(19) United States
(12) Patent Application Publication
(10) Pub. No.: US 2026/0136829 A1
(43) Pub. Date: May 14, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: So-Young JUNG, Gyeonggi-do (KR); Ji-Song JUN, Gyeonggi-do (KR); Su-Hyun LEE, Gyeonggi-do (KR); Sang-Hee CHO, Gyeonggi-do (KR)

(21) Appl. No.: 19/008,260

(22) Filed: Jan. 2, 2025

Related U.S. Application Data
(63) Continuation of application No. 17/432,359, now abandoned.

(30) Foreign Application Priority Data
Feb. 25, 2019 (KR) 10-2019-0021936
Dec. 19, 2019 (KR) 10-2019-0170385

(51) Int. Cl. H10K 85/60; C07D 487/06; C07D 491/06; C09K 11/02; C09K 11/06; H10K 50/11; H10K 101/00; H10K 101/10

(52) U.S. Cl. CPC H10K 85/6572; C07D 487/06; C07D 491/06; C09K 11/02; C09K 11/06; H10K 85/636; H10K 85/654; H10K 85/657; H10K 85/6574; C09K 2211/1007; C09K 2211/1029; C09K 2211/185; H10K 50/11; H10K 2101/10; H10K 2101/90

(57) ABSTRACT

The present disclosure relates to an organic electroluminescent compound represented by formula 1 and an organic electroluminescent device comprising the same. By comprising the organic electroluminescent compound of the present disclosure, it is possible to provide an organic electroluminescent device having improved operating voltage, luminous efficiency, lifetime, and/or power efficiency.

### CLAIM OF BENEFIT OF PRIOR APPLICATION

This application is a continuation of U.S. patent application Ser. No. 17/432,359 filed Aug. 19, 2021, which is the National Stage Entry of PCT/KR2020/000762, filed Jan. 16, 2020, both of which are incorporated by reference herein in their entirety.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

The most important factor determining luminous efficiency in the organic electroluminescent device is light-emitting materials. Until now, fluorescent materials have been widely used as light-emitting materials. However, in view of electroluminescent mechanisms, since phosphorescent light-emitting materials theoretically enhance luminous efficiency by four (4) times compared to fluorescent light-emitting materials, phosphorescent light-emitting materials have been widely researched. Iridium(III) complexes have been widely known as phosphorescent light-emitting materials, including bis(2-(2′-benzothienyl)-pyridinato-N,C-3′)iridium(acetylacetonate) [(acac)lr(btp)₂], tris(2-phenylpyridine)iridium [lr(ppy)₃] and bis(4,6-difluorophenylpyridinato-N,C2)picolinate iridium (Firpic) as red-, green-, and blue-emitting materials, respectively.

In the prior art, 4,4′-N,N′-dicarbazol-biphenyl (CBP) is the most widely known phosphorescent host material. Recently, Pioneer (Japan) et al., developed a high performance organic electroluminescent device using bathocuproine (BCP) and aluminum(III) bis(2-methyl-8-quinolinate)(4-phenylphenolate) (BAlq), etc., as host materials, which were known as hole blocking materials.

Although these materials provide good luminous characteristics, they have the following disadvantages: (1) Due to their low glass transition temperature and poor thermal stability, their degradation may occur during a high-temperature deposition process in a vacuum, and the lifetime of the device decreases. (2) The power efficiency of the organic electroluminescent device is given by [(π/voltage)×current efficiency], and the power efficiency is inversely proportional to the voltage. Although the organic electroluminescent device comprising phosphorescent host materials provides current efficiency [cd/A] higher than one comprising fluorescent materials, a significantly high operating voltage is necessary. Thus, there is no merit in terms of power efficiency [lm/W]. (3) In addition, when these materials are used in an organic electroluminescent device, the operational lifetime of an organic electroluminescent device is short and luminous efficiency is still required to be improved.

In order to improve luminous efficiency, operating voltage and/or lifetime, various materials or concepts for an organic layer of an organic electroluminescent device have been proposed, but they have not been satisfactory in practical use.

### DISCLOSURE OF INVENTION

### Technical Problem

The objective of the present disclosure is firstly, to provide an organic electroluminescent compound effective for producing an organic electroluminescent device having improved operating voltage, luminous efficiency, lifetime property and/or power efficiency, and secondly, to provide an organic electroluminescent device comprising the organic electroluminescent compound.

### Solution to Problem

The present inventors have found that the above objective can be achieved by a specific organic electroluminescent compound having a structure in which the residues of an 8-membered ring are multi-fused, and an organic electroluminescent device using the same. Specifically, the above objective can be achieved by an organic electroluminescent compound represented by the following formula 1:

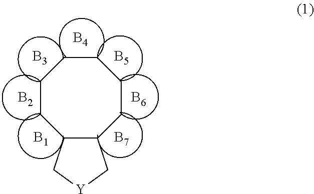

wherein
B₁ to B₇, each independently, are not present or represent a substituted or unsubstituted (C5-C20) ring, in which the carbon atom of the ring may be replaced with one or more heteroatoms selected from nitrogen, oxygen and sulfur; with the proviso that at least five of B₁ to B₇ are present, and the adjacent rings of B₁ to B₇ are fused with each other;
Y represents —N-L₁-(Ar₁)ₙ, —O—, —S—, or —CR₁R₂;
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ represents a substituted or unsubstituted (C6-C30)aryl, a substituted or unsubstituted (3- to 30-membered)heteroaryl, or —NR₃R₄;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to an adjacent substituent(s) to form a ring(s); and
n represents an integer of 1 or 2; where if n represents 2, each of Ar₁ may be the same as or different from each other.

### Advantageous Effects of Invention

By using the organic electroluminescent compound according to the present disclosure, it is possible to produce an organic electroluminescent device having improved operating voltage properties, improved luminous efficiency, excellent lifetime properties, and/or high power efficiency.

### MODE FOR THE INVENTION

Hereinafter, the present disclosure will be described in detail. However, the following description is intended to explain the invention, and is not meant in any way to restrict the scope of the invention.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, an electron injection material, etc.

The organic electroluminescent material of the present disclosure may comprise at least one compound represented by formula 1. The compound represented by formula 1 may be comprised in a light-emitting layer, an electron transport layer, and/or an electron buffer layer, but is not limited thereto. When comprised in the light-emitting layer, the compound represented by formula 1 may be comprised as a host material. Herein, the host material may be a host material of a green or red light-emitting organic electroluminescent device. In addition, when comprised in the electron transport layer, the compound represented by formula 1 may be comprised as an electron transport material. Further, when comprised in the electron buffer layer, the compound represented by formula 1 may be comprised as an electron buffer material.

The term “a plurality of organic electroluminescent materials” in the present disclosure means an organic electroluminescent material(s) comprising a combination of at least two compounds, which may be comprised in any organic layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. At least two compounds may be comprised in the same layer or different layers by means of the methods used in the art, for example, they may be mixture-evaporated or co-evaporated, or may be individually deposited.

The term “a plurality of host materials” in the present disclosure means a host material(s) comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure may be a combination of two or more host materials, and may optionally further include a conventional material comprised in organic electroluminescent materials.

The two or more compounds comprised in the plurality of host materials of the present disclosure may be included in one light-emitting layer or may be respectively included in different light-emitting layers. For example, the two or more host materials may be mixture-evaporated or co-evaporated, or individually deposited.

Herein, the term “(C1-C30)alkyl(ene)” is meant to be a linear or branched alkyl(ene) having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, preferably 6 to 25 ring backbone carbon atoms, and more preferably 6 to 18 ring backbone carbon atoms. The above aryl or arylene may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, etc.

The term “(3- to 30-membered)heteroaryl(ene)” is an aryl(ene) having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. In the present disclosure, the substituents of the substituted (C1-C30)alkyl(ene), the substituted (C6-C30)aryl(ene), the substituted (3- to 30-membered)heteroaryl(ene), the substituted (C3-C30)cycloalkyl(ene), the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di- (C1-C30)alkylamino, the substituted mono- or di- (C6-C30)arylamino, and the substituted (C1-C30)alkyl(C6-C30)arylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one selected from the group consisting of deuterium and a (3- to 30-membered)heteroaryl(s); a (3- to 30-membered)heteroaryl unsubstituted or substituted with a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of deuterium; a (C1-C20)alkyl; a (C6-C25)aryl unsubstituted or substituted with at least one selected from the group consisting of deuterium and a (5- to 30-membered)heteroaryl(s); a (5- to 30-membered)heteroaryl unsubstituted or substituted with a (C6-C25)aryl(s); and a (C1-C20)alkyl(C6-C25)aryl. According to another embodiment of the present disclosure, the substituents, each independently, are at least one selected from the group consisting of deuterium; a (C1-C20)alkyl; a (C6-C18)aryl unsubstituted or substituted with at least one selected from the group consisting of deuterium and a (5- to 26-membered)heteroaryl(s); a (6- to 26-membered)heteroaryl unsubstituted or substituted with a (C6-C18)aryl(s); and a (C1-C10)alkyl(C6-C18)aryl. For example, the substituents, each independently, may be at least one selected from the group consisting of deuterium, a methyl, an unsubstituted phenyl, a phenyl substituted with one or more deuterium, a phenyl substituted with a (26-membered)heteroaryl, a naphthyl, a biphenyl, a dimethylfluorenyl, a terphenyl, an unsubstituted pyridinyl, a pyridinyl substituted with a phenyl(s), a triazinyl substituted with a phenyl(s), a dibenzothiophenyl, a dibenzofuranyl, and a (26-membered)heteroaryl.

In the formulas of the present disclosure, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; and preferably, a substituted or unsubstituted mono- or polycyclic (5- to 26-membered) alicyclic or aromatic ring, or the combination thereof. In addition, the ring may contain at least one heteroatom selected from B, N, O, S, Si, and P, preferably at least one heteroatom selected from N, O, and S. For example, the ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, etc.

Herein, the heteroaryl(ene) and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, and a substituted or unsubstituted (C1-C30)alkyl(C6-C30)arylamino.

Hereinafter, the compound represented by formula 1 will be described in more detail.

In formula 1, B₁ to B₇, each independently, are not present or represent a substituted or unsubstituted (C5-C20) ring, preferably a substituted or unsubstituted (C5-C13) ring, in which the carbon atom(s) of the ring may be replaced with one or more heteroatoms selected from nitrogen, oxygen and sulfur, with the proviso that at least five of B₁ to B₇ are present, and the adjacent rings of B₁ to B₇ are fused with each other. Herein, the adjacent rings of B₁ to B₇ being fused with each other means ring B₁ and ring B₂, ring B₂ and ring B₃, ring B₃ and ring B₄, ring B₄ and ring B₅, ring B₅ and ring B₆, or ring B₆ and ring B₇ are fused with each other. According to one embodiment of the present disclosure, if any one of B₁ to B₇ represents a (C6-C20) ring, the adjacent ring may not be present or may represent a C5 ring, and the carbon atom(s) of the ring may be replaced with one or more heteroatoms selected from nitrogen, oxygen and sulfur. According to another embodiment of the present disclosure, B₁ to B₇, each independently, may not be present or may represent a substituted or unsubstituted benzene ring, a substituted or unsubstituted naphthalene ring, a substituted or unsubstituted pyrrole ring, a substituted or unsubstituted furan ring, a substituted or unsubstituted thiophene ring, a substituted or unsubstituted cyclopentadiene ring, a substituted or unsubstituted fluorene ring, a substituted or unsubstituted pyridine ring, or a substituted or unsubstituted dibenzofuran ring. For example, B₁ to B₇, each independently, may not be present or may represent a benzene ring unsubstituted or substituted with a phenyl(s), a naphthyl(s), and/or a diphenyltriazinyl(s); a naphthalene ring; a cyclopentadiene ring unsubstituted or substituted with a methyl(s); a fluorene ring substituted with a methyl(s); a pyrrole ring substituted with an unsubstituted phenyl(s), a phenyl substituted with one or more deuteriums, a biphenyl(s), and/or a pyridyl(s); a furan ring; a thiophene ring; a pyridine ring; or a dibenzofuran ring unsubstituted or substituted with a diphenyltriazinyl(s).

In formula 1, Y represents —N-L₁-(Ar₁)ₙ, —O—, —S—, or —CR₁R₂. According to one embodiment of the present disclosure, Y may represent —N-L₁-(Ar₁)ₙ.

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁ represents a single bond, a substituted or unsubstituted (C6-C25)arylene, or a substituted or unsubstituted (3- to 30-membered)heteroarylene. According to another embodiment of the present disclosure, L₁ represents a single bond, an unsubstituted (C6-C18)arylene, or an unsubstituted (5- to 25-membered)heteroarylene. For example, L₁ may represent a single bond, a phenylene, a naphthylene, a biphenylene, a pyridylene, a pyrimidinylene, a triazinylene, a quinoxalinylene, quinazolinylene, a dibenzofuranylene, a benzofuropyrimidinylene, a benzothienopyrimidinylene, an indolopyrimidinylene, or a benzoquinoxalinylene.

Ar₁ represents a substituted or unsubstituted (C6-C30)aryl, a substituted or unsubstituted (3- to 30-membered)heteroaryl, or —NR₃R₄. According to one embodiment of the present disclosure, Ar₁ represents a substituted or unsubstituted (C6-C25)aryl, a substituted or unsubstituted (5- to 25-membered)heteroaryl, or —NR₃R₄. According to another embodiment of the present disclosure, Ar₁ represents a (C6-C25)aryl unsubstituted or substituted with at least one selected from the group consisting of deuterium, a (C1-C6)alkyl and a (3- to 30-membered)heteroaryl; a (5- to 25-membered)heteroaryl unsubstituted or substituted with at least one selected from the group consisting of deuterium, a (C6-C18)alkyl and a (3- to 30-membered)heteroaryl; or —NR₃R₄. For example, Ar₁ may represent an unsubstituted phenyl, a phenyl substituted with one or more deuteriums, a phenyl substituted with a (26-membered)heteroaryl(s), a naphthyl, a biphenyl, a fluorenyl substituted with a methyl(s), a spirobifluorenyl, a terphenyl, a triphenylenyl, a pyridyl unsubstituted or substituted with a phenyl(s), a pyrimidinyl substituted with a phenyl(s), a substituted triazinyl, a substituted quinoxalinyl, a substituted quinazolinyl, a benzoquinoxalinyl substituted with a phenyl(s), a carbazolyl, a dibenzofuranyl, a dibenzothiophenyl, a benzofuropyrimidinyl substituted with a phenyl(s), a benzothienopyrimidinyl substituted with a phenyl(s), an indolopyrimidinyl substituted with a phenyl(s), or —NR₃R₄. The substituent of the substituted triazinyl, substituted quinoxalinyl, and substituted quinazolinyl, each independently, may be at least one selected from the group consisting of a phenyl unsubstituted or substituted with at least one of deuterium and a (26-membered)heteroaryl; a naphthyl; a biphenyl; a terphenyl; a dibenzofuranyl; a pyridyl substituted with a phenyl(s); a dimethylfluorenyl; and a dibenzothiophenyl.

R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to an adjacent substituent(s) to form a ring(s). According to one embodiment of the present disclosure, R₁ to R₄, each independently, represent hydrogen, deuterium, a substituted or unsubstituted (C1-C20)alkyl, or a substituted or unsubstituted (C6-C25)aryl. According to another embodiment of the present disclosure, R₁ and R₂, each independently, represent an unsubstituted (C1-C10)alkyl, and R₃ and R₄, each independently, represent an unsubstituted (C6-C18)aryl. For example, R₁ and R₂ may be a methyl, and R₃ and R₄ may be a phenyl.

The n above represents an integer of 1 or 2; where if n represents 2, each of Ar₁ may be the same as or different from each other.

The formula 1 may be represented by any one of the following formulas 1-1 to 1-5.

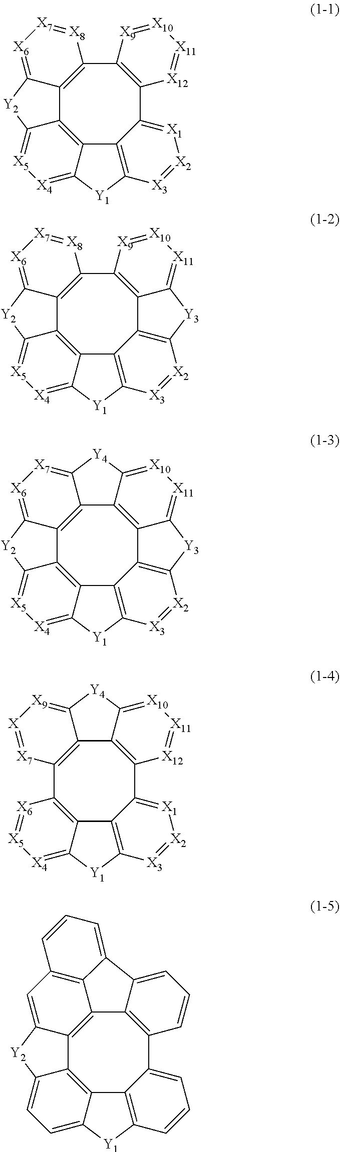

In formulas 1-1 to 1-5, Y₁, Y₂, Y₃, and Y₄, each independently, are the same as the definition of Y in formula 1, and where if a plurality of Ar₁ is present, each of Ar₁ may be the same as or different from each other; X₁ to X₁₂, each independently, represent —N═ or —C(Rₐ)═; and Rₐ, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or adjacent Rₐ's may be linked to each other to form a ring(s); and where if a plurality of Rₐ is present, each of Rₐ may be the same as or different from each other.

According to one embodiment of the present disclosure, Rₐ, each independently, represent hydrogen, deuterium, a substituted or unsubstituted (C6-C25)aryl, or a substituted or unsubstituted (5- to 25-membered)heteroaryl; or adjacent Rₐ's may be linked to each other to form a ring(s). According to another embodiment of the present disclosure, Rₐ, each independently, represent hydrogen, an unsubstituted (C6-C18)aryl, or a (5- to 25-membered)heteroaryl substituted with a (C6-C18)aryl(s); or adjacent Rₐ's may be linked to each other to form a benzene ring, an indene ring substituted with a methyl(s), or a benzofuran ring unsubstituted or substituted with a diphenyltriazinyl(s).

In any one of formulas 1-1 to 1-5, at least one of Ar₁(s) and Rₐ(s) may represent any one selected from those listed in the following Group 1.

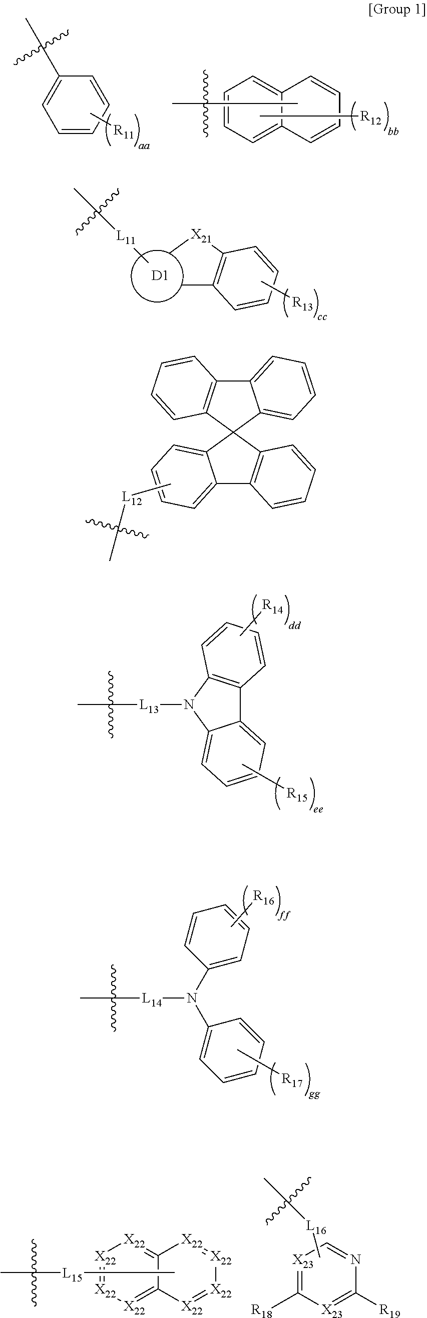

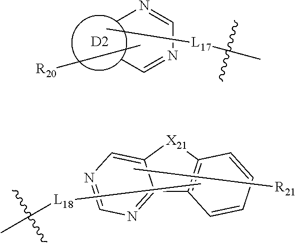

In Group 1, D1 and D2, each independently, represent a benzene ring or a naphthalene ring; X₂₁ represents O, S, NR₅, or CR₆R₇; X₂₂, each independently, represent CR₈ or N; with the proviso that at least one of X₂₂ represents N; X₂₃, each independently, represent CR₉ or N; L₁₁ to L₁₈, each independently, represent a single bond, a substituted or unsubstituted (C6-C30)arylene, or a substituted or unsubstituted (3- to 30-membered)heteroarylene; R₁₁ to R₂₁, and R₅ to R₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to an adjacent substituent(s) to form a ring(s); aa, ff, and gg, each independently, represent an integer of 1 to 5; bb represents an integer of 1 to 7; and cc, dd, and ee, each independently, represent an integer of 1 to 4.

According to one embodiment of the present disclosure, D1 may represent a benzene ring; X₂₁ may represent O, S, or CR₆R₇; L₁₁ to L₁₈, each independently, may represent a single bond; R₁ to R₂₁, and R₅ to R₉, each independently, may represent hydrogen, deuterium, a substituted or unsubstituted (C1-C20)alkyl, a substituted or unsubstituted (C6-C25)aryl, or a substituted or unsubstituted (5- to 25-membered)heteroaryl, or may be linked to an adjacent substituent(s) to form a ring(s); aa, bb, ff, and gg, each independently, may represent an integer of 1 to 5; and cc, dd, and ee, each independently, may represent an integer of 1 to 4. For example, R₁₁ may represent hydrogen, deuterium, a phenyl, a biphenyl, or a (26-membered)heteroaryl; R₁₂ may represent hydrogen or adjacent R₁₂'s may be linked to each other to form a benzene ring; R₁₃, R₁₆ and R₁₇ may represent hydrogen; R₁₈ and R₁₉ may represent hydrogen or a phenyl; R₂₁ may represent a phenyl; R₆ and R₇ may represent a methyl; R₈ may represent hydrogen, a phenyl, a biphenyl, a dibenzofuranyl, or a dibenzothiophenyl, or adjacent R₈'s may be linked to each other to form a benzene ring; R₉ may represent hydrogen, an unsubstituted phenyl, a phenyl substituted with one or more deuteriums, a phenyl substituted with a (26-membered)heteroaryl, a naphthyl, a biphenyl, a dimethylfluorenyl, a terphenyl, a pyridyl substituted with a phenyl(s), a dibenzofuranyl, or a dibenzothiophenyl; aa may represent an integer of 1 or 5; bb may represent an integer of 1 or 4; and cc may represent an integer of 1.

In any one of formulas 1-1 to 1-5, at least one of Ar₁(s) and Rₐ(s) may represent any one selected from those listed in the following Group 2.

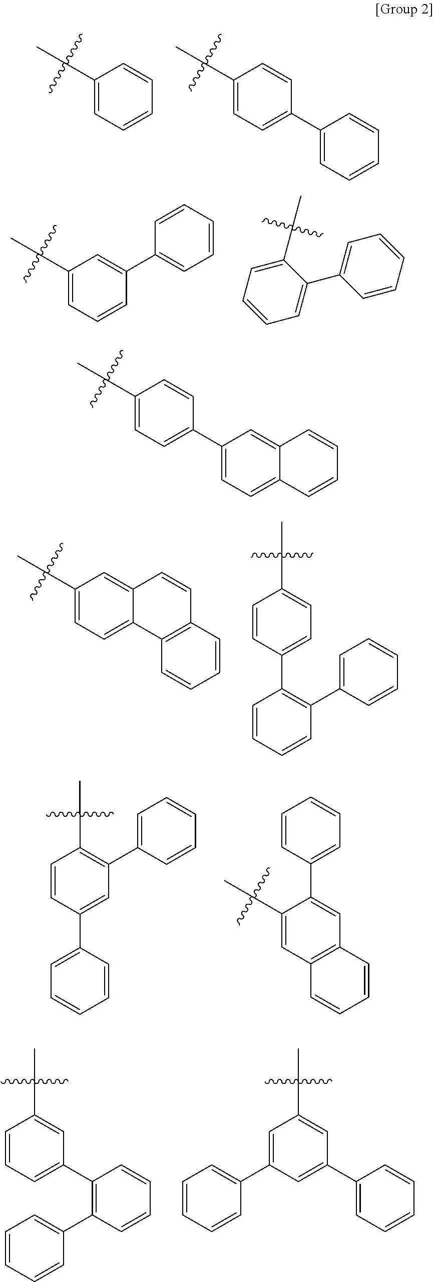

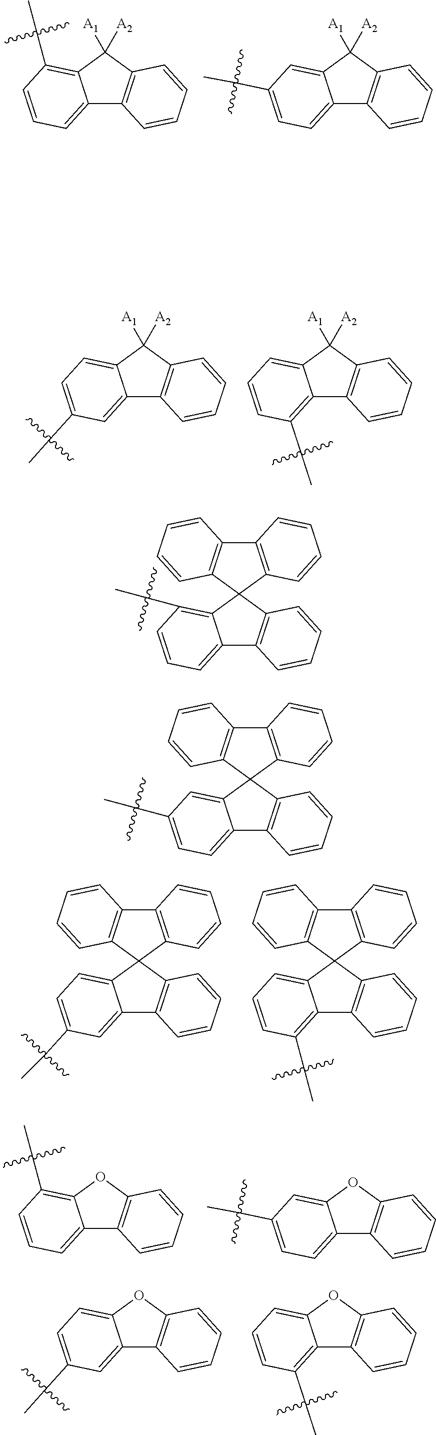

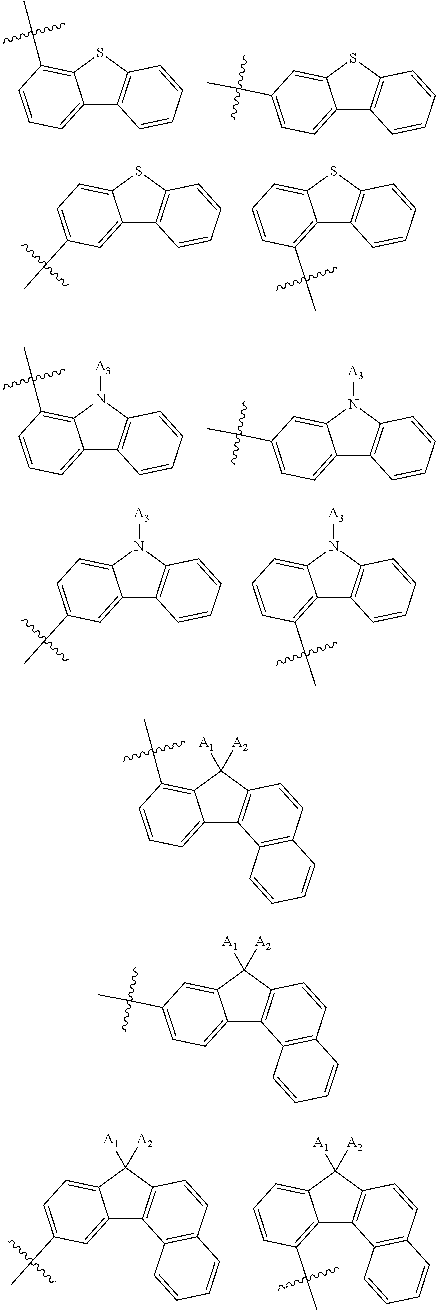

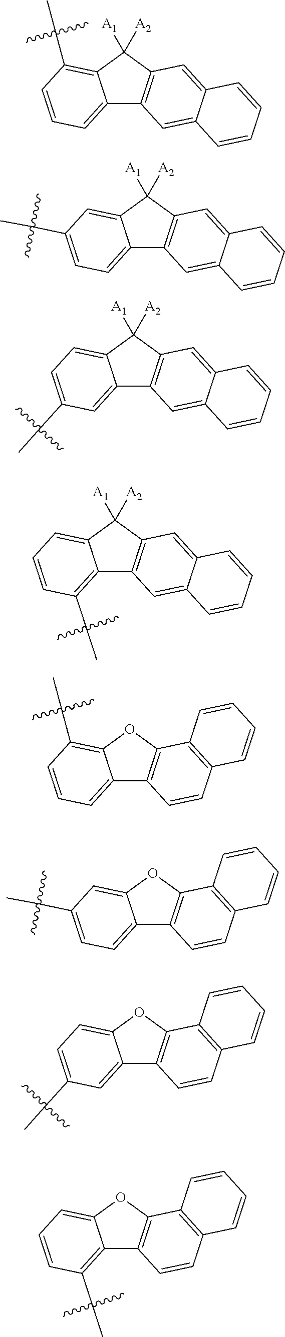

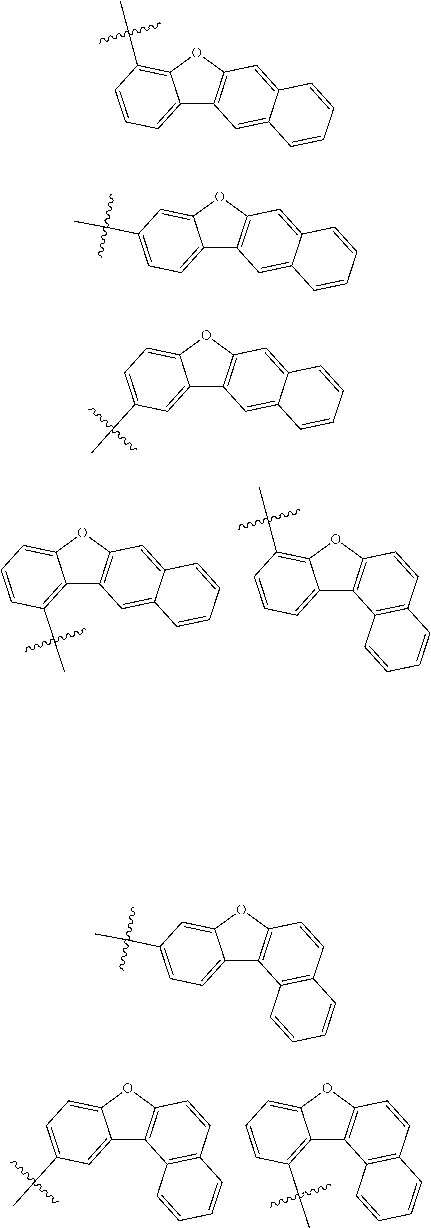

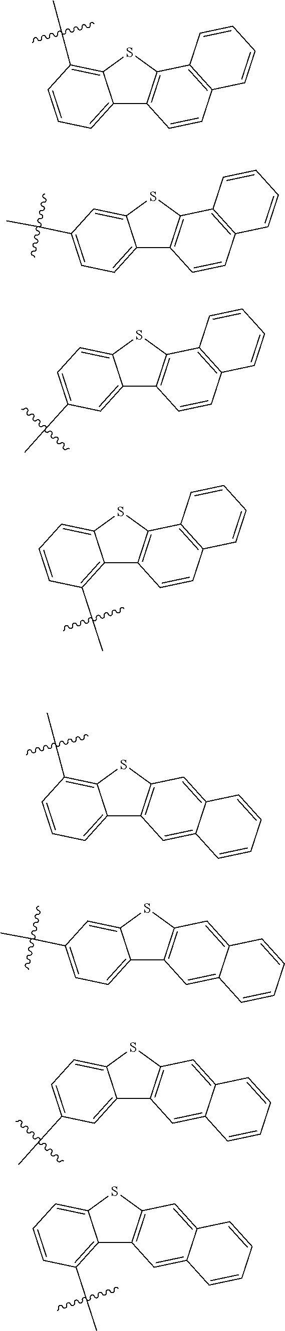

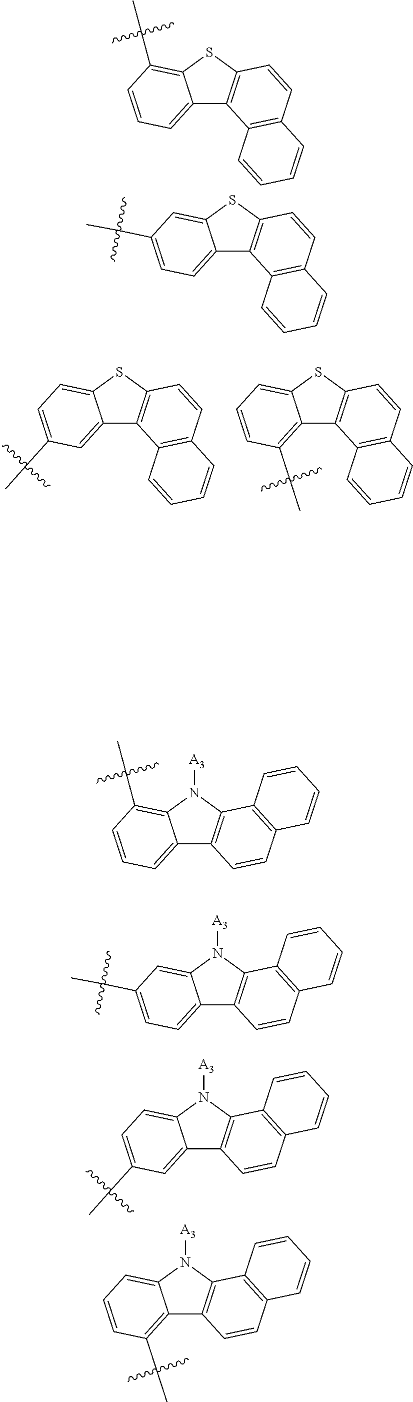

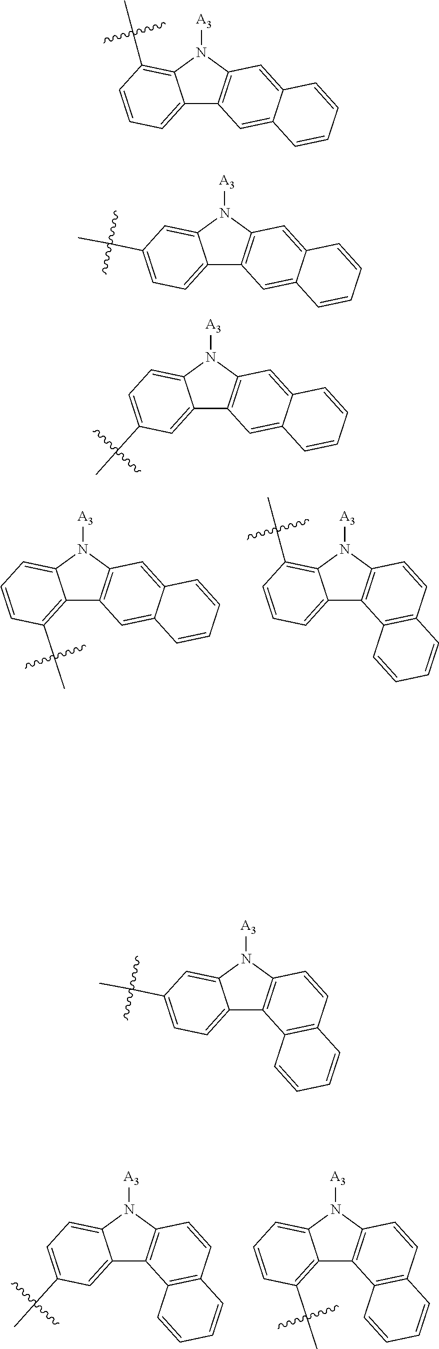

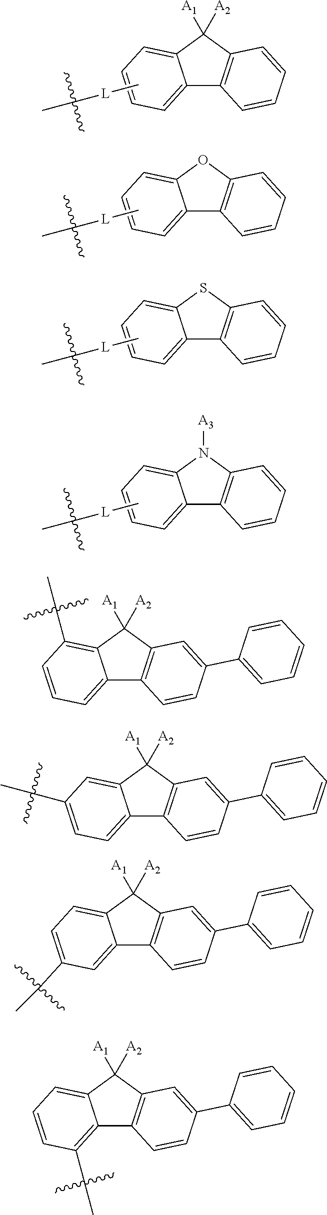

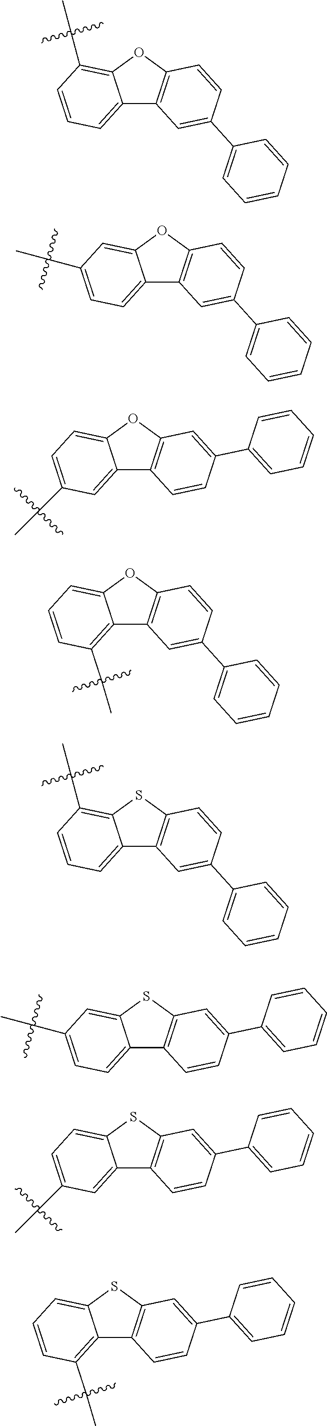

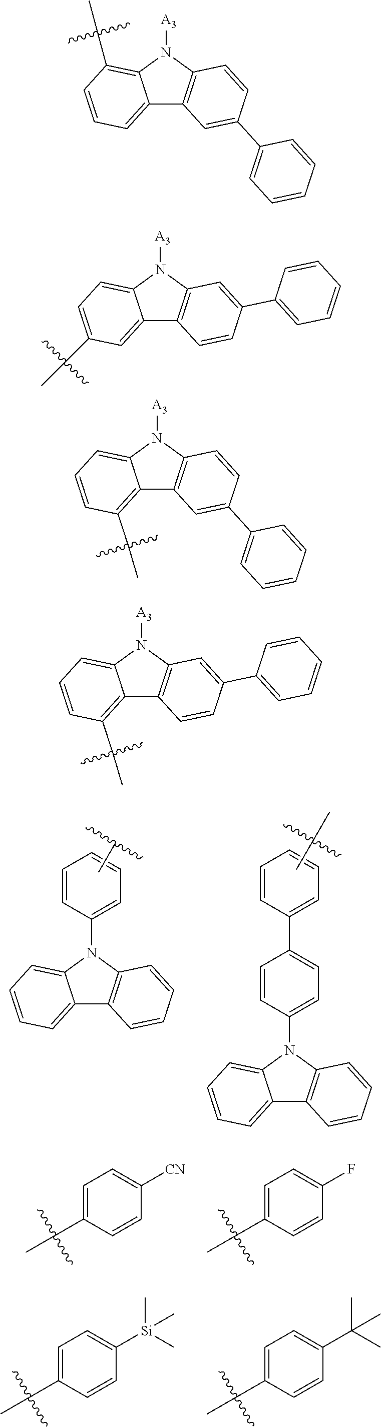

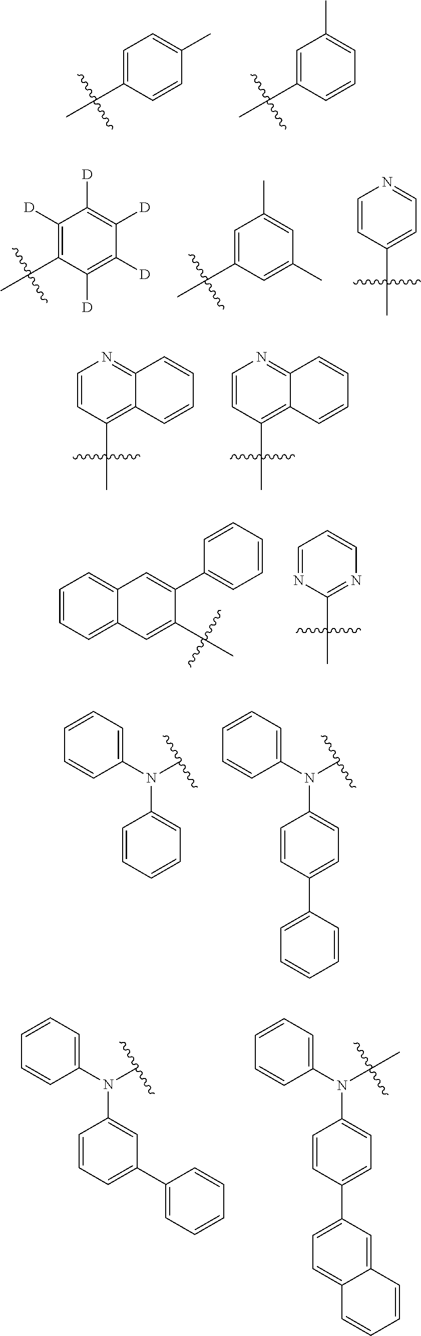

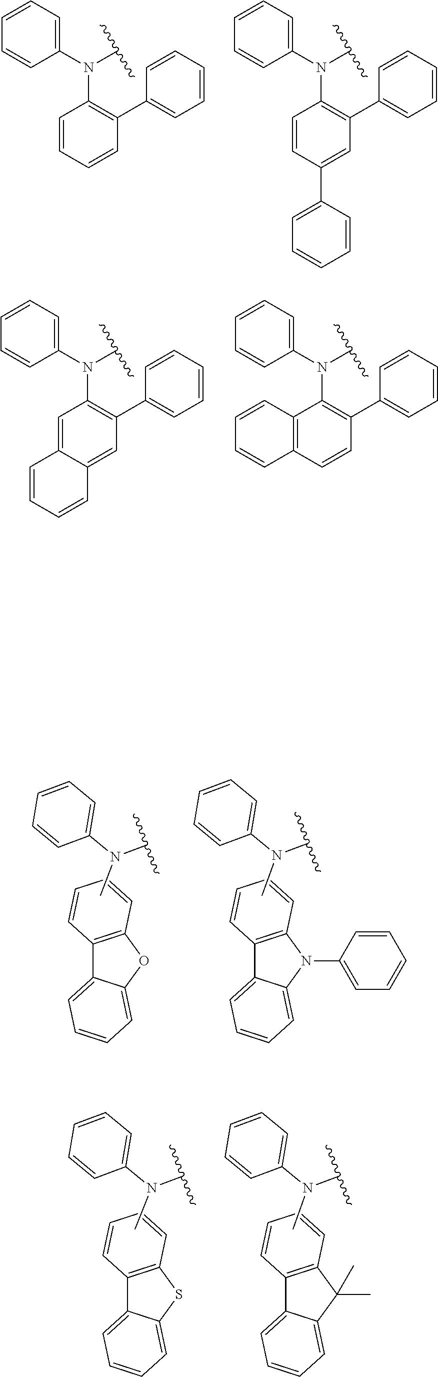

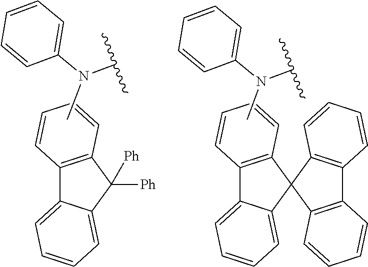

In Group 2, L represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; and A₁ to A₃, each independently, represent a substituted or unsubstituted (C1-C30)alkyl, or a substituted or unsubstituted (C6-C30)aryl.

In any one of formulas 1-1 to 1-5, at least one of Ar₁(s) and Rₐ(s) may represent any one selected from those listed in the following Group 3.

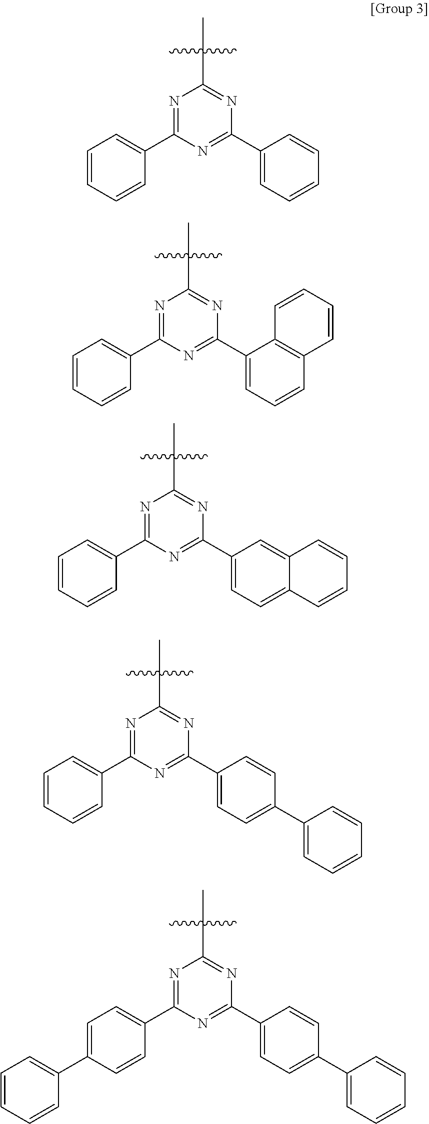 [SMILES: *c1nc(-c2ccccc2)nc(-c2ccccc2)n1 ; *c1nc(-c2ccccc2)nc(-c2cccc3ccccc23)n1 ; *c1nc(-c2ccccc2)nc(-c2ccc3ccccc3c2)n1 ; *c1nc(-c2ccccc2)nc(-c2ccc(-c3ccccc3)cc2)n1 ; *c1nc(-c2ccc(-c3ccccc3)cc2)nc(-c2ccc(-c3ccccc3)cc2)n1]

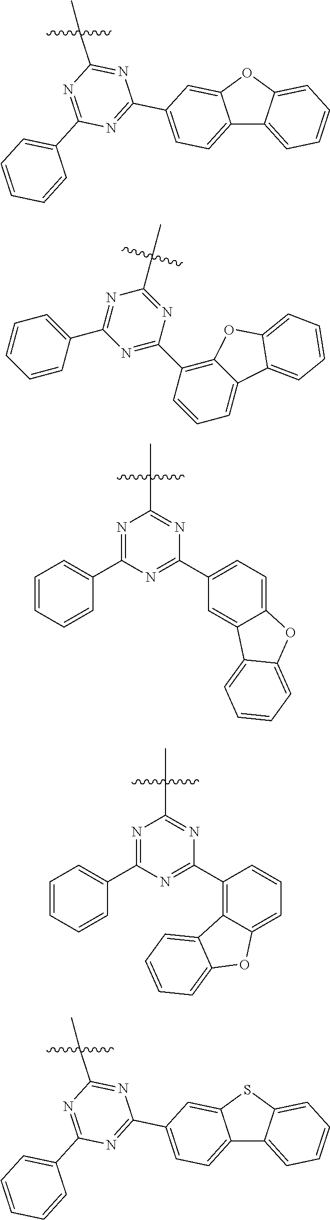 [SMILES: *c1nc(-c2ccccc2)nc(-c2ccc3c(c2)oc2ccccc23)n1 ; *c1nc(-c2ccccc2)nc(-c2cccc3c2oc2ccccc23)n1 ; *c1nc(-c2ccccc2)nc(-c2ccc3oc4ccccc4c3c2)n1 ; *c1nc(-c2ccccc2)nc(-c2cccc3oc4ccccc4c23)n1 ; *c1nc(-c2ccccc2)nc(-c2ccc3c(c2)sc2ccccc23)n1]

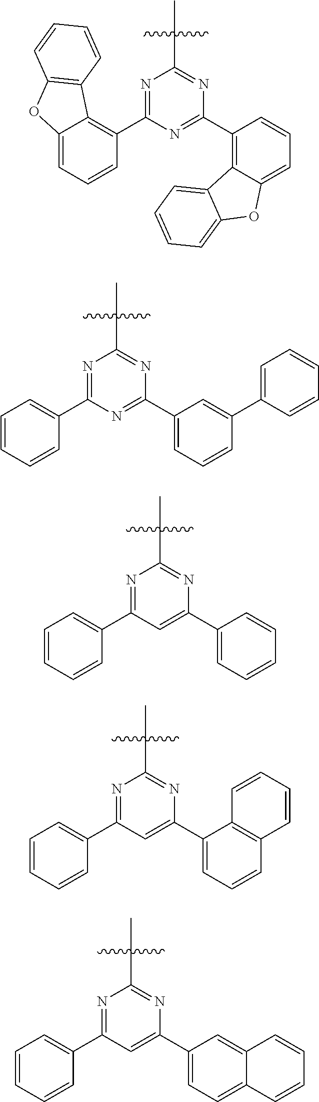 [SMILES: *c1nc(-c2cccc3oc4ccccc4c23)nc(-c2cccc3oc4ccccc4c23)n1 ; *c1nc(-c2ccccc2)nc(-c2cccc(-c3ccccc3)c2)n1 ; *c1nc(-c2ccccc2)cc(-c2ccccc2)n1 ; *c1nc(-c2ccccc2)cc(-c2cccc3ccccc23)n1 ; *c1nc(-c2ccccc2)cc(-c2ccc3ccccc3c2)n1]

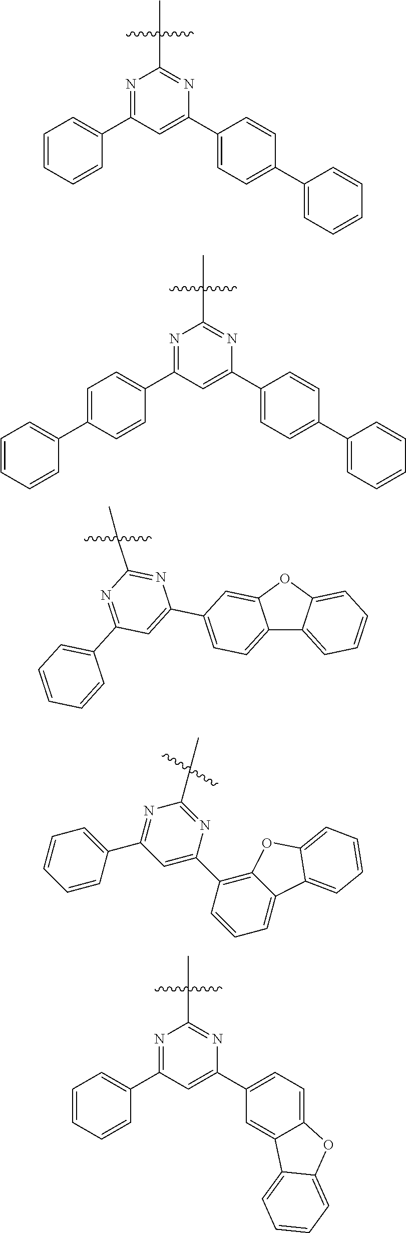 [SMILES: *c1nc(-c2ccccc2)cc(-c2ccc(-c3ccccc3)cc2)n1 ; *c1nc(-c2ccc(-c3ccccc3)cc2)cc(-c2ccc(-c3ccccc3)cc2)n1 ; *c1nc(-c2ccccc2)cc(-c2ccc3c(c2)oc2ccccc23)n1 ; *c1nc(-c2ccccc2)cc(-c2cccc3c2oc2ccccc23)n1 ; *c1nc(-c2ccccc2)cc(-c2ccc3oc4ccccc4c3c2)n1]

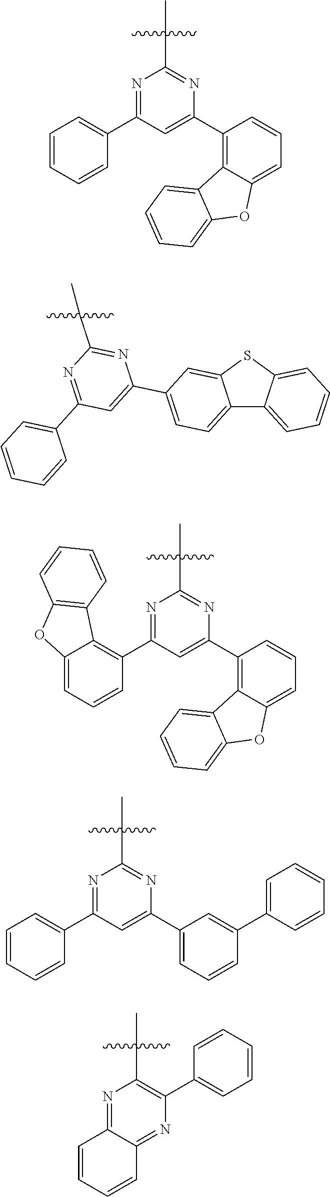 [SMILES: *c1nc(-c2ccccc2)cc(-c2cccc3oc4ccccc4c23)n1 ; *c1nc(-c2ccccc2)cc(-c2ccc3c(c2)sc2ccccc23)n1 ; *c1nc(-c2cccc3oc4ccccc4c23)cc(-c2cccc3oc4ccccc4c23)n1 ; *c1nc(-c2ccccc2)cc(-c2cccc(-c3ccccc3)c2)n1 ; *c1nc2ccccc2nc1-c1ccccc1]

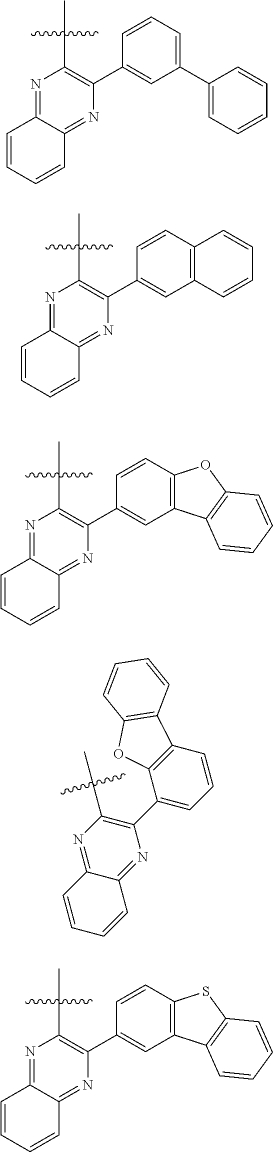 [SMILES: *c1nc2ccccc2nc1-c1cccc(-c2ccccc2)c1 ; *c1nc2ccccc2nc1-c1ccc2ccccc2c1 ; *c1nc2ccccc2nc1-c1ccc2oc3ccccc3c2c1 ; *c1nc2ccccc2nc1-c1cccc2c1oc1ccccc12 ; *c1nc2ccccc2nc1-c1ccc2sc3ccccc3c2c1]

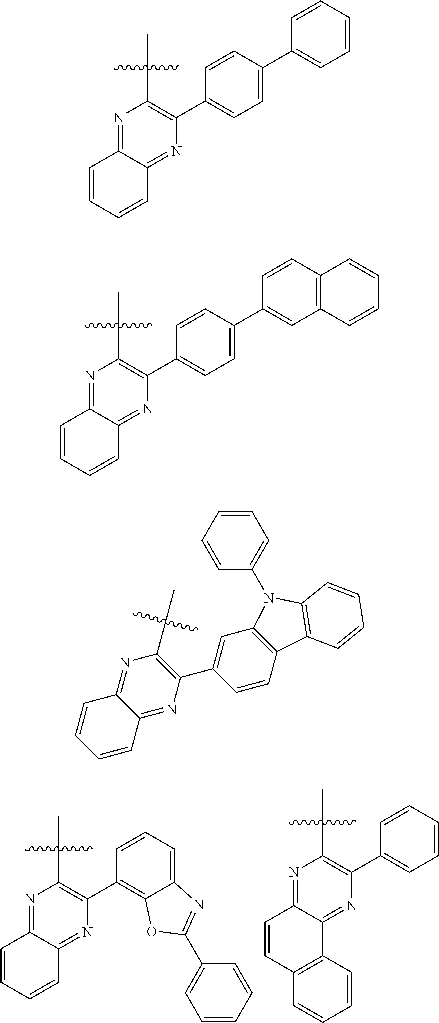

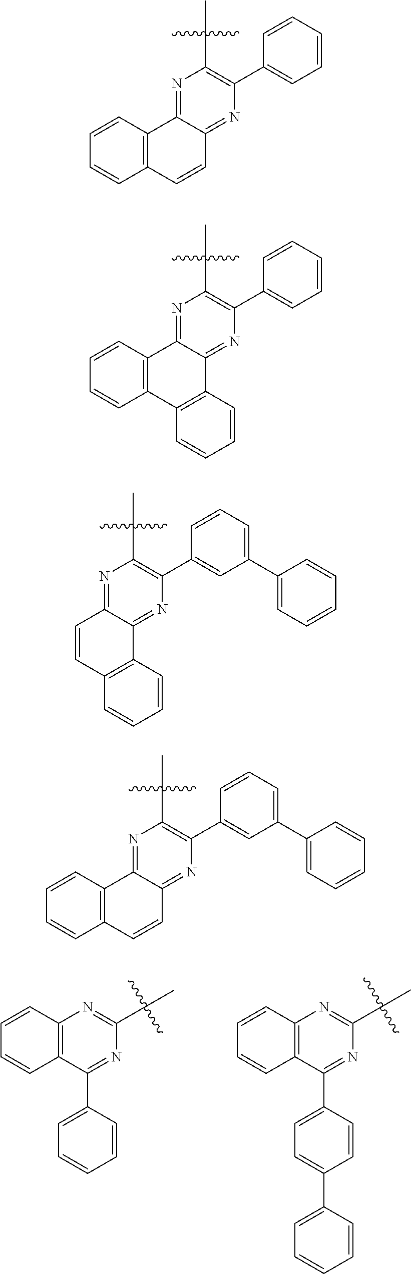 [SMILES: *c1nc2c(ccc3ccccc32)nc1-c1ccccc1 ; *c1nc2c3ccccc3c3ccccc3c2nc1-c1ccccc1 ; *c1nc2ccc3ccccc3c2nc1-c1cccc(-c2ccccc2)c1 ; *c1nc2c(ccc3ccccc32)nc1-c1cccc(-c2ccccc2)c1 ; *c1nc(-c2ccccc2)c2ccccc2n1 ; *c1nc(-c2ccc(-c3ccccc3)cc2)c2ccccc2n1]

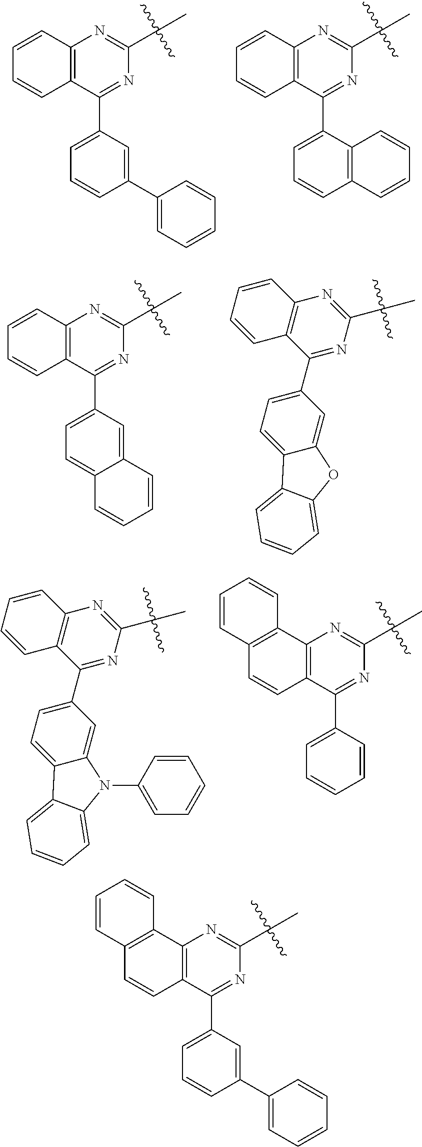

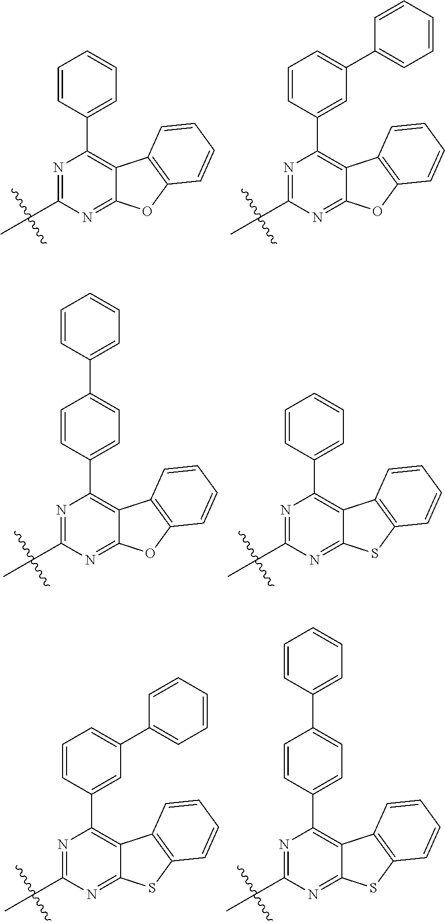 [SMILES: *c1nc(-c2ccccc2)c2c(n1)oc1ccccc12 ; *c1nc(-c2cccc(-c3ccccc3)c2)c2c(n1)oc1ccccc12 ; *c1nc(-c2ccc(-c3ccccc3)cc2)c2c(n1)oc1ccccc12 ; *c1nc(-c2ccccc2)c2c(n1)sc1ccccc12 ; *c1nc(-c2cccc(-c3ccccc3)c2)c2c(n1)sc1ccccc12 ; *c1nc(-c2ccc(-c3ccccc3)cc2)c2c(n1)sc1ccccc12]

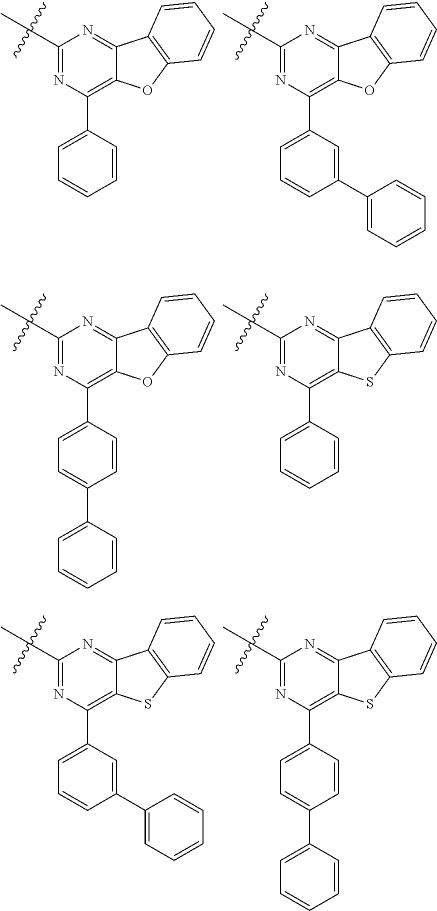

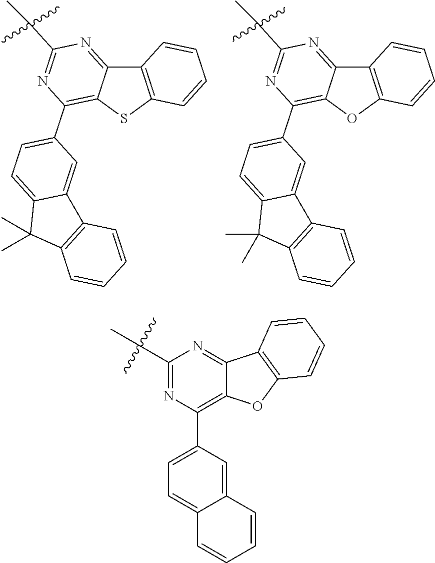

The compound represented by formula 1 may be specifically exemplified by the following compounds, but is not limited thereto.

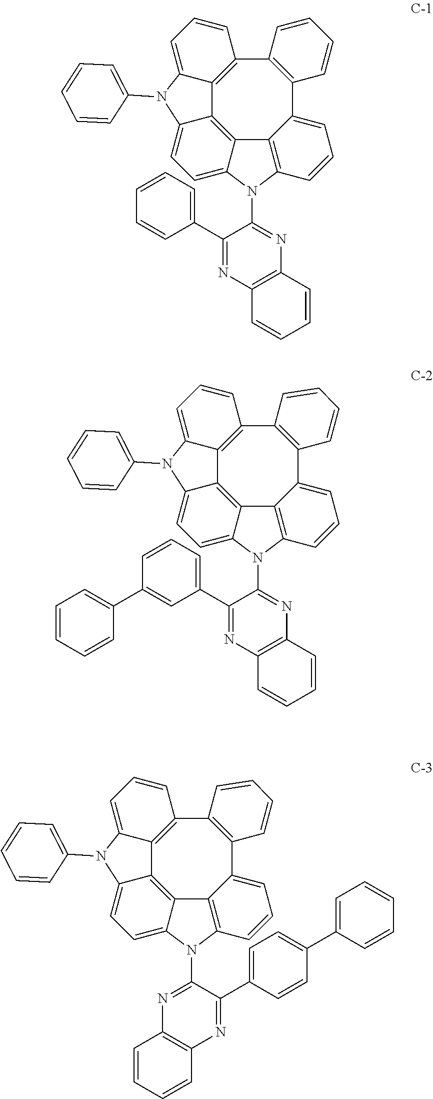 [SMILES: c1ccc(-c2nc3ccccc3nc2-n2c3cccc4c5ccccc5c5cccc6c5c5c(c43)c2ccc5n6-c2ccccc2)cc1 ; c1ccc(-c2cccc(-c3nc4ccccc4nc3-n3c4cccc5c6ccccc6c6cccc7c6c6c(c54)c3ccc6n7-c3ccccc3)c2)cc1 ; c1ccc(-c2ccc(-c3nc4ccccc4nc3-n3c4cccc5c6ccccc6c6cccc7c6c6c(c54)c3ccc6n7-c3ccccc3)cc2)cc1]

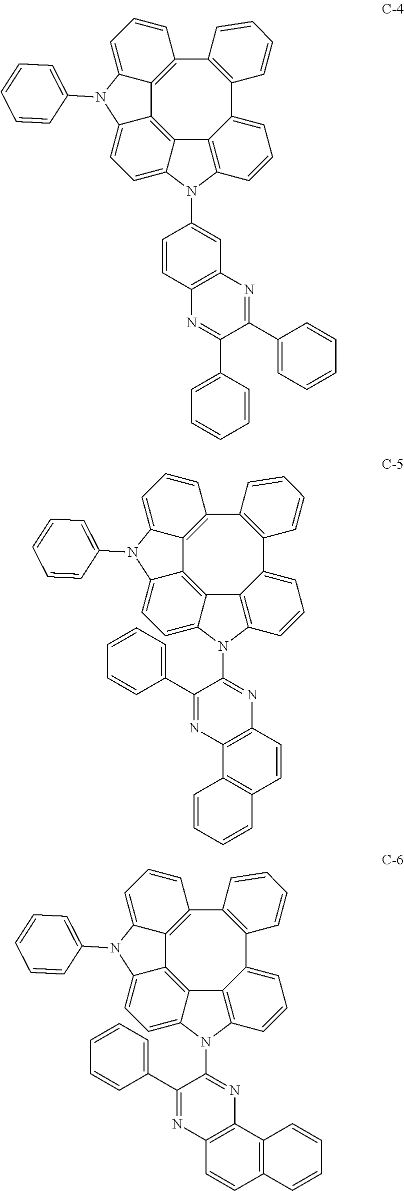

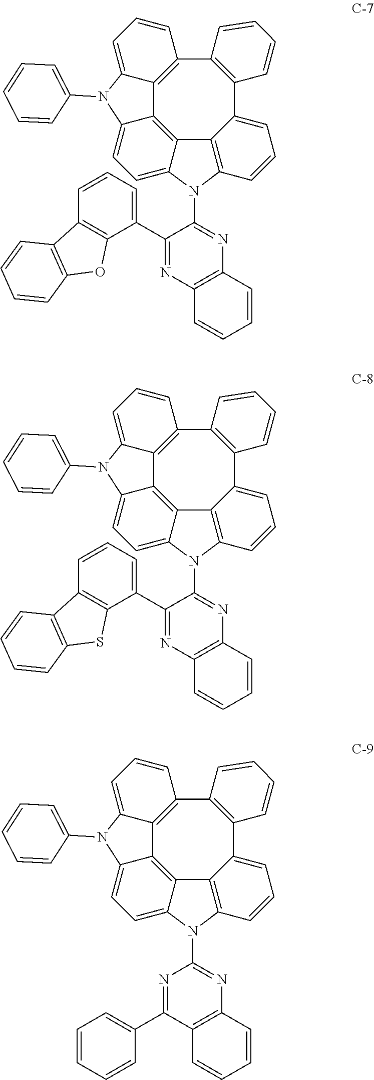 [SMILES: c1ccc(-n2c3cccc4c5ccccc5c5cccc6c5c5c(c43)c2ccc5n6-c2nc3ccccc3nc2-c2cccc3c2oc2ccccc23)cc1 ; c1ccc(-n2c3cccc4c5ccccc5c5cccc6c5c5c(c43)c2ccc5n6-c2nc3ccccc3nc2-c2cccc3c2sc2ccccc23)cc1 ; c1ccc(-c2nc(-n3c4cccc5c6ccccc6c6cccc7c6c6c(c54)c3ccc6n7-c3ccccc3)nc3ccccc23)cc1]

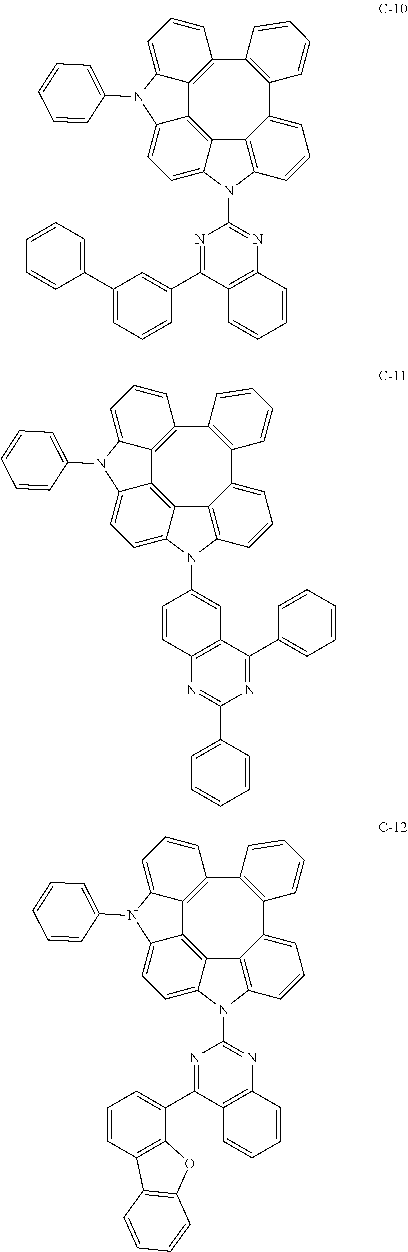

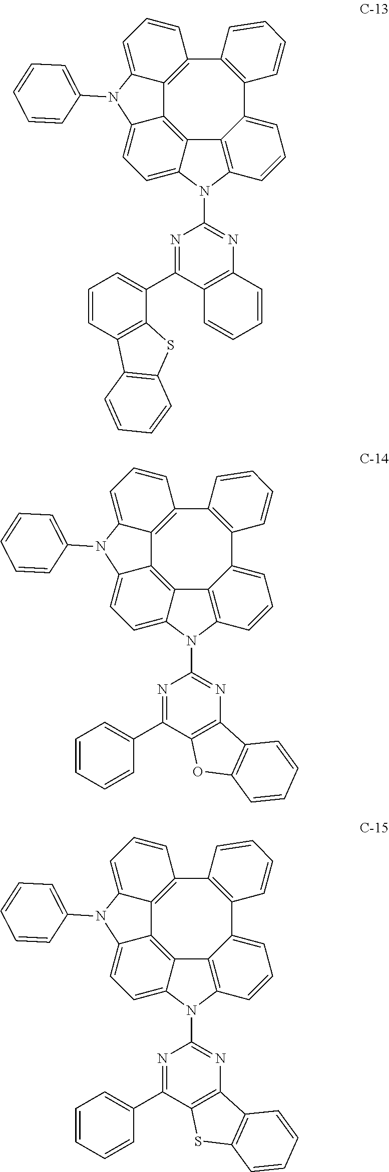

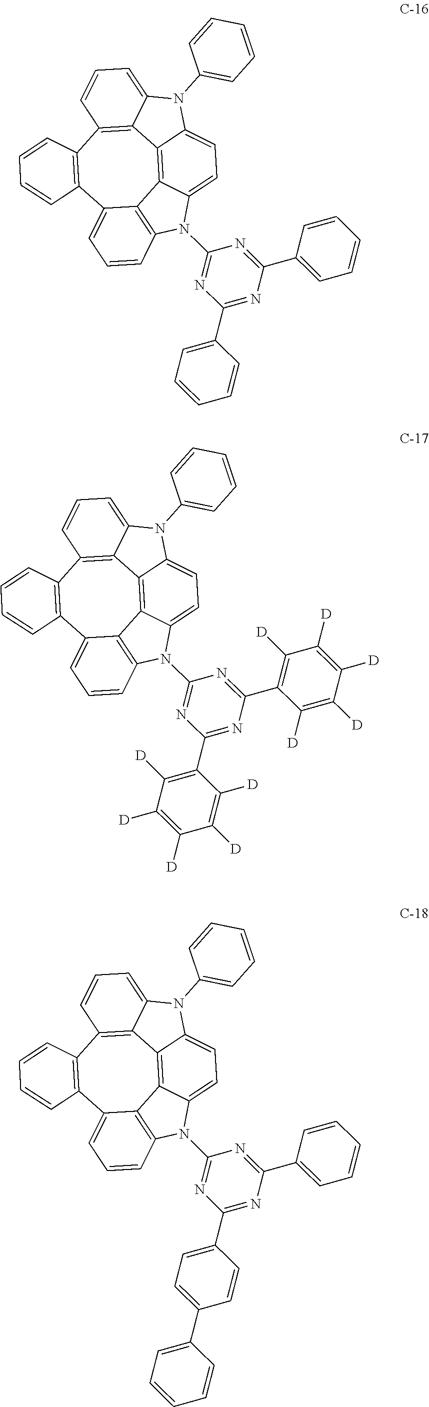

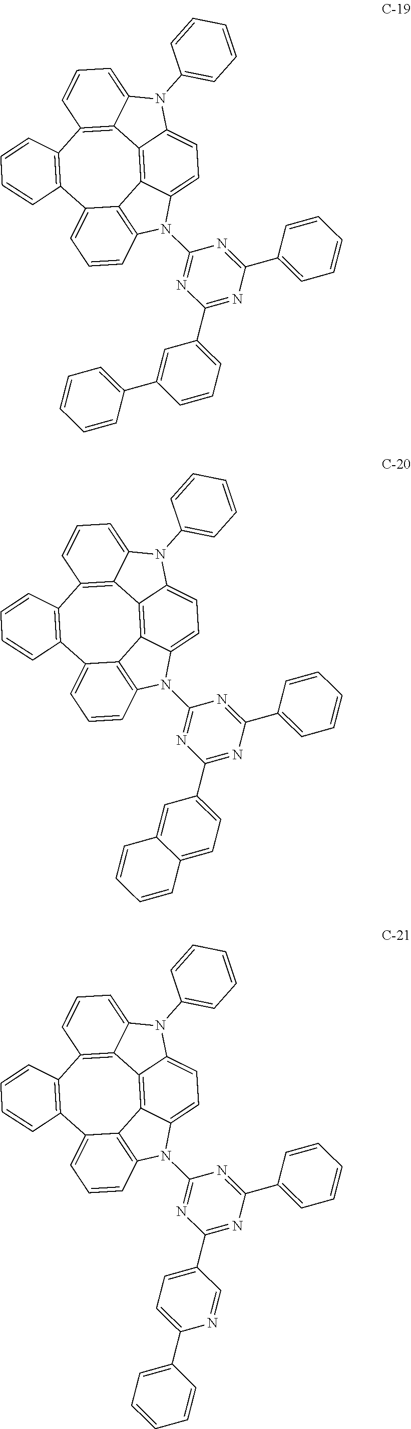 [SMILES: c1ccc(-c2cccc(-c3nc(-c4ccccc4)nc(-n4c5cccc6c7ccccc7c7cccc8c7c7c(c65)c4ccc7n8-c4ccccc4)n3)c2)cc1 ; c1ccc(-c2nc(-c3ccc4ccccc4c3)nc(-n3c4cccc5c6ccccc6c6cccc7c6c6c(c54)c3ccc6n7-c3ccccc3)n2)cc1 ; c1ccc(-c2ccc(-c3nc(-c4ccccc4)nc(-n4c5cccc6c7ccccc7c7cccc8c7c7c(c65)c4ccc7n8-c4ccccc4)n3)cn2)cc1]

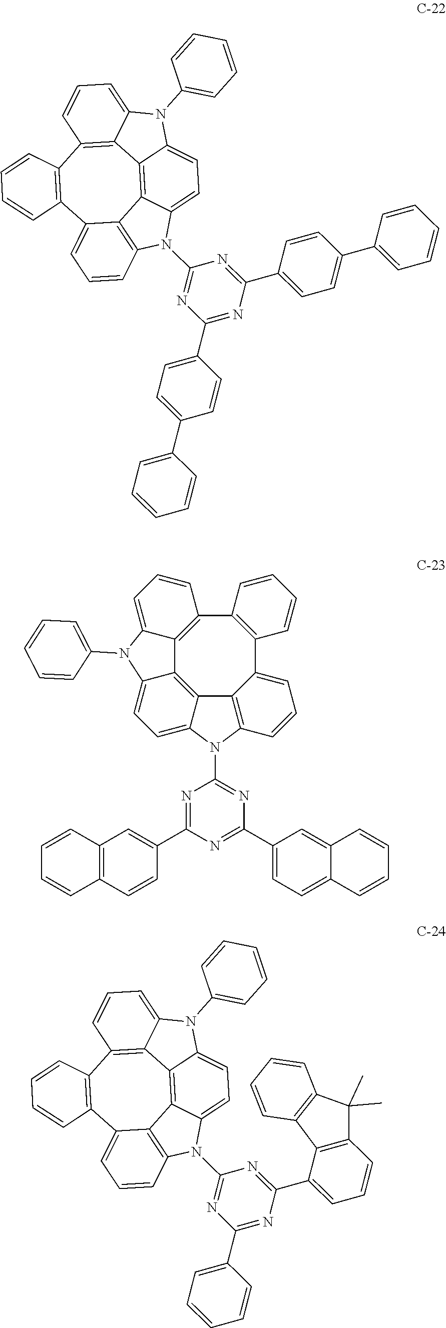 [SMILES: c1ccc(-c2ccc(-c3nc(-c4ccc(-c5ccccc5)cc4)nc(-n4c5cccc6c7ccccc7c7cccc8c7c7c(c65)c4ccc7n8-c4ccccc4)n3)cc2)cc1 ; c1ccc(-n2c3cccc4c5ccccc5c5cccc6c5c5c(c43)c2ccc5n6-c2nc(-c3ccc4ccccc4c3)nc(-c3ccc4ccccc4c3)n2)cc1 ; CC1(C)c2ccccc2-c2c(-c3nc(-c4ccccc4)nc(-n4c5cccc6c7ccccc7c7cccc8c7c7c(c65)c4ccc7n8-c4ccccc4)n3)cccc21]

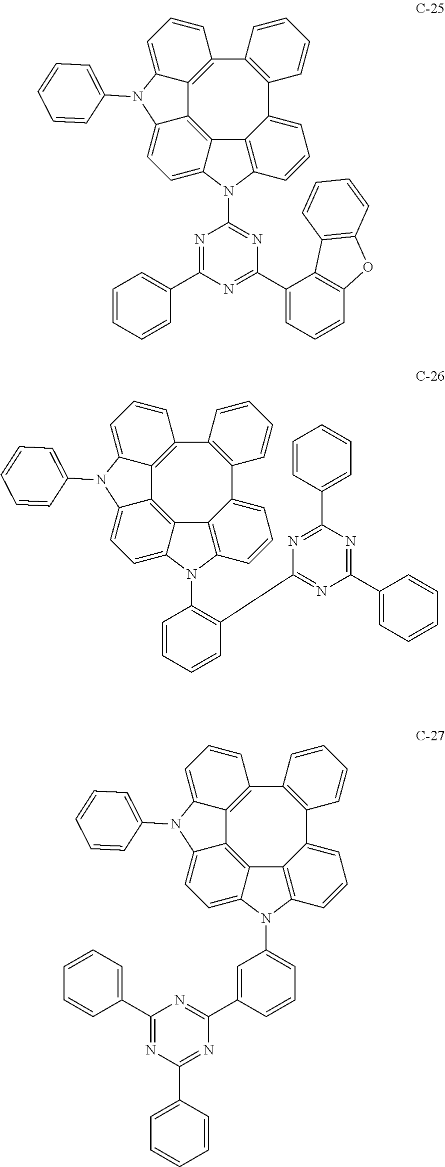 [SMILES: c1ccc(-c2nc(-c3cccc4oc5ccccc5c34)nc(-n3c4cccc5c6ccccc6c6cccc7c6c6c(c54)c3ccc6n7-c3ccccc3)n2)cc1 ; c1ccc(-c2nc(-c3ccccc3)nc(-c3ccccc3-n3c4cccc5c6ccccc6c6cccc7c6c6c(c54)c3ccc6n7-c3ccccc3)n2)cc1 ; c1ccc(-c2nc(-c3ccccc3)nc(-c3cccc(-n4c5cccc6c7ccccc7c7cccc8c7c7c(c65)c4ccc7n8-c4ccccc4)c3)n2)cc1]

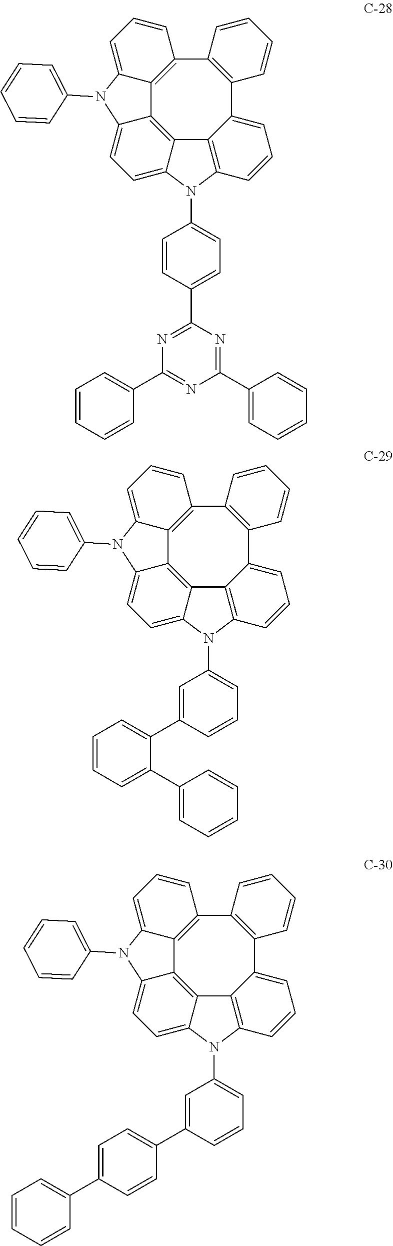

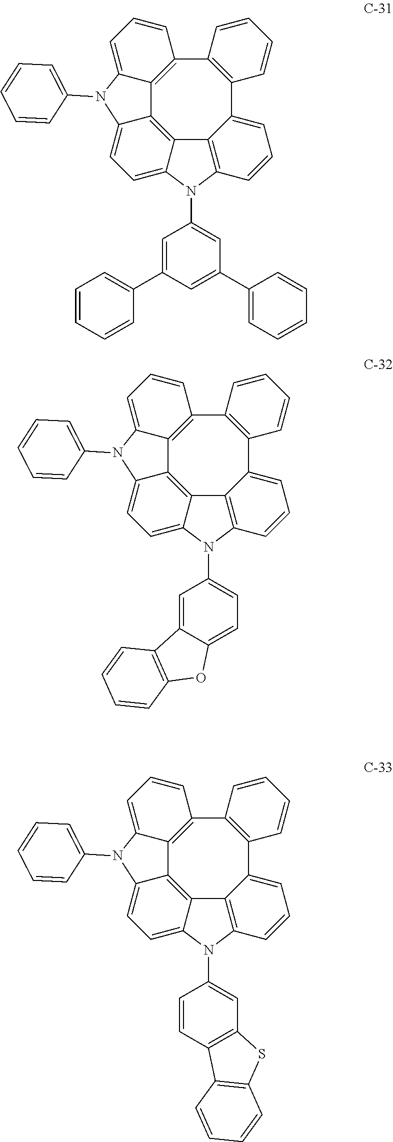

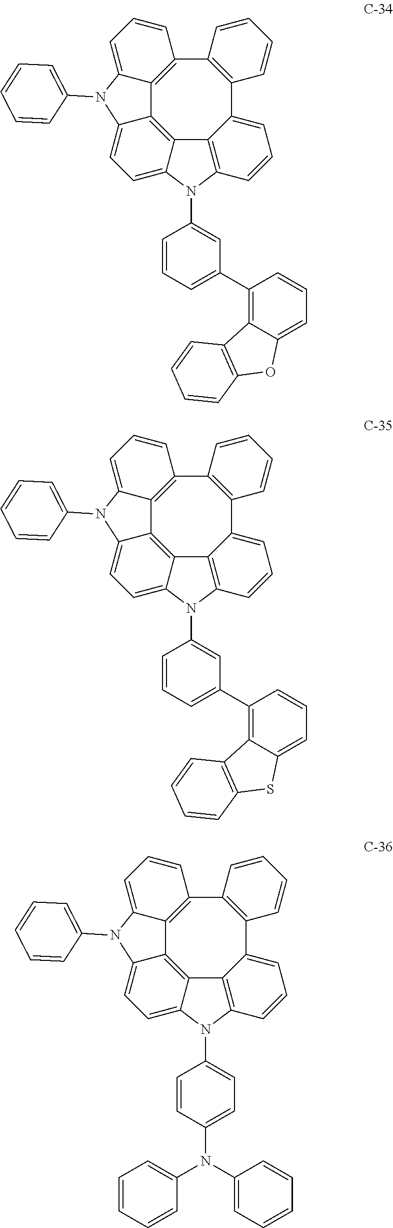

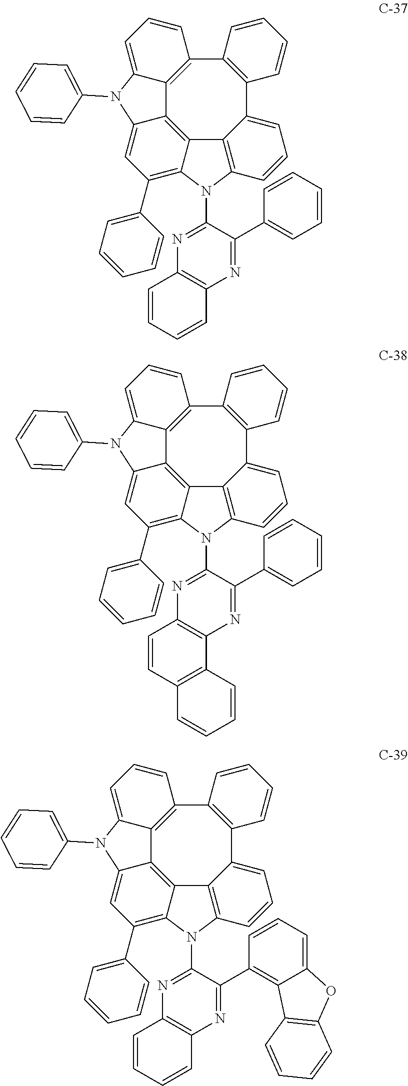

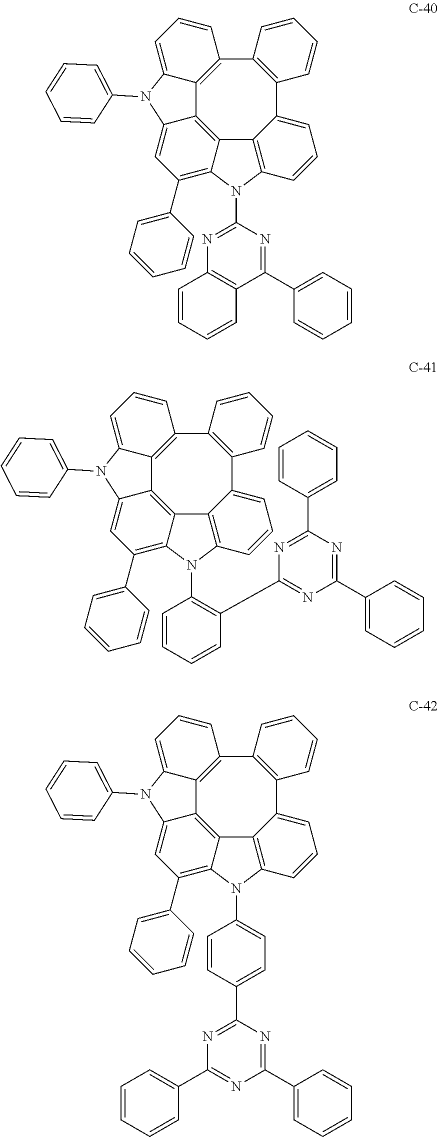 [SMILES: c1ccc(-c2nc(-n3c4cccc5c6ccccc6c6cccc7c6c6c(c54)c3c(-c3ccccc3)cc6n7-c3ccccc3)nc3ccccc23)cc1 ; c1ccc(-c2nc(-c3ccccc3)nc(-c3ccccc3-n3c4cccc5c6ccccc6c6cccc7c6c6c(c54)c3c(-c3ccccc3)cc6n7-c3ccccc3)n2)cc1 ; c1ccc(-c2nc(-c3ccccc3)nc(-c3ccc(-n4c5cccc6c7ccccc7c7cccc8c7c7c(c65)c4c(-c4ccccc4)cc7n8-c4ccccc4)cc3)n2)cc1]

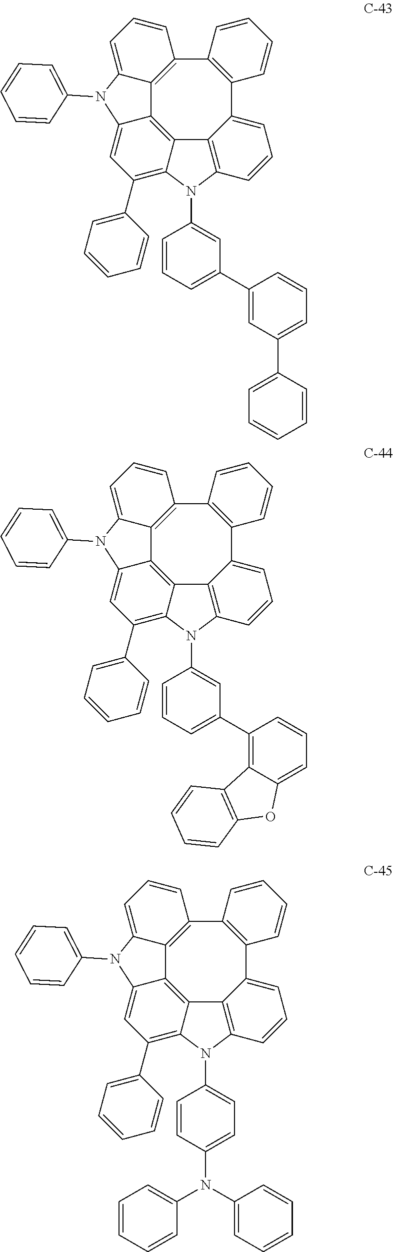

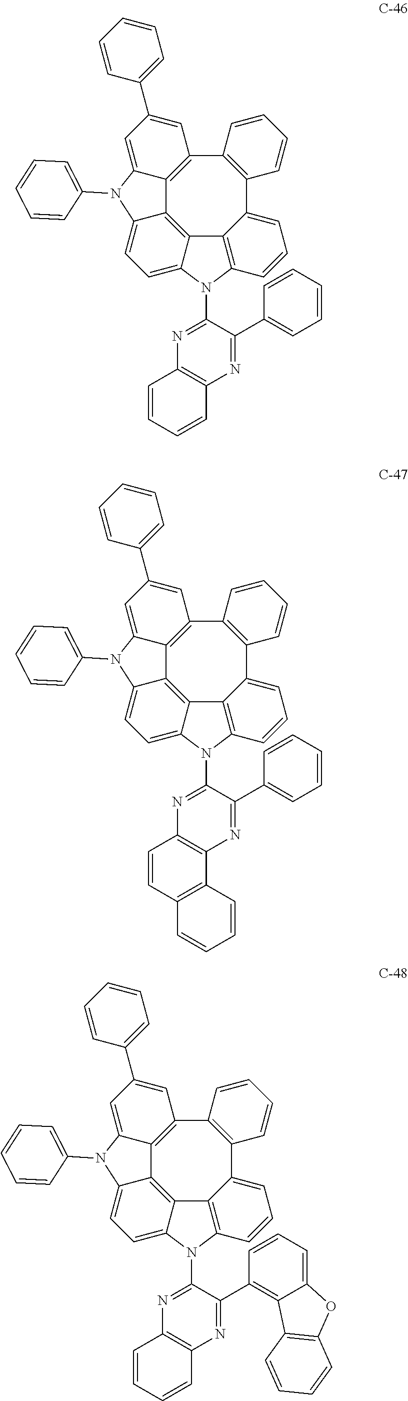 [SMILES: c1ccc(-c2cc3c4ccccc4c4cccc5c4c4c6c3c(c2)n(-c2ccccc2)c6ccc4n5-c2nc3ccccc3nc2-c2ccccc2)cc1 ; c1ccc(-c2cc3c4ccccc4c4cccc5c4c4c6c3c(c2)n(-c2ccccc2)c6ccc4n5-c2nc3ccc4ccccc4c3nc2-c2ccccc2)cc1 ; c1ccc(-c2cc3c4ccccc4c4cccc5c4c4c6c3c(c2)n(-c2ccccc2)c6ccc4n5-c2nc3ccccc3nc2-c2cccc3oc4ccccc4c23)cc1]

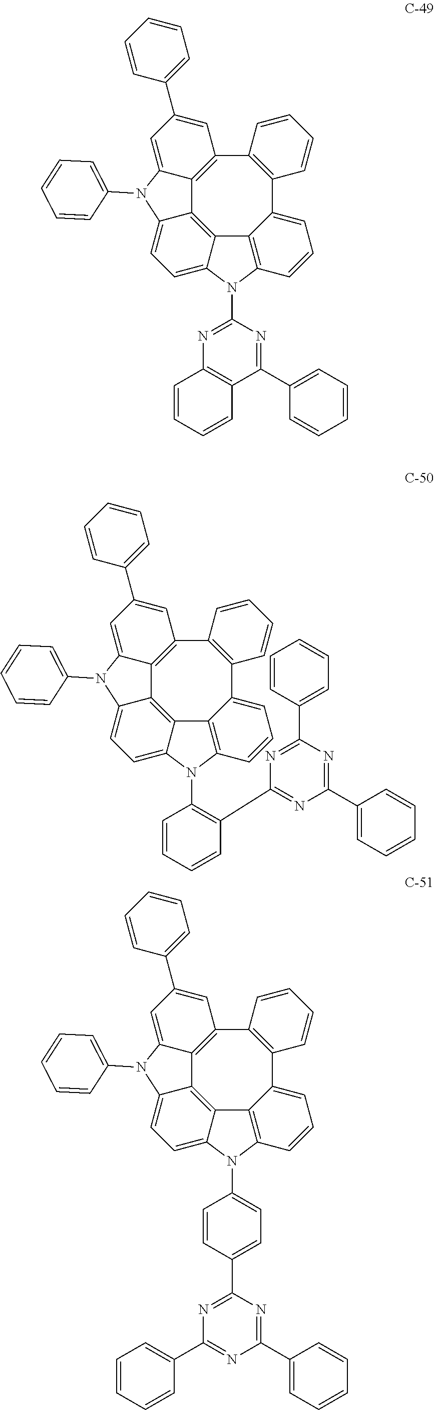

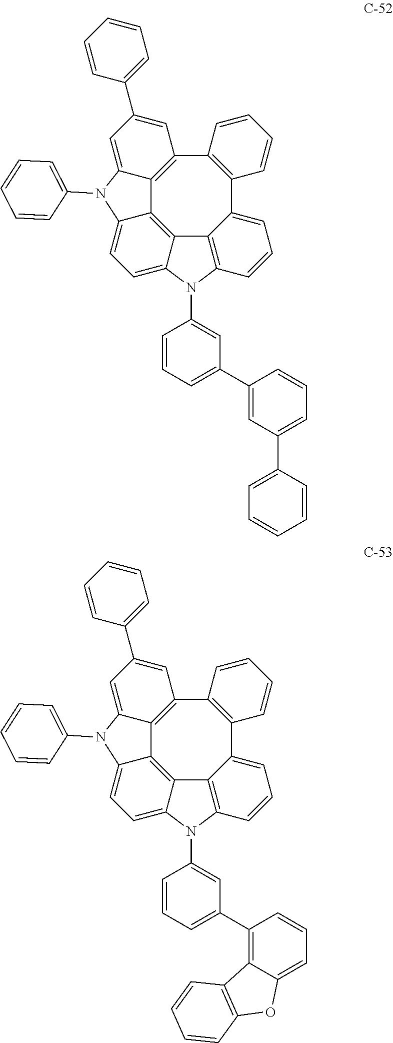 [SMILES: c1ccc(-c2cccc(-c3cccc(-n4c5cccc6c7ccccc7c7cc(-c8ccccc8)cc8c7c7c(c65)c4ccc7n8-c4ccccc4)c3)c2)cc1 ; c1ccc(-c2cc3c4ccccc4c4cccc5c4c4c6c3c(c2)n(-c2ccccc2)c6ccc4n5-c2cccc(-c3cccc4oc5ccccc5c34)c2)cc1]

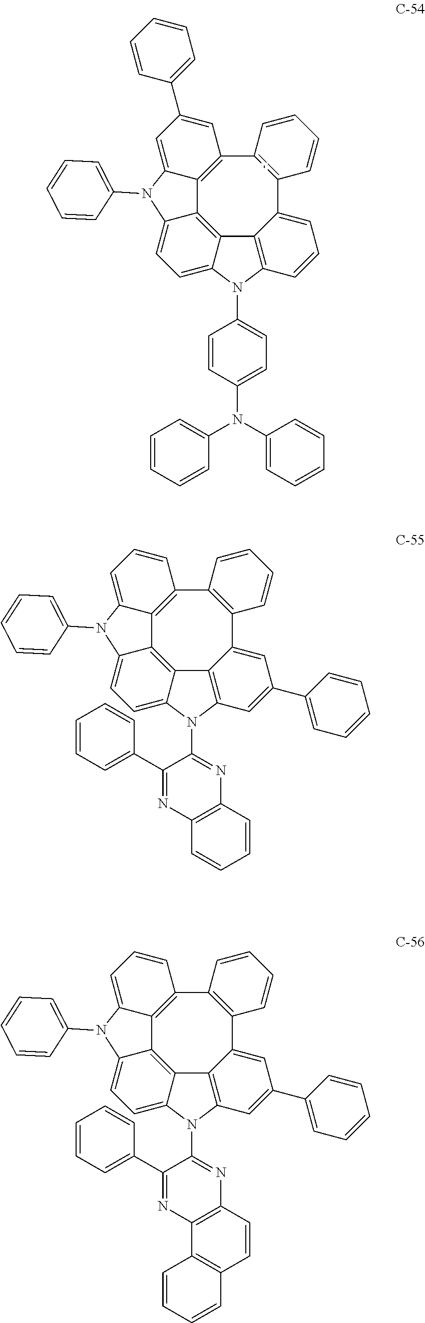

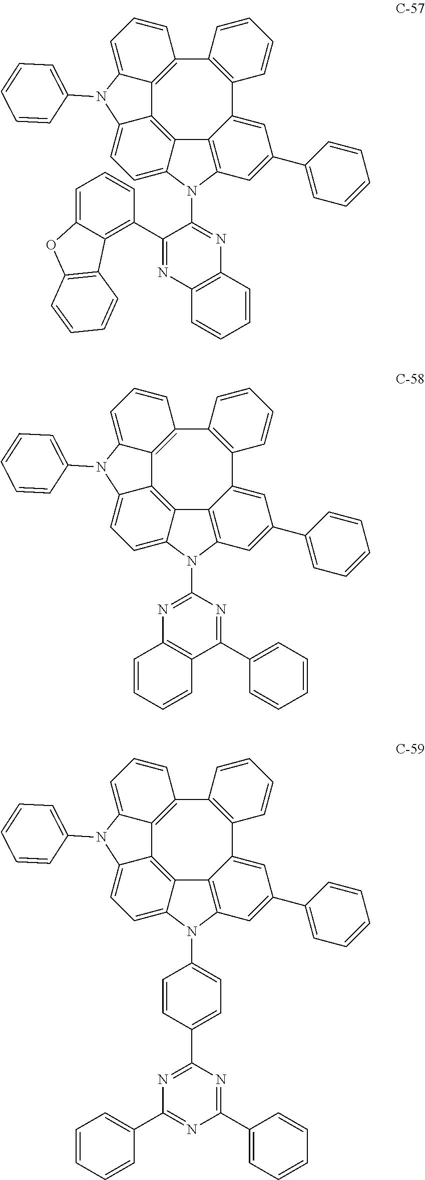 [SMILES: c1ccc(-c2cc3c4ccccc4c4cccc5c4c4c6c3c(c2)n(-c2nc3ccccc3nc2-c2cccc3oc7ccccc7c23)c6ccc4n5-c2ccccc2)cc1 ; c1ccc(-c2cc3c4ccccc4c4cccc5c4c4c6c3c(c2)n(-c2nc(-c3ccccc3)c3ccccc3n2)c6ccc4n5-c2ccccc2)cc1 ; c1ccc(-c2cc3c4ccccc4c4cccc5c4c4c6c3c(c2)n(-c2ccc(-c3nc(-c7ccccc7)nc(-c7ccccc7)n3)cc2)c6ccc4n5-c2ccccc2)cc1]

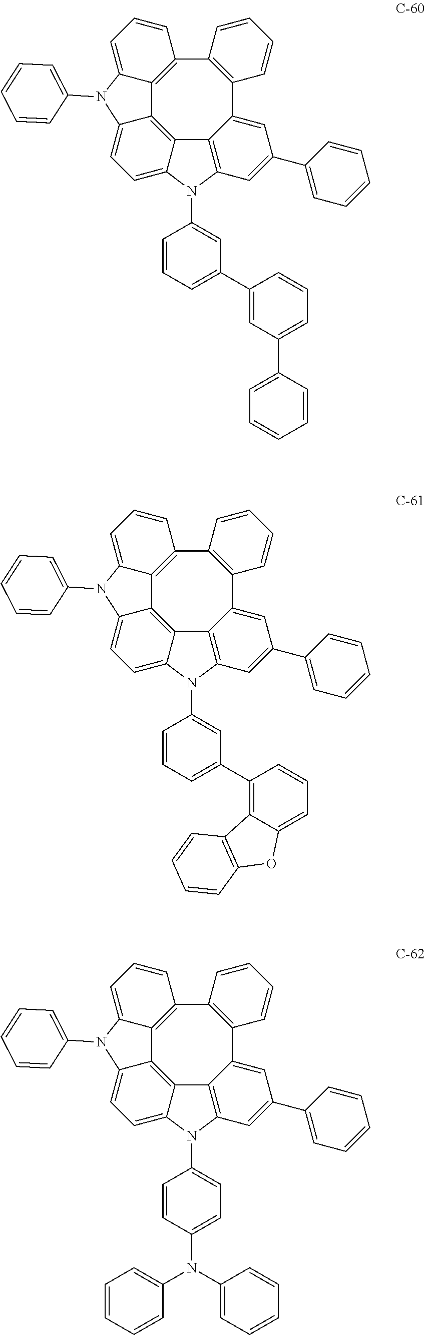 [SMILES: c1ccc(-c2cccc(-c3cccc(-n4c5cc(-c6ccccc6)cc6c7ccccc7c7cccc8c7c7c(c65)c4ccc7n8-c4ccccc4)c3)c2)cc1 ; c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1]

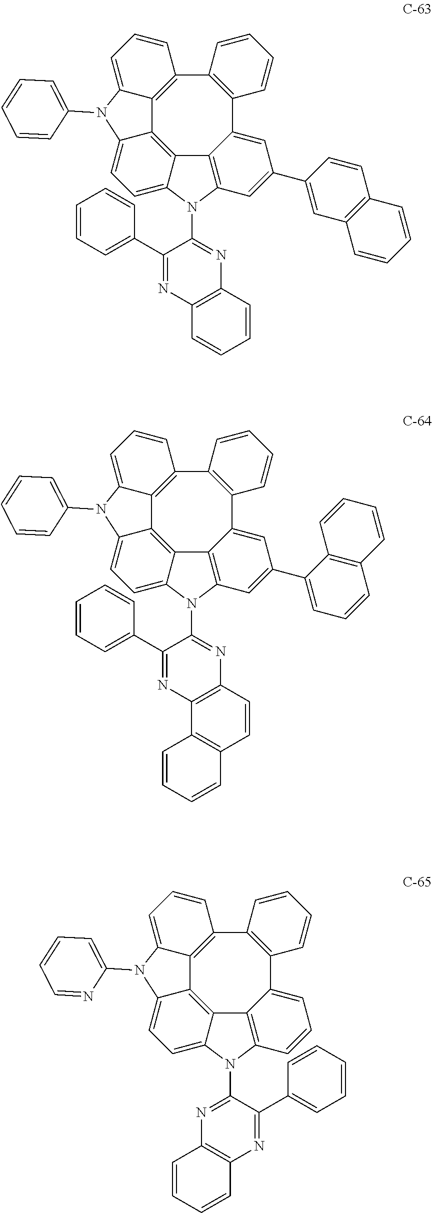 [SMILES: c1ccc(-c2nc3ccccc3nc2-n2c3cc(-c4ccc5ccccc5c4)cc4c5ccccc5c5cccc6c5c5c(c43)c2ccc5n6-c2ccccc2)cc1 ; c1ccc(-c2nc3c(ccc4ccccc43)nc2-n2c3cc(-c4cccc5ccccc45)cc4c5ccccc5c5cccc6c5c5c(c43)c2ccc5n6-c2ccccc2)cc1 ; c1ccc(-c2nc3ccccc3nc2-n2c3cccc4c5ccccc5c5cccc6c5c5c(c43)c2ccc5n6-c2ccccn2)cc1]

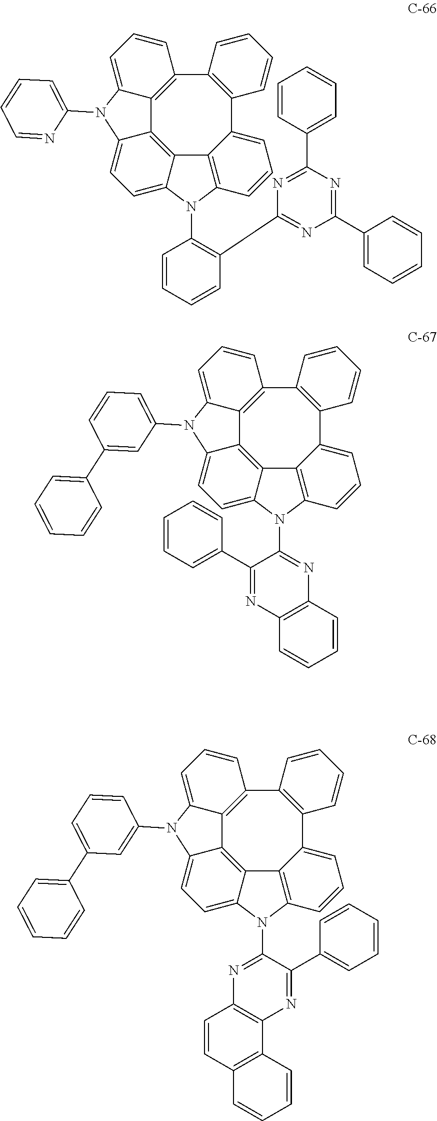 [SMILES: c1ccc(-c2nc(-c3ccccc3)nc(-c3ccccc3-n3c4cccc5c6ccccc6c6cccc7c6c6c(c54)c3ccc6n7-c3ccccn3)n2)cc1 ; c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3nc4ccc5ccccc5c4nc3-c3ccccc3)c2)cc1]

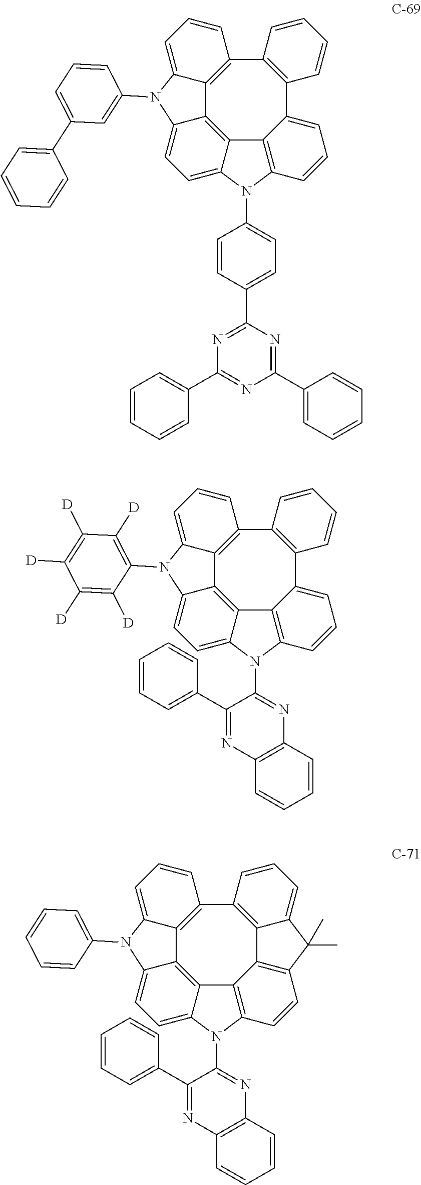 [SMILES: c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1 ; [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H] ; CC1(C)c2cccc3c4cccc5c4c4c6c7c(c1ccc7n(-c1nc7ccccc7nc1-c1ccccc1)c6ccc4n5-c1ccccc1)c23]

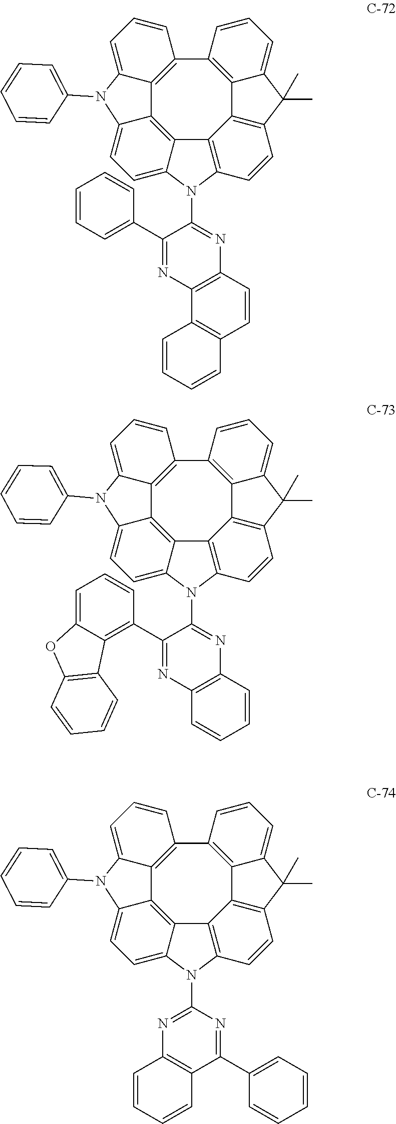

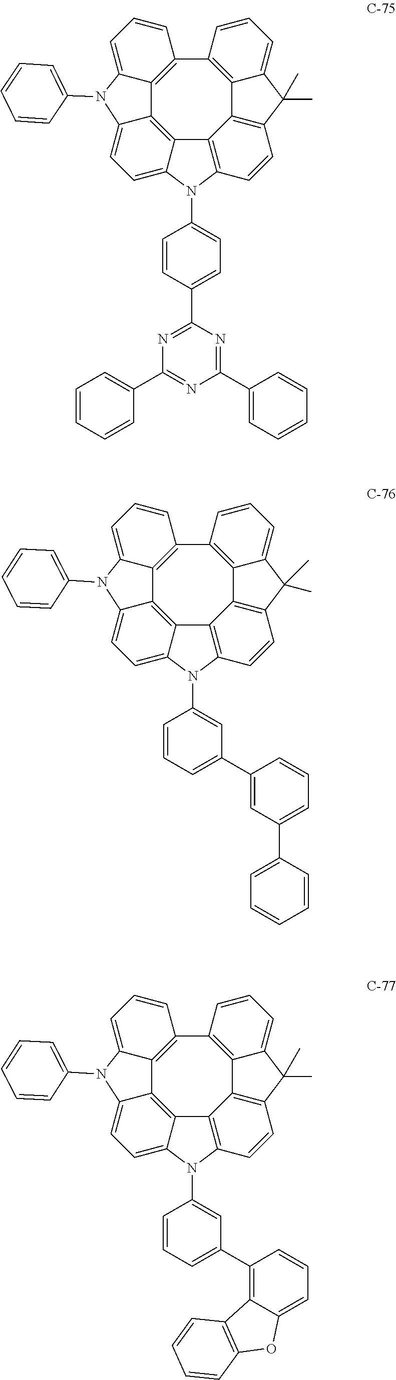 [SMILES: CC1(C)c2cccc3c4cccc5c4c4c6c7c(c1ccc7n(-c1ccc(-c7nc(-c8ccccc8)nc(-c8ccccc8)n7)cc1)c6ccc4n5-c1ccccc1)c23 ; CC1(C)c2cccc3c4cccc5c4c4c6c7c(c1ccc7n(-c1cccc(-c7cccc(-c8ccccc8)c7)c1)c6ccc4n5-c1ccccc1)c23 ; CC1(C)c2cccc3c4cccc5c4c4c6c7c(c1ccc7n(-c1cccc(-c7cccc8oc9ccccc9c78)c1)c6ccc4n5-c1ccccc1)c23]

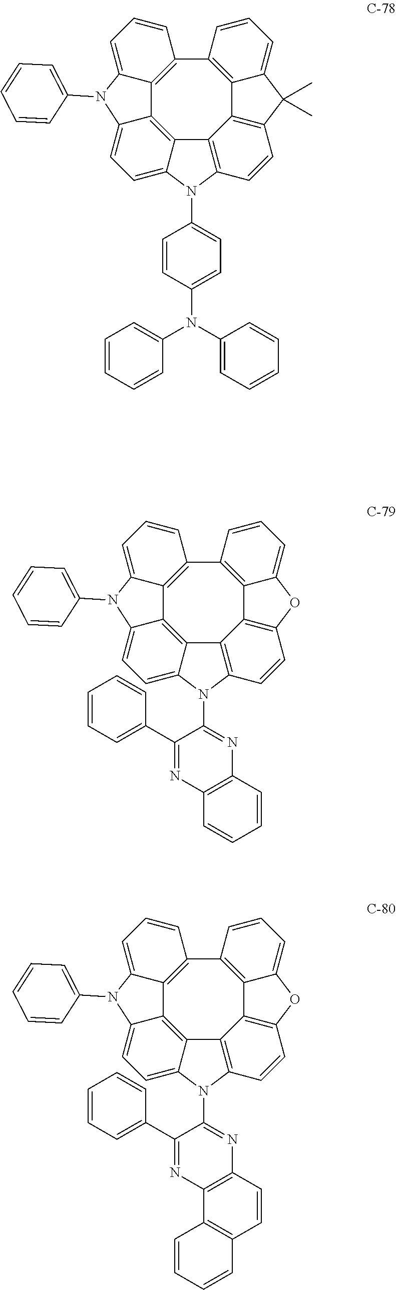 [SMILES: CC1(C)c2cccc3c4cccc5c4c4c6c7c(c1ccc7n(-c1ccc(N(c7ccccc7)c7ccccc7)cc1)c6ccc4n5-c1ccccc1)c23 ; c1ccc(-c2nc3ccccc3nc2-n2c3ccc4oc5cccc6c7cccc8c7c7c(c3c4c56)c2ccc7n8-c2ccccc2)cc1 ; c1ccc(-c2nc3c(ccc4ccccc43)nc2-n2c3ccc4oc5cccc6c7cccc8c7c7c(c3c4c56)c2ccc7n8-c2ccccc2)cc1]

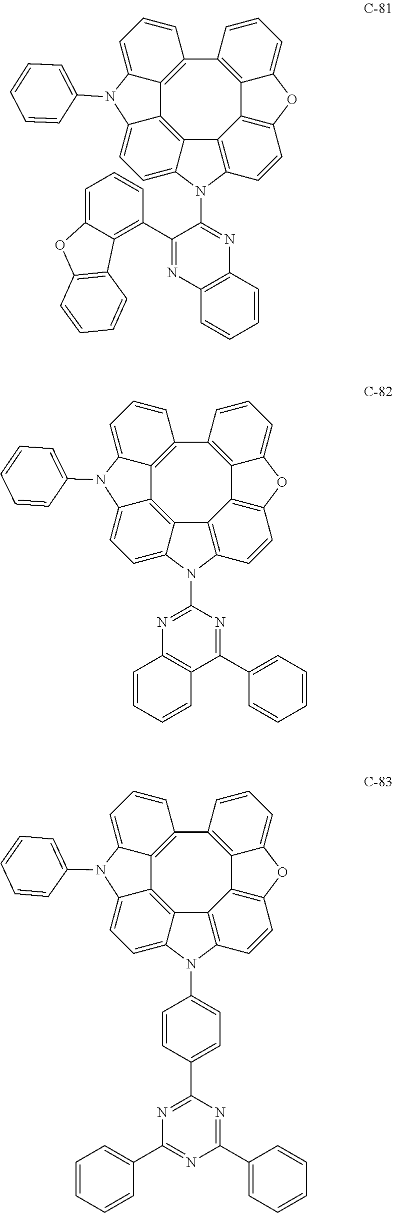 [SMILES: c1ccc(-n2c3cccc4c5cccc6oc7ccc8c(c7c65)c5c(c43)c2ccc5n8-c2nc3ccccc3nc2-c2cccc3oc4ccccc4c23)cc1 ; c1ccc(-c2nc(-n3c4ccc5oc6cccc7c8cccc9c8c8c(c4c5c67)c3ccc8n9-c3ccccc3)nc3ccccc23)cc1 ; c1ccc(-c2nc(-c3ccccc3)nc(-c3ccc(-n4c5ccc6oc7cccc8c9cccc%10c9c9c(c5c6c78)c4ccc9n%10-c4ccccc4)cc3)n2)cc1]

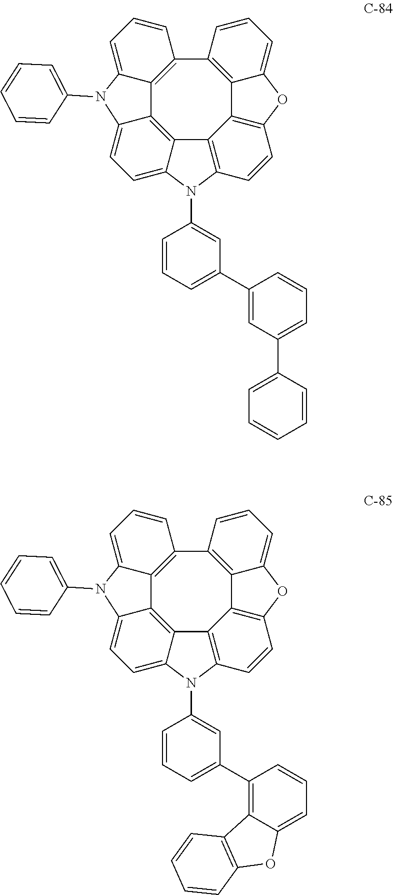 [SMILES: c1ccc(-c2cccc(-c3cccc(-n4c5ccc6oc7cccc8c9cccc%10c9c9c(c5c6c78)c4ccc9n%10-c4ccccc4)c3)c2)cc1 ; c1ccc(-n2c3cccc4c5cccc6oc7ccc8c(c7c65)c5c(c43)c2ccc5n8-c2cccc(-c3cccc4oc5ccccc5c34)c2)cc1]

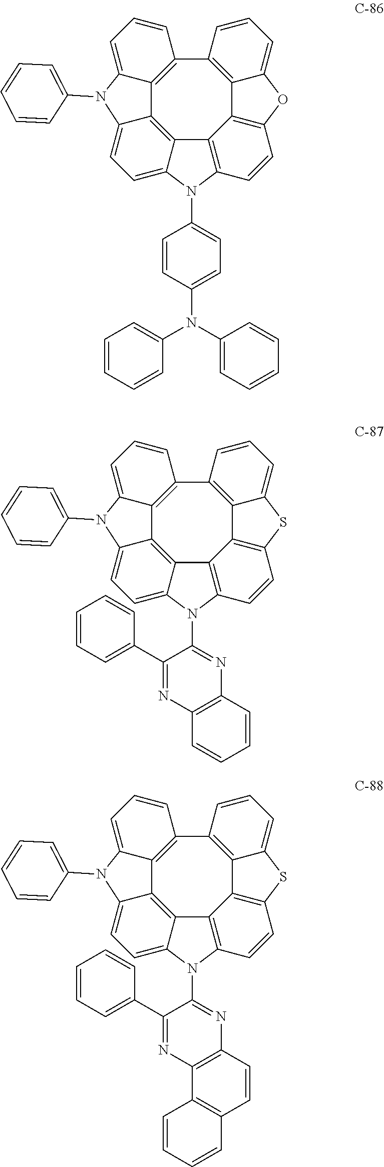

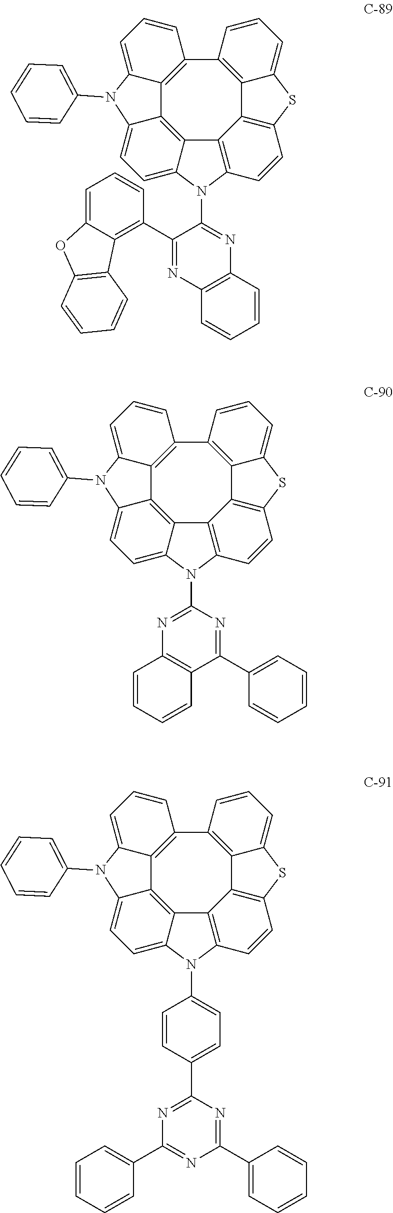 [SMILES: c1ccc(-n2c3cccc4c5cccc6sc7ccc8c(c7c65)c5c(c43)c2ccc5n8-c2nc3ccccc3nc2-c2cccc3oc4ccccc4c23)cc1 ; c1ccc(-c2nc(-n3c4ccc5sc6cccc7c8cccc9c8c8c(c4c5c67)c3ccc8n9-c3ccccc3)nc3ccccc23)cc1 ; c1ccc(-c2nc(-c3ccccc3)nc(-c3ccc(-n4c5ccc6sc7cccc8c9cccc%10c9c9c(c5c6c78)c4ccc9n%10-c4ccccc4)cc3)n2)cc1]

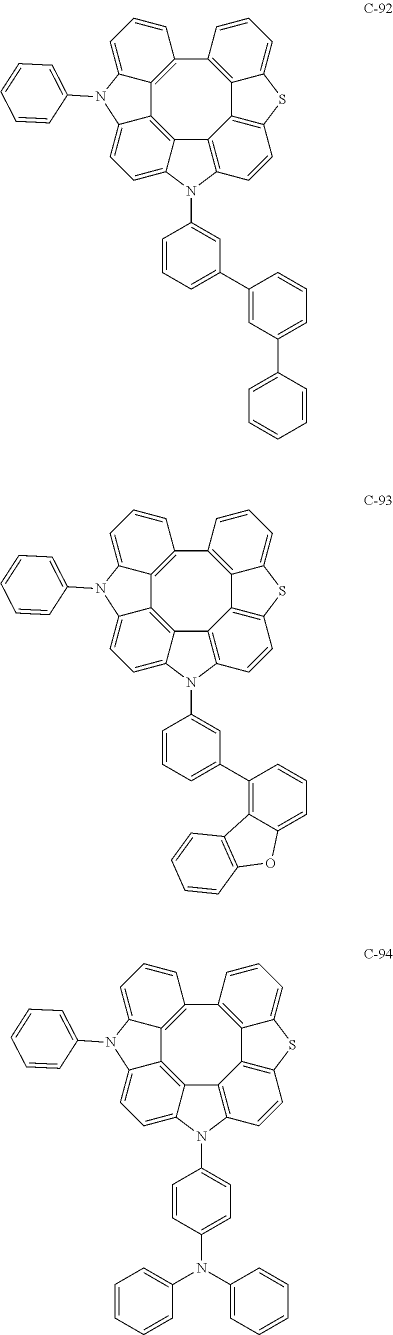 [SMILES: c1ccc(-c2cccc(-c3cccc(-n4c5ccc6sc7cccc8c9cccc%10c9c9c(c5c6c78)c4ccc9n%10-c4ccccc4)c3)c2)cc1 ; c1ccc(-n2c3cccc4c5cccc6sc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5sc6cccc7c8cccc9c8c8c(c4c5c67)c3ccc8n9-c3ccccc3)cc2)cc1]

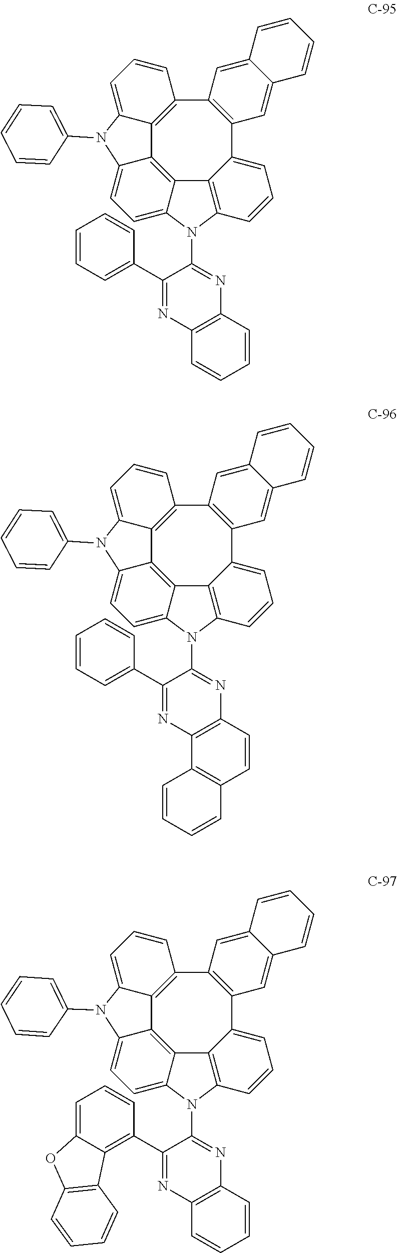 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

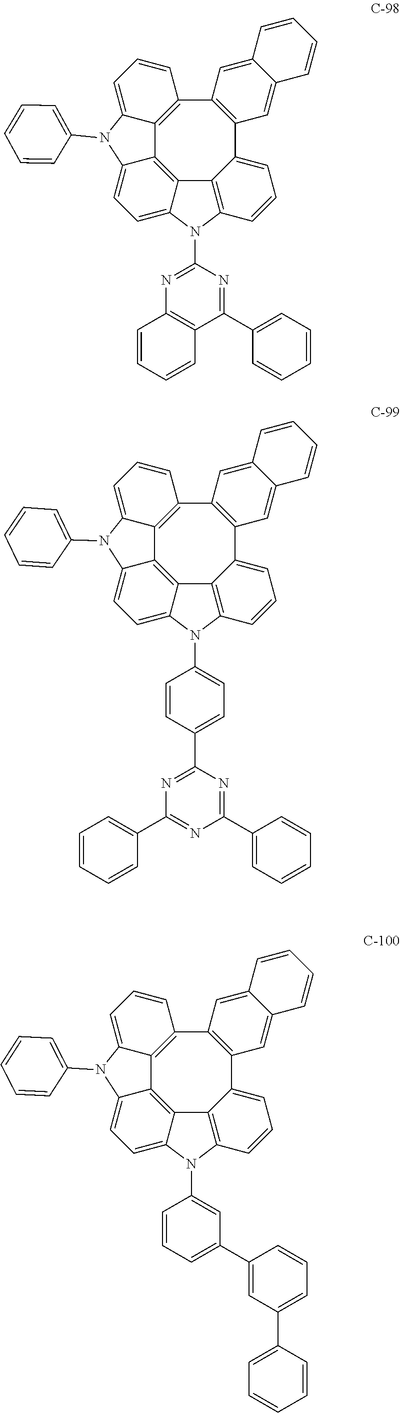

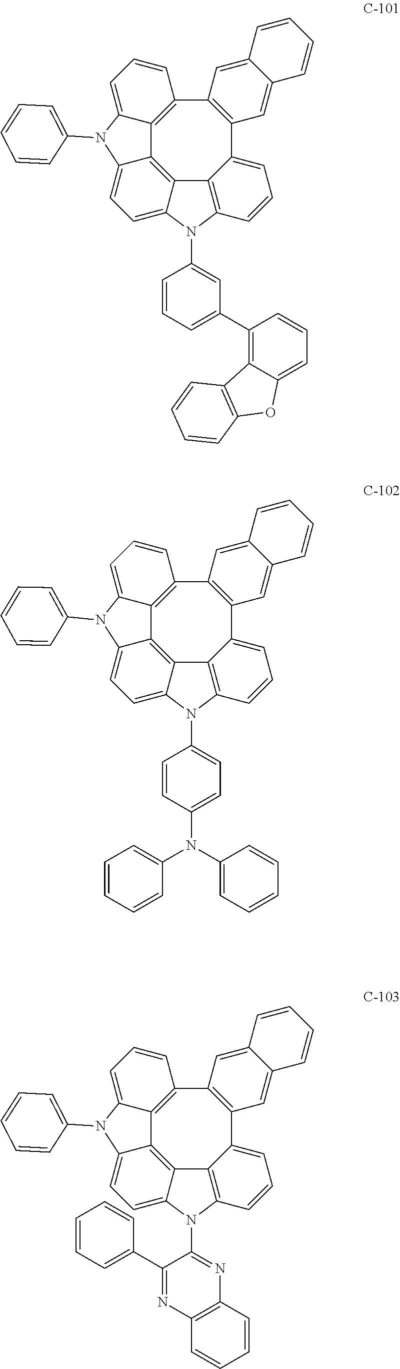 [SMILES: c1ccc(-n2c3cccc4c5cc6ccccc6cc5c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cc7ccccc7cc6c6cccc7c6c6c(c54)c3ccc6n7-c3ccccc3)cc2)cc1 ; c1ccc(-c2nc3ccccc3nc2-n2c3cccc4c5cc6ccccc6cc5c5cccc6c5c5c(c43)c2ccc5n6-c2ccccc2)cc1]

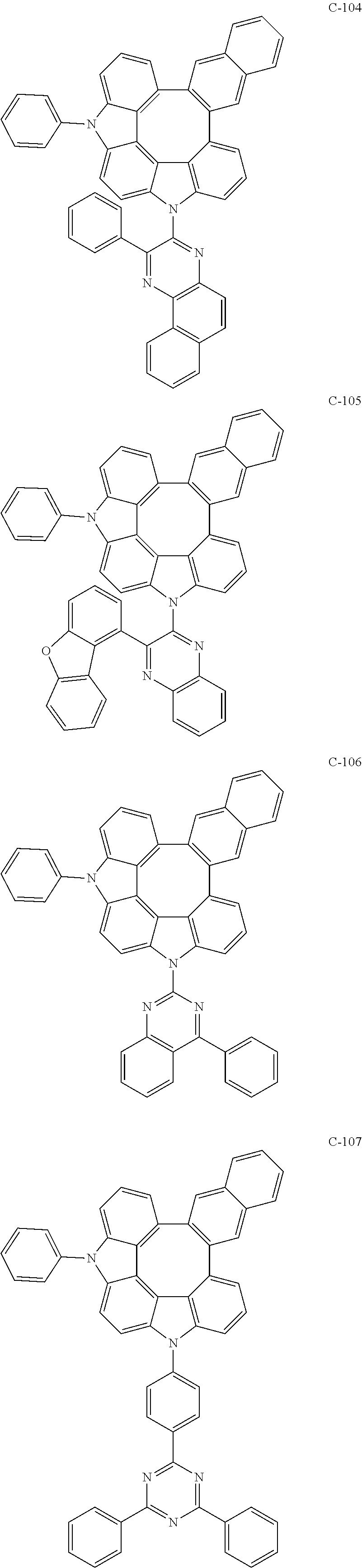 [SMILES: c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1 ; c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1]

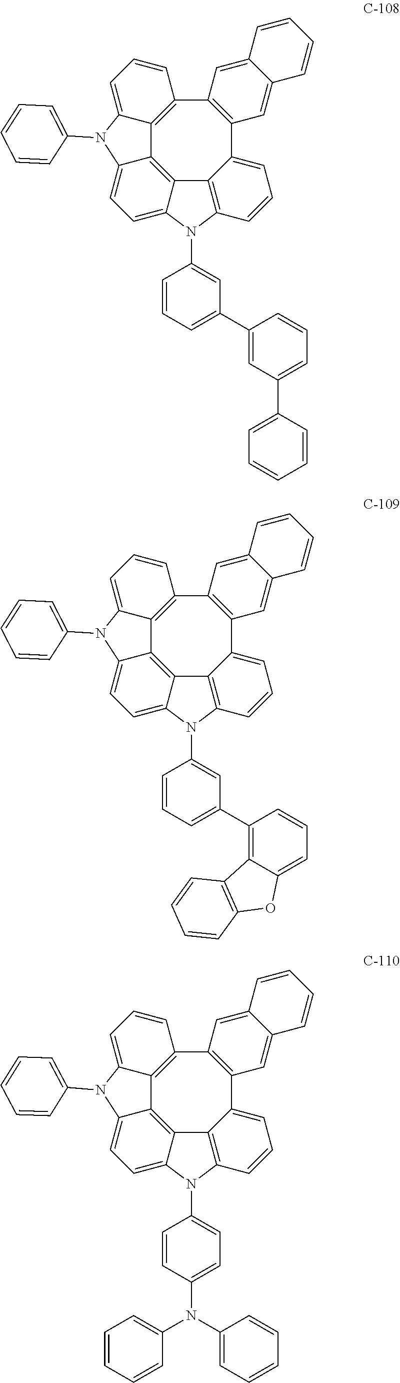

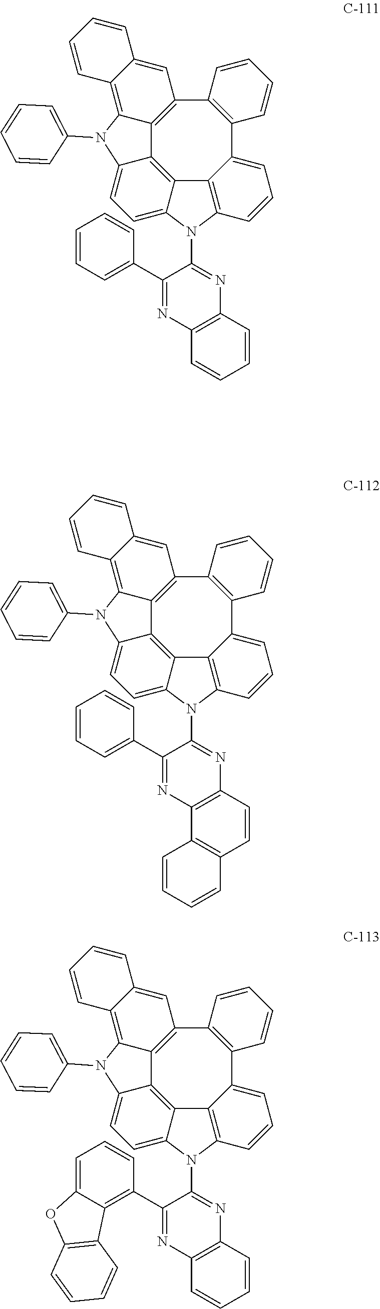 [SMILES: c1ccc(-c2nc3ccccc3nc2-n2c3cccc4c5ccccc5c5cc6ccccc6c6c5c5c(c43)c2ccc5n6-c2ccccc2)cc1 ; c1ccc(-c2nc3c(ccc4ccccc43)nc2-n2c3cccc4c5ccccc5c5cc6ccccc6c6c5c5c(c43)c2ccc5n6-c2ccccc2)cc1 ; c1ccc(-n2c3ccc4c5c6c(cccc6n4-c4nc6ccccc6nc4-c4cccc6oc7ccccc7c46)c4ccccc4c4cc6ccccc6c2c4c53)cc1]

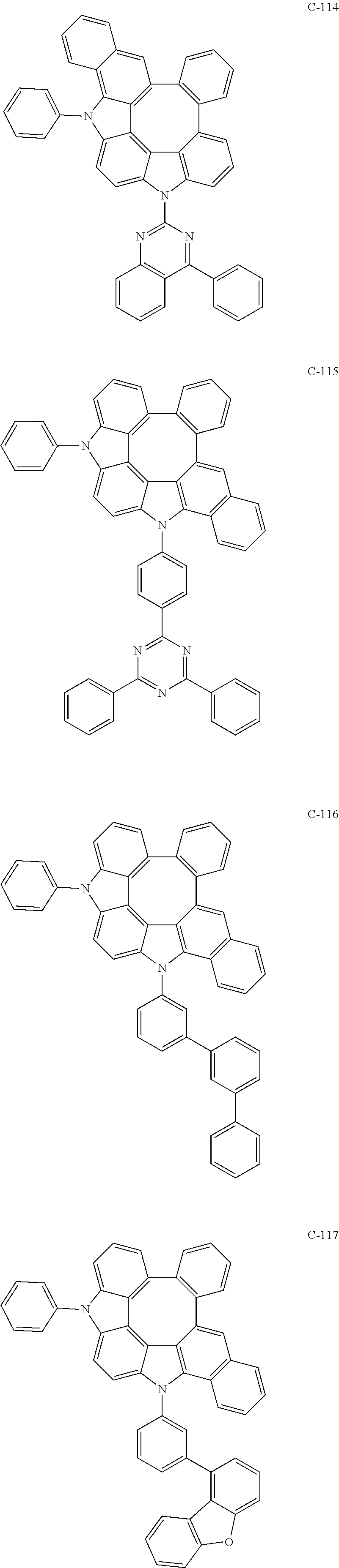 [SMILES: c1ccc(-c2nc(-n3c4cccc5c6ccccc6c6cc7ccccc7c7c6c6c(c54)c3ccc6n7-c3ccccc3)nc3ccccc23)cc1 ; c1ccc(-c2nc(-c3ccccc3)nc(-c3ccc(-n4c5ccc6c7c8c(cccc8n6-c6ccccc6)c6ccccc6c6cc8ccccc8c4c6c75)cc3)n2)cc1 ; c1ccc(-c2cccc(-c3cccc(-n4c5ccc6c7c8c(cccc8n6-c6ccccc6)c6ccccc6c6cc8ccccc8c4c6c75)c3)c2)cc1 ; c1ccc(-n2c3cccc4c5ccccc5c5cc6ccccc6c6c5c5c(c43)c2ccc5n6-c2cccc(-c3cccc4oc5ccccc5c34)c2)cc1]

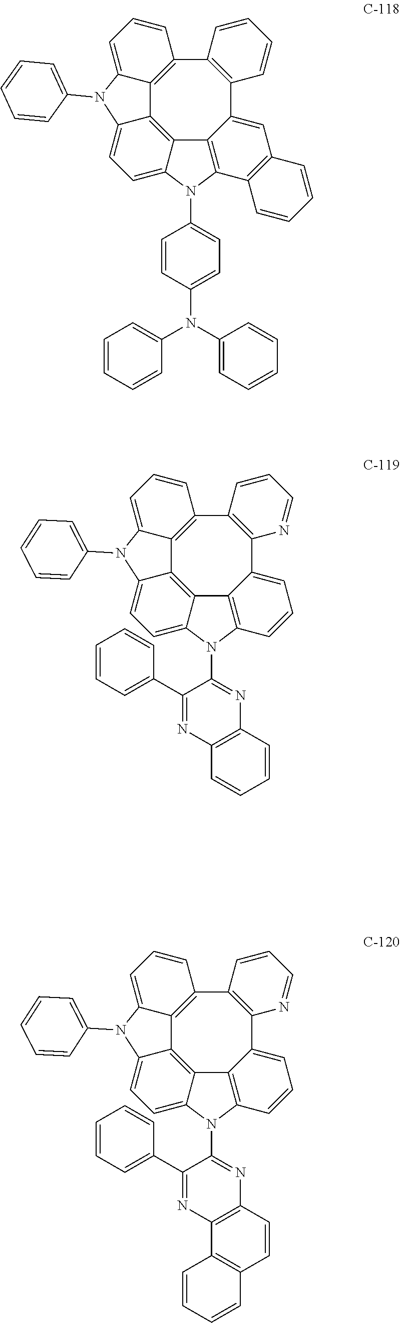 [SMILES: c1ccc(N(c2ccccc2)c2ccc(-n3c4ccc5c6c7c(cccc7n5-c5ccccc5)c5ccccc5c5cc7ccccc7c3c5c64)cc2)cc1 ; c1ccc(-c2nc3ccccc3nc2-n2c3cccc4c5ncccc5c5cccc6c5c5c(c43)c2ccc5n6-c2ccccc2)cc1 ; c1ccc(-c2nc3c(ccc4ccccc43)nc2-n2c3cccc4c5ncccc5c5cccc6c5c5c(c43)c2ccc5n6-c2ccccc2)cc1]

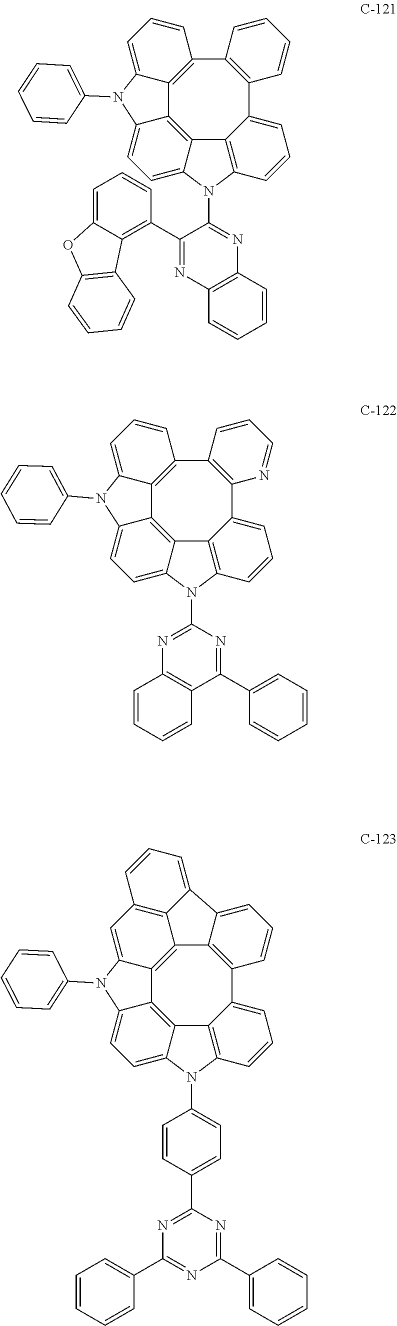 [SMILES: c1ccc(-n2c3cccc4c5ccccc5c5cccc6c5c5c(c43)c2ccc5n6-c2nc3ccccc3nc2-c2cccc3oc4ccccc4c23)cc1 ; c1ccc(-c2nc(-n3c4cccc5c6ncccc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cccc8c7c7c9c-8cccc9cc8c7c7c(c65)c4ccc7n8-c4ccccc4)cc3)n2)cc1]

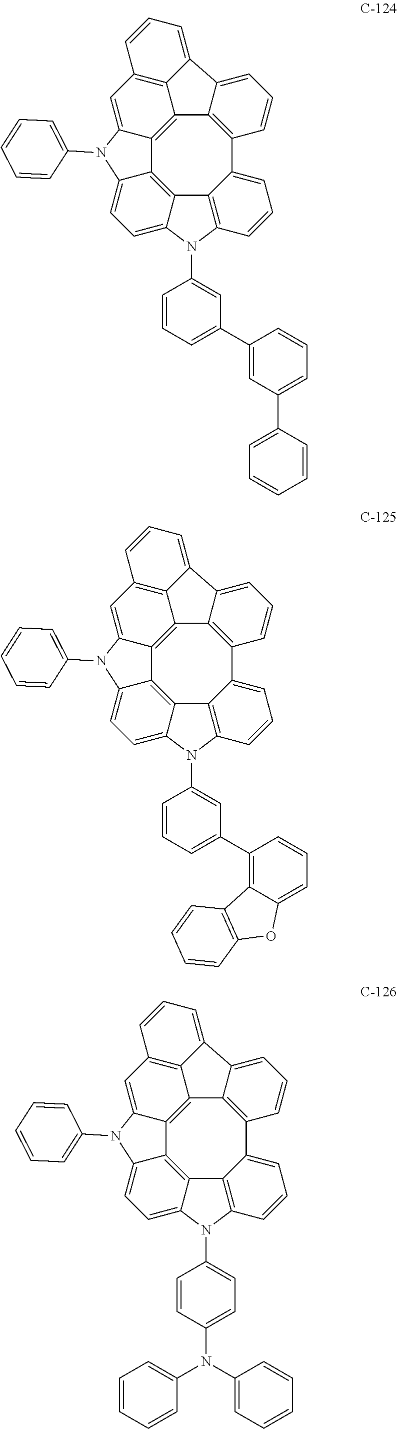

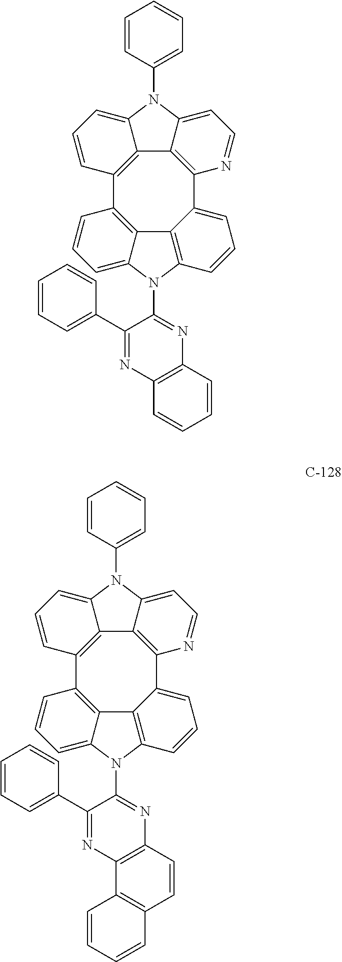 [SMILES: c1ccc(-c2nc3ccccc3nc2-n2c3cccc4c5cccc6c5c5c(nccc5n6-c5ccccc5)c5cccc2c5c43)cc1 ; c1ccc(-c2nc3c(ccc4ccccc43)nc2-n2c3cccc4c5cccc6c5c5c(nccc5n6-c5ccccc5)c5cccc2c5c43)cc1]

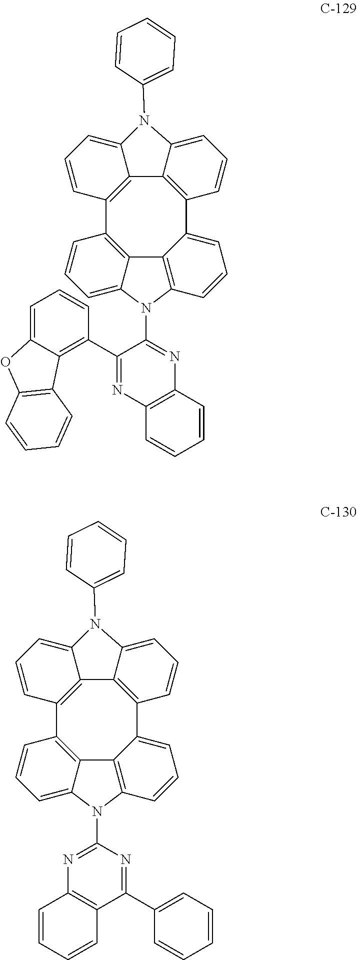 [SMILES: c1ccc(-n2c3cccc4c5cccc6c5c5c(cccc5n6-c5nc6ccccc6nc5-c5cccc6oc7ccccc7c56)c5cccc2c5c43)cc1 ; c1ccc(-c2nc(-n3c4cccc5c6cccc7c6c6c(cccc6n7-c6ccccc6)c6cccc3c6c54)nc3ccccc23)cc1]

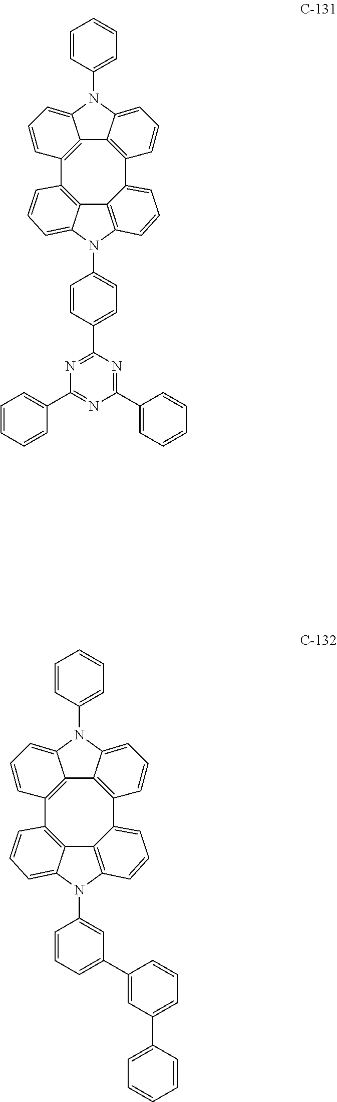 [SMILES: c1ccc(-c2nc(-c3ccccc3)nc(-c3ccc(-n4c5cccc6c7cccc8c7c7c(cccc7n8-c7ccccc7)c7cccc4c7c65)cc3)n2)cc1 ; c1ccc(-c2cccc(-c3cccc(-n4c5cccc6c7cccc8c7c7c(cccc7n8-c7ccccc7)c7cccc4c7c65)c3)c2)cc1]

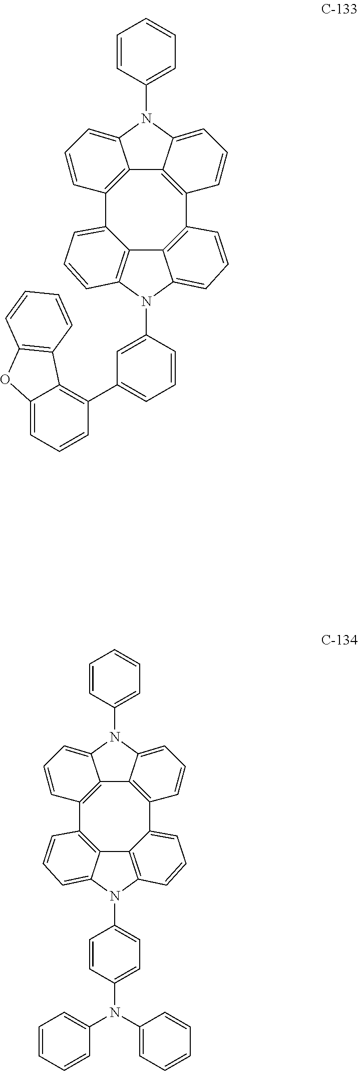 [SMILES: c1ccc(-n2c3cccc4c5cccc6c5c5c(cccc5n6-c5cccc(-c6cccc7oc8ccccc8c67)c5)c5cccc2c5c43)cc1 ; c1ccc(N(c2ccccc2)c2ccc(-n3c4cccc5c6cccc7c6c6c(cccc6n7-c6ccccc6)c6cccc3c6c54)cc2)cc1]

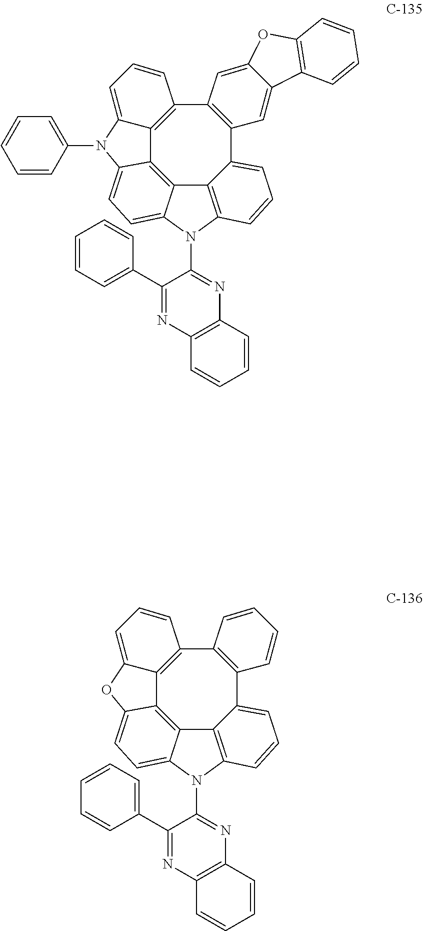 [SMILES: c1ccc(-c2nc3ccccc3nc2-n2c3cccc4c5cc6c(cc5c5cccc7c5c5c(c43)c2ccc5n7-c2ccccc2)oc2ccccc26)cc1 ; c1ccc(-c2nc3ccccc3nc2-n2c3cccc4c5ccccc5c5cccc6oc7ccc2c(c7c65)c43)cc1]

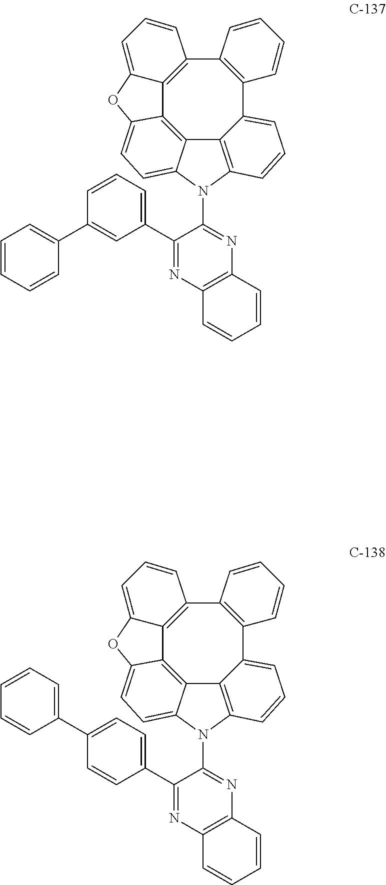 [SMILES: c1ccc(-c2cccc(-c3nc4ccccc4nc3-n3c4cccc5c6ccccc6c6cccc7oc8ccc3c(c8c76)c54)c2)cc1 ; c1ccc(-c2ccc(-c3nc4ccccc4nc3-n3c4cccc5c6ccccc6c6cccc7oc8ccc3c(c8c76)c54)cc2)cc1]

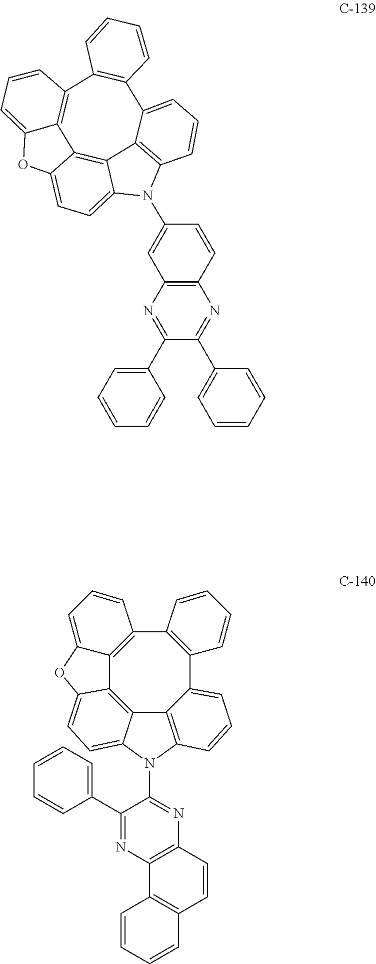 [SMILES: c1ccc(-c2nc3ccc(-n4c5cccc6c7ccccc7c7cccc8oc9ccc4c(c9c87)c65)cc3nc2-c2ccccc2)cc1 ; c1ccc(-c2nc3c(ccc4ccccc43)nc2-n2c3cccc4c5ccccc5c5cccc6oc7ccc2c(c7c65)c43)cc1]

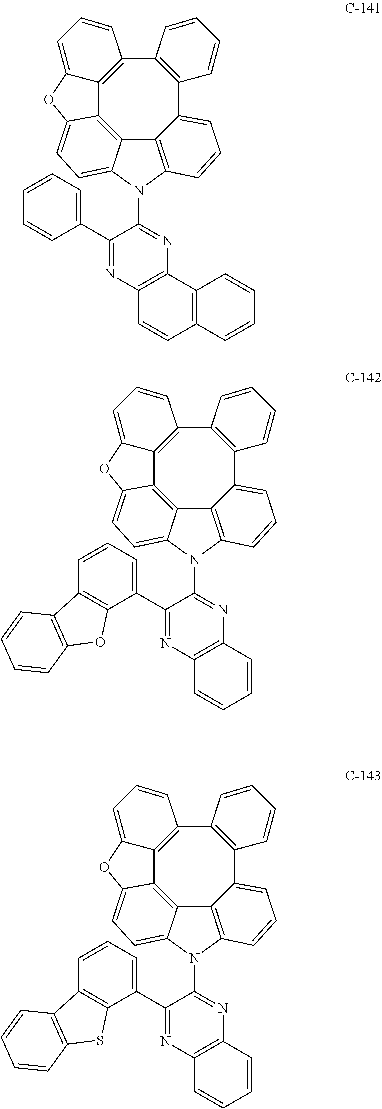 [SMILES: c1ccc(-c2nc3ccc4ccccc4c3nc2-n2c3cccc4c5ccccc5c5cccc6oc7ccc2c(c7c65)c43)cc1 ; c1ccc2nc(-n3c4cccc5c6ccccc6c6cccc7oc8ccc3c(c8c76)c54)c(-c3cccc4c3oc3ccccc34)nc2c1 ; c1ccc2nc(-n3c4cccc5c6ccccc6c6cccc7oc8ccc3c(c8c76)c54)c(-c3cccc4c3sc3ccccc34)nc2c1]

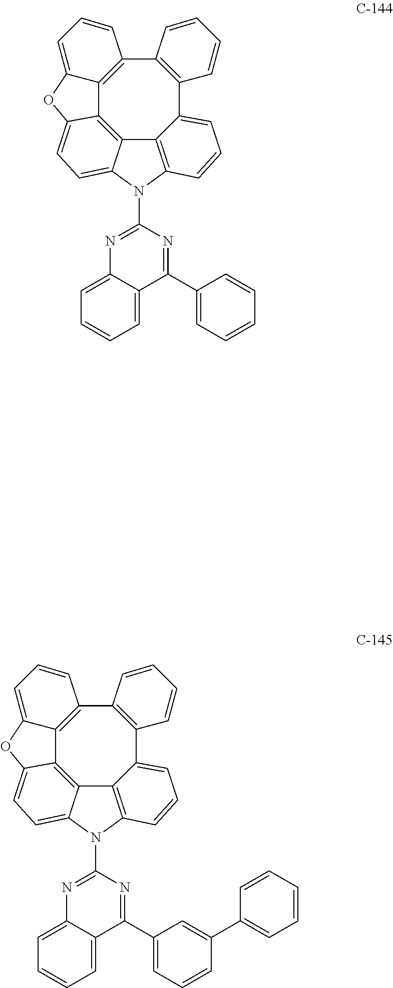 [SMILES: c1ccc(-c2nc(-n3c4cccc5c6ccccc6c6cccc7oc8ccc3c(c8c76)c54)nc3ccccc23)cc1 ; c1ccc(-c2cccc(-c3nc(-n4c5cccc6c7ccccc7c7cccc8oc9ccc4c(c9c87)c65)nc4ccccc34)c2)cc1]

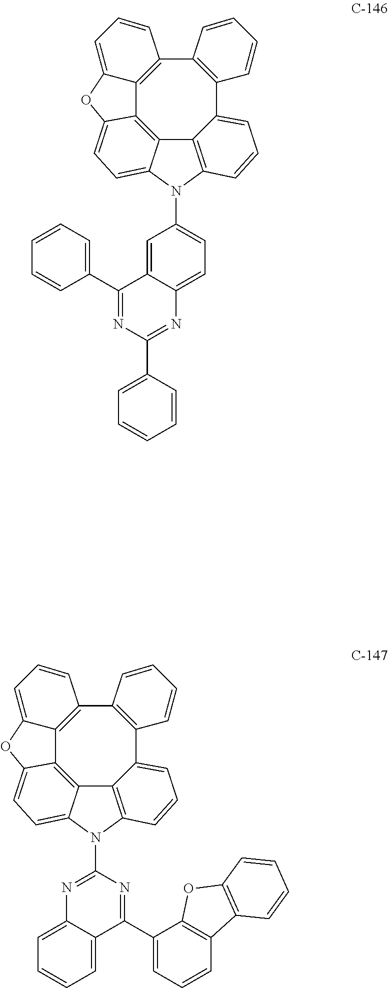 [SMILES: c1ccc(-c2nc(-c3ccccc3)c3cc(-n4c5cccc6c7ccccc7c7cccc8oc9ccc4c(c9c87)c65)ccc3n2)cc1 ; c1ccc2c(-c3cccc4c3oc3ccccc34)nc(-n3c4cccc5c6ccccc6c6cccc7oc8ccc3c(c8c76)c54)nc2c1]

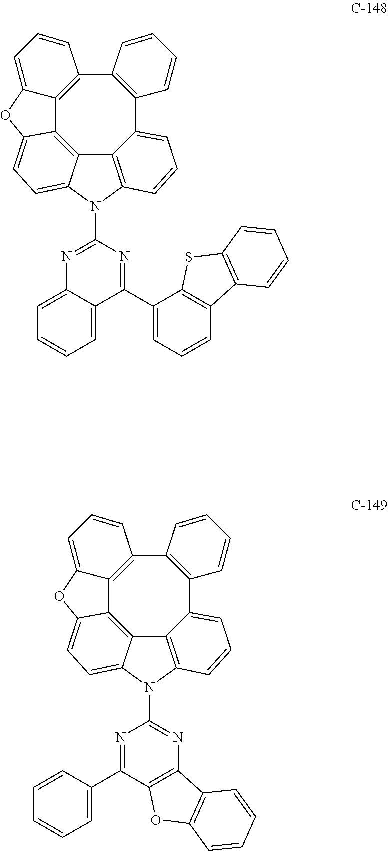 [SMILES: c1ccc2c(-c3cccc4c3sc3ccccc34)nc(-n3c4cccc5c6ccccc6c6cccc7oc8ccc3c(c8c76)c54)nc2c1 ; c1ccc(-c2nc(-n3c4cccc5c6ccccc6c6cccc7oc8ccc3c(c8c76)c54)nc3c2oc2ccccc23)cc1]

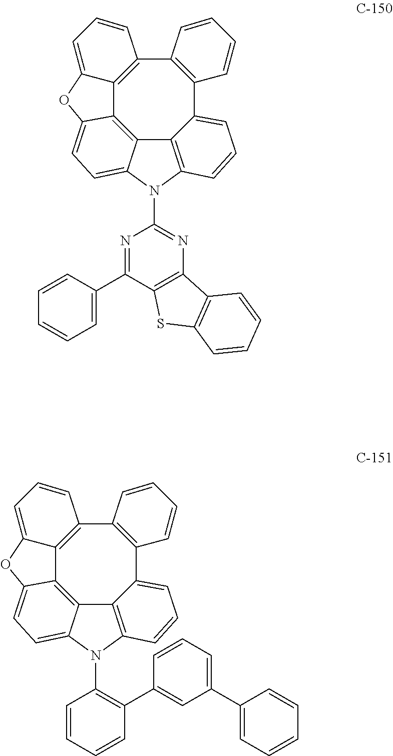 [SMILES: c1ccc(-c2nc(-n3c4cccc5c6ccccc6c6cccc7oc8ccc3c(c8c76)c54)nc3c2sc2ccccc23)cc1 ; c1ccc(-c2cccc(-c3ccccc3-n3c4cccc5c6ccccc6c6cccc7oc8ccc3c(c8c76)c54)c2)cc1]

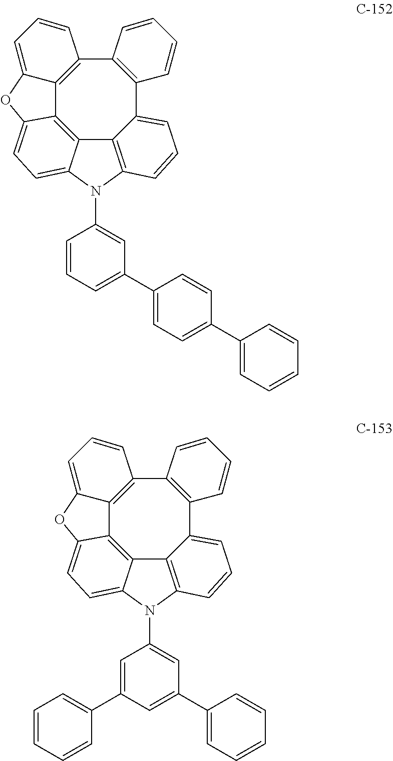 [SMILES: c1ccc(-c2ccc(-c3cccc(-n4c5cccc6c7ccccc7c7cccc8oc9ccc4c(c9c87)c65)c3)cc2)cc1 ; c1ccc(-c2cc(-c3ccccc3)cc(-n3c4cccc5c6ccccc6c6cccc7oc8ccc3c(c8c76)c54)c2)cc1]

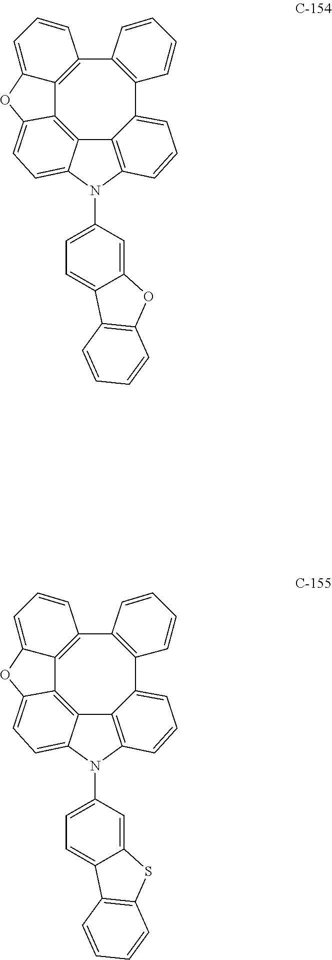 [SMILES: c1ccc2c(c1)oc1cc(-n3c4cccc5c6ccccc6c6cccc7oc8ccc3c(c8c76)c54)ccc12 ; c1ccc2c(c1)sc1cc(-n3c4cccc5c6ccccc6c6cccc7oc8ccc3c(c8c76)c54)ccc12]

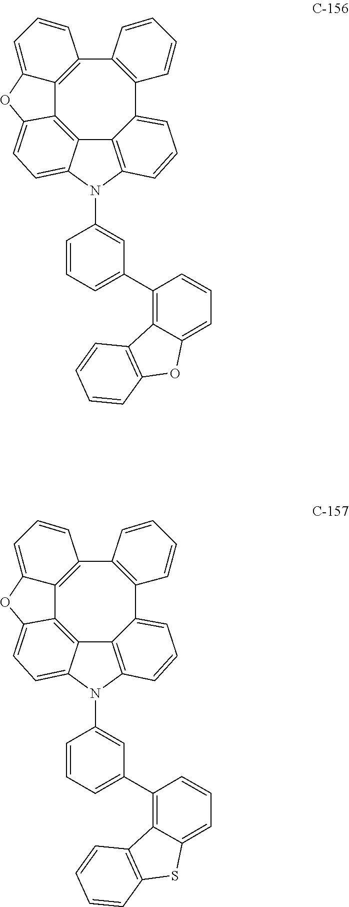 [SMILES: c1cc(-c2cccc3oc4ccccc4c23)cc(-n2c3cccc4c5ccccc5c5cccc6oc7ccc2c(c7c65)c43)c1 ; c1cc(-c2cccc3sc4ccccc4c23)cc(-n2c3cccc4c5ccccc5c5cccc6oc7ccc2c(c7c65)c43)c1]

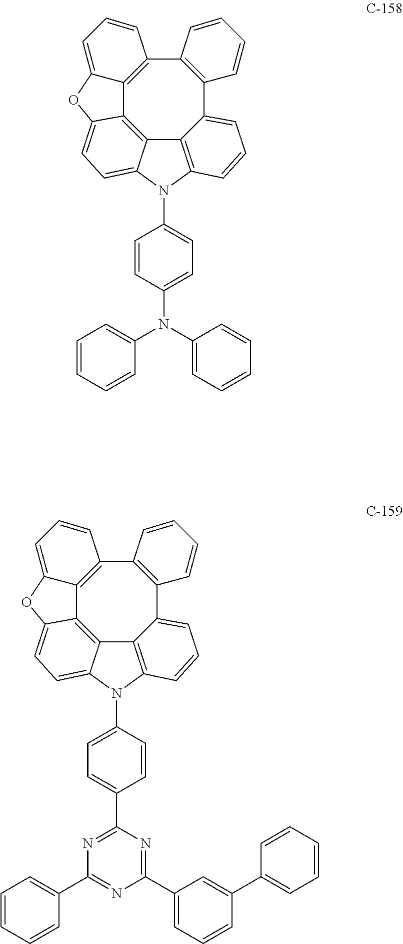 [SMILES: c1ccc(N(c2ccccc2)c2ccc(-n3c4cccc5c6ccccc6c6cccc7oc8ccc3c(c8c76)c54)cc2)cc1 ; c1ccc(-c2cccc(-c3nc(-c4ccccc4)nc(-c4ccc(-n5c6cccc7c8ccccc8c8cccc9oc%10ccc5c(c%10c98)c76)cc4)n3)c2)cc1]

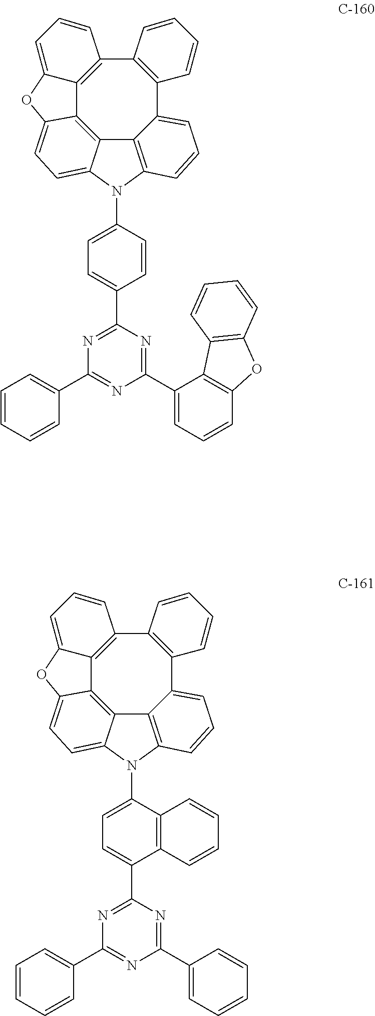 [SMILES: c1ccc(-c2nc(-c3ccc(-n4c5cccc6c7ccccc7c7cccc8oc9ccc4c(c9c87)c65)cc3)nc(-c3cccc4oc5ccccc5c34)n2)cc1 ; c1ccc(-c2nc(-c3ccccc3)nc(-c3ccc(-n4c5cccc6c7ccccc7c7cccc8oc9ccc4c(c9c87)c65)c4ccccc34)n2)cc1]

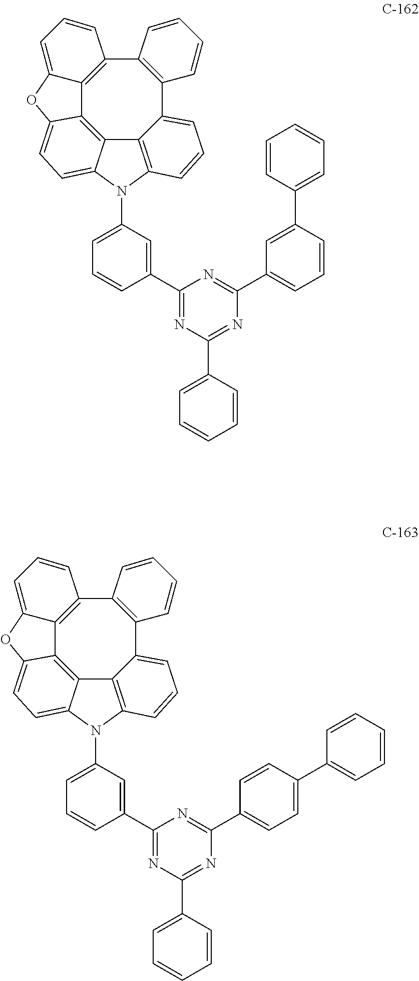 [SMILES: c1ccc(-c2cccc(-c3nc(-c4ccccc4)nc(-c4cccc(-n5c6cccc7c8ccccc8c8cccc9oc%10ccc5c(c%10c98)c76)c4)n3)c2)cc1 ; c1ccc(-c2ccc(-c3nc(-c4ccccc4)nc(-c4cccc(-n5c6cccc7c8ccccc8c8cccc9oc%10ccc5c(c%10c98)c76)c4)n3)cc2)cc1]

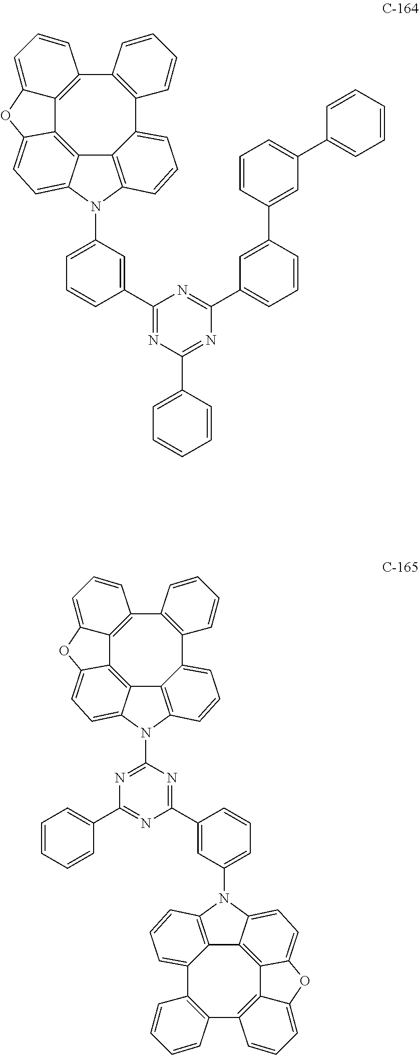 [SMILES: c1ccc(-c2cccc(-c3cccc(-c4nc(-c5ccccc5)nc(-c5cccc(-n6c7cccc8c9ccccc9c9cccc%10oc%11ccc6c(c%11c%109)c87)c5)n4)c3)c2)cc1 ; c1ccc(-c2nc(-c3cccc(-n4c5cccc6c7ccccc7c7cccc8oc9ccc4c(c9c87)c65)c3)nc(-n3c4cccc5c6ccccc6c6cccc7oc8ccc3c(c8c76)c54)n2)cc1]

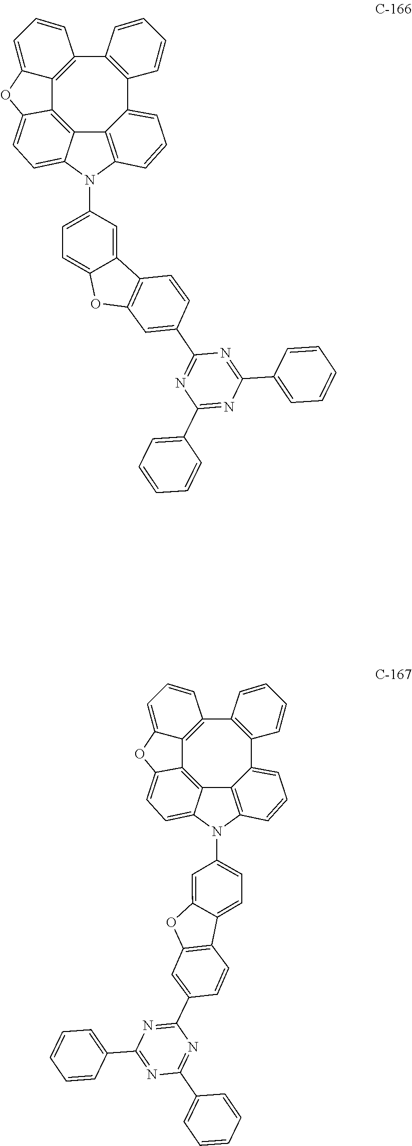 [SMILES: c1ccc(-c2nc(-c3ccccc3)nc(-c3ccc4c(c3)oc3ccc(-n5c6cccc7c8ccccc8c8cccc9oc%10ccc5c(c%10c98)c76)cc34)n2)cc1 ; c1ccc(-c2nc(-c3ccccc3)nc(-c3ccc4c(c3)oc3cc(-n5c6cccc7c8ccccc8c8cccc9oc%10ccc5c(c%10c98)c76)ccc34)n2)cc1]

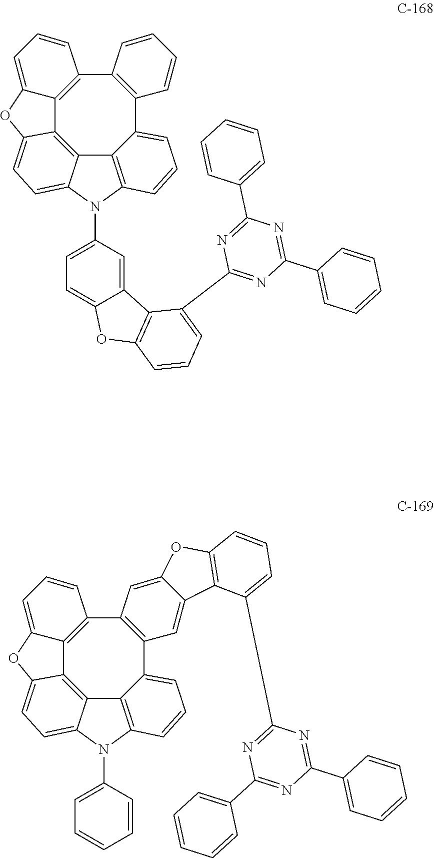 [SMILES: c1ccc(-c2nc(-c3ccccc3)nc(-c3cccc4oc5ccc(-n6c7cccc8c9ccccc9c9cccc%10oc%11ccc6c(c%11c%109)c87)cc5c34)n2)cc1 ; c1ccc(-c2nc(-c3ccccc3)nc(-c3cccc4oc5cc6c(cc5c34)c3cccc4c3c3c5c(ccc3n4-c3ccccc3)oc3cccc6c35)n2)cc1]

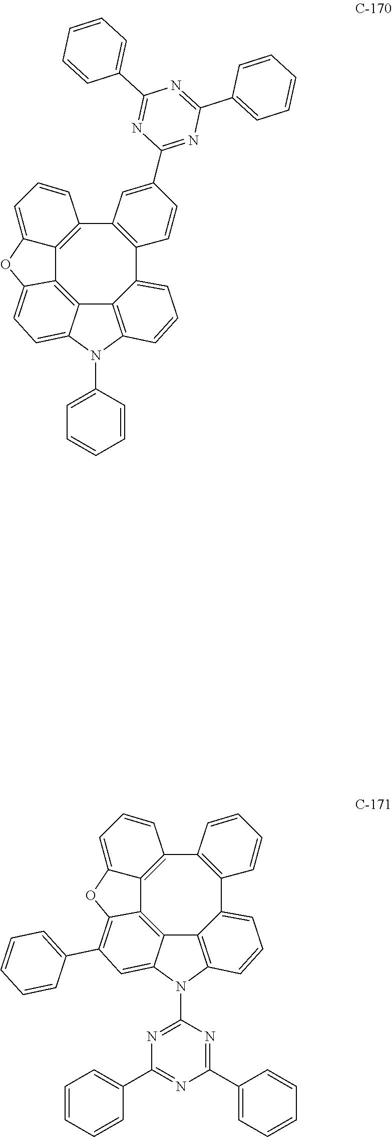 [SMILES: c1ccc(-c2nc(-c3ccccc3)nc(-c3ccc4c(c3)c3cccc5oc6ccc7c(c6c53)c3c4cccc3n7-c3ccccc3)n2)cc1 ; c1ccc(-c2nc(-c3ccccc3)nc(-n3c4cccc5c6ccccc6c6cccc7oc8c(-c9ccccc9)cc3c(c8c76)c54)n2)cc1]

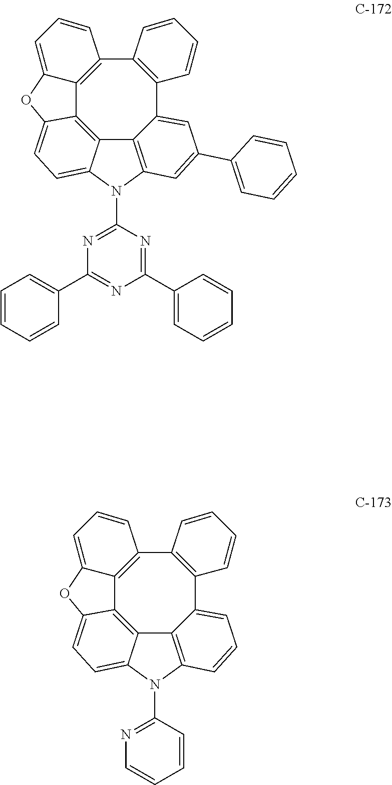 [SMILES: c1ccc(-c2cc3c4ccccc4c4cccc5oc6ccc7c(c6c54)c3c(c2)n7-c2nc(-c3ccccc3)nc(-c3ccccc3)n2)cc1 ; c1ccc(-n2c3cccc4c5ccccc5c5cccc6oc7ccc2c(c7c65)c43)nc1]

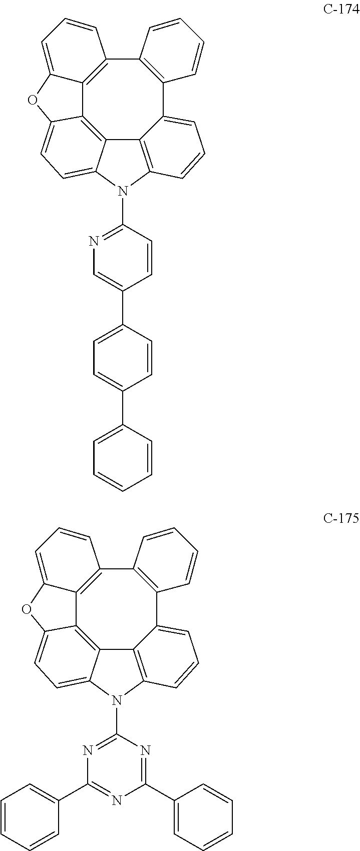 [SMILES: c1ccc(-c2ccc(-c3ccc(-n4c5cccc6c7ccccc7c7cccc8oc9ccc4c(c9c87)c65)nc3)cc2)cc1 ; c1ccc(-c2nc(-c3ccccc3)nc(-n3c4cccc5c6ccccc6c6cccc7oc8ccc3c(c8c76)c54)n2)cc1]

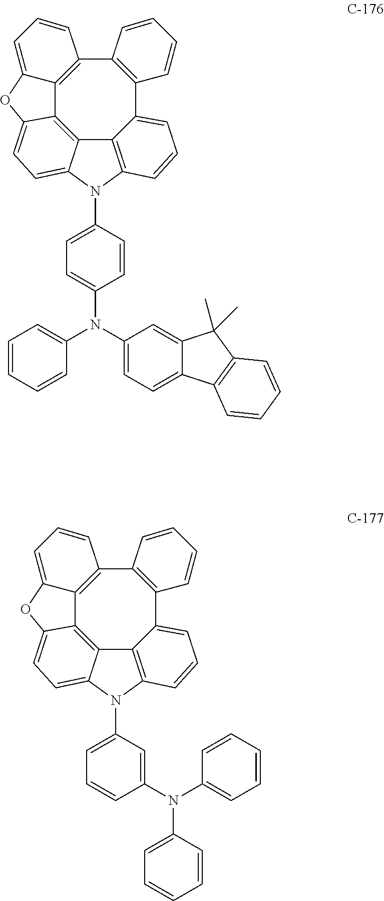 [SMILES: CC1(C)c2ccccc2-c2ccc(N(c3ccccc3)c3ccc(-n4c5cccc6c7ccccc7c7cccc8oc9ccc4c(c9c87)c65)cc3)cc21 ; c1ccc(N(c2ccccc2)c2cccc(-n3c4cccc5c6ccccc6c6cccc7oc8ccc3c(c8c76)c54)c2)cc1]

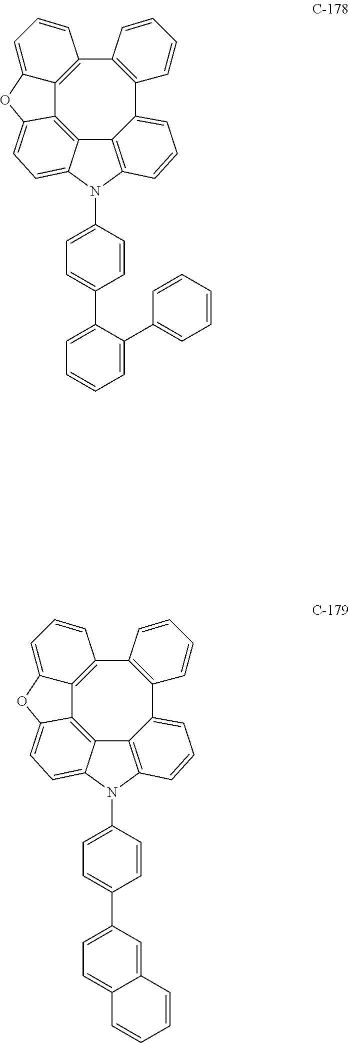 [SMILES: c1ccc(-c2ccccc2-c2ccc(-n3c4cccc5c6ccccc6c6cccc7oc8ccc3c(c8c76)c54)cc2)cc1 ; c1ccc2cc(-c3ccc(-n4c5cccc6c7ccccc7c7cccc8oc9ccc4c(c9c87)c65)cc3)ccc2c1]

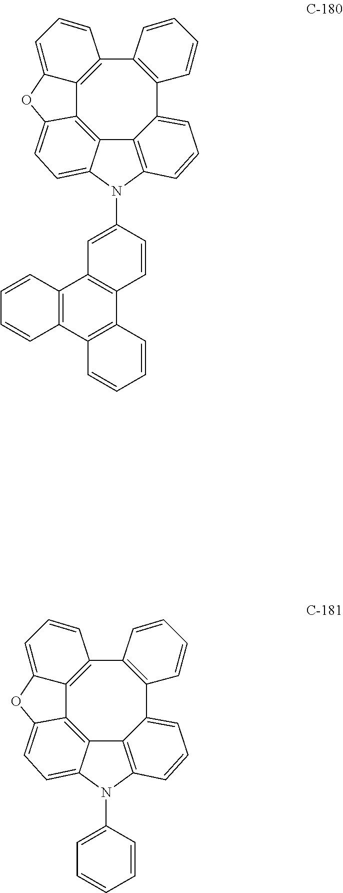 [SMILES: c1ccc2c(c1)c1ccccc1c1cc(-n3c4cccc5c6ccccc6c6cccc7oc8ccc3c(c8c76)c54)ccc21 ; c1ccc(-n2c3cccc4c5ccccc5c5cccc6oc7ccc2c(c7c65)c43)cc1]

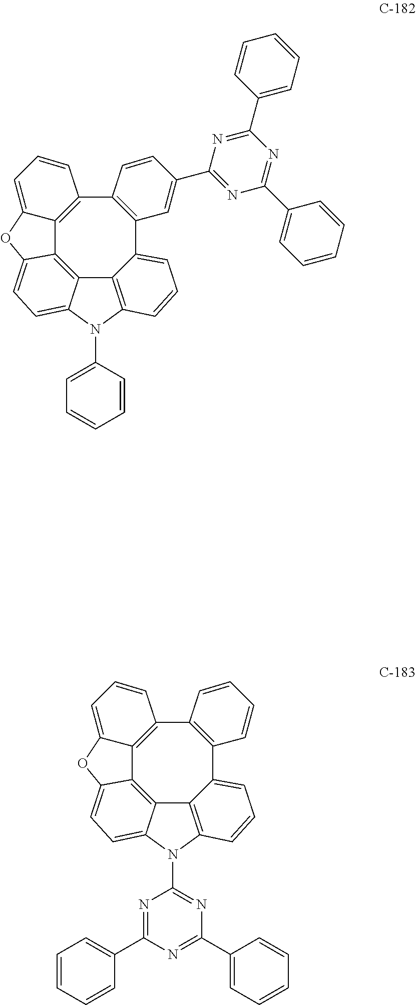 [SMILES: c1ccc(-c2nc(-c3ccccc3)nc(-c3ccc4c(c3)c3cccc5c3c3c6c(ccc3n5-c3ccccc3)oc3cccc4c36)n2)cc1 ; c1ccc(-c2nc(-c3ccccc3)nc(-n3c4cccc5c6ccccc6c6cccc7oc8ccc3c(c8c76)c54)n2)cc1]

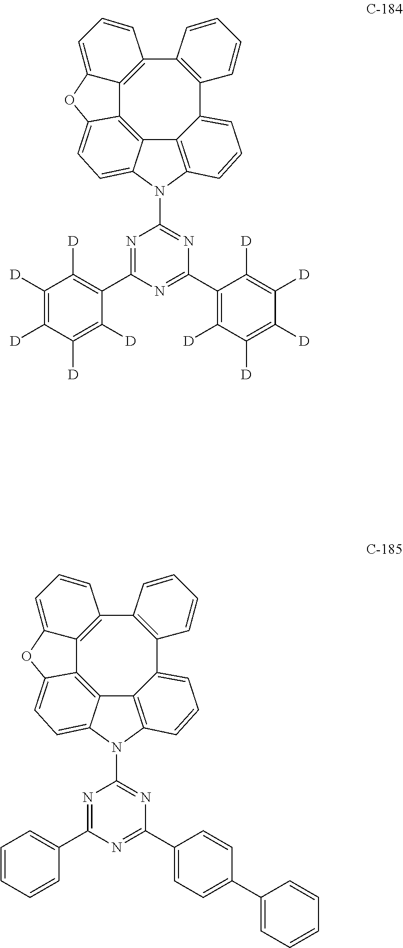 [SMILES: [2H]c1c([2H])c([2H])c(-c2nc(-c3c([2H])c([2H])c([2H])c([2H])c3[2H])nc(-n3c4cccc5c6ccccc6c6cccc7oc8ccc3c(c8c76)c54)n2)c([2H])c1[2H] ; c1ccc(-c2ccc(-c3nc(-c4ccccc4)nc(-n4c5cccc6c7ccccc7c7cccc8oc9ccc4c(c9c87)c65)n3)cc2)cc1]

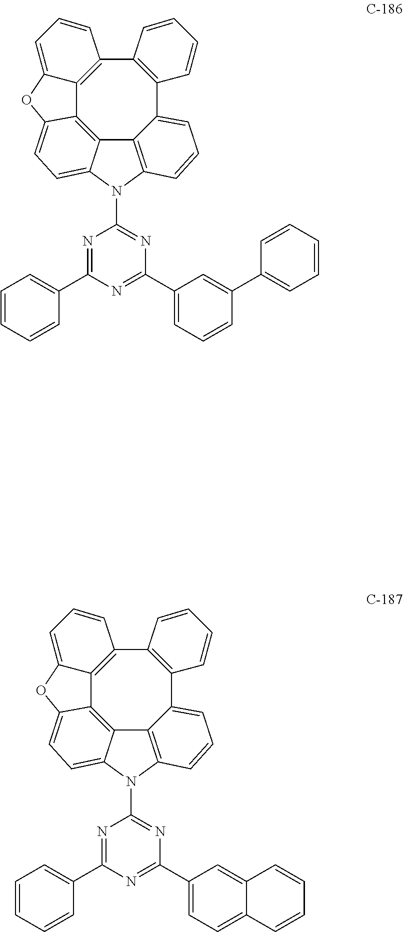 [SMILES: c1ccc(-c2cccc(-c3nc(-c4ccccc4)nc(-n4c5cccc6c7ccccc7c7cccc8oc9ccc4c(c9c87)c65)n3)c2)cc1 ; c1ccc(-c2nc(-c3ccc4ccccc4c3)nc(-n3c4cccc5c6ccccc6c6cccc7oc8ccc3c(c8c76)c54)n2)cc1]

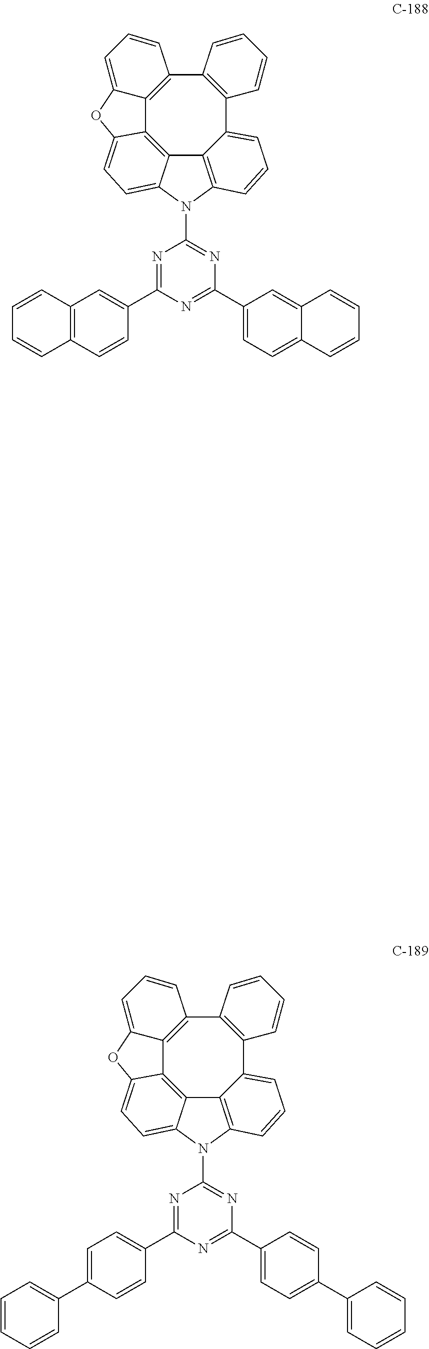 [SMILES: c1ccc2cc(-c3nc(-c4ccc5ccccc5c4)nc(-n4c5cccc6c7ccccc7c7cccc8oc9ccc4c(c9c87)c65)n3)ccc2c1 ; c1ccc(-c2ccc(-c3nc(-c4ccc(-c5ccccc5)cc4)nc(-n4c5cccc6c7ccccc7c7cccc8oc9ccc4c(c9c87)c65)n3)cc2)cc1]

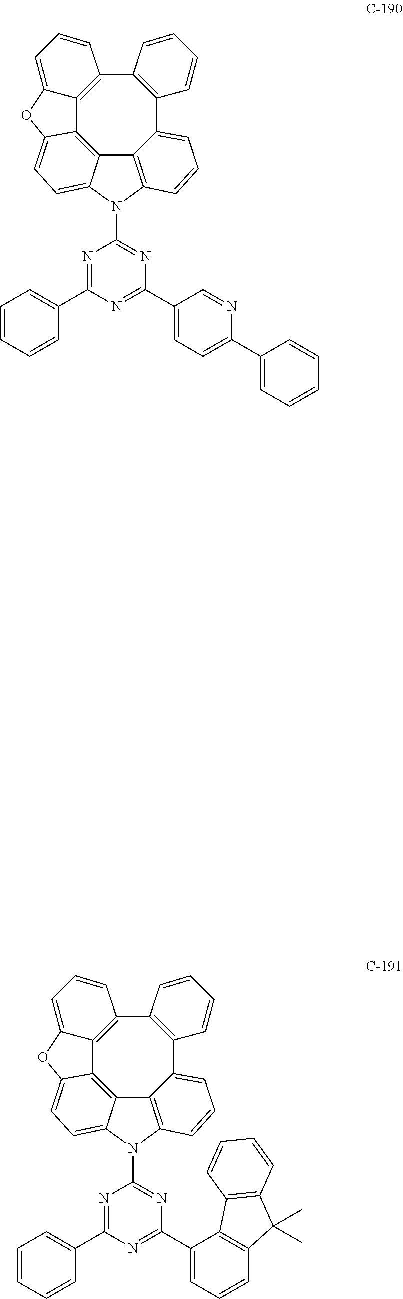 [SMILES: c1ccc(-c2ccc(-c3nc(-c4ccccc4)nc(-n4c5cccc6c7ccccc7c7cccc8oc9ccc4c(c9c87)c65)n3)cn2)cc1 ; CC1(C)c2ccccc2-c2c(-c3nc(-c4ccccc4)nc(-n4c5cccc6c7ccccc7c7cccc8oc9ccc4c(c9c87)c65)n3)cccc21]

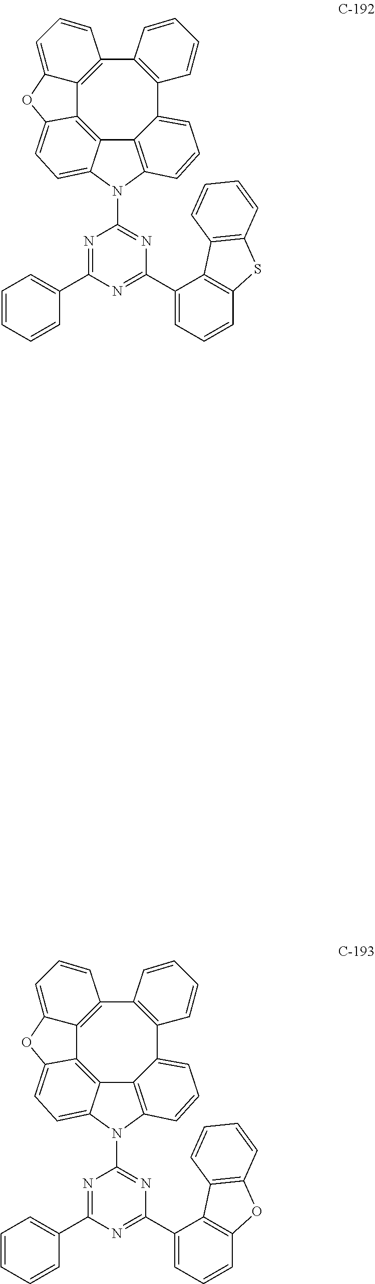 [SMILES: c1ccc(-c2nc(-c3cccc4sc5ccccc5c34)nc(-n3c4cccc5c6ccccc6c6cccc7oc8ccc3c(c8c76)c54)n2)cc1 ; c1ccc(-c2nc(-c3cccc4oc5ccccc5c34)nc(-n3c4cccc5c6ccccc6c6cccc7oc8ccc3c(c8c76)c54)n2)cc1]

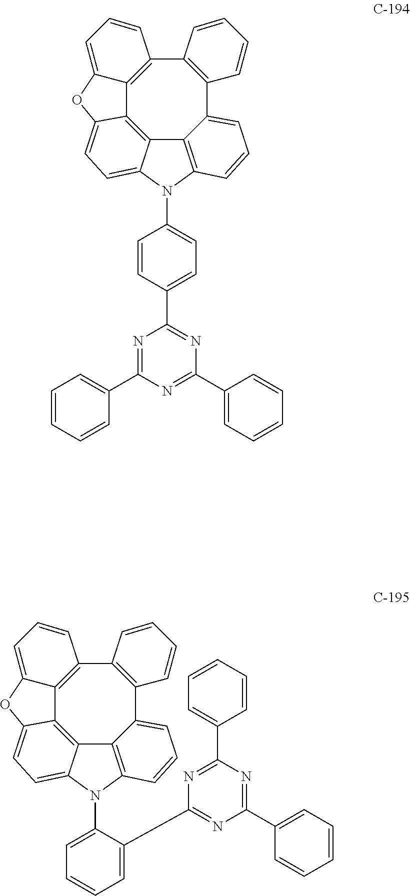 [SMILES: c1ccc(-c2nc(-c3ccccc3)nc(-c3ccc(-n4c5cccc6c7ccccc7c7cccc8oc9ccc4c(c9c87)c65)cc3)n2)cc1 ; c1ccc(-c2nc(-c3ccccc3)nc(-c3ccccc3-n3c4cccc5c6ccccc6c6cccc7oc8ccc3c(c8c76)c54)n2)cc1]

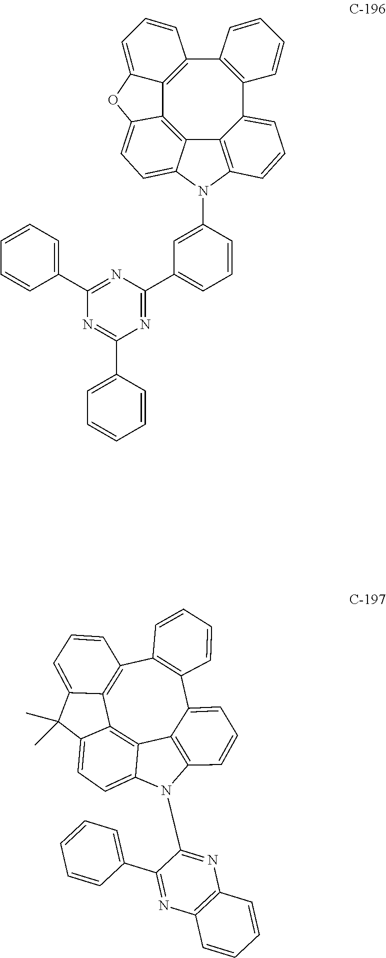 [SMILES: c1ccc(-c2nc(-c3ccccc3)nc(-c3cccc(-n4c5cccc6c7ccccc7c7cccc8oc9ccc4c(c9c87)c65)c3)n2)cc1 ; CC1(C)c2cccc3c4ccccc4c4cccc5c4c4c(c1ccc4n5-c1nc4ccccc4nc1-c1ccccc1)c23]

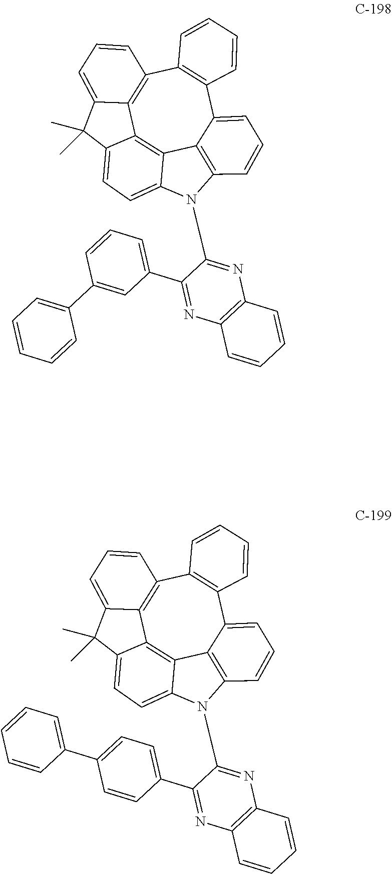 [SMILES: CC1(C)c2cccc3c4ccccc4c4cccc5c4c4c(c1ccc4n5-c1nc4ccccc4nc1-c1cccc(-c4ccccc4)c1)c23 ; CC1(C)c2cccc3c4ccccc4c4cccc5c4c4c(c1ccc4n5-c1nc4ccccc4nc1-c1ccc(-c4ccccc4)cc1)c23]

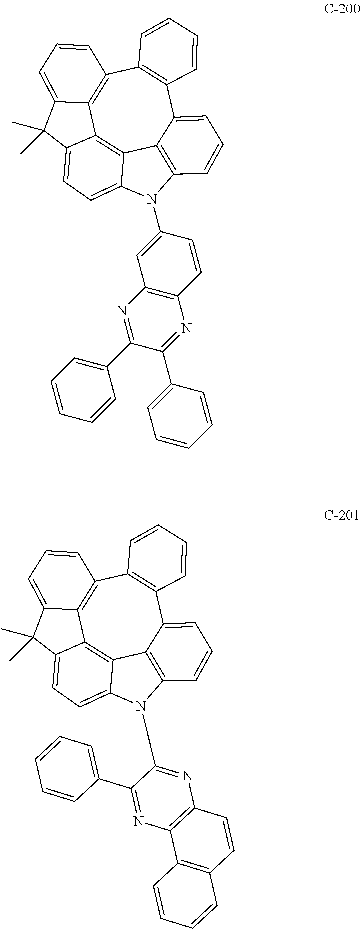 [SMILES: CC1(C)c2cccc3c4ccccc4c4cccc5c4c4c(c1ccc4n5-c1ccc4nc(-c5ccccc5)c(-c5ccccc5)nc4c1)c23 ; CC1(C)c2cccc3c4ccccc4c4cccc5c4c4c(c1ccc4n5-c1nc4ccc5ccccc5c4nc1-c1ccccc1)c23]

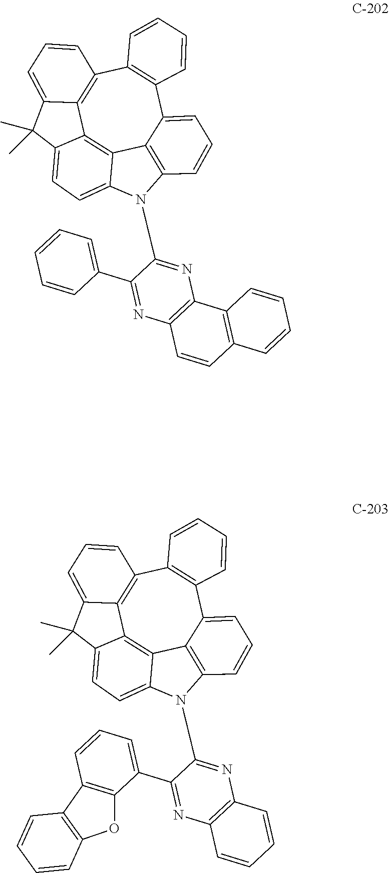 [SMILES: CC1(C)c2cccc3c4ccccc4c4cccc5c4c4c(c1ccc4n5-c1nc4c(ccc5ccccc54)nc1-c1ccccc1)c23 ; CC1(C)c2cccc3c4ccccc4c4cccc5c4c4c(c1ccc4n5-c1nc4ccccc4nc1-c1cccc4c1oc1ccccc14)c23]

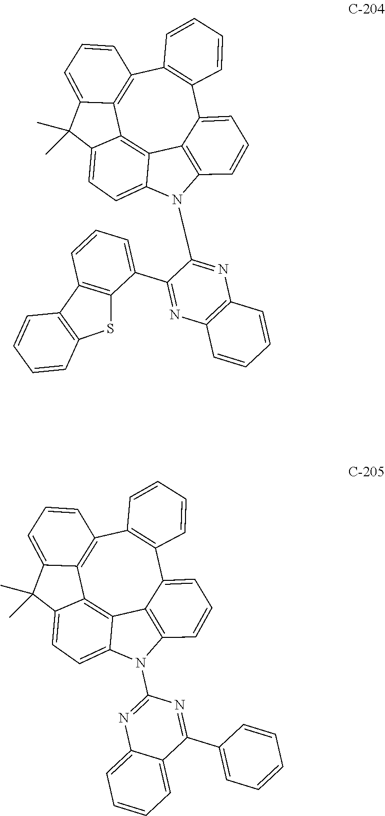 [SMILES: CC1(C)c2cccc3c4ccccc4c4cccc5c4c4c(c1ccc4n5-c1nc4ccccc4nc1-c1cccc4c1sc1ccccc14)c23 ; CC1(C)c2cccc3c4ccccc4c4cccc5c4c4c(c1ccc4n5-c1nc(-c4ccccc4)c4ccccc4n1)c23]

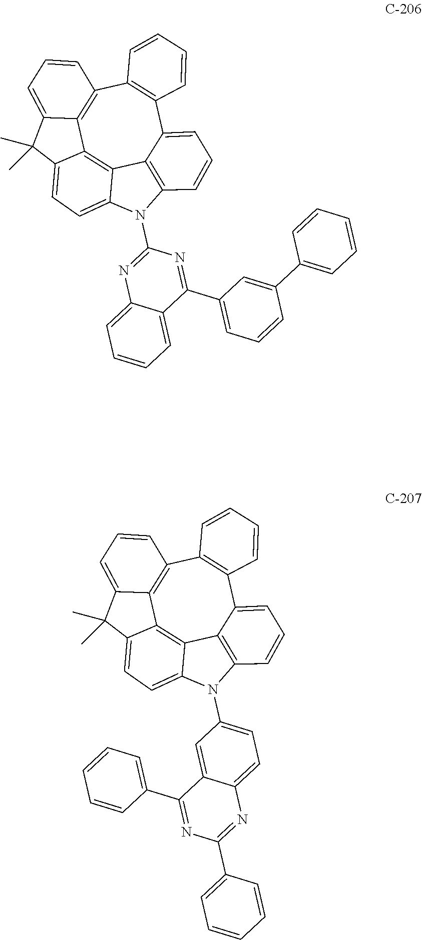 [SMILES: CC1(C)c2cccc3c4ccccc4c4cccc5c4c4c(c1ccc4n5-c1nc(-c4cccc(-c5ccccc5)c4)c4ccccc4n1)c23 ; CC1(C)c2cccc3c4ccccc4c4cccc5c4c4c(c1ccc4n5-c1ccc4nc(-c5ccccc5)nc(-c5ccccc5)c4c1)c23]

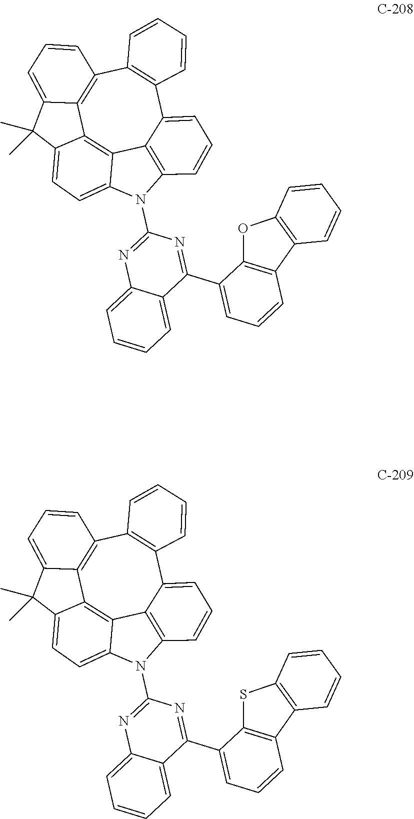 [SMILES: CC1(C)c2cccc3c4ccccc4c4cccc5c4c4c(c1ccc4n5-c1nc(-c4cccc5c4oc4ccccc45)c4ccccc4n1)c23 ; CC1(C)c2cccc3c4ccccc4c4cccc5c4c4c(c1ccc4n5-c1nc(-c4cccc5c4sc4ccccc45)c4ccccc4n1)c23]

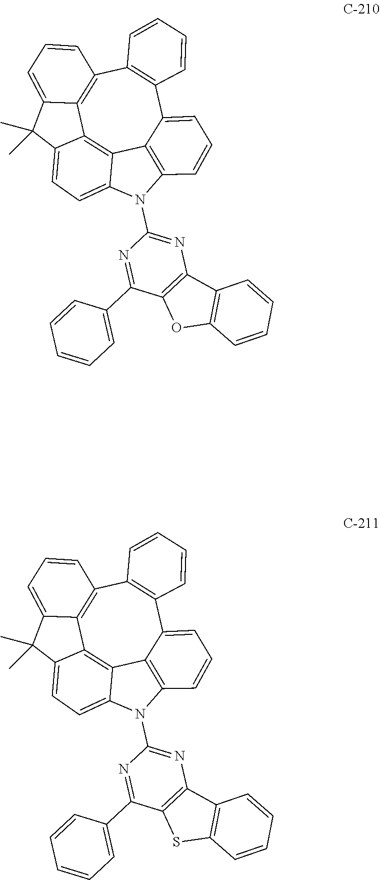 [SMILES: CC1(C)c2cccc3c4ccccc4c4cccc5c4c4c(c1ccc4n5-c1nc(-c4ccccc4)c4oc5ccccc5c4n1)c23 ; CC1(C)c2cccc3c4ccccc4c4cccc5c4c4c(c1ccc4n5-c1nc(-c4ccccc4)c4sc5ccccc5c4n1)c23]

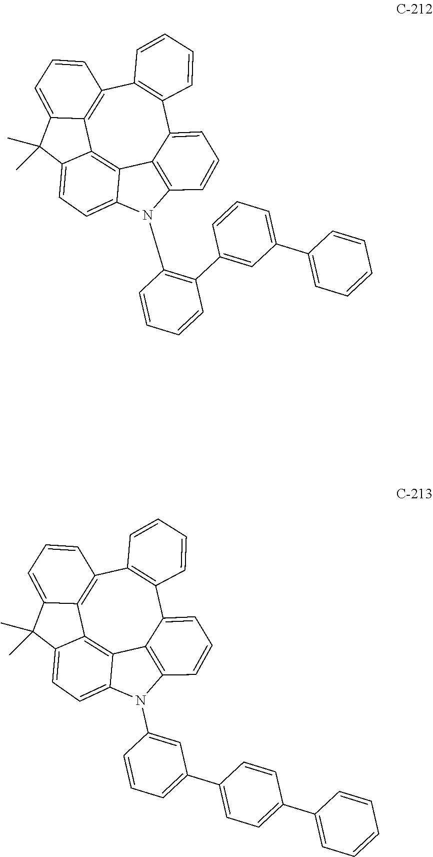 [SMILES: CC1(C)c2cccc3c4ccccc4c4cccc5c4c4c(c1ccc4n5-c1ccccc1-c1cccc(-c4ccccc4)c1)c23 ; CC1(C)c2cccc3c4ccccc4c4cccc5c4c4c(c1ccc4n5-c1cccc(-c4ccc(-c5ccccc5)cc4)c1)c23]

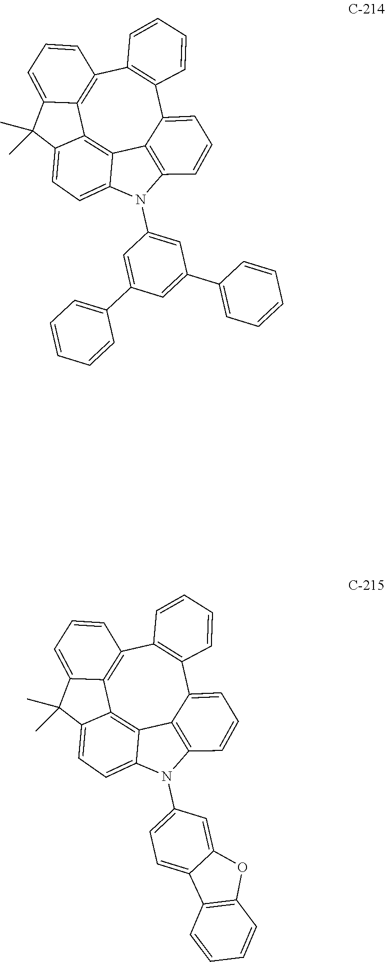 [SMILES: CC1(C)c2cccc3c4ccccc4c4cccc5c4c4c(c1ccc4n5-c1cc(-c4ccccc4)cc(-c4ccccc4)c1)c23 ; CC1(C)c2cccc3c4ccccc4c4cccc5c4c4c(c1ccc4n5-c1ccc4c(c1)oc1ccccc14)c23]

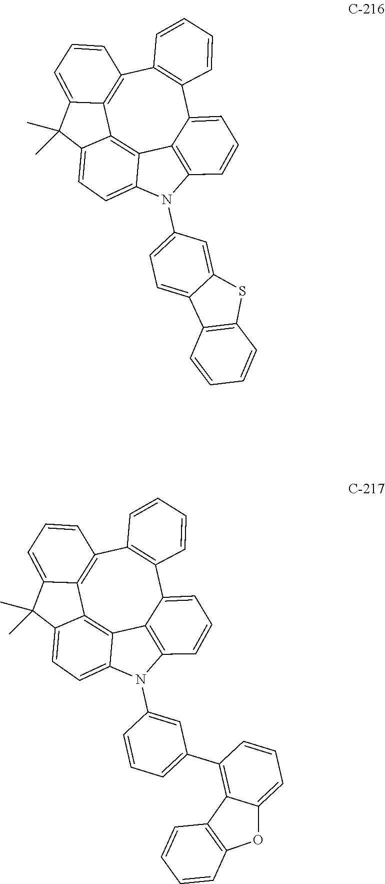 [SMILES: CC1(C)c2cccc3c4ccccc4c4cccc5c4c4c(c1ccc4n5-c1ccc4c(c1)sc1ccccc14)c23 ; CC1(C)c2cccc3c4ccccc4c4cccc5c4c4c(c1ccc4n5-c1cccc(-c4cccc5oc6ccccc6c45)c1)c23]

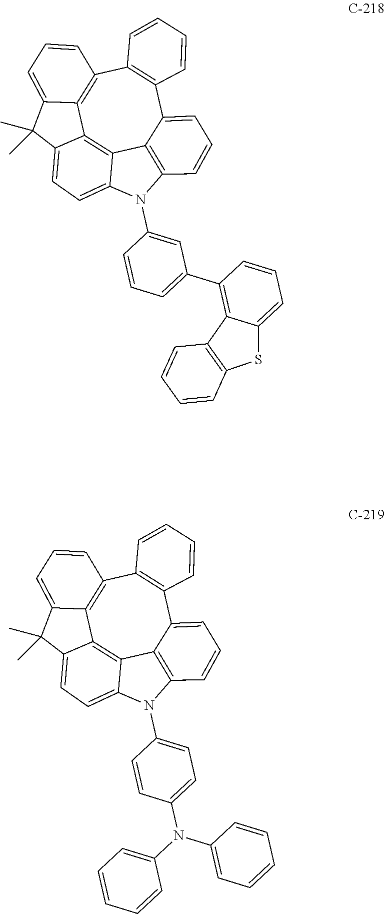 [SMILES: CC1(C)c2cccc3c4ccccc4c4cccc5c4c4c(c1ccc4n5-c1cccc(-c4cccc5sc6ccccc6c45)c1)c23 ; CC1(C)c2cccc3c4ccccc4c4cccc5c4c4c(c1ccc4n5-c1ccc(N(c4ccccc4)c4ccccc4)cc1)c23]

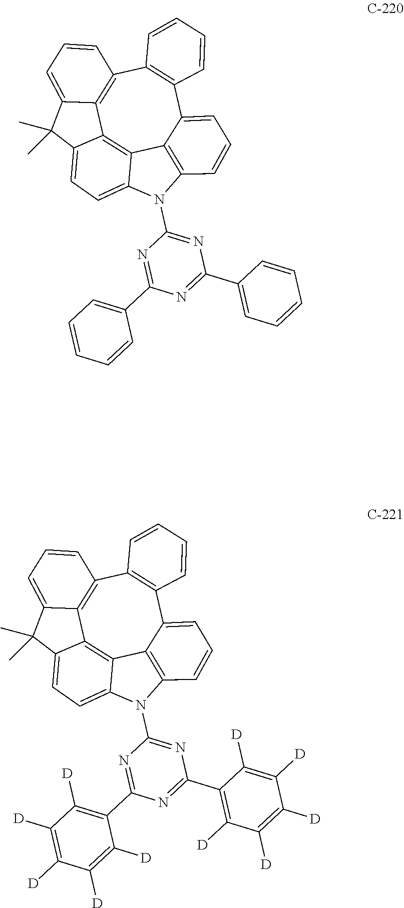 [SMILES: CC1(C)c2cccc3c4ccccc4c4cccc5c4c4c(c1ccc4n5-c1nc(-c4ccccc4)nc(-c4ccccc4)n1)c23 ; [2H]c1c([2H])c([2H])c(-c2nc(-c3c([2H])c([2H])c([2H])c([2H])c3[2H])nc(-n3c4cccc5c6ccccc6c6cccc7c6c6c(ccc3c6c54)C7(C)C)n2)c([2H])c1[2H]]

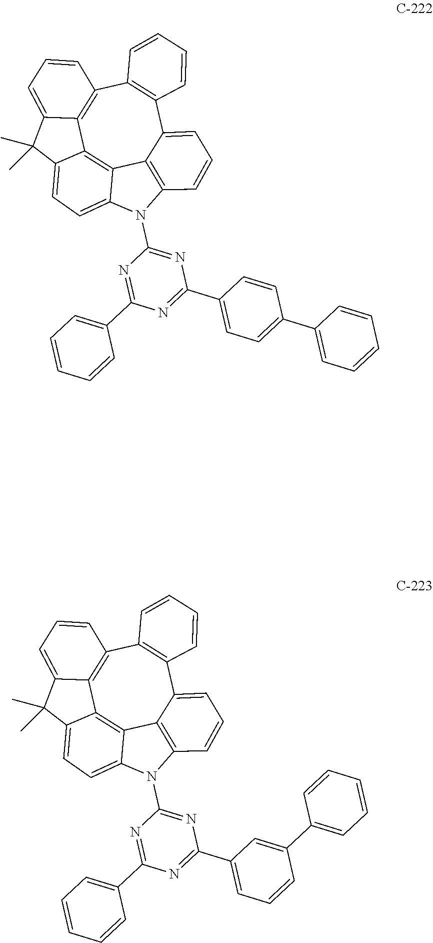 [SMILES: CC1(C)c2cccc3c4ccccc4c4cccc5c4c4c(c1ccc4n5-c1nc(-c4ccccc4)nc(-c4ccc(-c5ccccc5)cc4)n1)c23 ; CC1(C)c2cccc3c4ccccc4c4cccc5c4c4c(c1ccc4n5-c1nc(-c4ccccc4)nc(-c4cccc(-c5ccccc5)c4)n1)c23]

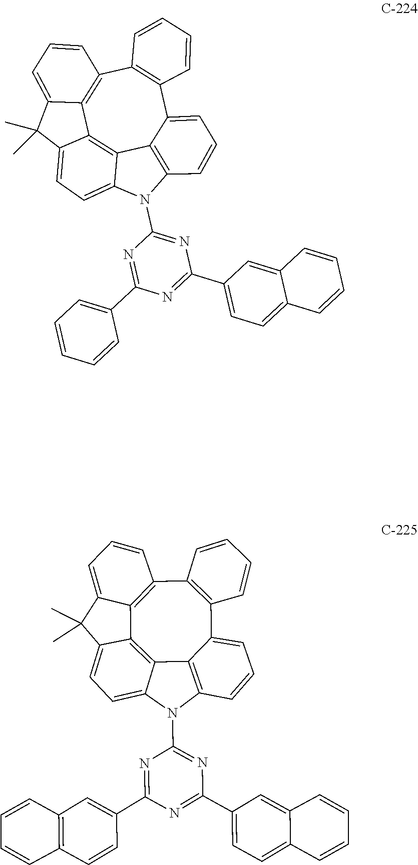 [SMILES: CC1(C)c2cccc3c4ccccc4c4cccc5c4c4c(c1ccc4n5-c1nc(-c4ccccc4)nc(-c4ccc5ccccc5c4)n1)c23 ; CC1(C)c2cccc3c4ccccc4c4cccc5c4c4c(c1ccc4n5-c1nc(-c4ccc5ccccc5c4)nc(-c4ccc5ccccc5c4)n1)c23]

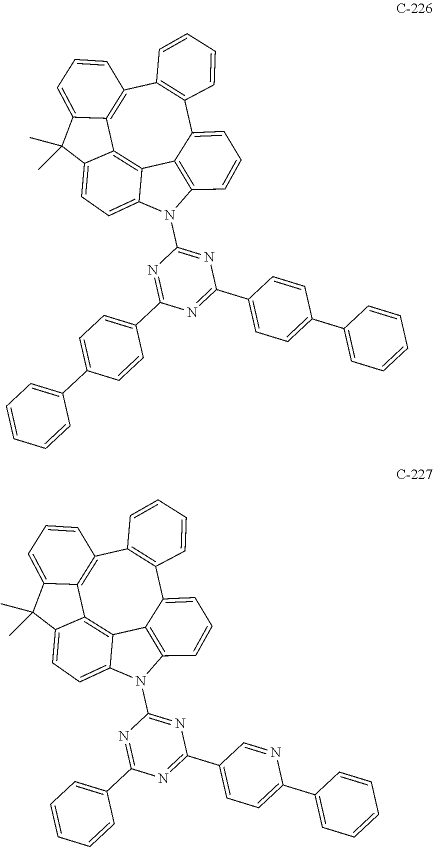 [SMILES: CC1(C)c2cccc3c4ccccc4c4cccc5c4c4c(c1ccc4n5-c1nc(-c4ccc(-c5ccccc5)cc4)nc(-c4ccc(-c5ccccc5)cc4)n1)c23 ; CC1(C)c2cccc3c4ccccc4c4cccc5c4c4c(c1ccc4n5-c1nc(-c4ccccc4)nc(-c4ccc(-c5ccccc5)nc4)n1)c23]

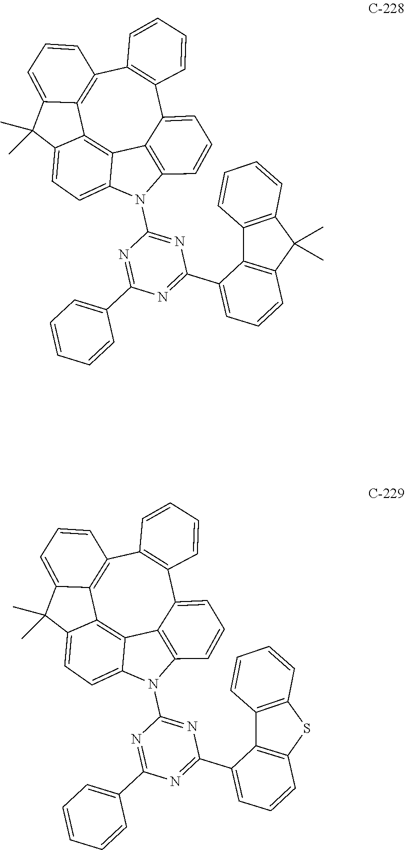

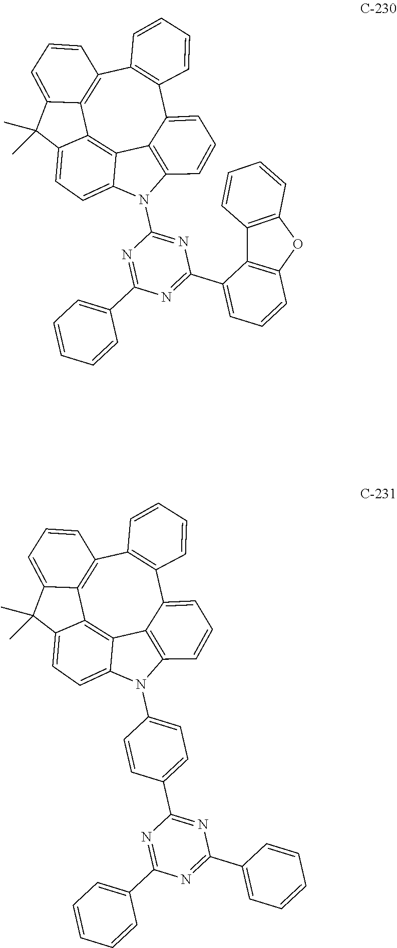 [SMILES: CC1(C)c2cccc3c4ccccc4c4cccc5c4c4c(c1ccc4n5-c1nc(-c4ccccc4)nc(-c4cccc5oc6ccccc6c45)n1)c23 ; CC1(C)c2cccc3c4ccccc4c4cccc5c4c4c(c1ccc4n5-c1ccc(-c4nc(-c5ccccc5)nc(-c5ccccc5)n4)cc1)c23]

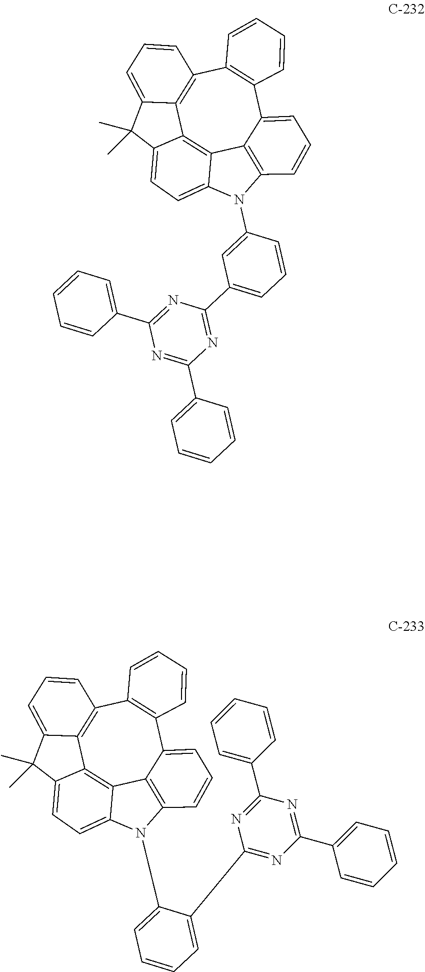 [SMILES: CC1(C)c2cccc3c4ccccc4c4cccc5c4c4c(c1ccc4n5-c1cccc(-c4nc(-c5ccccc5)nc(-c5ccccc5)n4)c1)c23 ; CC1(C)c2cccc3c4ccccc4c4cccc5c4c4c(c1ccc4n5-c1ccccc1-c1nc(-c4ccccc4)nc(-c4ccccc4)n1)c23]

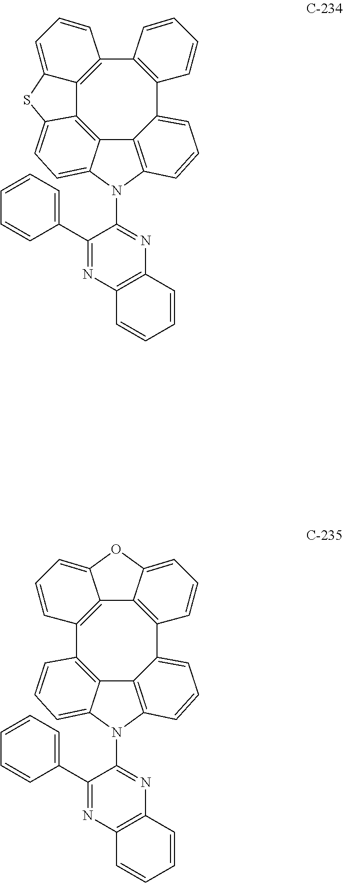 [SMILES: c1ccc(-c2nc3ccccc3nc2-n2c3cccc4c5ccccc5c5cccc6sc7ccc2c(c7c65)c43)cc1 ; c1ccc(-c2nc3ccccc3nc2-n2c3cccc4c5cccc6oc7cccc(c8cccc2c8c43)c7c65)cc1]

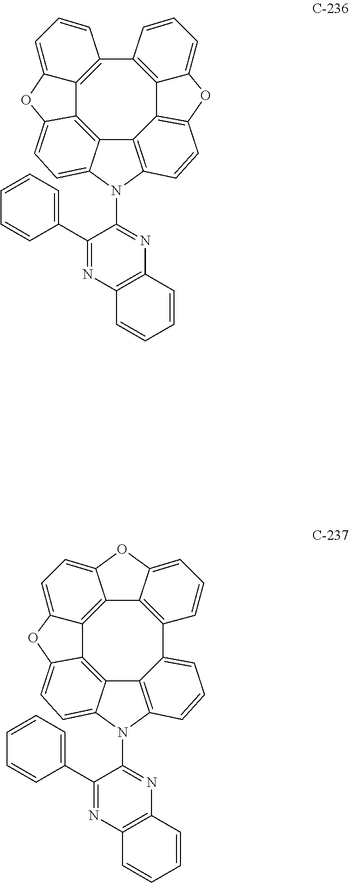 [SMILES: c1ccc(-c2nc3ccccc3nc2-n2c3ccc4oc5cccc6c7cccc8oc9ccc2c(c9c87)c3c4c56)cc1 ; c1ccc(-c2nc3ccccc3nc2-n2c3cccc4c5cccc6oc7ccc8oc9ccc2c(c9c8c7c65)c43)cc1]

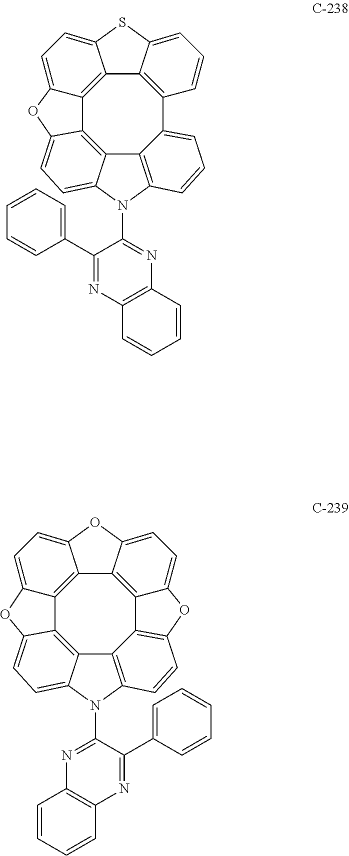 [SMILES: c1ccc(-c2nc3ccccc3nc2-n2c3cccc4c5cccc6sc7ccc8oc9ccc2c(c9c8c7c65)c43)cc1 ; c1ccc(-c2nc3ccccc3nc2-n2c3ccc4oc5ccc6oc7ccc8oc9ccc2c2c9c8c7c6c5c4c23)cc1]

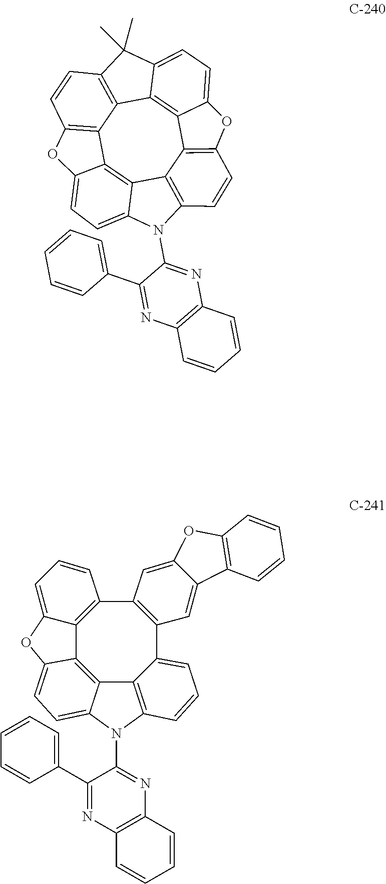 [SMILES: CC1(C)c2ccc3oc4ccc5c6c4c3c2c2c1ccc1oc3ccc(c6c3c12)n5-c1nc2ccccc2nc1-c1ccccc1 ; c1ccc(-c2nc3ccccc3nc2-n2c3cccc4c5cc6c(cc5c5cccc7oc8ccc2c(c8c75)c43)oc2ccccc26)cc1]

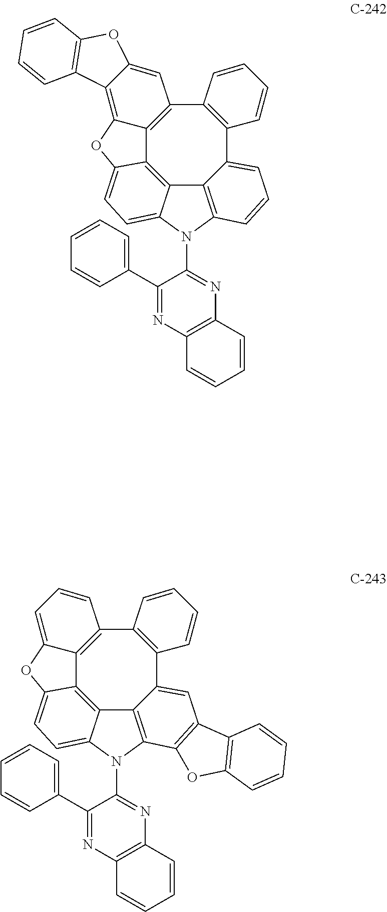 [SMILES: c1ccc(-c2nc3ccccc3nc2-n2c3cccc4c5ccccc5c5cc6oc7ccccc7c6c6oc7ccc2c(c7c56)c43)cc1 ; c1ccc(-c2nc3ccccc3nc2-n2c3ccc4oc5cccc6c7ccccc7c7cc8c9ccccc9oc8c2c7c3c4c56)cc1]

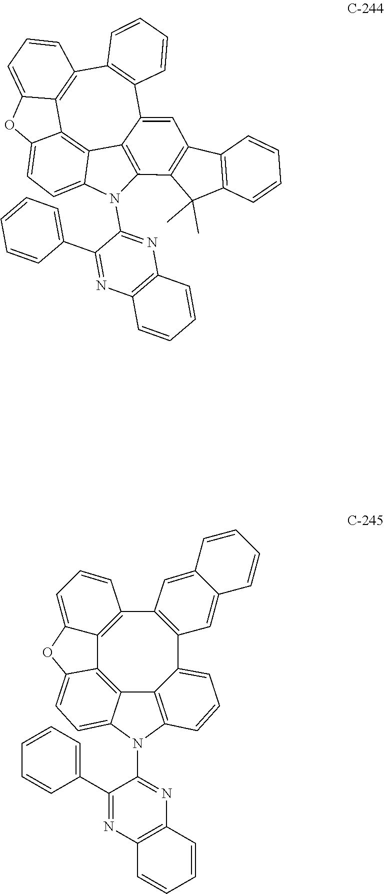 [SMILES: CC1(C)c2ccccc2-c2cc3c4ccccc4c4cccc5oc6ccc7c(c6c54)c3c(c21)n7-c1nc2ccccc2nc1-c1ccccc1 ; c1ccc(-c2nc3ccccc3nc2-n2c3cccc4c5cc6ccccc6cc5c5cccc6oc7ccc2c(c7c65)c43)cc1]

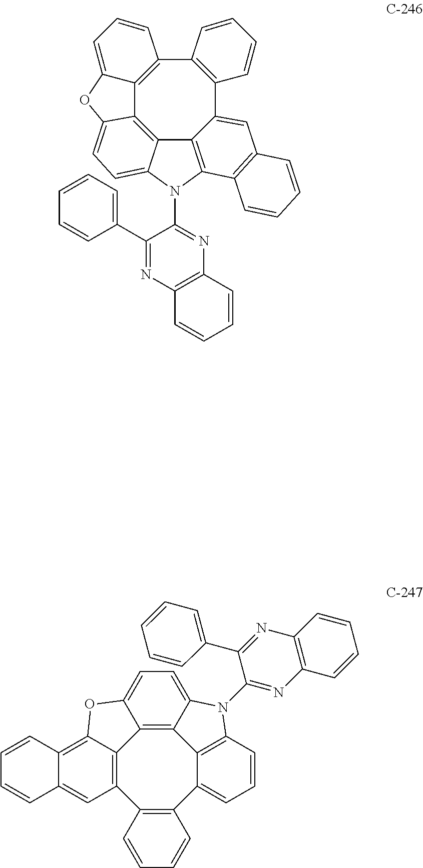 [SMILES: c1ccc(-c2nc3ccccc3nc2-n2c3ccc4oc5cccc6c7ccccc7c7cc8ccccc8c2c7c3c4c56)cc1 ; c1ccc(-c2nc3ccccc3nc2-n2c3cccc4c5ccccc5c5cc6ccccc6c6oc7ccc2c(c7c56)c43)cc1]

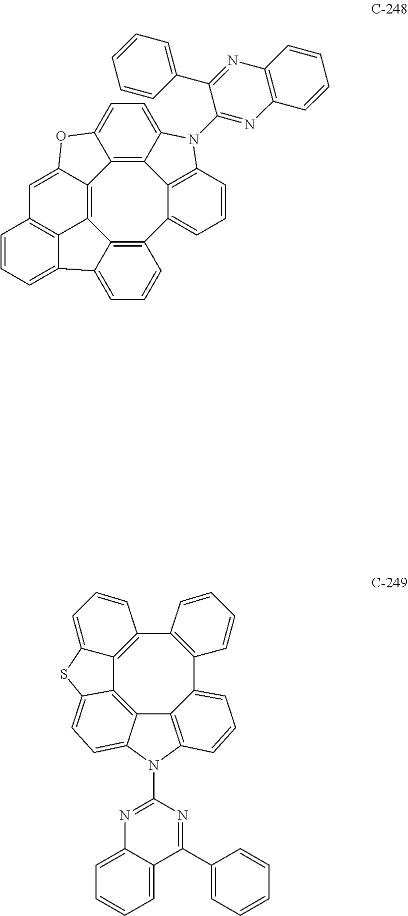 [SMILES: c1ccc(-c2nc3ccccc3nc2-n2c3cccc4c5cccc6c5c5c7c-6cccc7cc6oc7ccc2c(c7c65)c43)cc1 ; c1ccc(-c2nc(-n3c4cccc5c6ccccc6c6cccc7sc8ccc3c(c8c76)c54)nc3ccccc23)cc1]

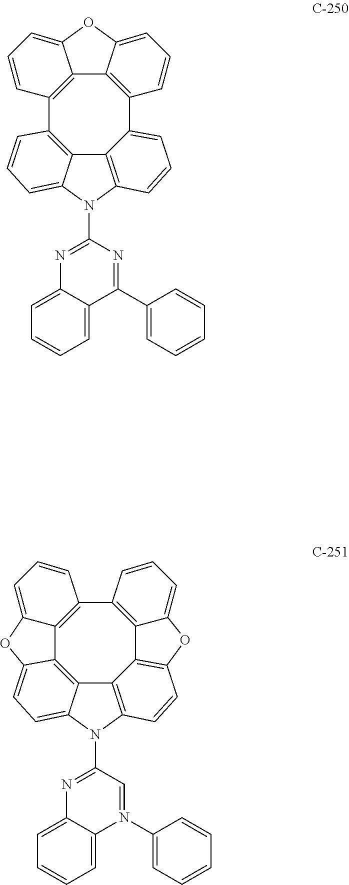

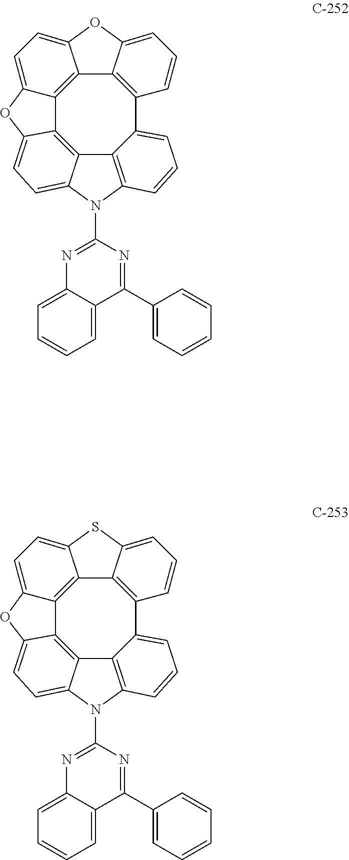 [SMILES: c1ccc(-c2nc(-n3c4cccc5c6cccc7oc8ccc9oc%10ccc3c(c%10c9c8c76)c54)nc3ccccc23)cc1 ; c1ccc(-c2nc(-n3c4cccc5c6cccc7sc8ccc9oc%10ccc3c(c%10c9c8c76)c54)nc3ccccc23)cc1]

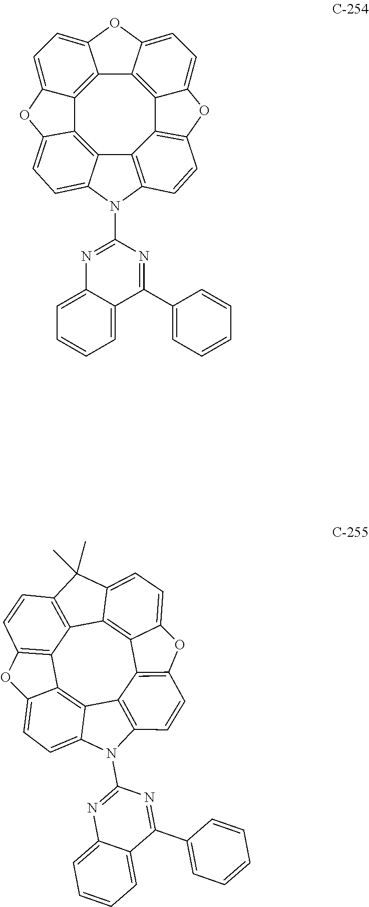 [SMILES: c1ccc(-c2nc(-n3c4ccc5oc6ccc7oc8ccc9oc%10ccc3c3c%10c9c8c7c6c5c34)nc3ccccc23)cc1 ; CC1(C)c2ccc3oc4ccc5c6c4c3c2c2c1ccc1oc3ccc(c6c3c12)n5-c1nc(-c2ccccc2)c2ccccc2n1]

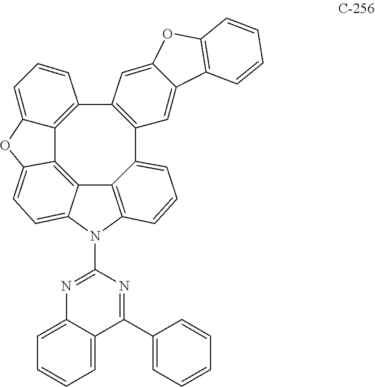 [SMILES: c1ccc(-c2nc(-n3c4cccc5c6cc7c(cc6c6cccc8oc9ccc3c(c9c86)c54)oc3ccccc37)nc3ccccc23)cc1]

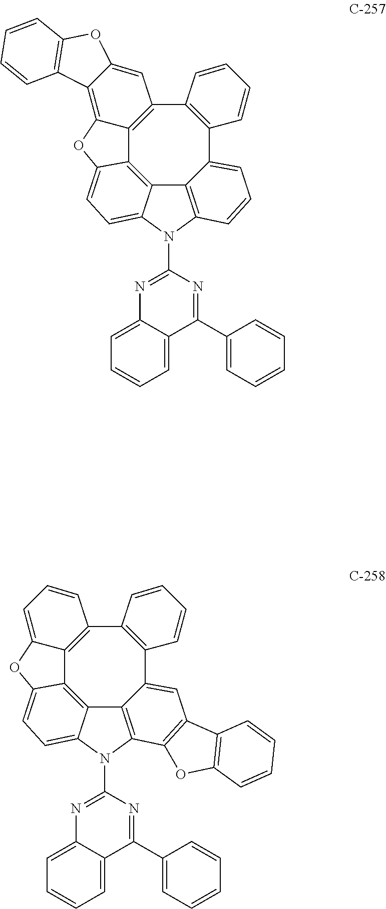 [SMILES: c1ccc(-c2nc(-n3c4cccc5c6ccccc6c6cc7oc8ccccc8c7c7oc8ccc3c(c8c67)c54)nc3ccccc23)cc1 ; c1ccc(-c2nc(-n3c4ccc5oc6cccc7c8ccccc8c8cc9c%10ccccc%10oc9c3c8c4c5c67)nc3ccccc23)cc1]

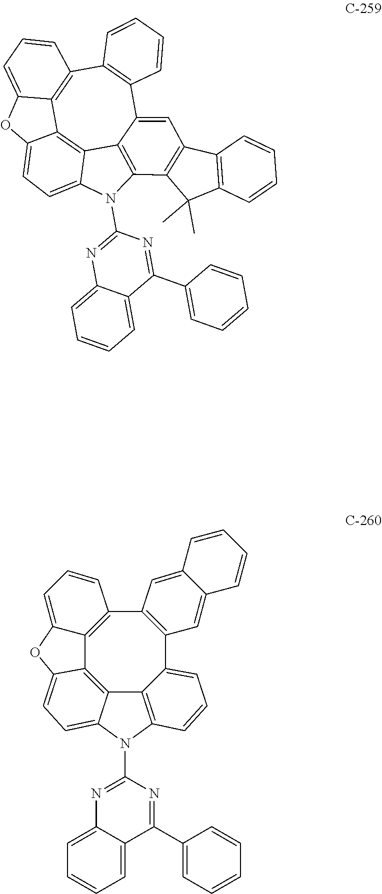 [SMILES: CC1(C)c2ccccc2-c2cc3c4ccccc4c4cccc5oc6ccc7c(c6c54)c3c(c21)n7-c1nc(-c2ccccc2)c2ccccc2n1 ; c1ccc(-c2nc(-n3c4cccc5c6cc7ccccc7cc6c6cccc7oc8ccc3c(c8c76)c54)nc3ccccc23)cc1]

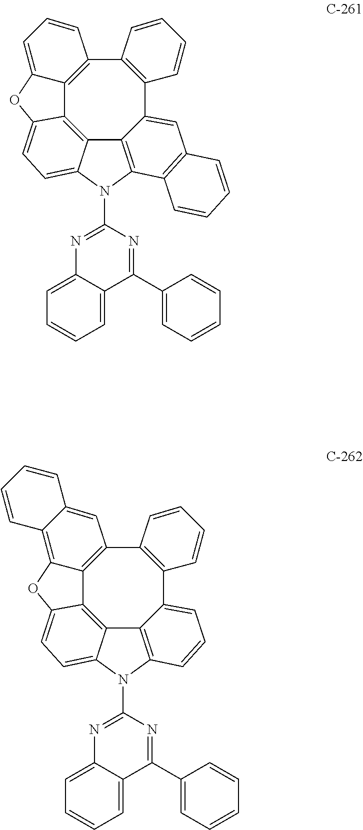 [SMILES: c1ccc(-c2nc(-n3c4ccc5oc6cccc7c8ccccc8c8cc9ccccc9c3c8c4c5c67)nc3ccccc23)cc1 ; c1ccc(-c2nc(-n3c4cccc5c6ccccc6c6cc7ccccc7c7oc8ccc3c(c8c67)c54)nc3ccccc23)cc1]

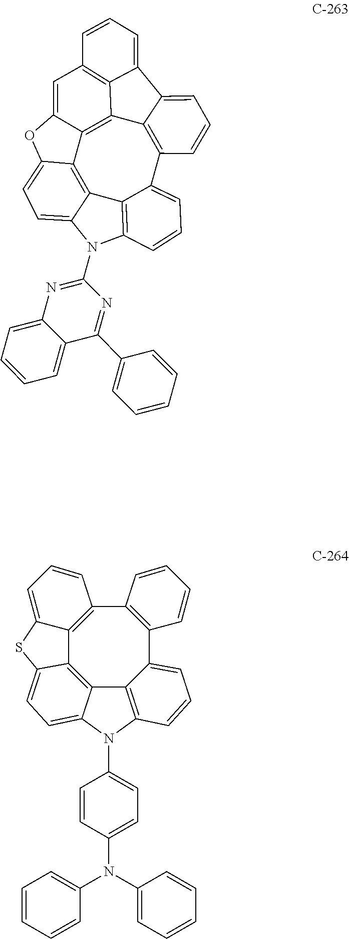 [SMILES: c1ccc(-c2nc(-n3c4cccc5c6cccc7c6c6c8c-7cccc8cc7oc8ccc3c(c8c76)c54)nc3ccccc23)cc1 ; c1ccc(N(c2ccccc2)c2ccc(-n3c4cccc5c6ccccc6c6cccc7sc8ccc3c(c8c76)c54)cc2)cc1]

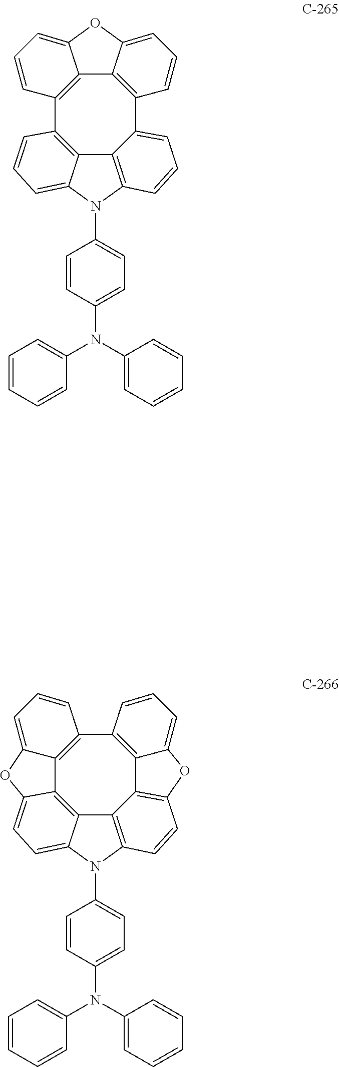 [SMILES: c1ccc(N(c2ccccc2)c2ccc(-n3c4cccc5c6cccc7oc8cccc(c9cccc3c9c54)c8c76)cc2)cc1 ; c1ccc(N(c2ccccc2)c2ccc(-n3c4ccc5oc6cccc7c8cccc9oc%10ccc3c(c%10c98)c4c5c67)cc2)cc1]

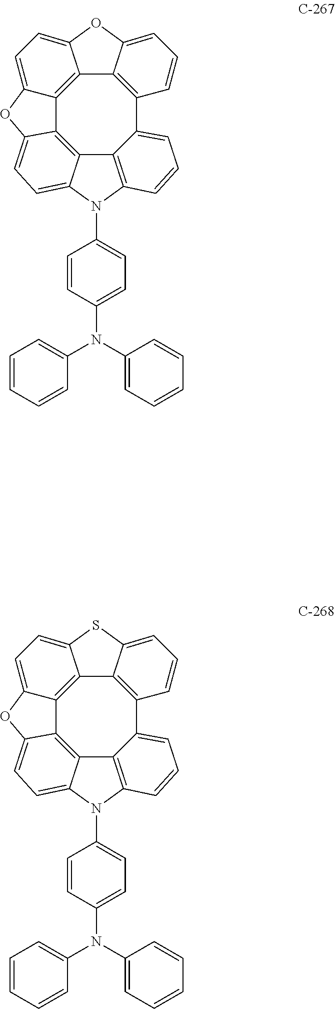 [SMILES: c1ccc(N(c2ccccc2)c2ccc(-n3c4cccc5c6cccc7oc8ccc9oc%10ccc3c(c%10c9c8c76)c54)cc2)cc1 ; c1ccc(N(c2ccccc2)c2ccc(-n3c4cccc5c6cccc7sc8ccc9oc%10ccc3c(c%10c9c8c76)c54)cc2)cc1]

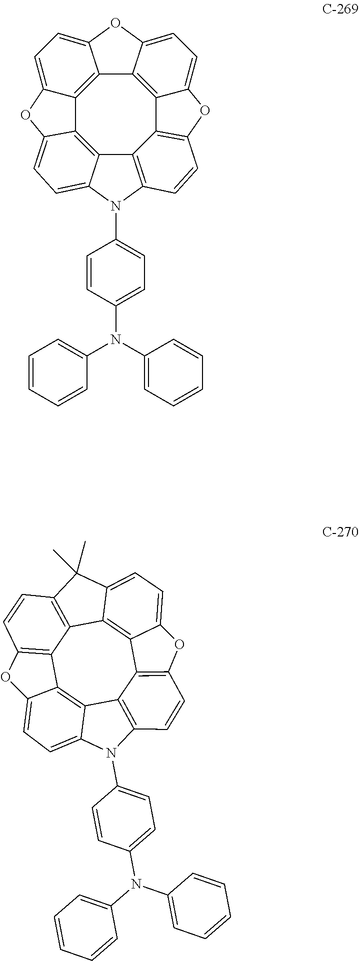 [SMILES: c1ccc(N(c2ccccc2)c2ccc(-n3c4ccc5oc6ccc7oc8ccc9oc%10ccc3c3c%10c9c8c7c6c5c34)cc2)cc1 ; CC1(C)c2ccc3oc4ccc5c6c4c3c2c2c1ccc1oc3ccc(c6c3c12)n5-c1ccc(N(c2ccccc2)c2ccccc2)cc1]

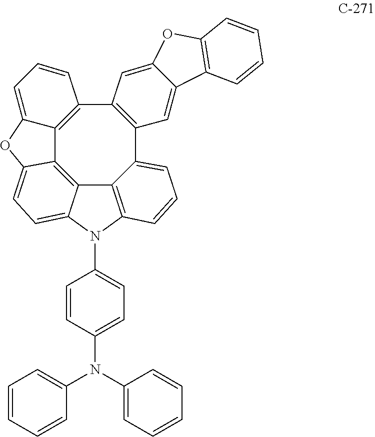 [SMILES: c1ccc(N(c2ccccc2)c2ccc(-n3c4cccc5c6cc7c(cc6c6cccc8oc9ccc3c(c9c86)c54)oc3ccccc37)cc2)cc1]

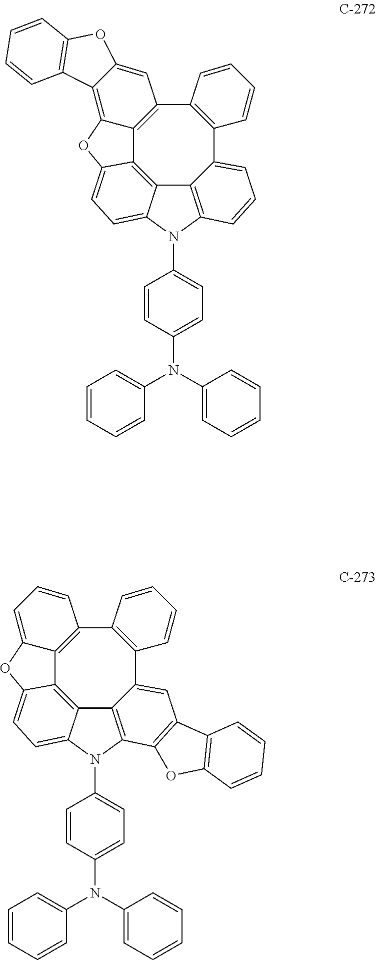 [SMILES: c1ccc(N(c2ccccc2)c2ccc(-n3c4cccc5c6ccccc6c6cc7oc8ccccc8c7c7oc8ccc3c(c8c67)c54)cc2)cc1 ; c1ccc(N(c2ccccc2)c2ccc(-n3c4ccc5oc6cccc7c8ccccc8c8cc9c%10ccccc%10oc9c3c8c4c5c67)cc2)cc1]

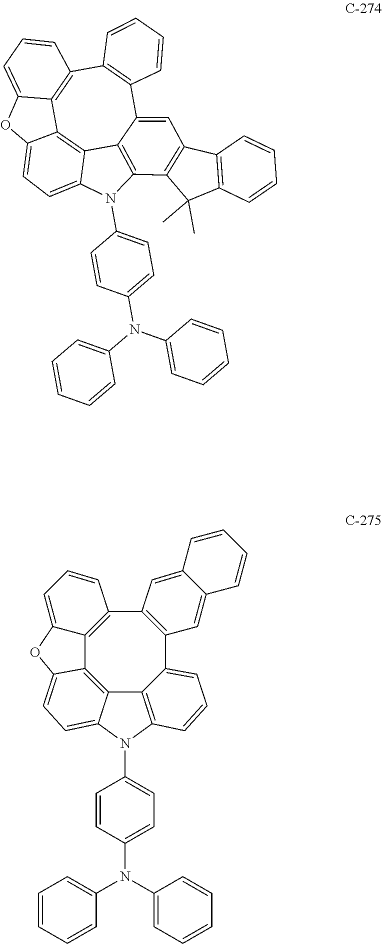 [SMILES: CC1(C)c2ccccc2-c2cc3c4ccccc4c4cccc5oc6ccc7c(c6c54)c3c(c21)n7-c1ccc(N(c2ccccc2)c2ccccc2)cc1 ; c1ccc(N(c2ccccc2)c2ccc(-n3c4cccc5c6cc7ccccc7cc6c6cccc7oc8ccc3c(c8c76)c54)cc2)cc1]

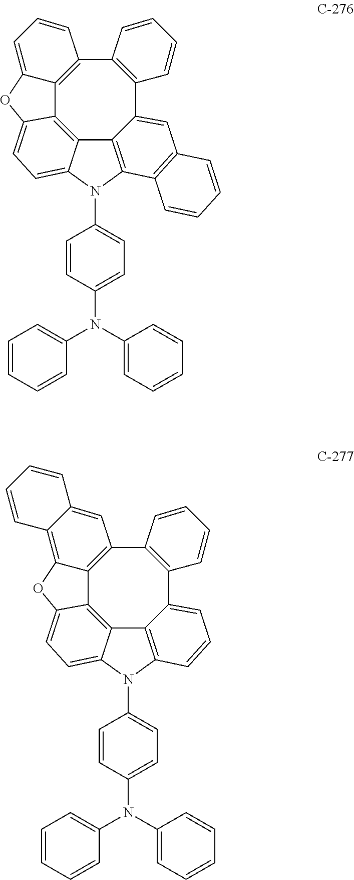 [SMILES: c1ccc(N(c2ccccc2)c2ccc(-n3c4ccc5oc6cccc7c8ccccc8c8cc9ccccc9c3c8c4c5c67)cc2)cc1 ; c1ccc(N(c2ccccc2)c2ccc(-n3c4cccc5c6ccccc6c6cc7ccccc7c7oc8ccc3c(c8c67)c54)cc2)cc1]

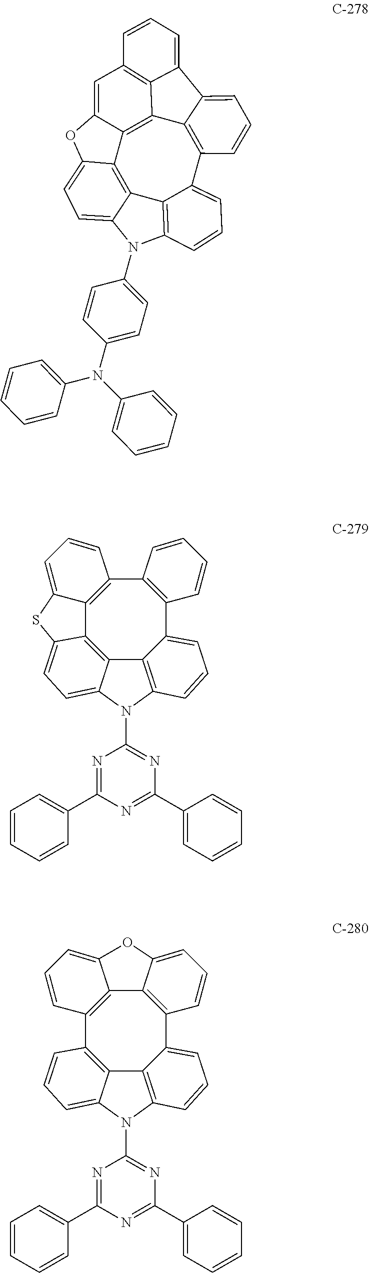 [SMILES: c1ccc(N(c2ccccc2)c2ccc(-n3c4cccc5c6cccc7c6c6c8c-7cccc8cc7oc8ccc3c(c8c76)c54)cc2)cc1 ; c1ccc(-c2nc(-c3ccccc3)nc(-n3c4cccc5c6ccccc6c6cccc7sc8ccc3c(c8c76)c54)n2)cc1 ; c1ccc(-c2nc(-c3ccccc3)nc(-n3c4cccc5c6cccc7oc8cccc(c9cccc3c9c54)c8c76)n2)cc1]

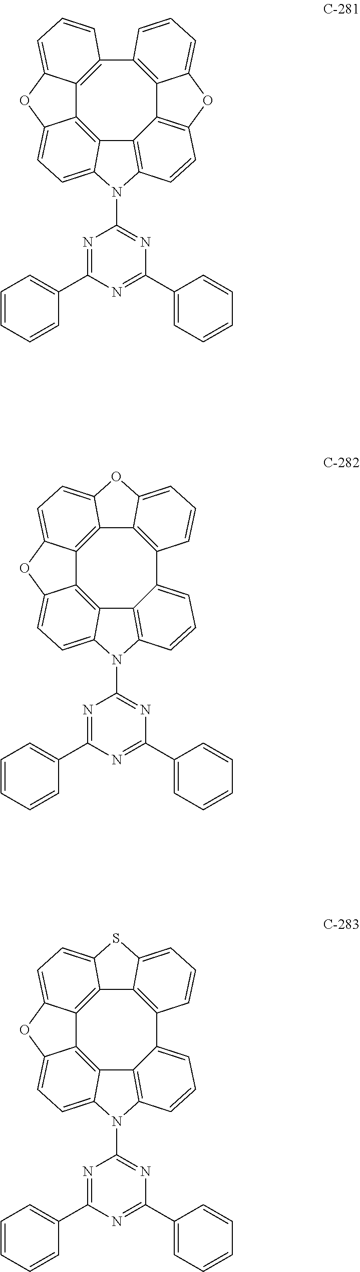 [SMILES: c1ccc(-c2nc(-c3ccccc3)nc(-n3c4ccc5oc6cccc7c8cccc9oc%10ccc3c(c%10c98)c4c5c67)n2)cc1 ; c1ccc(-c2nc(-c3ccccc3)nc(-n3c4cccc5c6cccc7oc8ccc9oc%10ccc3c(c%10c9c8c76)c54)n2)cc1 ; c1ccc(-c2nc(-c3ccccc3)nc(-n3c4cccc5c6cccc7sc8ccc9oc%10ccc3c(c%10c9c8c76)c54)n2)cc1]

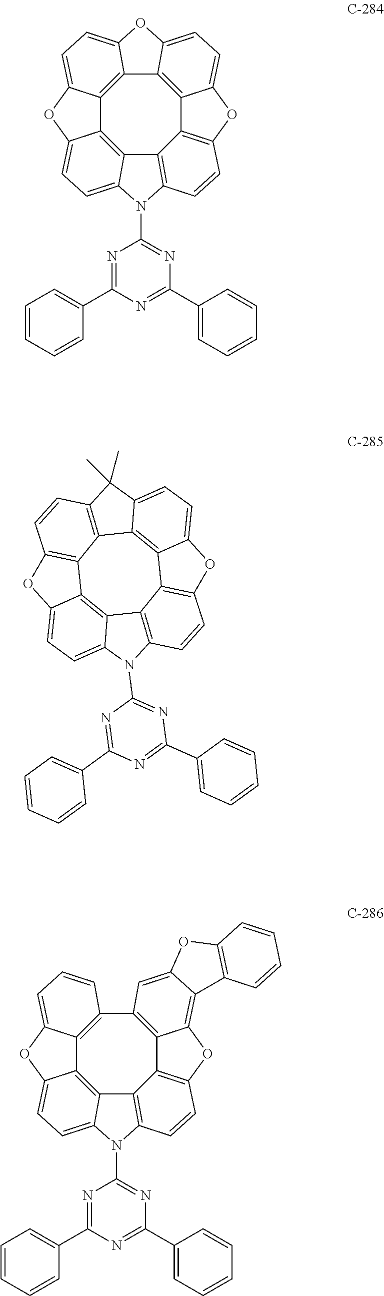 [SMILES: c1ccc(-c2nc(-c3ccccc3)nc(-n3c4ccc5oc6ccc7oc8ccc9oc%10ccc3c3c%10c9c8c7c6c5c34)n2)cc1 ; CC1(C)c2ccc3oc4ccc5c6c4c3c2c2c1ccc1oc3ccc(c6c3c12)n5-c1nc(-c2ccccc2)nc(-c2ccccc2)n1 ; c1ccc(-c2nc(-c3ccccc3)nc(-n3c4ccc5oc6cccc7c8cc9oc%10ccccc%10c9c9oc%10ccc3c(c%10c89)c4c5c67)n2)cc1]

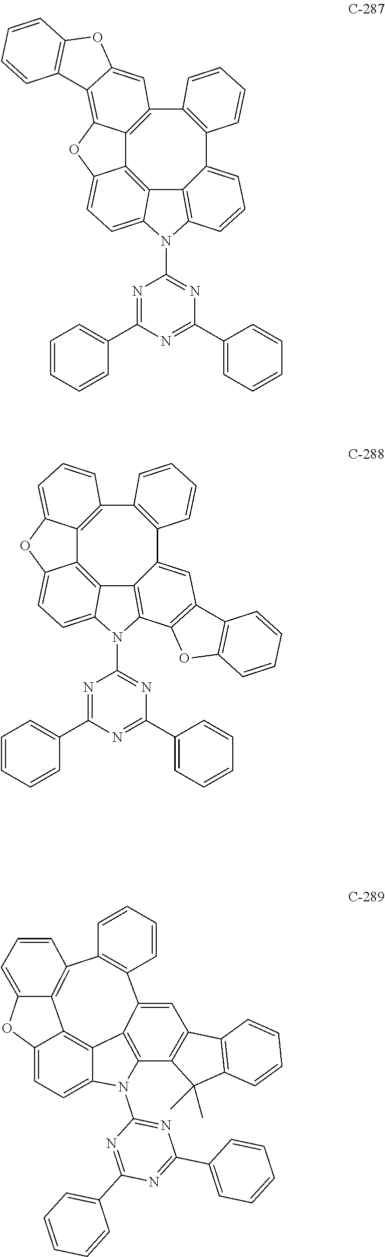 [SMILES: c1ccc(-c2nc(-c3ccccc3)nc(-n3c4cccc5c6ccccc6c6cc7oc8ccccc8c7c7oc8ccc3c(c8c67)c54)n2)cc1 ; c1ccc(-c2nc(-c3ccccc3)nc(-n3c4ccc5oc6cccc7c8ccccc8c8cc9c%10ccccc%10oc9c3c8c4c5c67)n2)cc1 ; CC1(C)c2ccccc2-c2cc3c4ccccc4c4cccc5oc6ccc7c(c6c54)c3c(c21)n7-c1nc(-c2ccccc2)nc(-c2ccccc2)n1]

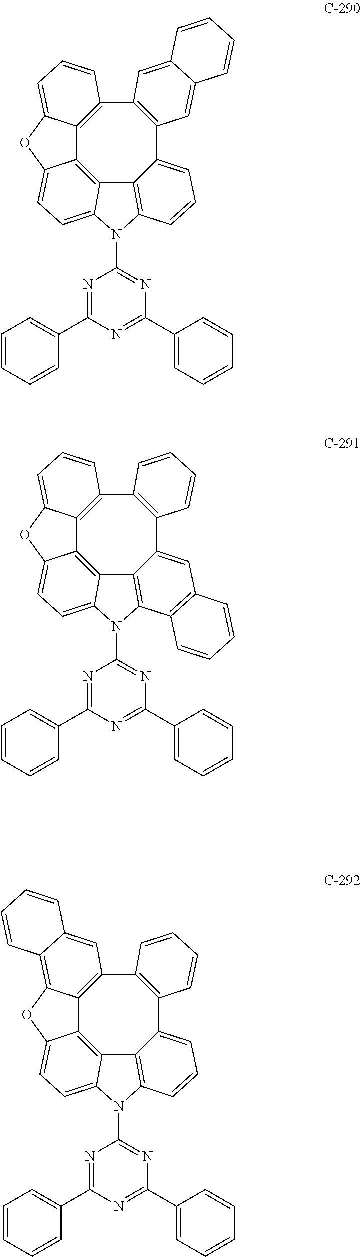 [SMILES: c1ccc(-c2nc(-c3ccccc3)nc(-n3c4cccc5c6cc7ccccc7cc6c6cccc7oc8ccc3c(c8c76)c54)n2)cc1 ; c1ccc(-c2nc(-c3ccccc3)nc(-n3c4ccc5oc6cccc7c8ccccc8c8cc9ccccc9c3c8c4c5c67)n2)cc1 ; c1ccc(-c2nc(-c3ccccc3)nc(-n3c4cccc5c6ccccc6c6cc7ccccc7c7oc8ccc3c(c8c67)c54)n2)cc1]

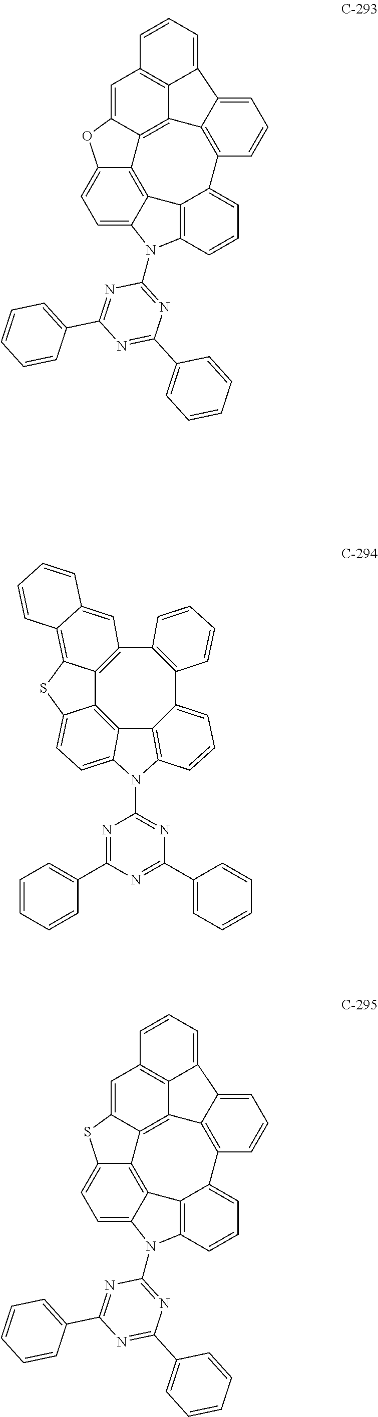 [SMILES: c1ccc(-c2nc(-c3ccccc3)nc(-n3c4cccc5c6cccc7c6c6c8c-7cccc8cc7oc8ccc3c(c8c76)c54)n2)cc1 ; c1ccc(-c2nc(-c3ccccc3)nc(-n3c4cccc5c6ccccc6c6cc7ccccc7c7sc8ccc3c(c8c67)c54)n2)cc1 ; c1ccc(-c2nc(-c3ccccc3)nc(-n3c4cccc5c6cccc7c6c6c8c-7cccc8cc7sc8ccc3c(c8c76)c54)n2)cc1]

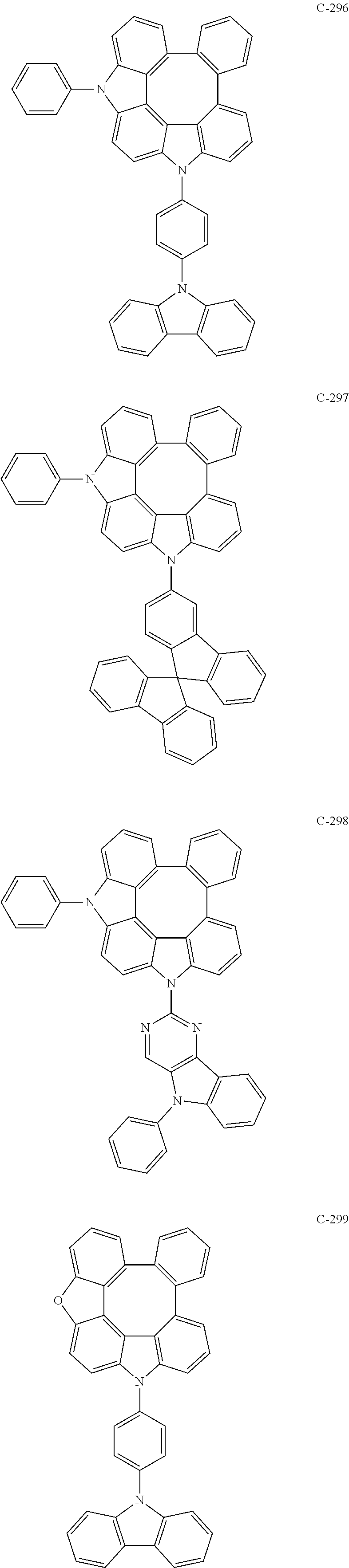 [SMILES: c1ccc(-n2c3cccc4c5ccccc5c5cccc6c5c5c(c43)c2ccc5n6-c2ccc(-n3c4ccccc4c4ccccc43)cc2)cc1 ; c1ccc(-n2c3cccc4c5ccccc5c5cccc6c5c5c(c43)c2ccc5n6-c2ccc3c(c2)-c2ccccc2C32c3ccccc3-c3ccccc32)cc1 ; c1ccc(-n2c3ccccc3c3nc(-n4c5cccc6c7ccccc7c7cccc8c7c7c(c65)c4ccc7n8-c4ccccc4)ncc32)cc1 ; c1ccc2c(c1)c1cccc3oc4ccc5c(c4c31)c1c2cccc1n5-c1ccc(-n2c3ccccc3c3ccccc32)cc1]

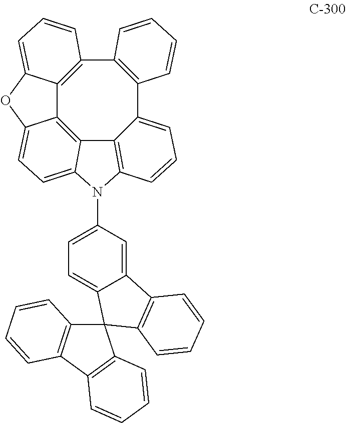 [SMILES: c1ccc2c(c1)-c1ccccc1C21c2ccccc2-c2cc(-n3c4cccc5c6ccccc6c6cccc7oc8ccc3c(c8c76)c54)ccc21]

The scaffolds of formula 1 according to the present disclosure may be prepared by a synthetic method known to one skilled in the art, and for example may be prepared as shown in the following reaction schemes, but is not limited thereto.

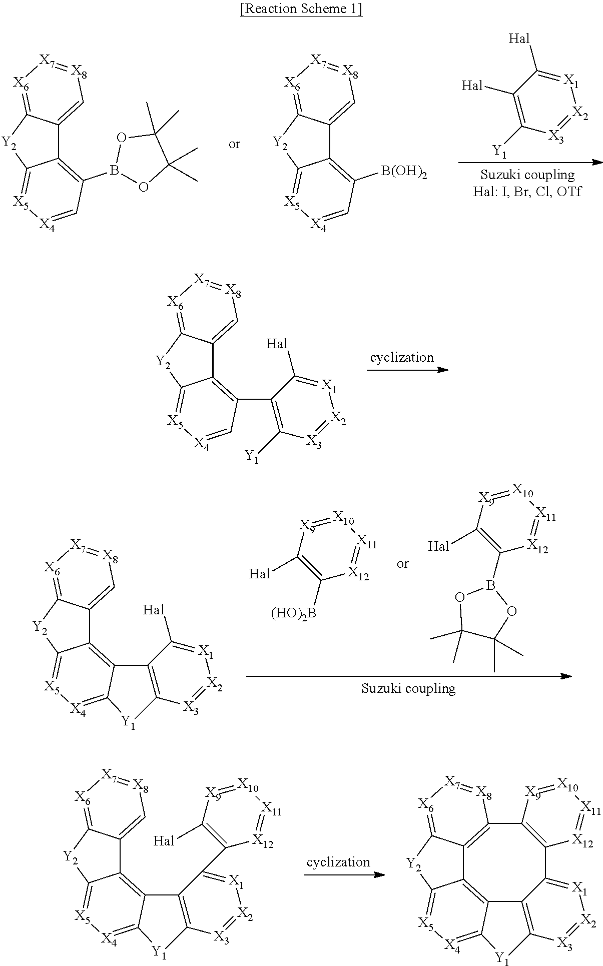

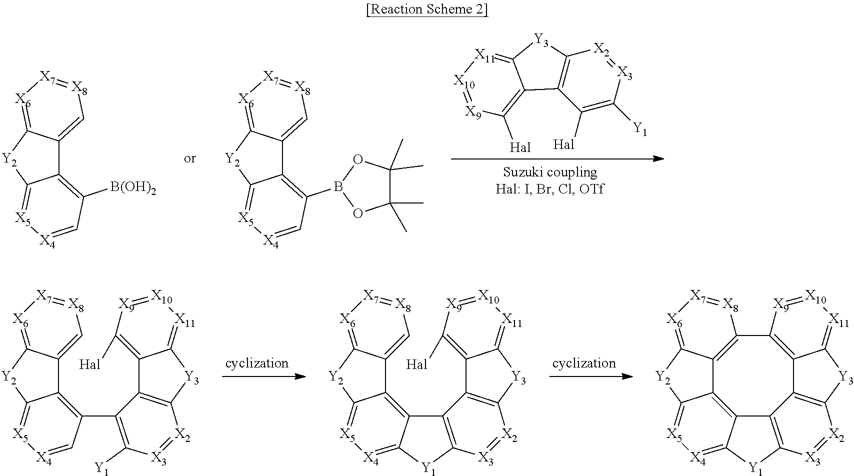

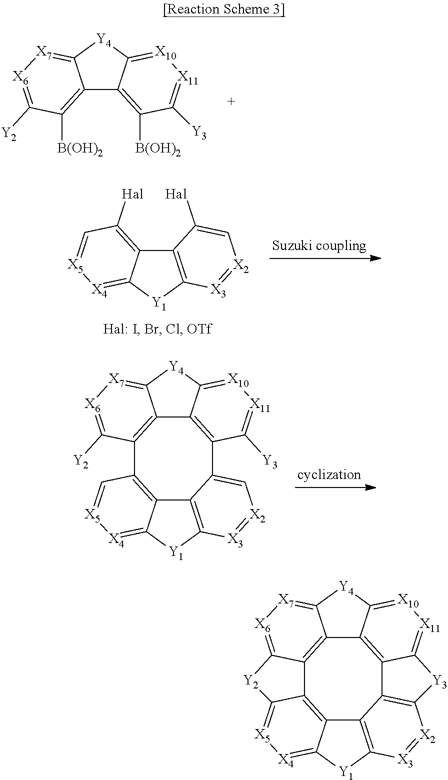

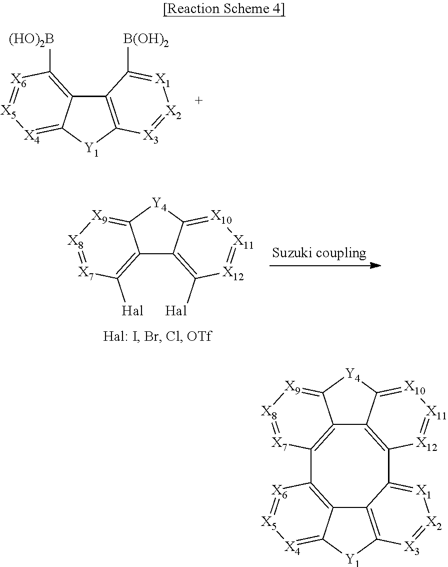

In reaction schemes 1 to 4, Y₁ to Y₄, and X₁ to X₁₂ are as defined in formulas 1-1 to 1-5.

Although illustrative synthesis examples of the compound represented by formula 1 are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 1 above but are not specified in the specific synthesis examples, are bonded.

The present disclosure provides an organic electroluminescent material comprising the organic electroluminescent compound represented by formula 1, and an organic electroluminescent device comprising the organic electroluminescent material. The organic electroluminescent material may consist of the compound according to the present disclosure alone, or may further comprise conventional materials included in the organic electroluminescent material.

The organic electroluminescent compound represented by formula 1 of the present disclosure may be comprised in at least one of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, preferably, may be comprised in the light-emitting layer. When used in the light-emitting layer, the organic electroluminescent compound represented by formula 1 of the present disclosure may be comprised as a host material. Preferably, the light-emitting layer may further comprise at least one dopant. If necessary, the organic electroluminescent compound of the present disclosure may be used as a co-host material. That is, the light-emitting layer may further include an organic electroluminescent compound other than the organic electroluminescent compound represented by formula 1 of the present disclosure (first host material) as a second host material. The weight ratio between the first host material and the second host material is in the range of 1:99 to 99:1. When two or more materials are included in one layer, mixed deposition may be performed to form a layer, or co-deposition may be performed separately at the same time to form a layer.

The second host material may be selected from any of the known host materials. For example, the second host material may comprise a compound represented by the following formula 11, but is not limited thereto.

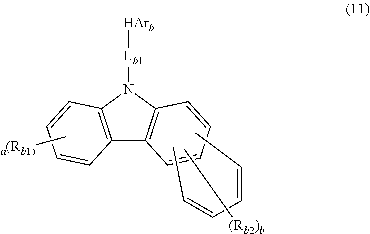

wherein
HAr_{b} represents a substituted or unsubstituted (3- to 30-membered)heteroaryl;
L_{b1} represents a single bond, a substituted or unsubstituted (C6-C30)arylene, or a substituted or unsubstituted (3- to 30-membered)heteroarylene;
R_{b1} and R_{b2}, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent(s) to form a ring(s);
a represents an integer of 1 to 4; and b represents an integer of 1 to 6; where if a and b, each independently, represent an integer of 2 or more, each of R_{b1} and each of R_{b2} may be the same as or different from each other.

Specifically, the formula 11 may be represented by any one of the following formulas 11-1 and 11-2.

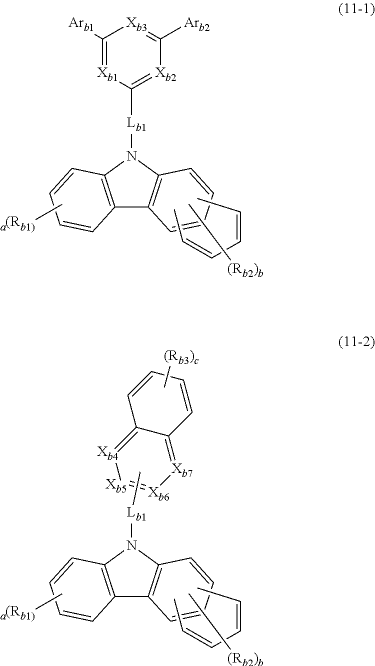

In the formulas 11-1 and 11-2, X_{b1} to X_{b7}, each independently, represent CR_{b4} or N; at least one of X_{b1} to X_{b3} represents N; at least one of X_{b4} to X_{b7} represents N; and R_{b3} and R_{b4}, each independently, are the same as the definition of R_{b1}.

In the formulas 11, 11-1 and 11-2,

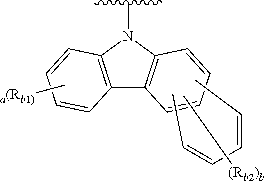

may be specifically represented as follows.

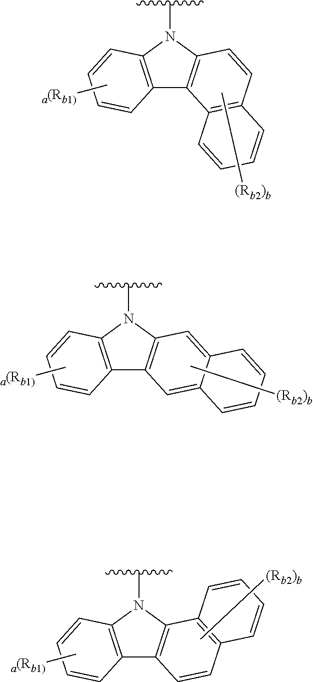

The compound represented by formula 11 may be specifically exemplified by the following compounds, but is not limited thereto.

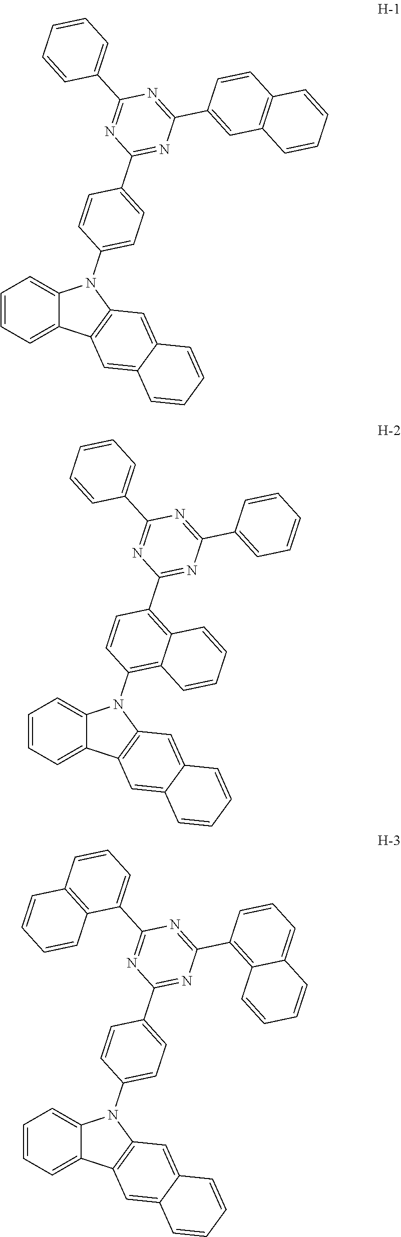 [SMILES: c1ccc(-c2nc(-c3ccc(-n4c5ccccc5c5cc6ccccc6cc54)cc3)nc(-c3ccc4ccccc4c3)n2)cc1 ; c1ccc(-c2nc(-c3ccccc3)nc(-c3ccc(-n4c5ccccc5c5cc6ccccc6cc54)c4ccccc34)n2)cc1 ; c1ccc2cc3c(cc2c1)c1ccccc1n3-c1ccc(-c2nc(-c3cccc4ccccc34)nc(-c3cccc4ccccc34)n2)cc1]

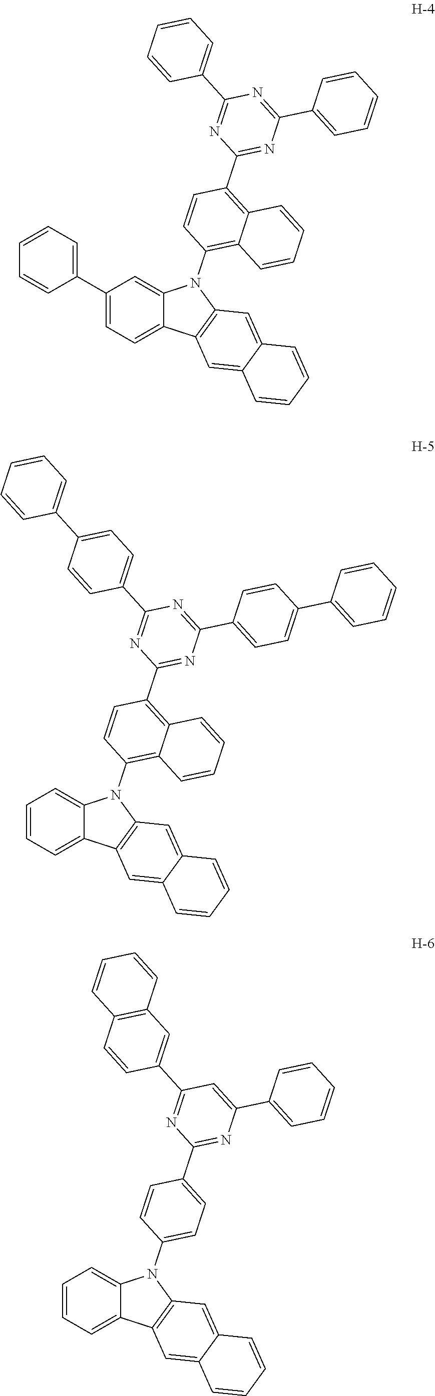 [SMILES: c1ccc(-c2ccc3c4cc5ccccc5cc4n(-c4ccc(-c5nc(-c6ccccc6)nc(-c6ccccc6)n5)c5ccccc45)c3c2)cc1 ; c1ccc(-c2ccc(-c3nc(-c4ccc(-c5ccccc5)cc4)nc(-c4ccc(-n5c6ccccc6c6cc7ccccc7cc65)c5ccccc45)n3)cc2)cc1 ; c1ccc(-c2cc(-c3ccc4ccccc4c3)nc(-c3ccc(-n4c5ccccc5c5cc6ccccc6cc54)cc3)n2)cc1]

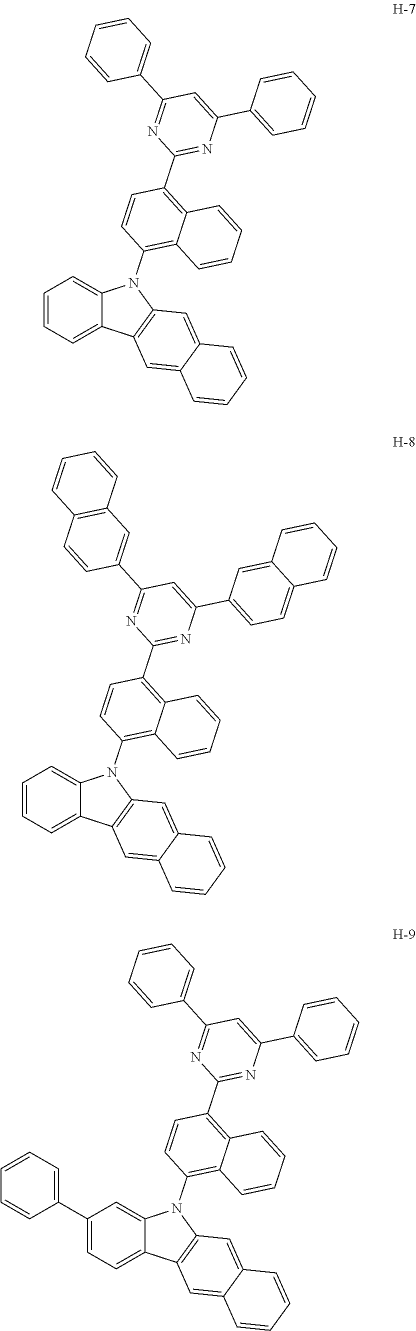 [SMILES: c1ccc(-c2cc(-c3ccccc3)nc(-c3ccc(-n4c5ccccc5c5cc6ccccc6cc54)c4ccccc34)n2)cc1 ; c1ccc2cc(-c3cc(-c4ccc5ccccc5c4)nc(-c4ccc(-n5c6ccccc6c6cc7ccccc7cc65)c5ccccc45)n3)ccc2c1 ; c1ccc(-c2ccc3c4cc5ccccc5cc4n(-c4ccc(-c5nc(-c6ccccc6)cc(-c6ccccc6)n5)c5ccccc45)c3c2)cc1]

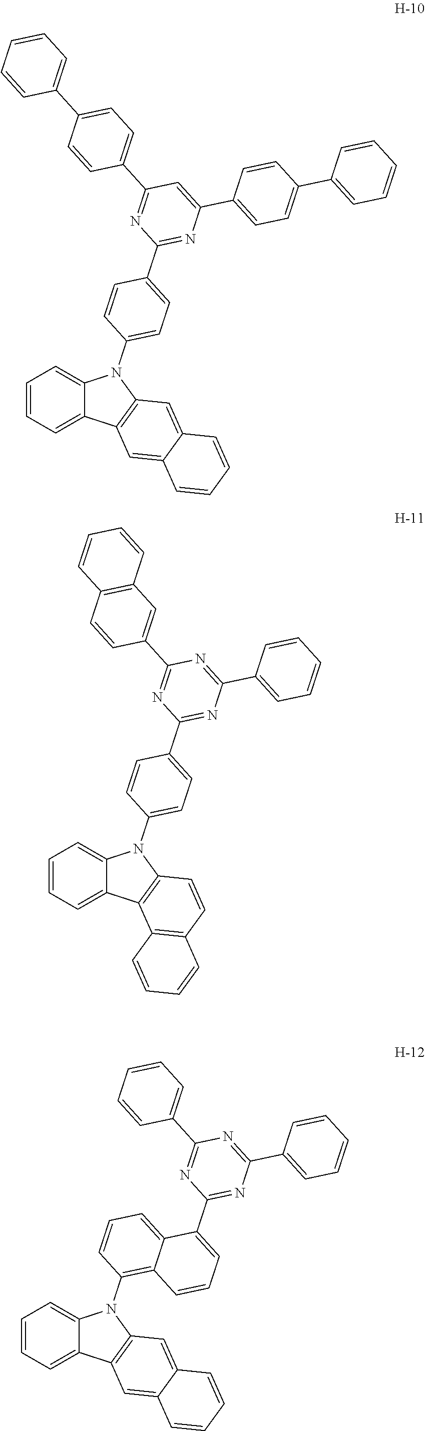 [SMILES: c1ccc(-c2ccc(-c3cc(-c4ccc(-c5ccccc5)cc4)nc(-c4ccc(-n5c6ccccc6c6cc7ccccc7cc65)cc4)n3)cc2)cc1 ; c1ccc(-c2nc(-c3ccc(-n4c5ccccc5c5c6ccccc6ccc54)cc3)nc(-c3ccc4ccccc4c3)n2)cc1 ; c1ccc(-c2nc(-c3ccccc3)nc(-c3cccc4c(-n5c6ccccc6c6cc7ccccc7cc65)cccc34)n2)cc1]

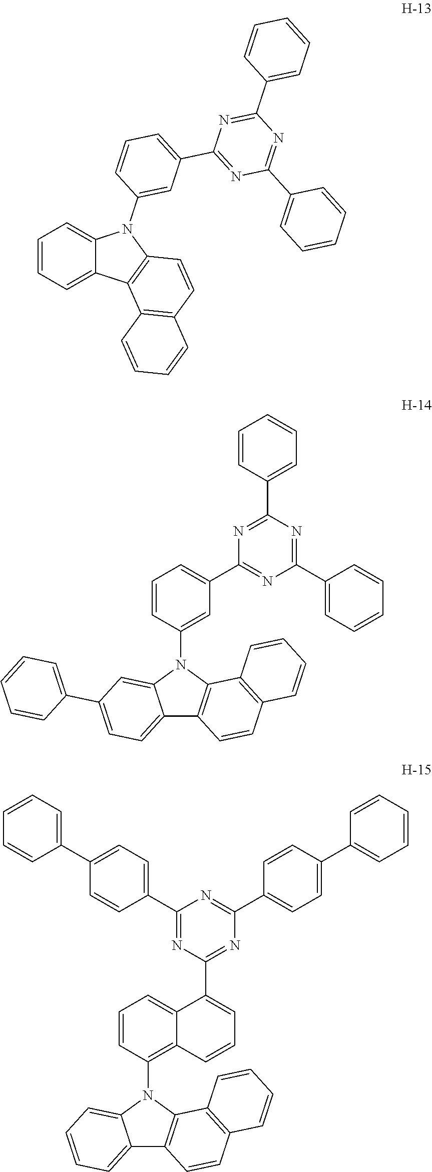 [SMILES: c1ccc(-c2nc(-c3ccccc3)nc(-c3cccc(-n4c5ccccc5c5c6ccccc6ccc54)c3)n2)cc1 ; c1ccc(-c2ccc3c4ccc5ccccc5c4n(-c4cccc(-c5nc(-c6ccccc6)nc(-c6ccccc6)n5)c4)c3c2)cc1 ; c1ccc(-c2ccc(-c3nc(-c4ccc(-c5ccccc5)cc4)nc(-c4cccc5c(-n6c7ccccc7c7ccc8ccccc8c76)cccc45)n3)cc2)cc1]

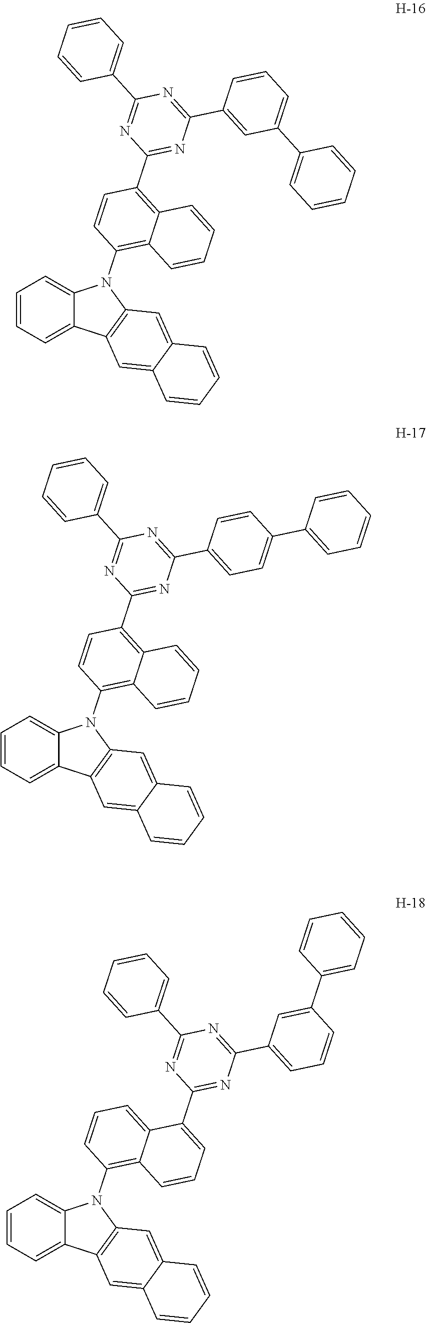 [SMILES: c1ccc(-c2cccc(-c3nc(-c4ccccc4)nc(-c4ccc(-n5c6ccccc6c6cc7ccccc7cc65)c5ccccc45)n3)c2)cc1 ; c1ccc(-c2ccc(-c3nc(-c4ccccc4)nc(-c4ccc(-n5c6ccccc6c6cc7ccccc7cc65)c5ccccc45)n3)cc2)cc1 ; c1ccc(-c2cccc(-c3nc(-c4ccccc4)nc(-c4cccc5c(-n6c7ccccc7c7cc8ccccc8cc76)cccc45)n3)c2)cc1]

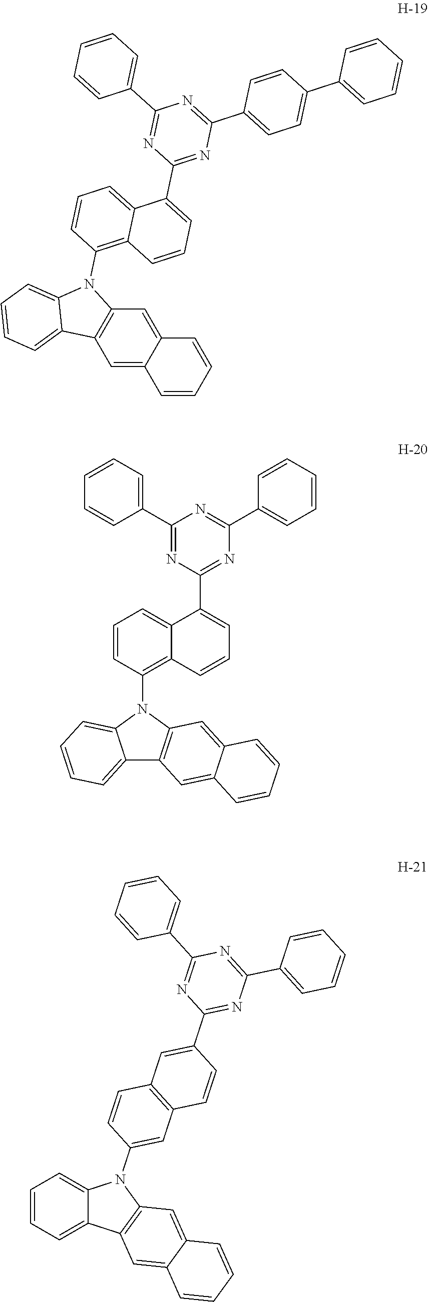 [SMILES: c1ccc(-c2ccc(-c3nc(-c4ccccc4)nc(-c4cccc5c(-n6c7ccccc7c7cc8ccccc8cc76)cccc45)n3)cc2)cc1 ; c1ccc(-c2nc(-c3ccccc3)nc(-c3cccc4c(-n5c6ccccc6c6cc7ccccc7cc65)cccc34)n2)cc1 ; c1ccc(-c2nc(-c3ccccc3)nc(-c3ccc4cc(-n5c6ccccc6c6cc7ccccc7cc65)ccc4c3)n2)cc1]

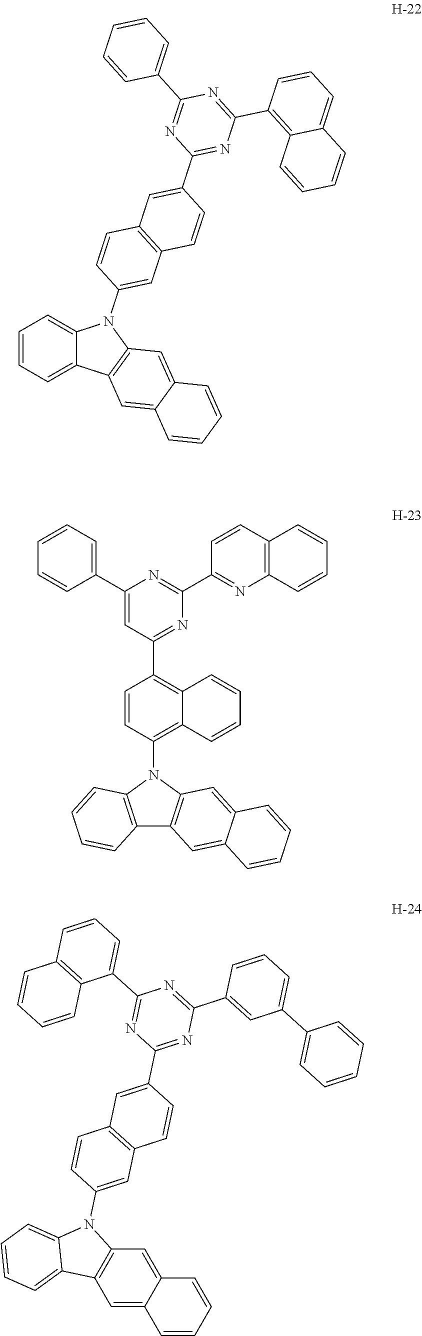 [SMILES: c1ccc(-c2nc(-c3ccc4cc(-n5c6ccccc6c6cc7ccccc7cc65)ccc4c3)nc(-c3cccc4ccccc34)n2)cc1 ; c1ccc(-c2cc(-c3ccc(-n4c5ccccc5c5cc6ccccc6cc54)c4ccccc34)nc(-c3ccc4ccccc4n3)n2)cc1 ; c1ccc(-c2cccc(-c3nc(-c4ccc5cc(-n6c7ccccc7c7cc8ccccc8cc76)ccc5c4)nc(-c4cccc5ccccc45)n3)c2)cc1]

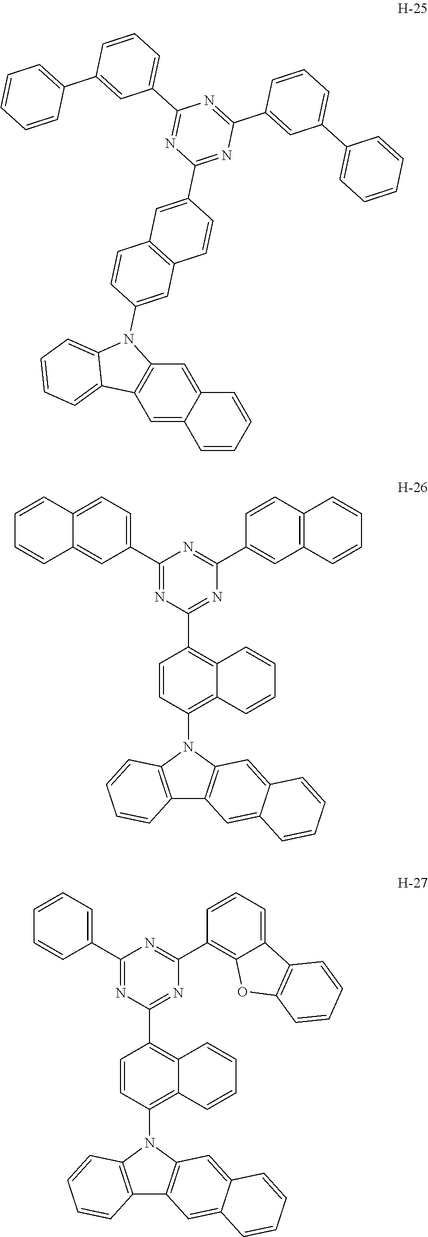 [SMILES: c1ccc(-c2cccc(-c3nc(-c4cccc(-c5ccccc5)c4)nc(-c4ccc5cc(-n6c7ccccc7c7cc8ccccc8cc76)ccc5c4)n3)c2)cc1 ; c1ccc2cc(-c3nc(-c4ccc5ccccc5c4)nc(-c4ccc(-n5c6ccccc6c6cc7ccccc7cc65)c5ccccc45)n3)ccc2c1 ; c1ccc(-c2nc(-c3ccc(-n4c5ccccc5c5cc6ccccc6cc54)c4ccccc34)nc(-c3cccc4c3oc3ccccc34)n2)cc1]

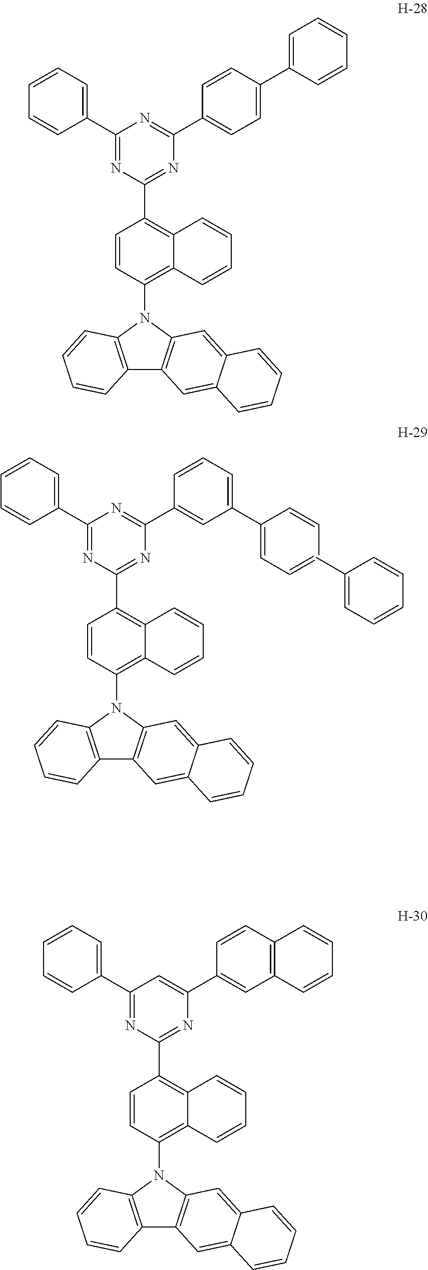

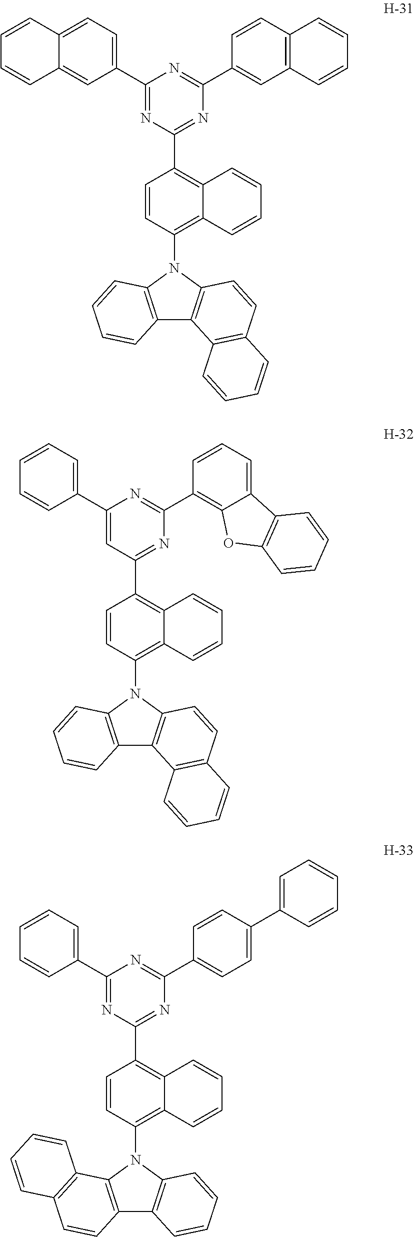

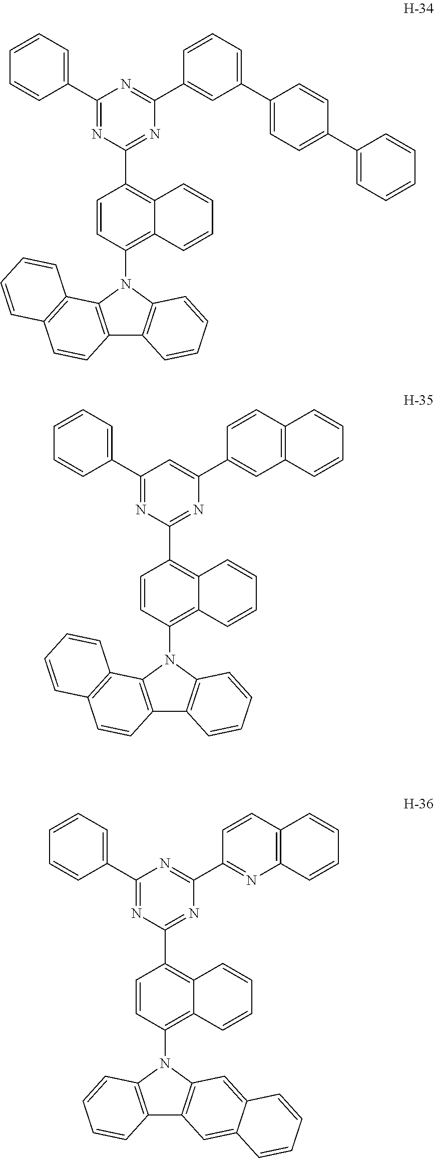 [SMILES: c1ccc(-c2ccc(-c3cccc(-c4nc(-c5ccccc5)nc(-c5ccc(-n6c7ccccc7c7ccc8ccccc8c76)c6ccccc56)n4)c3)cc2)cc1 ; c1ccc(-c2cc(-c3ccc4ccccc4c3)nc(-c3ccc(-n4c5ccccc5c5ccc6ccccc6c54)c4ccccc34)n2)cc1 ; c1ccc(-c2nc(-c3ccc4ccccc4n3)nc(-c3ccc(-n4c5ccccc5c5cc6ccccc6cc54)c4ccccc34)n2)cc1]

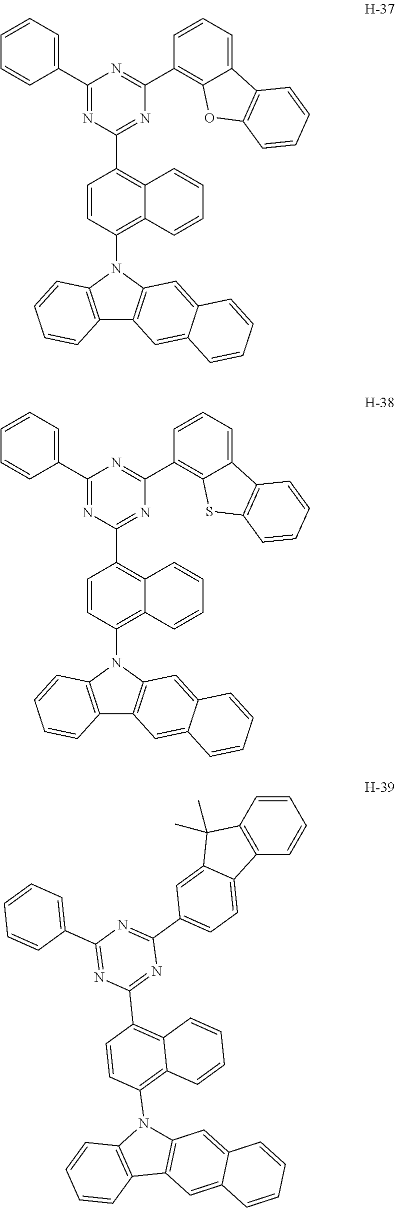

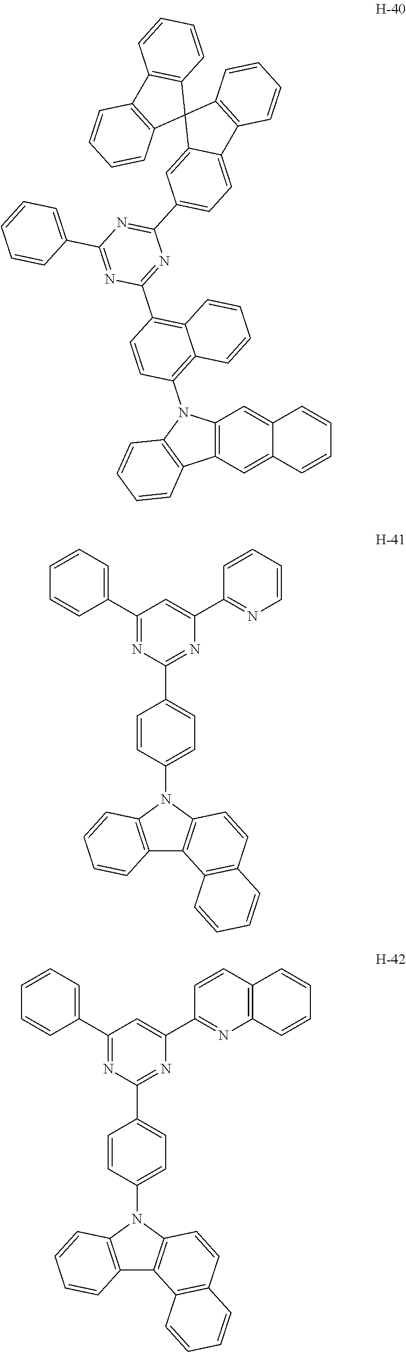

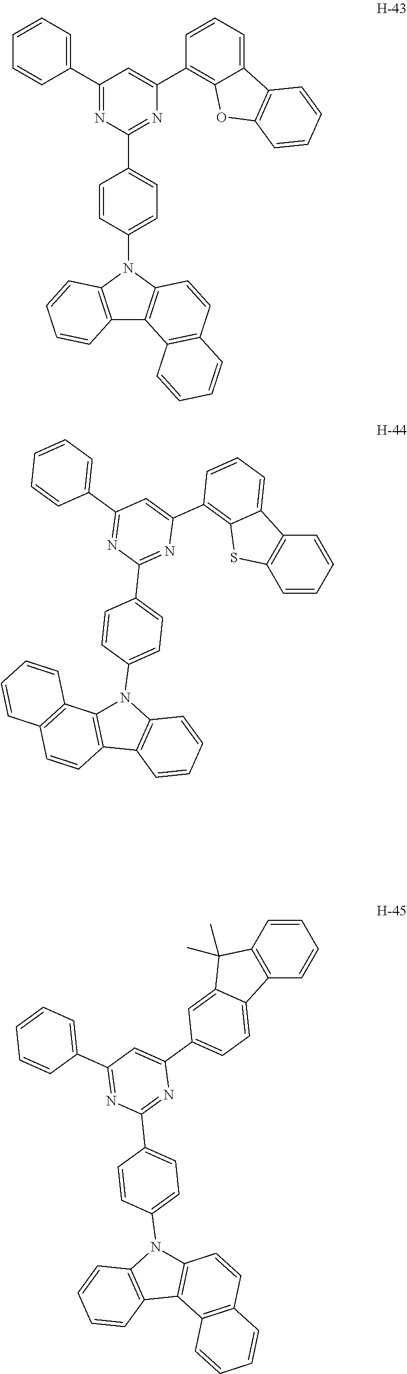 [SMILES: c1ccc(-c2cc(-c3cccc4c3oc3ccccc34)nc(-c3ccc(-n4c5ccccc5c5c6ccccc6ccc54)cc3)n2)cc1 ; c1ccc(-c2cc(-c3cccc4c3sc3ccccc34)nc(-c3ccc(-n4c5ccccc5c5ccc6ccccc6c54)cc3)n2)cc1 ; CC1(C)c2ccccc2-c2ccc(-c3cc(-c4ccccc4)nc(-c4ccc(-n5c6ccccc6c6c7ccccc7ccc65)cc4)n3)cc21]

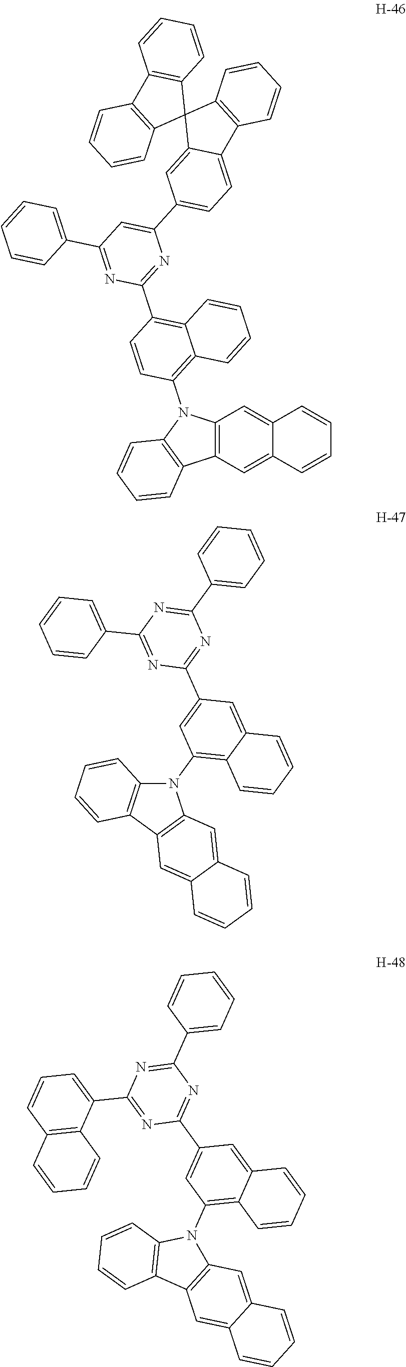

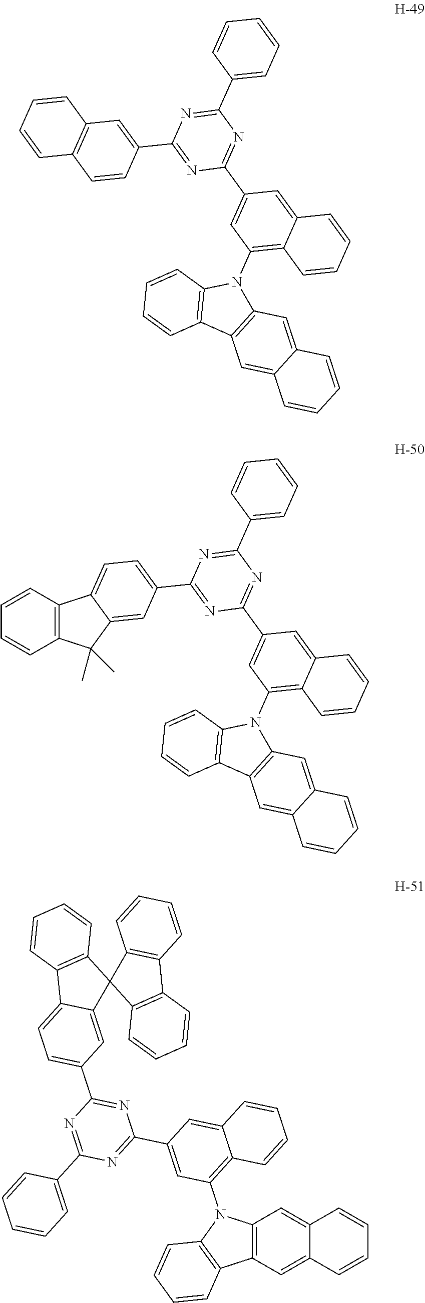 [SMILES: c1ccc(-c2nc(-c3ccc4ccccc4c3)nc(-c3cc(-n4c5ccccc5c5cc6ccccc6cc54)c4ccccc4c3)n2)cc1 ; CC1(C)c2ccccc2-c2ccc(-c3nc(-c4ccccc4)nc(-c4cc(-n5c6ccccc6c6cc7ccccc7cc65)c5ccccc5c4)n3)cc21 ; c1ccc(-c2nc(-c3ccc4c(c3)C3(c5ccccc5-c5ccccc53)c3ccccc3-4)nc(-c3cc(-n4c5ccccc5c5cc6ccccc6cc54)c4ccccc4c3)n2)cc1]

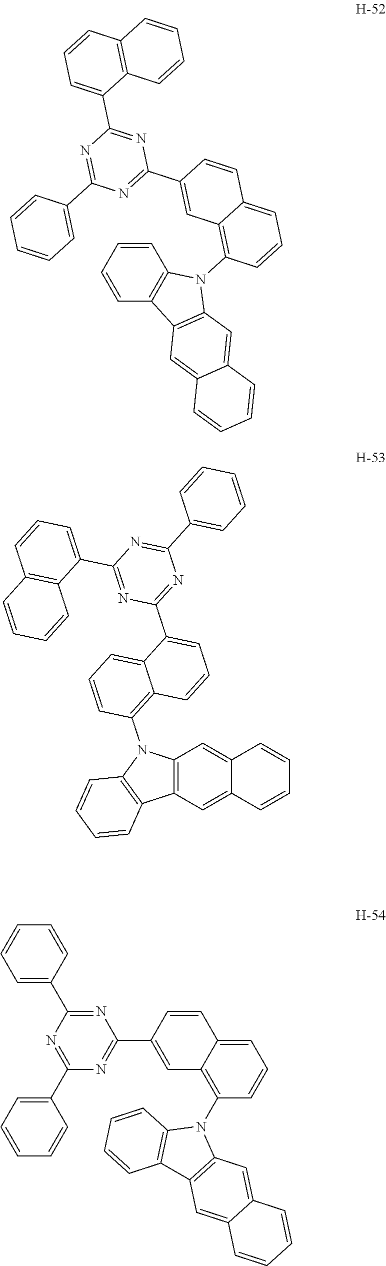

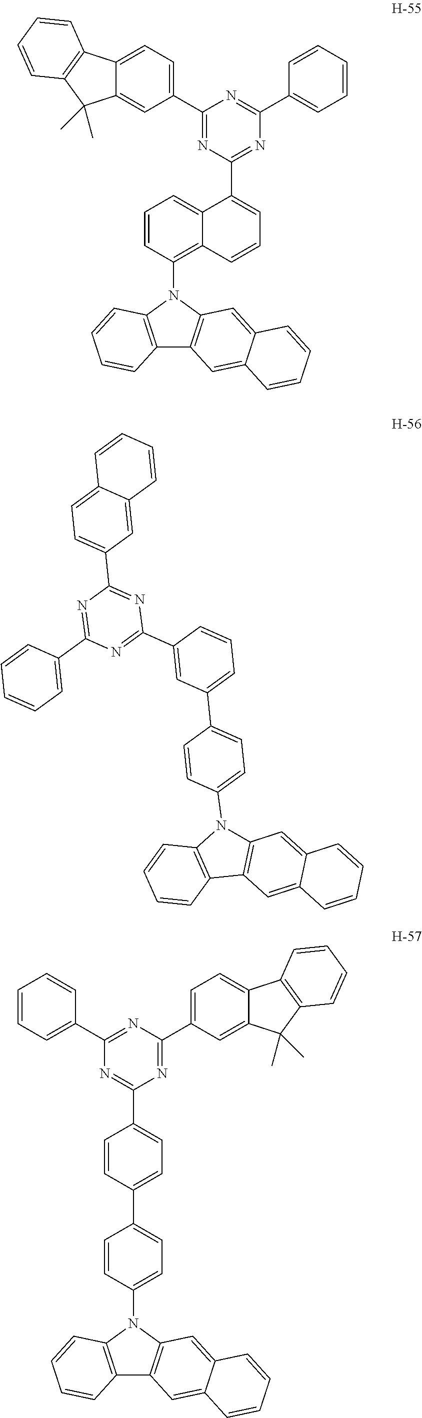

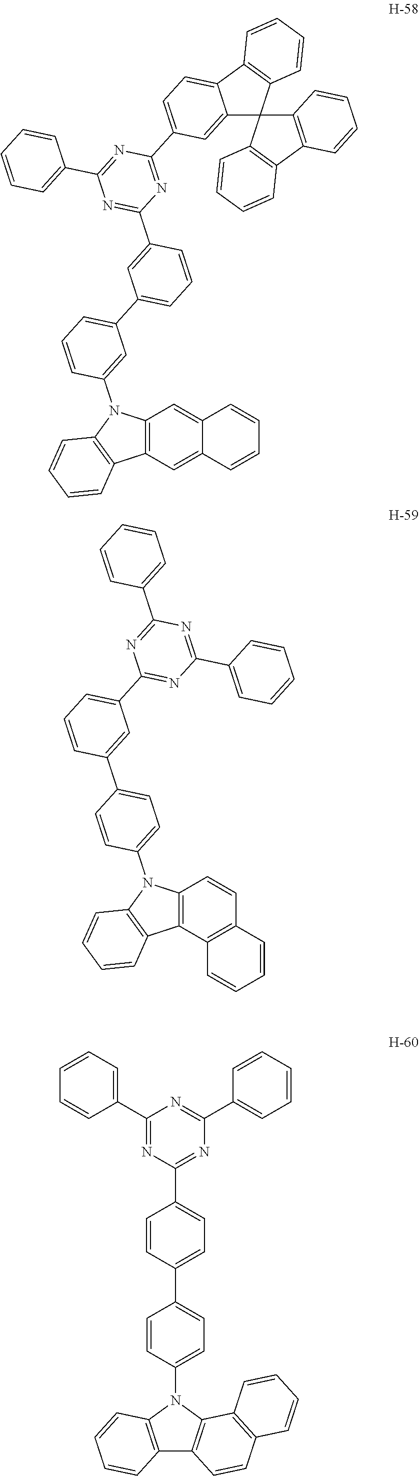

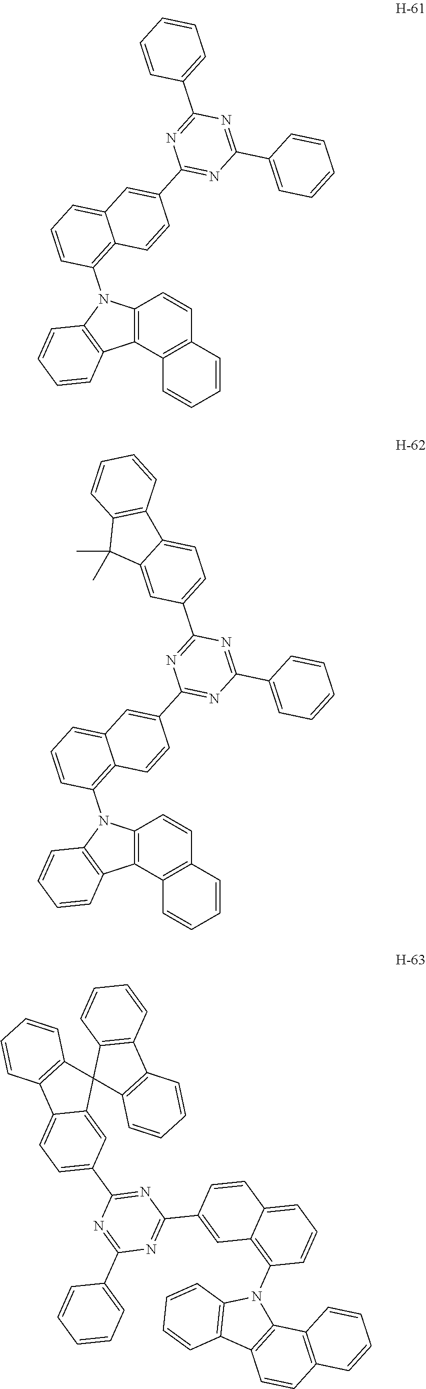 [SMILES: c1ccc(-c2nc(-c3ccccc3)nc(-c3ccc4c(-n5c6ccccc6c6c7ccccc7ccc65)cccc4c3)n2)cc1 ; CC1(C)c2ccccc2-c2ccc(-c3nc(-c4ccccc4)nc(-c4ccc5c(-n6c7ccccc7c7c8ccccc8ccc76)cccc5c4)n3)cc21 ; c1ccc(-c2nc(-c3ccc4c(c3)C3(c5ccccc5-c5ccccc53)c3ccccc3-4)nc(-c3ccc4cccc(-n5c6ccccc6c6ccc7ccccc7c65)c4c3)n2)cc1]

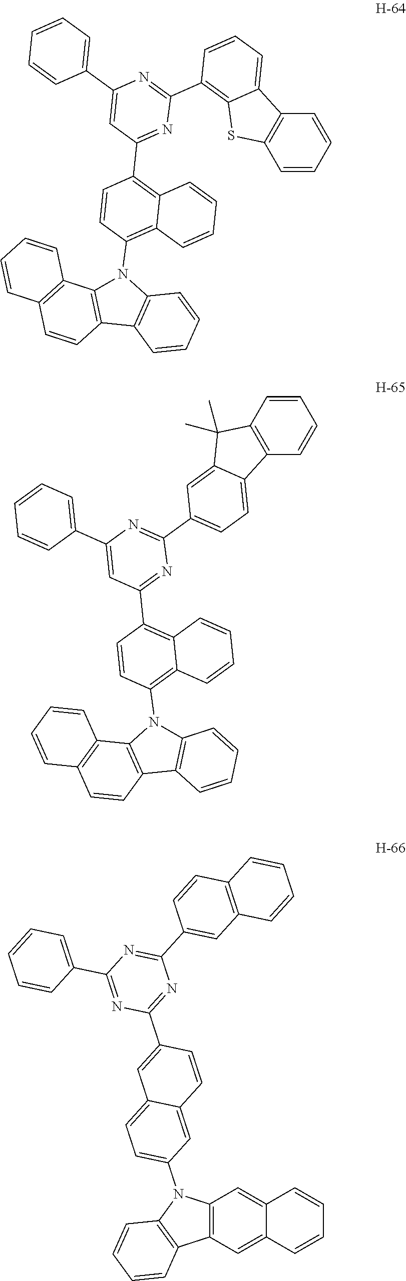 [SMILES: c1ccc(-c2cc(-c3ccc(-n4c5ccccc5c5ccc6ccccc6c54)c4ccccc34)nc(-c3cccc4c3sc3ccccc34)n2)cc1 ; CC1(C)c2ccccc2-c2ccc(-c3nc(-c4ccccc4)cc(-c4ccc(-n5c6ccccc6c6ccc7ccccc7c65)c5ccccc45)n3)cc21 ; c1ccc(-c2nc(-c3ccc4ccccc4c3)nc(-c3ccc4cc(-n5c6ccccc6c6cc7ccccc7cc65)ccc4c3)n2)cc1]

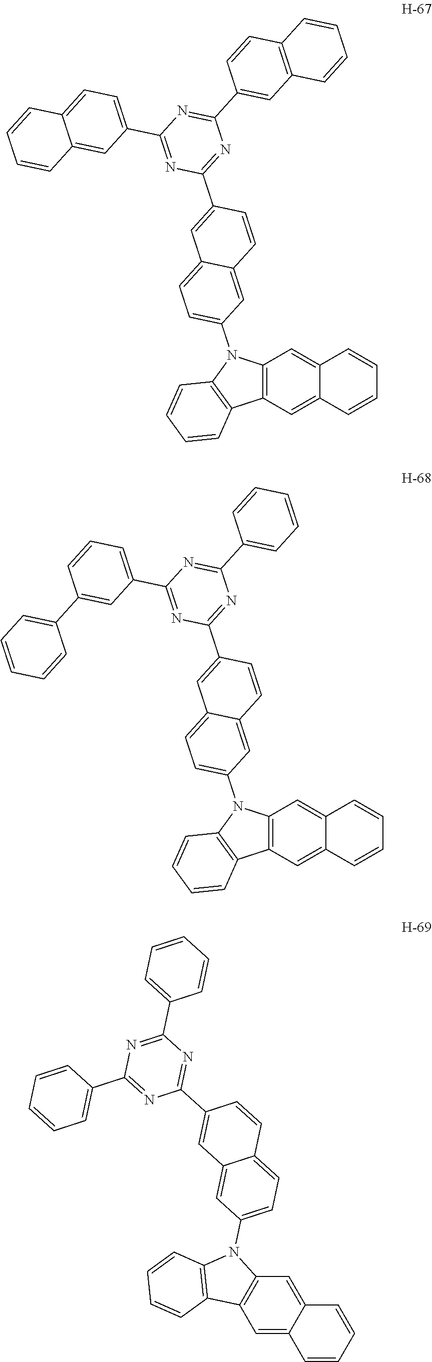

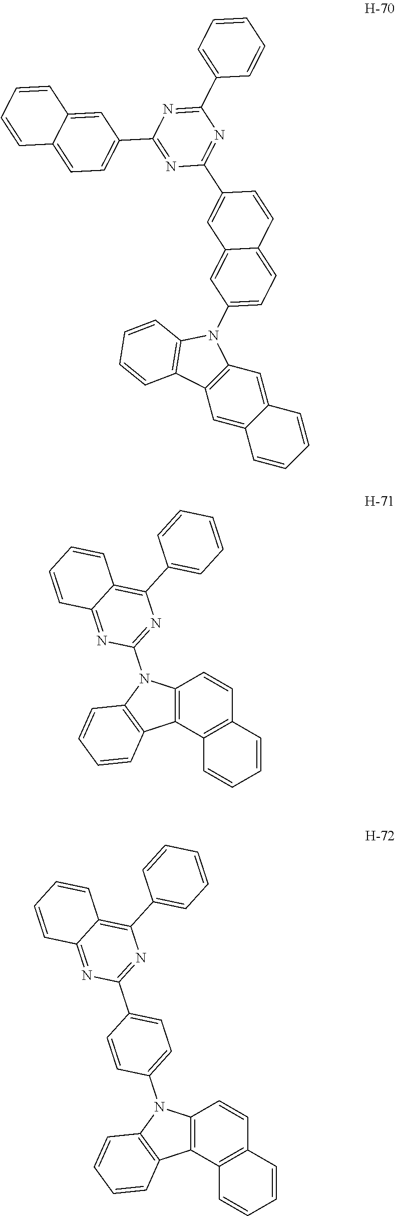 [SMILES: c1ccc(-c2nc(-c3ccc4ccccc4c3)nc(-c3ccc4ccc(-n5c6ccccc6c6cc7ccccc7cc65)cc4c3)n2)cc1 ; c1ccc(-c2nc(-n3c4ccccc4c4c5ccccc5ccc43)nc3ccccc23)cc1 ; c1ccc(-c2nc(-c3ccc(-n4c5ccccc5c5c6ccccc6ccc54)cc3)nc3ccccc23)cc1]

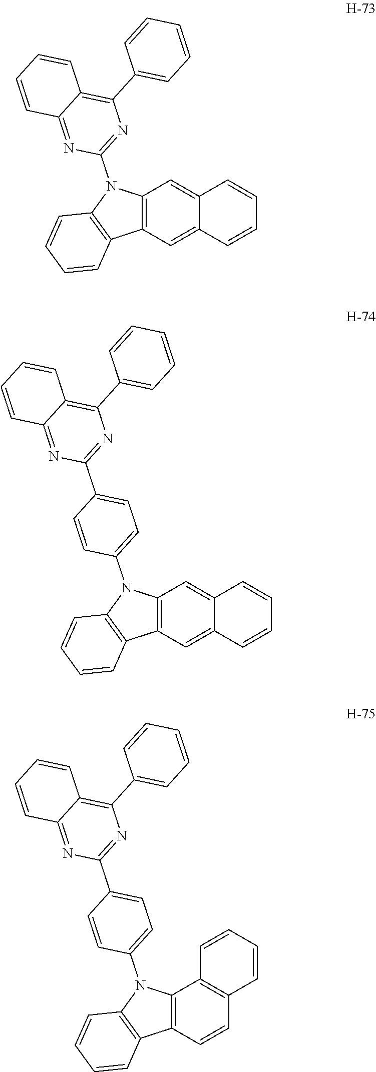 [SMILES: c1ccc(-c2nc(-n3c4ccccc4c4cc5ccccc5cc43)nc3ccccc23)cc1 ; c1ccc(-c2nc(-c3ccc(-n4c5ccccc5c5cc6ccccc6cc54)cc3)nc3ccccc23)cc1 ; c1ccc(-c2nc(-c3ccc(-n4c5ccccc5c5ccc6ccccc6c54)cc3)nc3ccccc23)cc1]

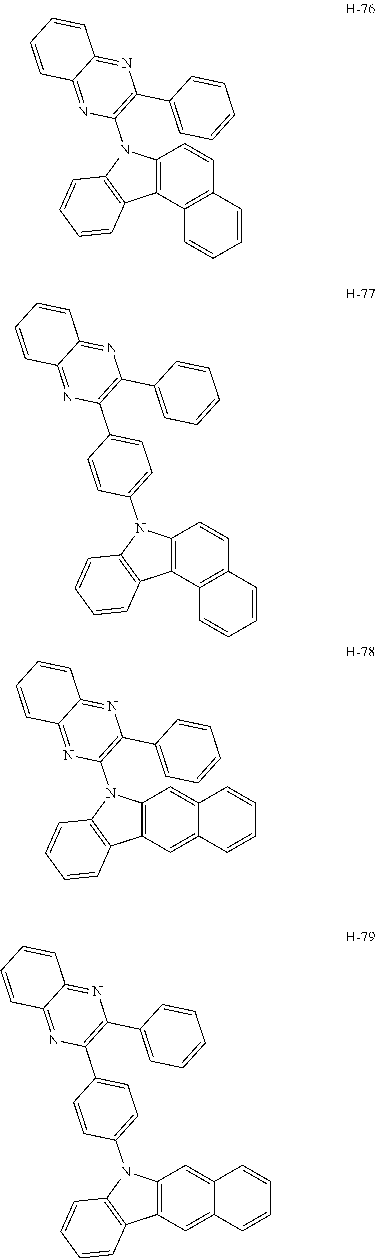 [SMILES: c1ccc(-c2nc3ccccc3nc2-n2c3ccccc3c3c4ccccc4ccc32)cc1 ; c1ccc(-c2nc3ccccc3nc2-c2ccc(-n3c4ccccc4c4c5ccccc5ccc43)cc2)cc1 ; c1ccc(-c2nc3ccccc3nc2-n2c3ccccc3c3cc4ccccc4cc32)cc1 ; c1ccc(-c2nc3ccccc3nc2-c2ccc(-n3c4ccccc4c4cc5ccccc5cc43)cc2)cc1]

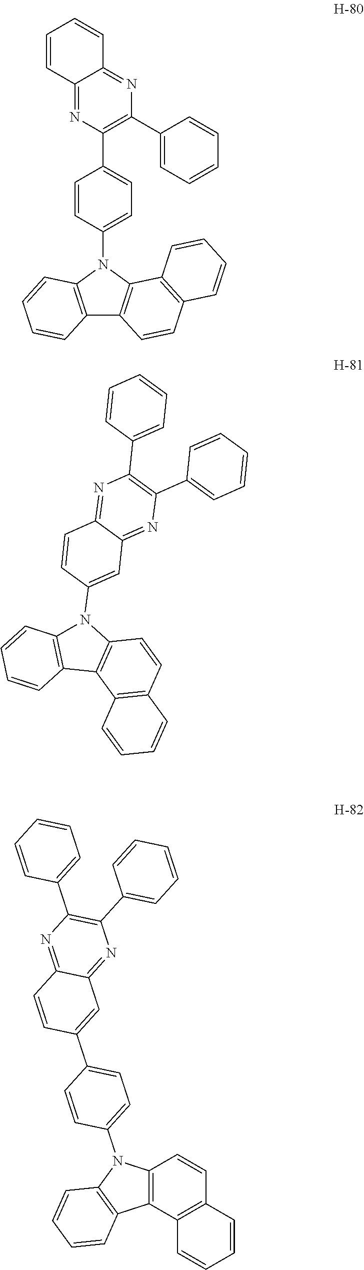

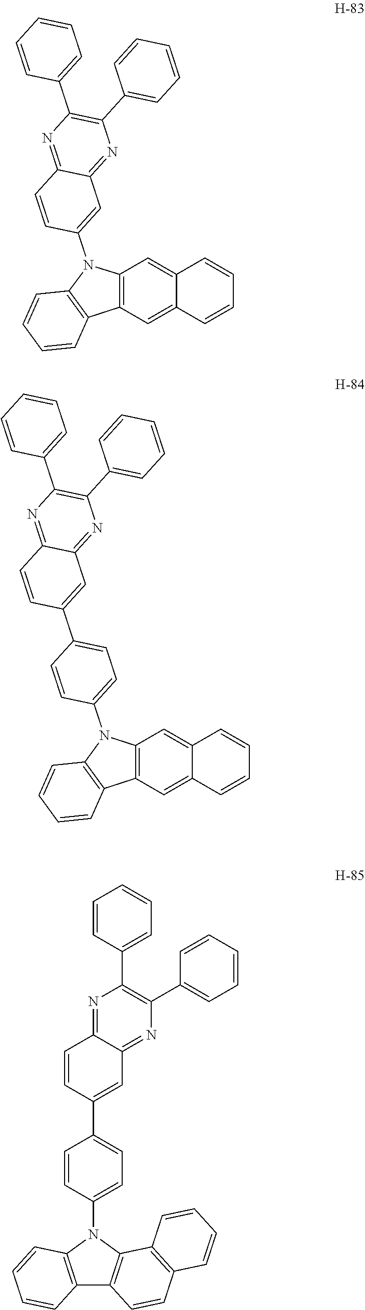 [SMILES: c1ccc(-c2nc3ccc(-n4c5ccccc5c5cc6ccccc6cc54)cc3nc2-c2ccccc2)cc1 ; c1ccc(-c2nc3ccc(-c4ccc(-n5c6ccccc6c6cc7ccccc7cc65)cc4)cc3nc2-c2ccccc2)cc1 ; c1ccc(-c2nc3ccc(-c4ccc(-n5c6ccccc6c6ccc7ccccc7c65)cc4)cc3nc2-c2ccccc2)cc1]

The dopant comprised in the organic electroluminescent device of the present disclosure may be at least one phosphorescent or fluorescent dopant, preferably at least one phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise the compound represented by the following formula 101, but is not limited thereto.

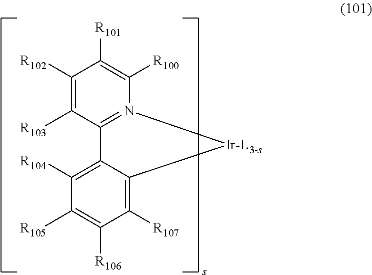

In formula 101, L is any one selected from the following structures 1 to 3:

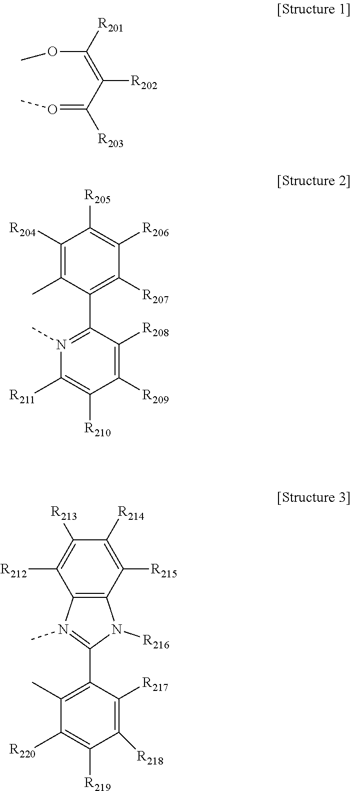

R₁₀₀ to R₁₀₃, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to adjacent one(s) of R₁₀₀ to R₁₀₃, to form a substituted or unsubstituted fused ring with a pyridine, e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline or a substituted or unsubstituted indenoquinoline;
R₁₀₄ to R₁₀₇, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to adjacent one(s) of R₁₀₄ to R₁₀₇ to form a substituted or unsubstituted fused ring with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀, each independently, represent hydrogen, deuterium, a halogen, a (C1-C30)alkyl unsubstituted or substituted with deuterium or a halogen, a substituted or unsubstituted (C3-C30)cycloalkyl, or a substituted or unsubstituted (C6-C30)aryl; or may be linked to adjacent one(s) of adjacent R₂₀₁ to R₂₂₀ to form a substituted or unsubstituted fused ring; and
n represents an integer of 1 to 3.

The specific examples of the dopant compound are as follows, but are not limited thereto.

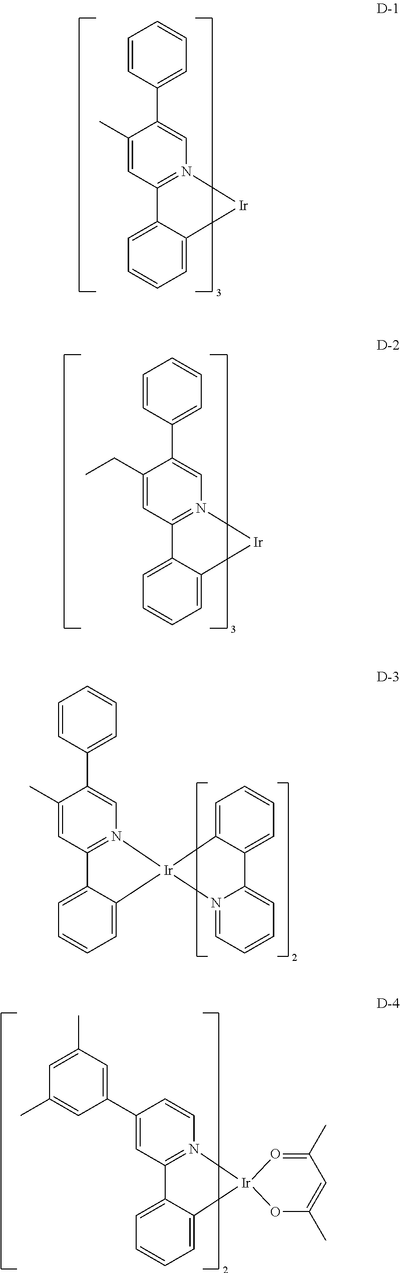

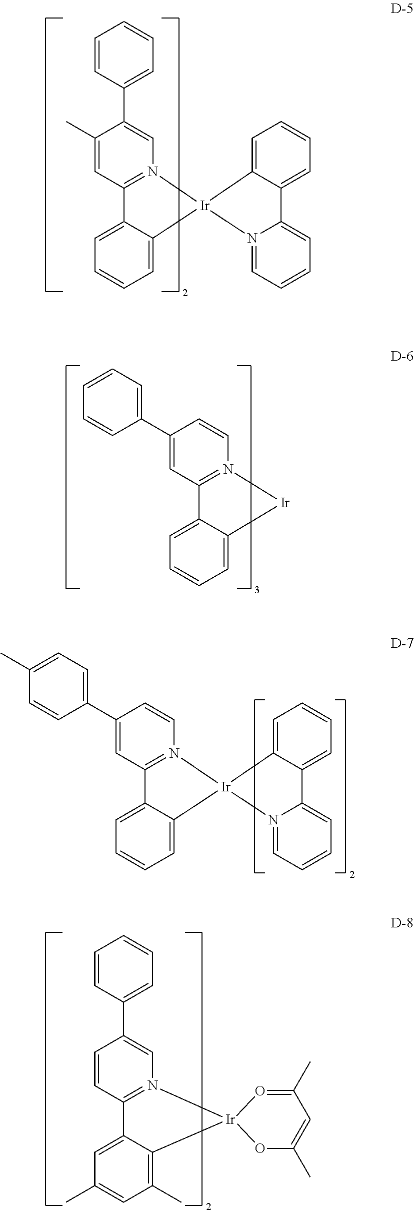

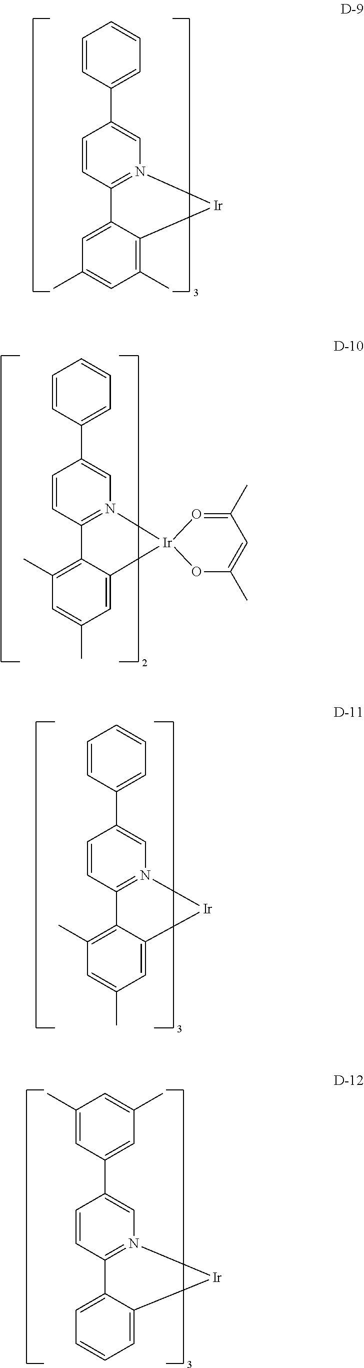

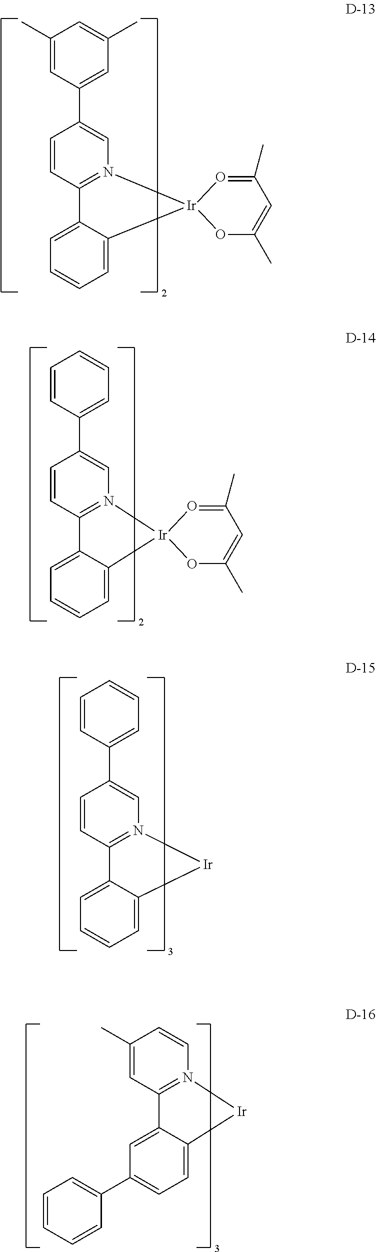

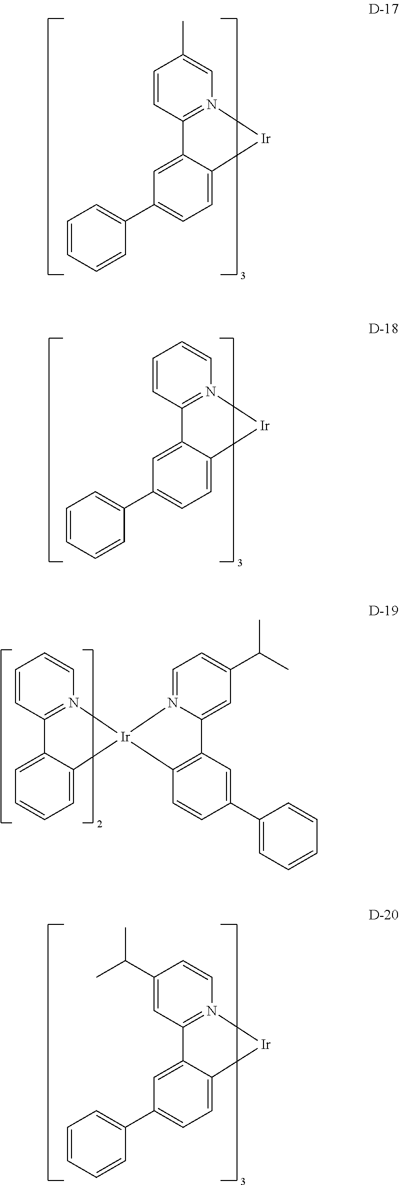

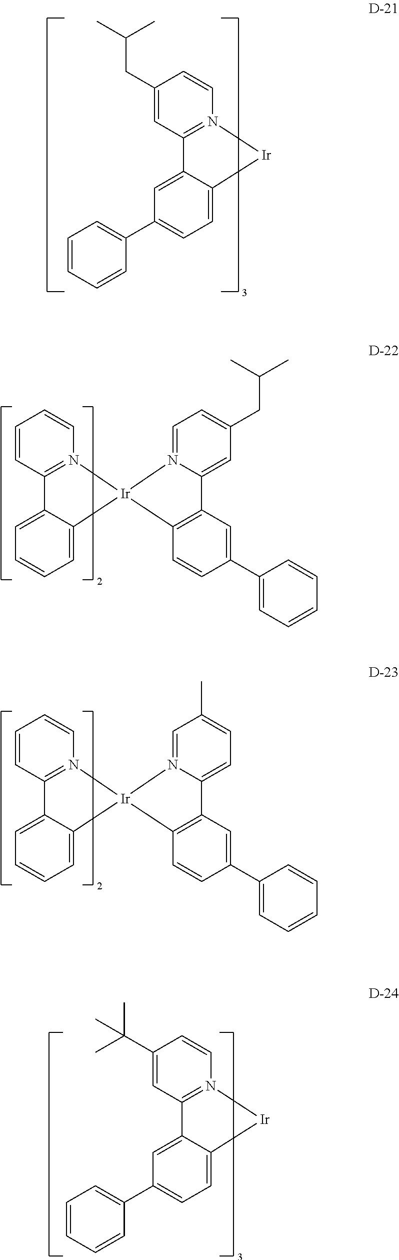

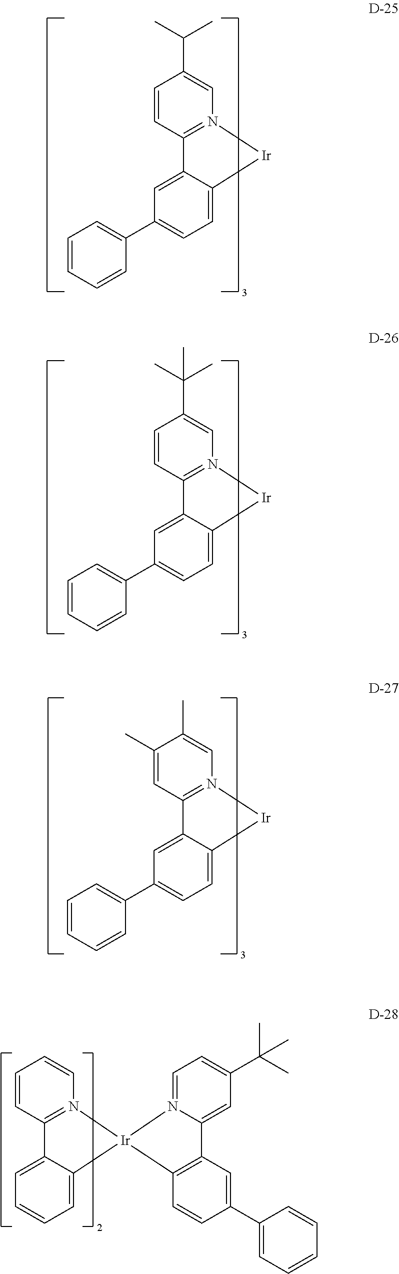

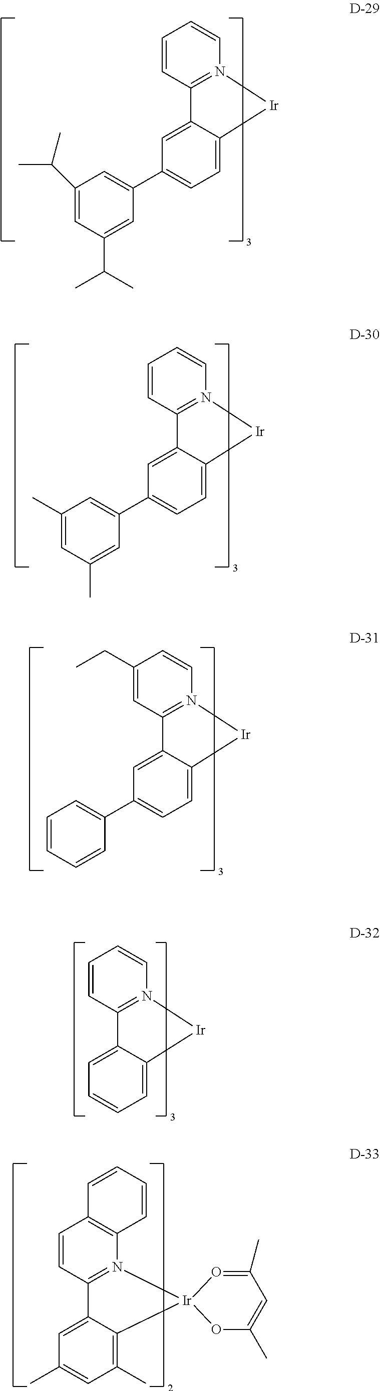

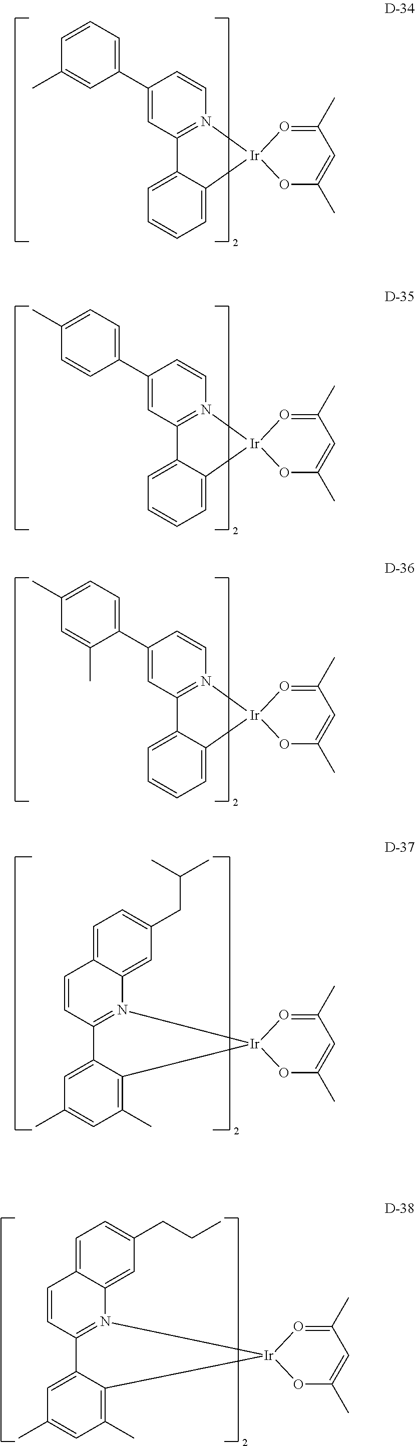

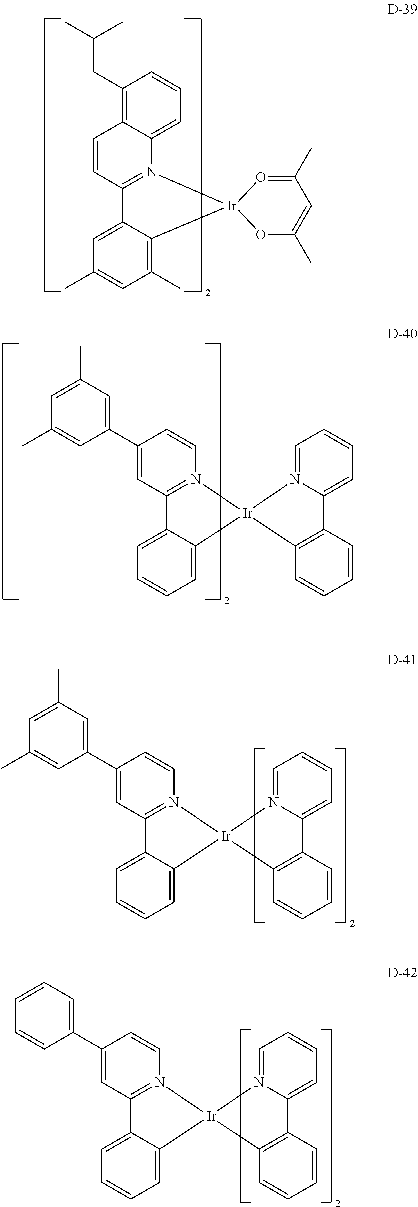

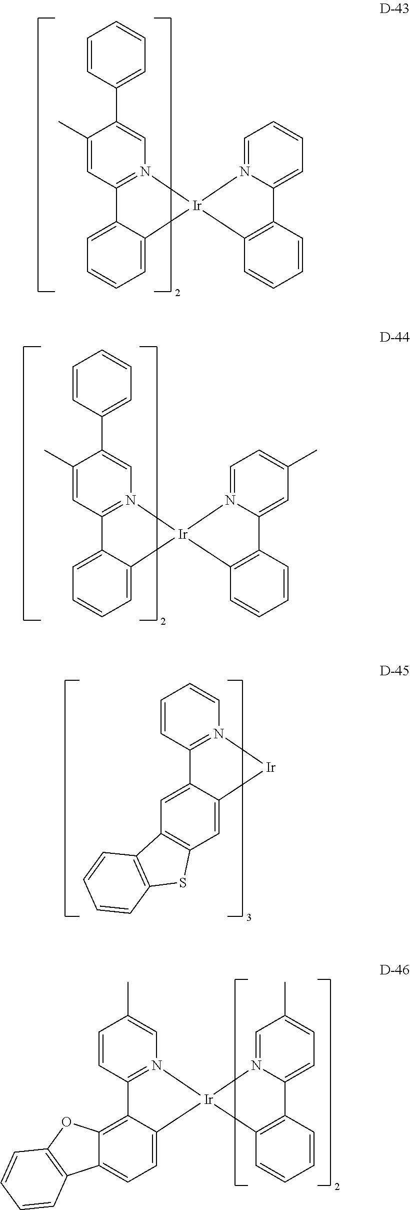

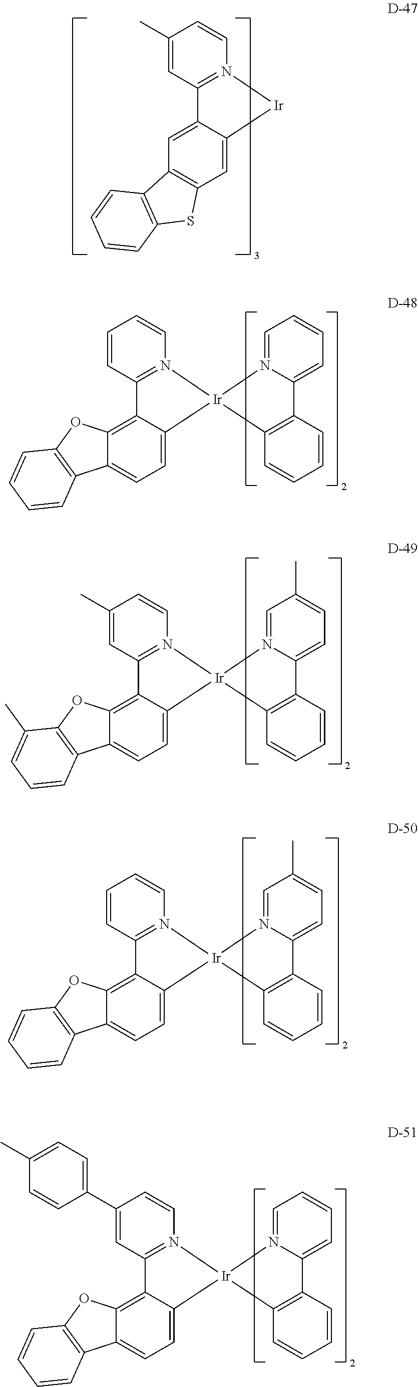

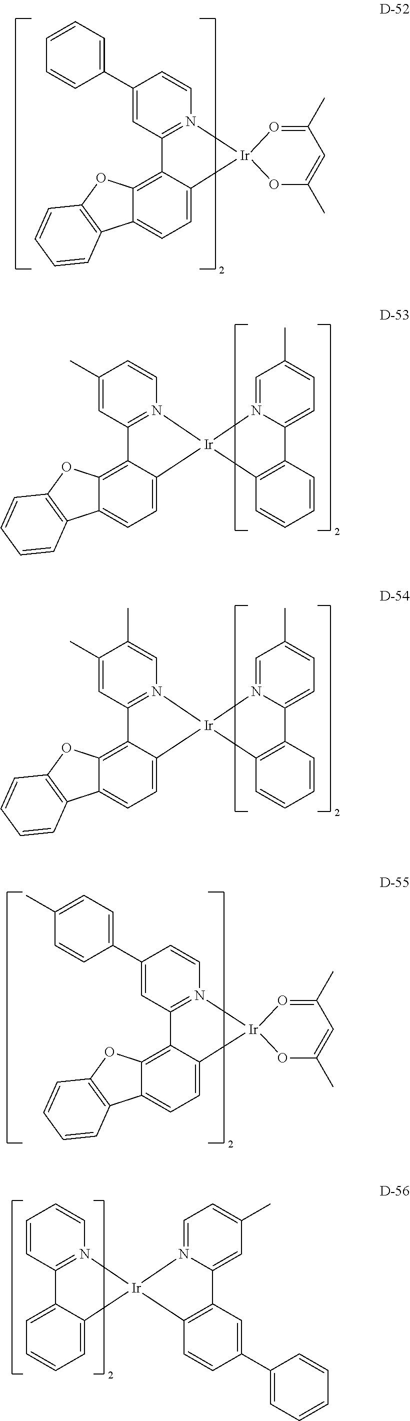

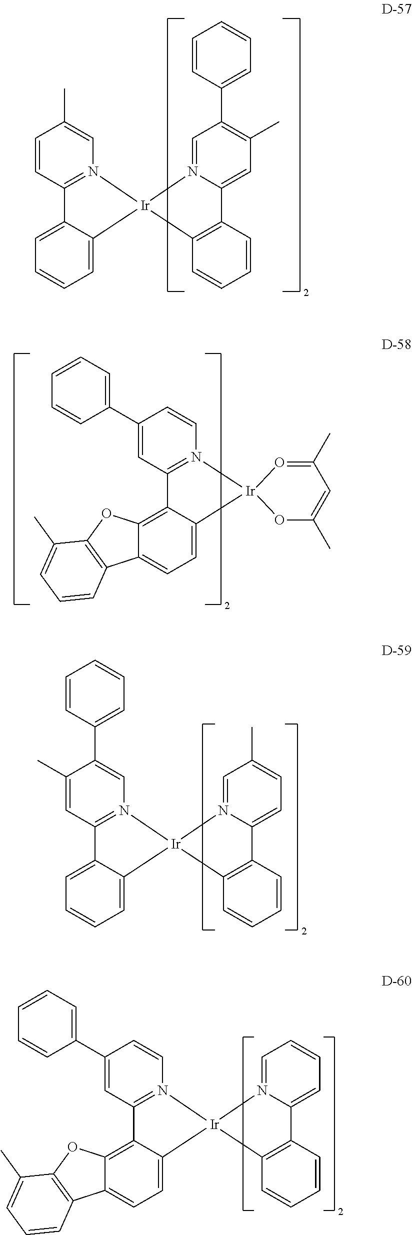

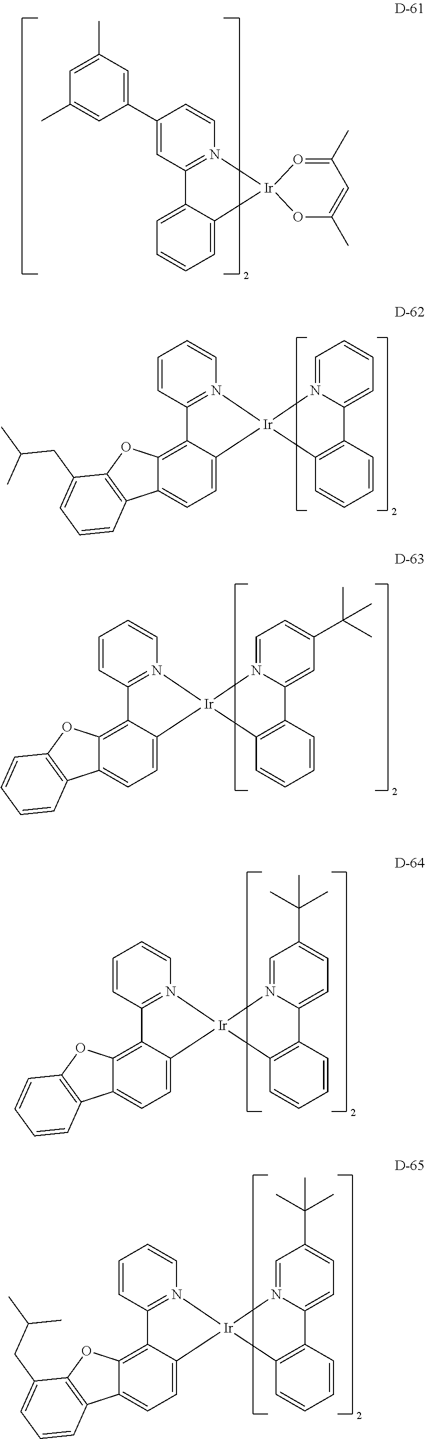

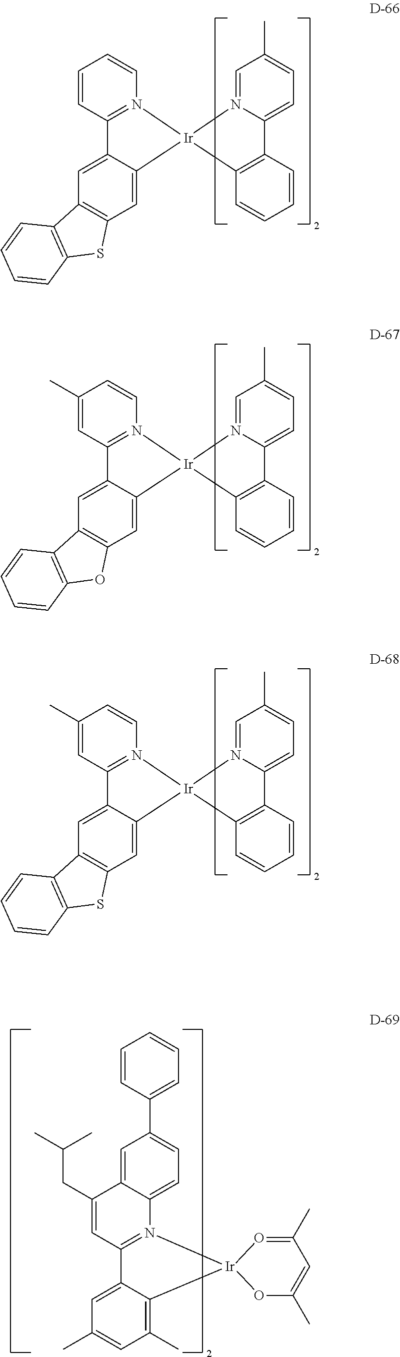

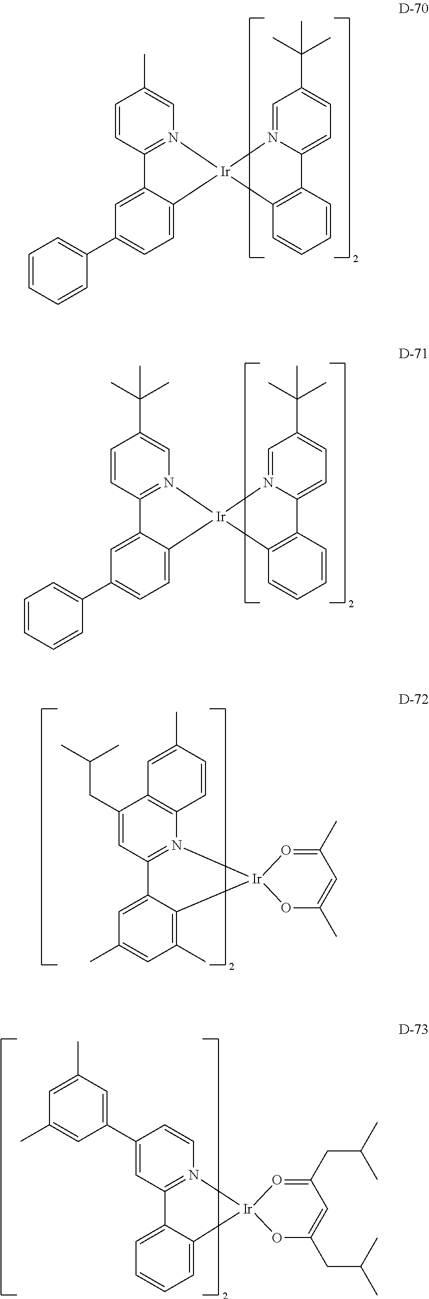

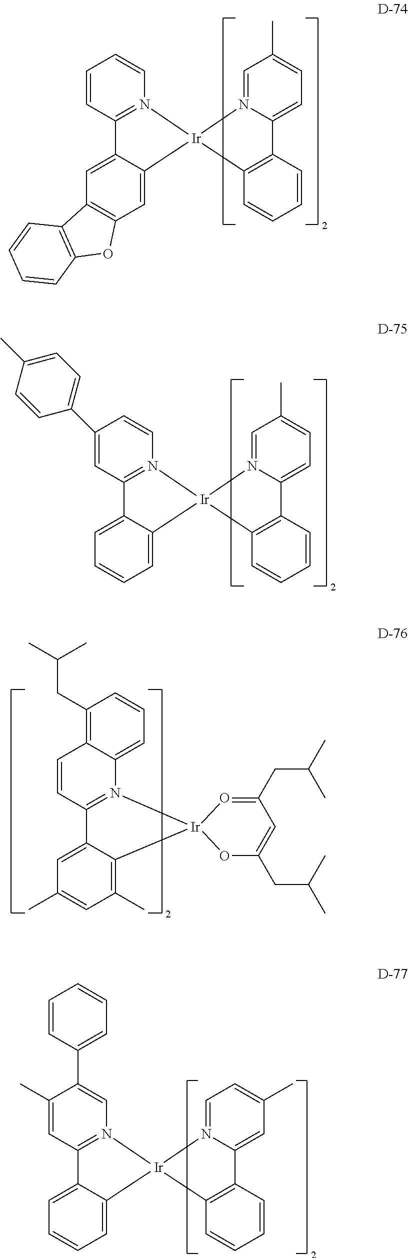

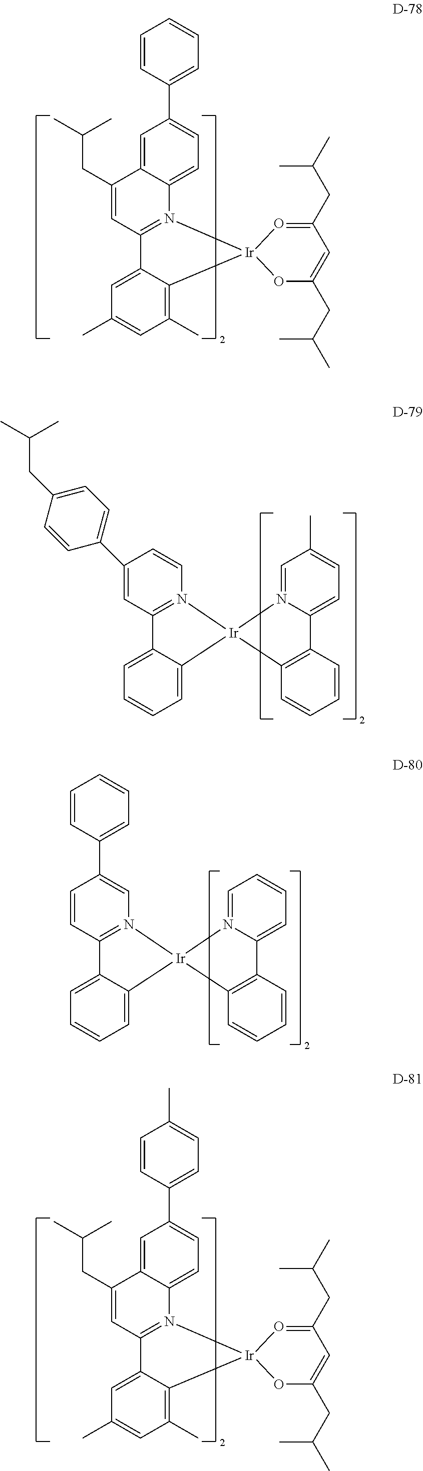

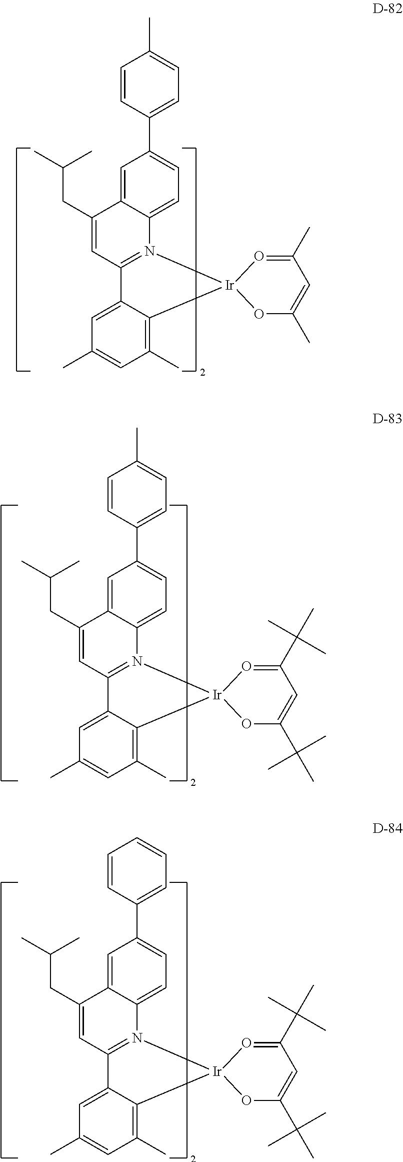

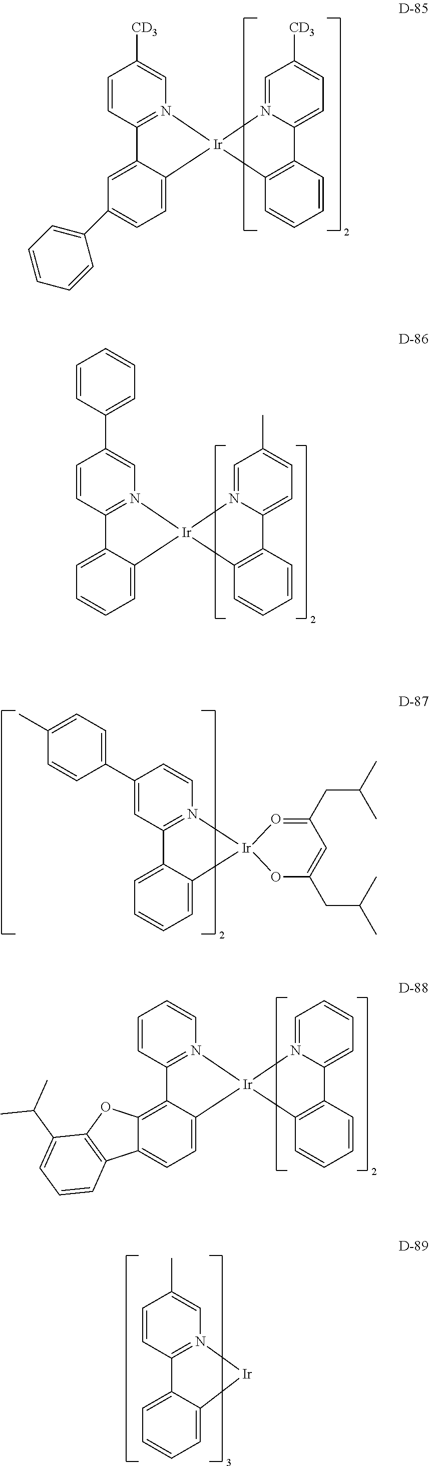

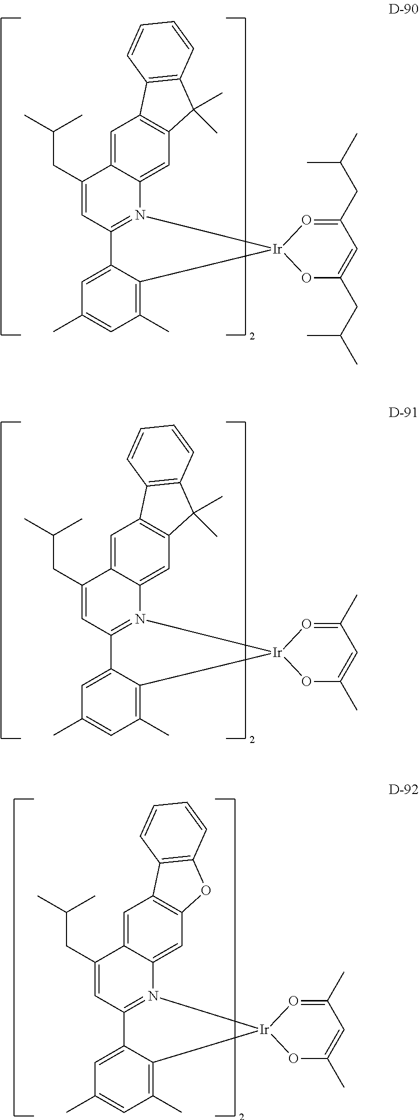

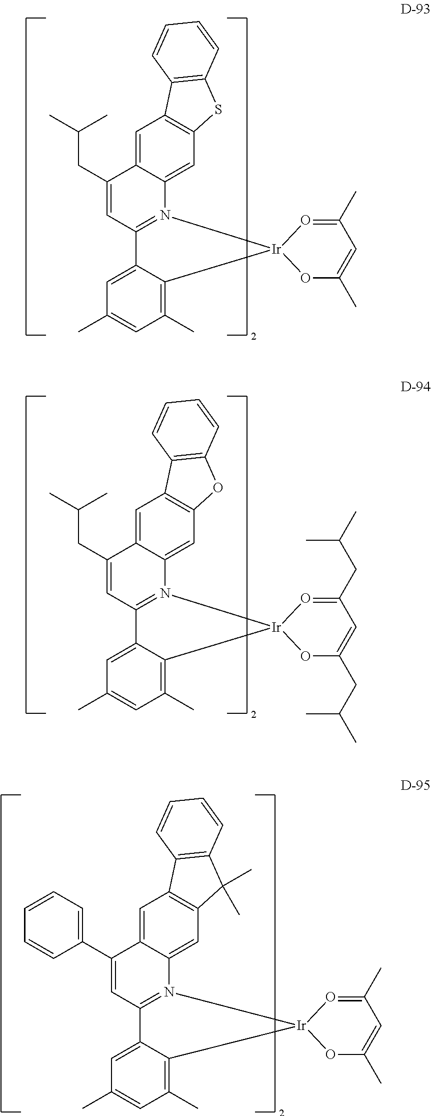

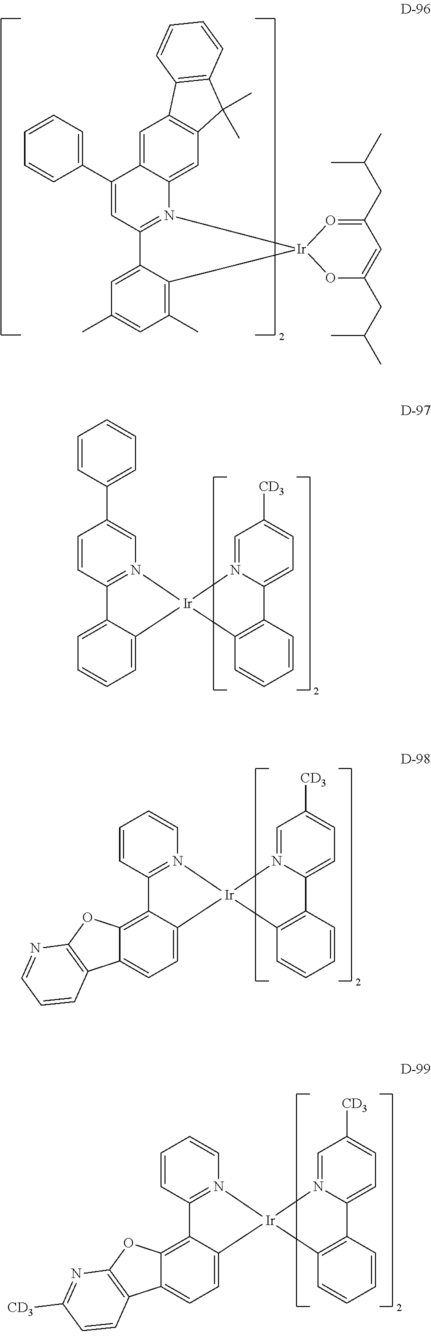

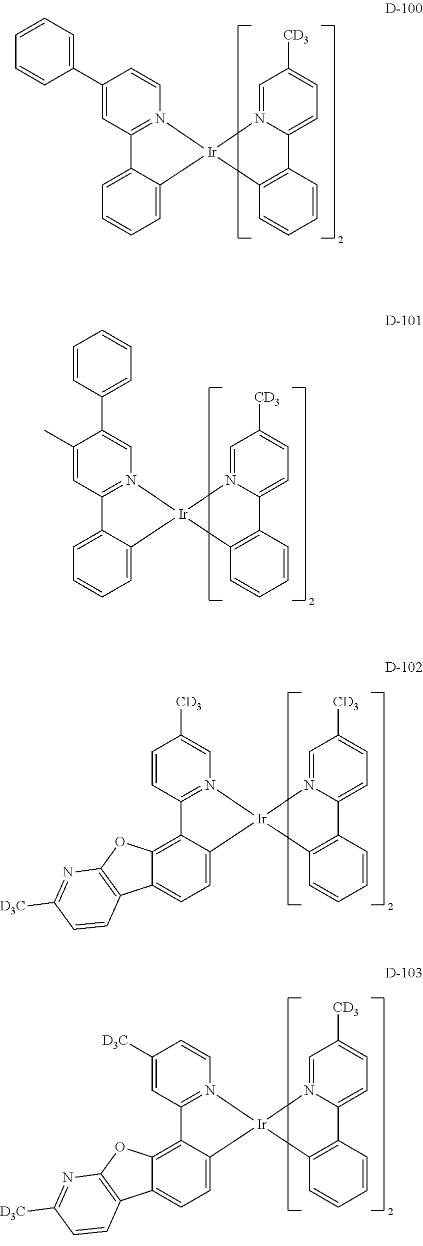

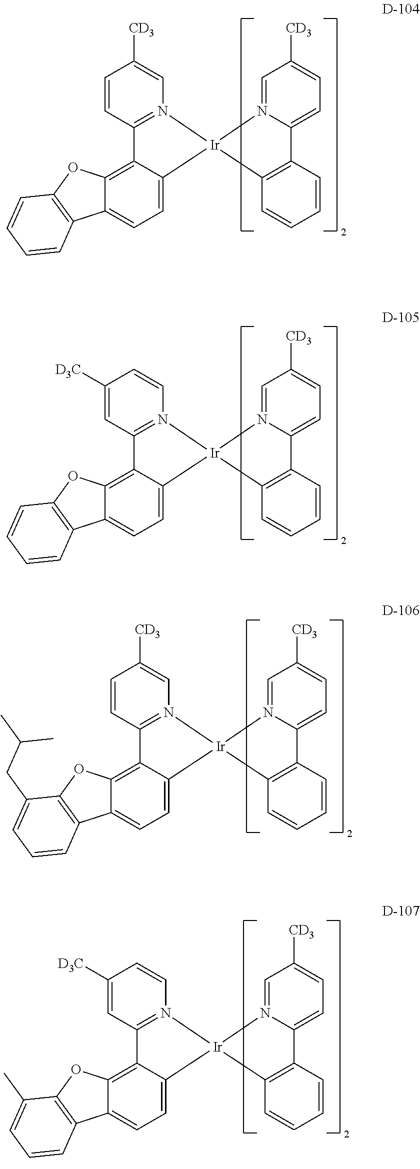

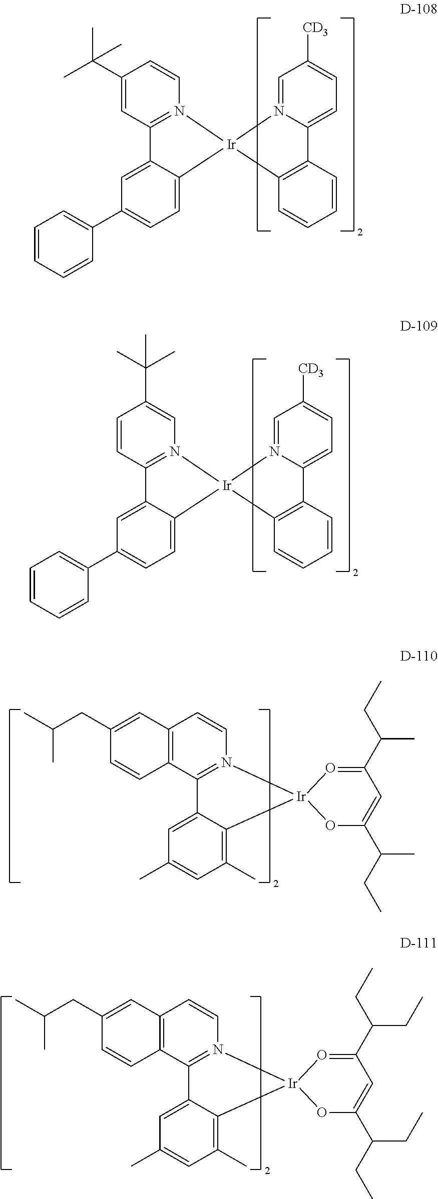

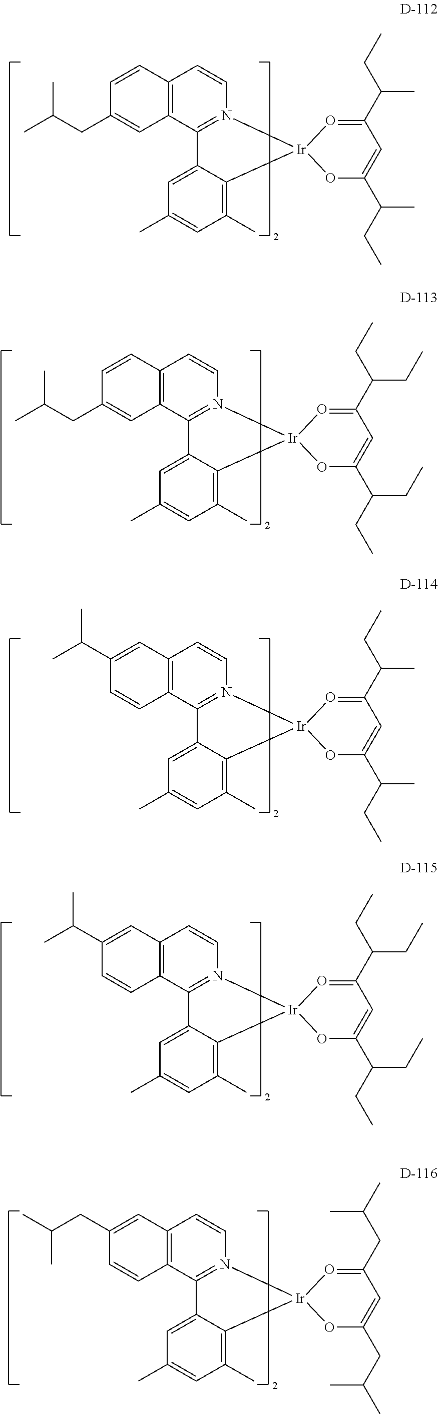

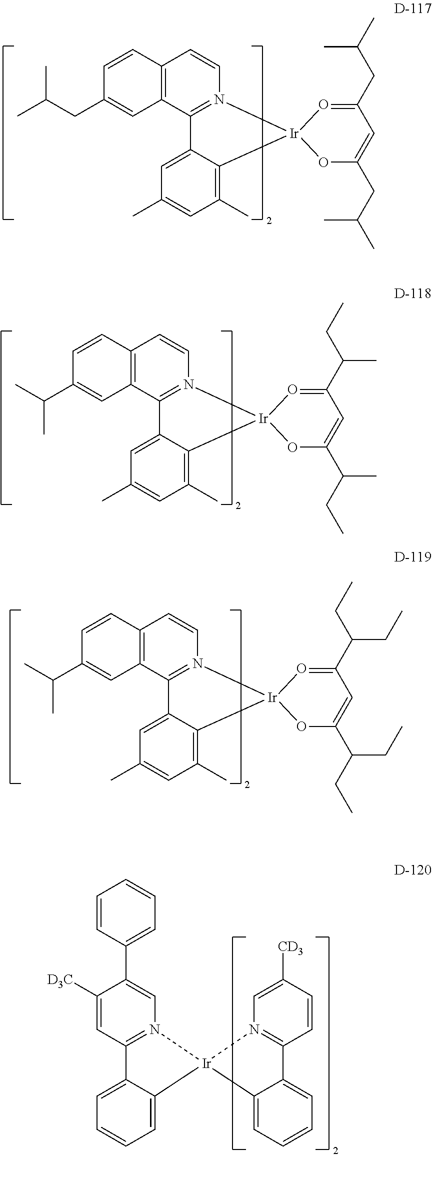

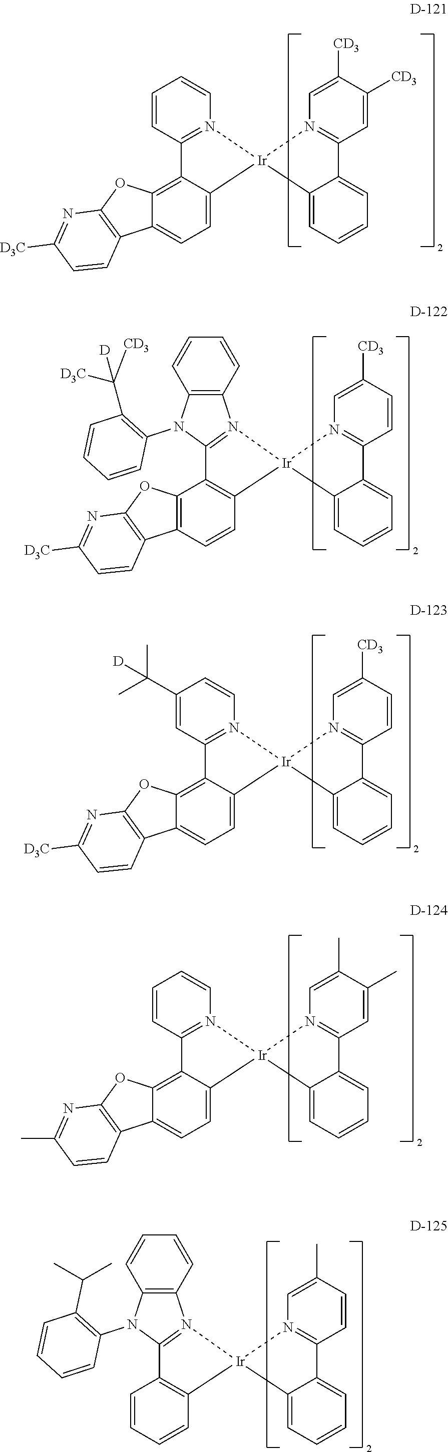

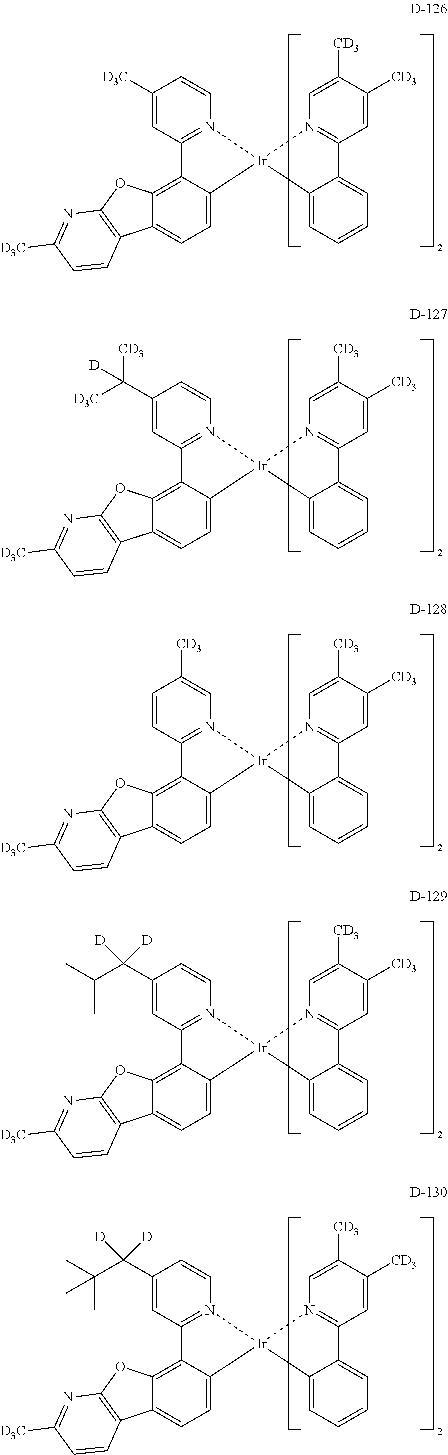

The organic electroluminescent device according to the present disclosure comprises a first electrode, a second electrode, and at least one organic layer between the first and second electrodes.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may further consist of multi-layers.

The first electrode and the second electrode may each be formed with a transmissive conductive material, a transflective conductive material, or a reflective conductive material. The organic electroluminescent device may be a top emission type, a bottom emission type, or both-sides emission type according to the kinds of the material forming the first electrode and the second electrode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

In addition, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal.

The organic electroluminescent device of the present disclosure may emit white light by further including at least one light-emitting layer containing a blue, red or green light-emitting compound, which is known in the art. In addition, it may further include a yellow or orange light-emitting layer, if necessary.

In the organic electroluminescent device of the present disclosure, at least one layer selected from a chalcogenide layer, a metal halide layer and a metal oxide layer (hereinafter, “a surface layer”) may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. The surface layer may provide operating stability for the organic electroluminescent device. Preferably, the chalcogenide includes SiOₓ (1≤X≤2), AlOₓ (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, or an electron blocking layer, or a combination thereof may be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The hole transport layer or the electron blocking layer may also be multilayers.

An electron buffer layer, a hole blocking layer, an electron transport layer, or an electron injection layer, or a combination thereof can be used between the light-emitting layer and the cathode. The electron buffer layer may be multilayers in order to control the injection of the electron and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. Further, the electron blocking layer may be placed between the hole transport layer (or hole injection layer) and the light-emitting layer, and may block overflowing electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to prepare an organic electroluminescent device having two or more light-emitting layers, which emits white light.

An organic electroluminescent material according to one embodiment of the present disclosure may be used as light-emitting materials for a white organic light-emitting device. The white organic light-emitting device has been suggested to have various structures such as a parallel arrangement (side-by-side) method, a stacking method, or a color conversion material (CCM) method, etc., according to the arrangement of R (red), G (green), YG (yellowish green), or B (blue) light-emitting units. In addition, the organic electroluminescent material according to one embodiment of the present disclosure may also be applied to the organic electroluminescent device comprising QD (quantum dot).

In order to form each layer of the organic electroluminescent device of the present disclosure, dry film-forming methods such as vacuum evaporation, sputtering, plasma, ion plating, etc., or wet film-forming methods such as ink jet printing, nozzle printing, slot coating, spin coating, dip coating, flow coating, etc., can be used. The first and second host compounds of the present disclosure may be co-evaporated or mixture-evaporated.

When using a wet film-forming method, a thin film can be formed by dissolving or diffusing the materials forming each layer into any suitable solvent such as ethanol, chloroform, tetrahydrofuran, dioxane, etc. The solvent is not specifically limited as long as the material constituting each layer is soluble or dispersible in the solvents, which do not cause any problems in forming a film.

It is possible to produce a display system, e.g., a display system for smartphones, tablets, notebooks, PCs, TVs, or cars, or a lighting system, e.g., an outdoor or indoor lighting system, by using the organic electroluminescent device of the present disclosure.

Hereinafter, the preparation method of the compound of the present disclosure, and the properties thereof will be explained in detail with reference to the representative compounds of the present disclosure. However, the present disclosure is not limited to the following examples.

### Example 1: Preparation of Compound C-1

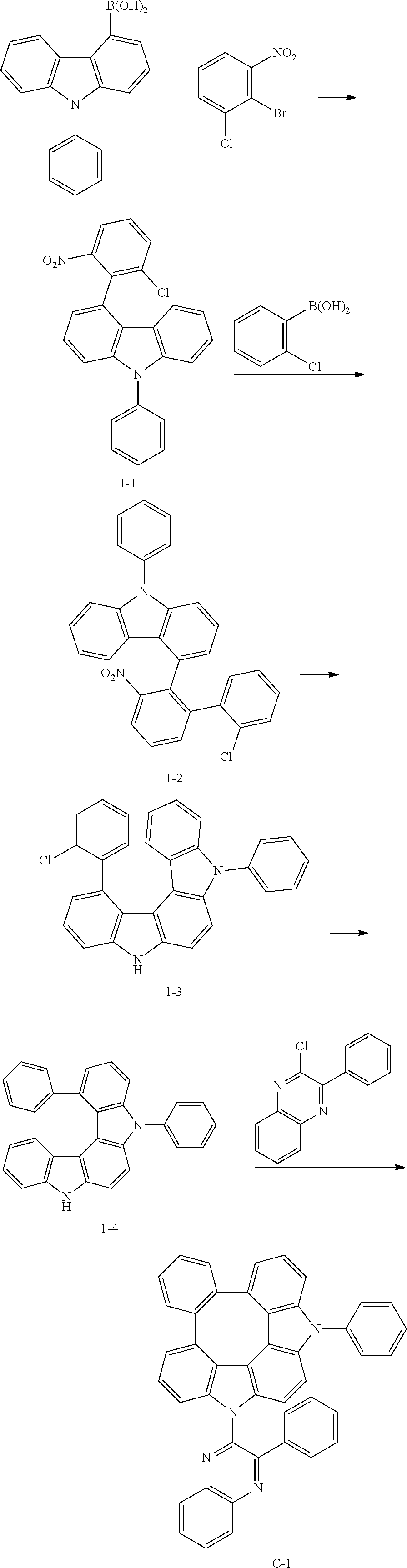

### 1) Synthesis of Compound 1-1

In a flask, 96 g of (9-phenyl-9H-carbazol-4-yl)boronic acid (334.3 mmol), 71.8 g of 2-bromo-1-chloro-3-nitrobenzene (304 mmol), 15 g of Pd₂(dba)₃ (16.71 mmol), 10.9 g of S-Phos (26.76 mmol), and 315 g of K₃PO₄ (1.64 mol) were dissolved in 1500 mL of toluene, and the mixture was stirred at 130° C. for 4 hours. After completion of the reaction, the organic layer was extracted with ethyl acetate and the residual moisture was removed by using magnesium sulfate. The residue was dried and separated by column chromatography to obtain 67 g of compound 1-1 (yield: 56.6%).

### 2) Synthesis of Compound 1-2

In a flask, 23.5 g of compound 1-1 (58.9 mmol), 18.4 g of (2-chlorophenyl)boronic acid (117.8 mmol), 2.7 g of Pd₂(dba)₃ (2.95 mmol), 2.4 g of S-Phos (5.89 mmol), and 63 g of K₃PO₄ (294.5 mmol) were dissolved in 300 mL of toluene, and the mixture was stirred at 130° C. for 12 hours. After completion of the reaction, the organic layer was extracted with ethyl acetate, and the residual moisture was removed by using magnesium sulfate. The residue was dried and separated by column chromatography to obtain 14 g of compound 1-2 (yield: 50%).

### 3) Synthesis of Compound 1-3

In a flask, 13 g of compound 1-2 (27.4 mmol) and 21.5 g of triphenylphosphine (82.1 mmol) were dissolved in 140 mL of o-DCB, and the mixture was stirred at 220° C. for 7 hours. After completion of the reaction, the reaction was removed by distillation and the residue was separated by column chromatography to obtain 4 g of compound 1-3 (yield: 32%).

### 4) Synthesis of Compound 1-4

In a flask, 10 g of compound 1-3 (22.5 mmol), 505 mg of Pd(OAc)₂ (2.25 mmol), 1.63 g of Pcy₃-HBF₄ (4.5 mmol), and 22 g of Cs₂CO₃ (67.5 mmol) were dissolved in 113 mL of o-Xylene, and the mixture was stirred at 160° C. for 4 hours. After completion of the reaction, the organic layer was extracted with ethyl acetate and the residual moisture was removed by using magnesium sulfate. The residue was dried and separated by column chromatography to obtain 1 g of compound 1-4 (yield: 11%).

### 5) Synthesis of Compound C-1

In a flask, 4.5 g of compound 1-4 (11.06 mmol), 4 g of 2-chloro-3-phenylquinoxaline (16.6 mmol), 67 mg of DMAP (0.553 mmol), and 10.8 g of Cs₂CO₃ (331.8 mmol) were dissolved in 60 mL of DMSO, and the mixture was refluxed at 140° C. for 4 hours. After completion of the reaction, the organic layer was extracted with ethyl acetate, and the residual moisture was removed by using magnesium sulfate. The residue was dried and separated by column chromatography to obtain 2.5 g of compound C-1 (yield: 37%).

| | MW | M.P. |
|---|---|---|
| C-1 | 610.22 | 246° C. |

### Example 2: Preparation of Compound C-29

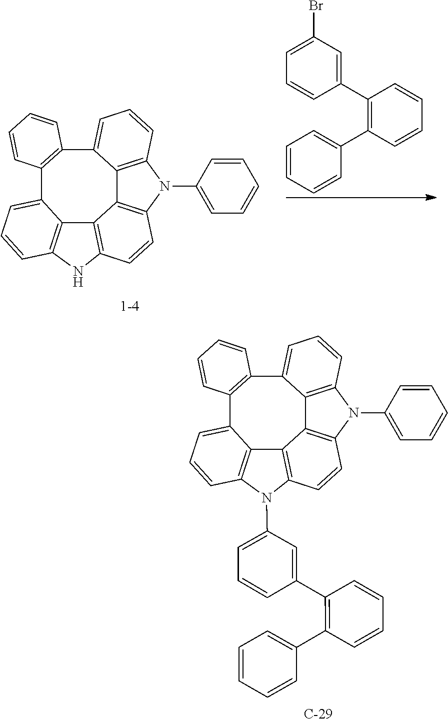

In a flask, 4 g of compound 1-4 (9.84 mmol), 3.65 g of 3-bromo-1,1′:2′,1″-terphenyl (11.8 mmol), 448 mg of Pd₂(dba)₃ (0.492 mmol), 448 mg of S-Phos (0.984 mmol), and 2.84 g of NaOtBu (29.52 mmol) were dissolved in 50 mL of o-Xylene, and the mixture was stirred at 170° C. for 4 hours. After completion of the reaction, the organic layer was extracted with ethyl acetate and the residual moisture was removed by using magnesium sulfate. The residue was dried and separated by column chromatography to obtain 1.5 g of compound C-29 (yield: 24%).

| | MW | M.P. |
|---|---|---|
| C-29 | 643.78 | 282° C. |

### Example 3: Preparation of Compound C-196

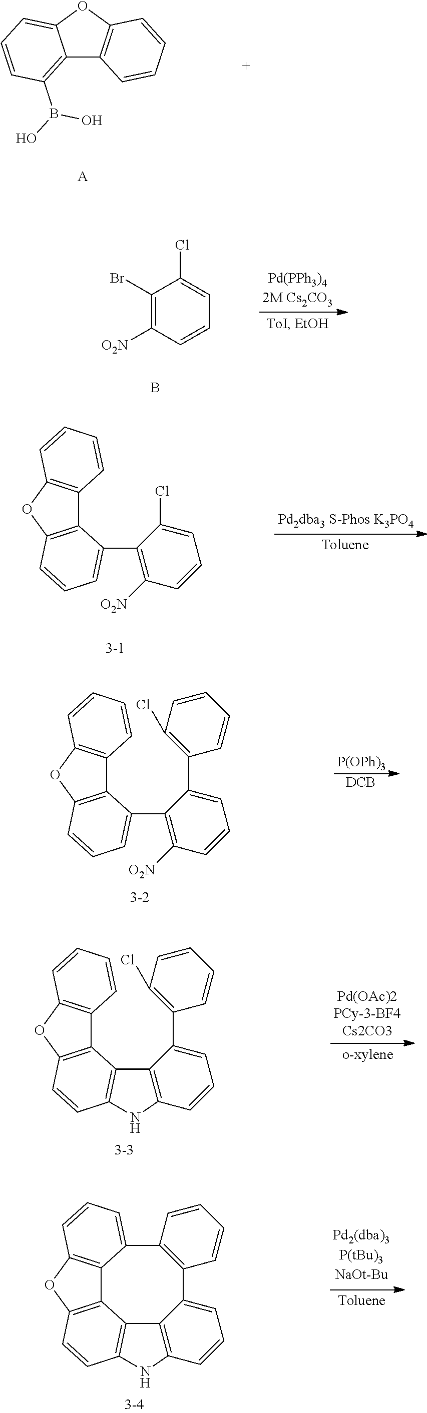

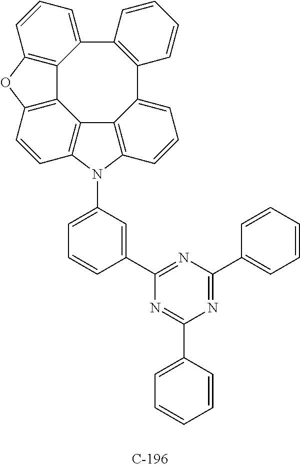 [SMILES: c1ccc(-c2nc(-c3ccccc3)nc(-c3cccc(-n4c5cccc6c7ccccc7c7cccc8oc9ccc4c(c9c87)c65)c3)n2)cc1]

### 1) Synthesis of Compound 3-1

In a reaction vessel, 60 g of compound A (283 mmol), 100 g of compound B (424 mmol), 16.3 g of tetrakis(triphenylphosphine)palladium (14.1 mmol), 276 g of cesium carbonate (849 mmol), 1400 mL of toluene, 350 mL of ethanol, and 350 mL of distilled water were added, and the mixture was stirred at 130° C. for 12 hours. After completion of the reaction, the reaction mixture was cooled to room temperature and an organic layer was extracted with ethyl acetate. The extracted organic layer was dried with magnesium sulfate and the solvent was removed by a rotary evaporator. The residue was separated by column chromatography to obtain 38 g of compound 3-1 (yield: 41%).

### 2) Synthesis of Compound 3-2

In a reaction vessel, 38 g of compound 3-1 (117 mmol), 35 g of phenylboronic acid (234 mmol), 5.3 g of tris(dibenzylideneacetone)dipalladium (5.86 mmol), 4.8 g of S-Phos (11.7 mmol), 62 g of tripotassium phosphate (293 mmol), and 600 mL of toluene were added, and the mixture was stirred under reflux for 2 hours. After completion of the reaction, the reaction mixture was washed with distilled water and an organic layer was extracted with ethyl acetate. The extracted organic layer was dried with magnesium sulfate and the solvent was removed by a rotary evaporator. The residue was separated by column chromatography to obtain 31 g of compound 3-2 (yield: 67%).

### 3) Synthesis of Compound 3-3

In a reaction vessel, 21 g of compound 3-2 (53.7 mmol), 70 mL of triphenyl phosphite (268 mmol), and 180 mL of DCB were added, and the mixture was stirred at 200° C. for 12 hours. After completion of the reaction, the reaction mixture was distilled under reduced pressure to remove DCB. The reaction mixture was washed with distilled water and an organic layer was extracted with ethyl acetate. The organic layer was dried with magnesium sulfate and the solvent was removed by a rotary evaporator. The residue was separated by column chromatography to obtain 10 g of compound 3-3 (yield: 55%).

### 4) Synthesis of Compound 3-4

In a reaction vessel, 6.6 g of compound 3-3 (17.9 mmol), 0.2 g of palladium(II) acetate (0.89 mmol), 1.3 g of PCy3-BF4 (3.58 mmol), 17 g of cesium carbonate (53.7 mmol), and 90 mL of o-xylene were added, and the mixture was stirred under reflux at 160° C. for 4 hours. After completion of the reaction, the reaction mixture was washed with distilled water and an organic layer was extracted with ethyl acetate. The extracted organic layer was dried with magnesium sulfate and the solvent was removed by a rotary evaporator. The residue was separated by column chromatography to obtain 1.8 g of compound 3-4 (yield: 32%).

### 5) Synthesis of Compound C-196

In a reaction vessel, 1.8 g of compound 3-4 (5.43 mmol), 2.3 g of 2-(3-bromophenyl)-4,6-diphenyl-1,3,5-triazine (5.97 mmol), 0.2 g of tris(dibenzylideneacetone)dipalladium (0.27 mmol), 0.3 mL of tri-tert-butylphosphine (0.54 mmol), 1.3 g of sodium tert-butoxide (13.5 mmol), and 30 mL of toluene were added, and the mixture was stirred under reflux for 3 hours. After completion of the reaction, the reaction mixture was washed with distilled water and an organic layer was extracted with ethyl acetate. The organic layer was dried with magnesium sulfate and the solvent was removed by a rotary evaporator. The residue was separated by column chromatography to obtain 3.3 g of compound C-196 (yield: 95%).

| | MW | UV | PL | M.P. |
|---|---|---|---|---|
| C-196 | 638.21 | 410 nm | 522 nm | 240° C. |

### Example 4: Preparation of Compound C-36

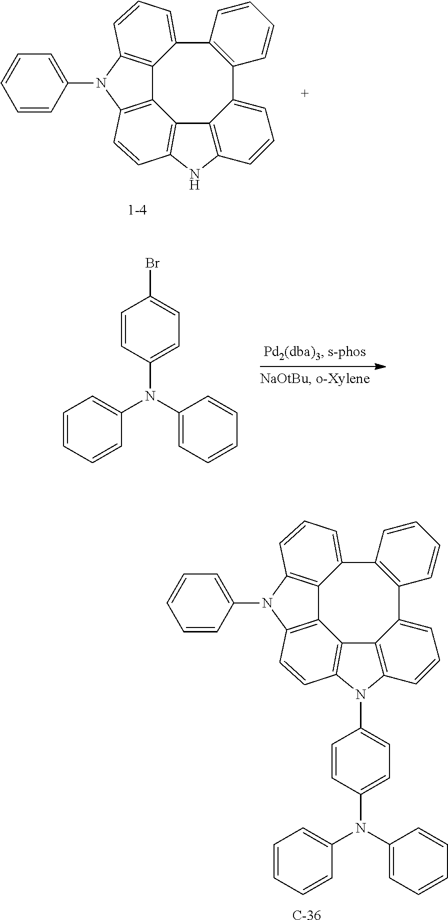

In a flask, 4.0 g of compound 1-4 (9.84 mmol), 3.2 g of 4-bromo-N,N-diphenylaniline (9.84 mmol), 0.45 g of Pd₂(dba)₃ (0.5 mmol), 0.4 g of s-phos (0.98 mmol), and 1.9 g of NaOtBu (19.7 mmol) were dissolved in 50 mL of o-Xylene, and the mixture was stirred under reflux for 5 hours. After completion of the reaction, the organic layer was extracted with ethyl acetate. The residue was separated by column chromatography to obtain 2.67 g of compound C-36 (yield: 42%).

| | MW | M.P. |
|---|---|---|
| C-36 | 649.78 | 312° C. |

### Example 5: Preparation of Compound C-32

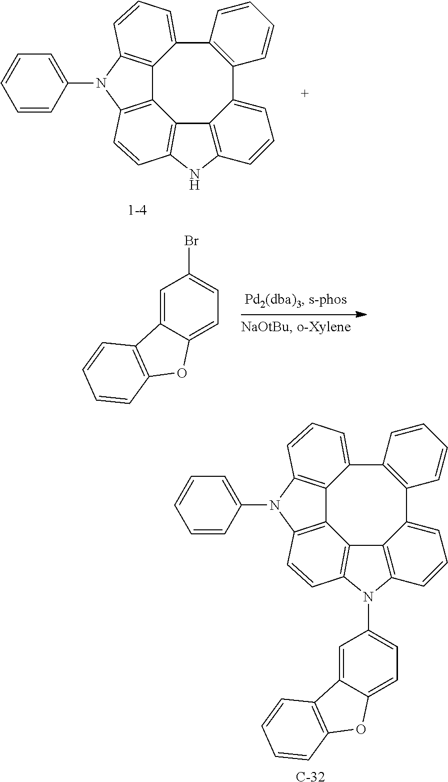

In a flask, 4.0 g of compound 1-4 (9.84 mmol), 1.7 g of 2-bromodibenzo[b,d]furan (9.84 mmol), 0.45 g of Pd₂(dba)₃ (0.5 mmol), 0.4 g of s-phos (0.98 mmol), and 1.9 g of NaOtBu (19.7 mmol) were dissolved in 50 mL of o-Xylene, and the mixture was stirred under reflux for 5 hours. After completion of the reaction, the organic layer was extracted with ethyl acetate. The residue was separated by column chromatography to obtain 1.68 g of compound C-32 (yield: 30%).

| | MW | M.P. |
|---|---|---|
| C-32 | 572.65 | 291° C. |

### Device Examples 1-1 and 1-2: Producing an OLED Deposited with a Compound According to the Present Disclosure as a Host

An OLED according to the present disclosure was produced as follows: A transparent electrode indium tin oxide (ITO) thin film (10 Ω/sq) on a glass substrate for an OLED (GEOMATEC CO., LTD., Japan) was subjected to an ultrasonic washing with acetone, ethanol and distilled water, sequentially, and then was stored in isopropanol. The ITO substrate was mounted on a substrate holder of a vacuum vapor deposition apparatus. Compound HI-1 was introduced into a cell of the vacuum vapor deposition apparatus, and the pressure in the chamber of the apparatus was then controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into another cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The host material shown in Table 1 was introduced into one cell of the vacuum vapor depositing apparatus as a host, and compound D-39 was introduced into another cell as a dopant. The two materials were evaporated at different rates and the dopant was deposited in a doping amount of 3 wt % based on the total amount of the host and dopant to form a light-emitting layer having a thickness of 40 nm on the second hole transport layer. Next, compound ET-1 and compound El-1 were evaporated at a rate of 1:1 in two other cells to deposit an electron transport layer having a thickness of 35 nm on the light-emitting layer. After depositing compound El-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Comparative Example 1-1: Producing an OLED Deposited with a Comparative Compound as a Host

An OLED was produced in the same manner as in Device Example 1-1, except that compound A was used as a host of the light-emitting layer.

The results of the operating voltage, luminous efficiency, and CIE color coordinates at a luminance of 1,000 nit, and the time taken for luminance to decrease from 100% to 95% at a luminance of 5,500 nit (lifetime, T95) of the OLEDs produced in Device Examples 1-1 and 1-2 and Comparative Example 1-1 are provided in Table 1 below.

TABLE 1

| | | Operating | Luminous | | | |
|---|---|---|---|---|---|---|
| | | Voltage | Efficiency | CIE | | Lifetime |
| | Host | [V] | [cd/A] | x | y | (T95) [hr] |
| Comparative | A | 9.0 | 12.5 | 0.651 | 0.342 | 0.3 |
| Example 1-1 | | | | | | |
| Device | C-196 | 3.1 | 18.2 | 0.657 | 0.342 | 13.9 |
| Example 1-1 | | | | | | |
| Device | C-1 | 3.2 | 27.1 | 0.659 | 0.341 | 91.6 |
| Example 1-2 | | | | | | |

From Table 1, it can be confirmed that the OLED comprising the organic electroluminescent compound according to the present disclosure as a host has lower operating voltage, higher luminous efficiency, and longer lifetime than the OLED comprising the comparative compound of the Comparative Example.

Without intending to be limited by theory, it is understood that the compound of the present disclosure has a rigid planar structure, thereby reducing steric hindrance energy. In addition, it is understood that the compound of the present disclosure can not only increase hole stability in an OLED, but also increase hole mobility by increasing HOMO energy levels, thereby achieving a charge balance.

### Device Example 2-1: Producing an OLED Deposited with a Plurality of Host Materials According to the Present Disclosure

An OLED was produced in the same manner as in Device Example 1-1, except that a transparent electrode indium tin oxide (ITO) thin film (10 Ω/sq) on a glass substrate for an OLED (GEOMATEC CO., LTD., Japan) was subjected to an ultrasonic washing with acetone, trichloroethylene, ethanol and distilled water, sequentially, and then was stored in isopropanol; and a light-emitting layer was formed as follows: The first and second host compounds shown in Table 2 below were introduced into two cells of the vacuum vapor depositing apparatus as hosts, and compound D-39 was introduced into another cell as a dopant. The two host materials were evaporated at a rate of 1:1 and the dopant material was simultaneously evaporated at a different rate and the dopant was deposited in a doping amount of 3 wt % based on the total amount of the hosts and dopant to form a light-emitting layer having a thickness of 40 nm on the second hole transport layer.

### Comparative Example 2-1: Producing an OLED Deposited with a Comparative Compound as a Host

An OLED was produced in the same manner as in Device Example 2-1, except that the compound shown in Table 2 was used as a host of the light-emitting layer.

The results of the luminous efficiency and its increase rate, and the time taken for luminance to decrease from 100% to 97% (lifetime, T97) at a luminance of 5,000 nit of the OLEDs produced in Device Example 2-1 and Comparative Example 2-1 are provided in Table 2 below.

TABLE 2

| | | | Luminous | | |
|---|---|---|---|---|---|
| | First | Second | Efficiency | Increase Rate in | Lifetime |
| | Host | Host | [cd/A] | Efficiency [%] | (T97) [hr] |
| Comparative | — | H-2 | 17.8 | — | 24 |
| Example 2-1 | | | | | |
| Device | C-29 | H-2 | 29.6 | 66 | 267 |
| Example 2-1 | | | | | |

### Device Examples 3-1 and 3-2: Producing a Red OLED Deposited with a Plurality of Host Materials According to the Present Disclosure

An OLED according to the present disclosure was produced as follows: A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-3 shown in Table 4 below was introduced into a cell of the vacuum vapor deposition apparatus, and compound HT-1 shown in Table 4 below was introduced into another cell of the vacuum vapor deposition apparatus. The two materials were evaporated at different rates and compound HI-3 was deposited in a doping amount of 3 wt % based on the total amount of compound HI-3 and compound HT-1 to form a first hole injection layer having a thickness of 10 nm on the ITO substrate. Next, compound HT-1 was deposited on the first hole injection layer to form a first hole transport layer having a thickness of 80 nm. Subsequently, compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first and second host compounds shown in Table 3 below were introduced into two cells of the vacuum vapor depositing apparatus as hosts, and compound D-39 was introduced into another cell. The two host materials were evaporated at a rate of 1:1 and the dopant material was simultaneously evaporated at a different rate and the dopant was deposited in a doping amount of 3 wt % based on the total amount of the hosts and dopant to form a light-emitting layer having a thickness of 40 nm on the second hole transport layer. Next, compound ET-1 and compound El-1 as electron transport materials were evaporated at a weight ratio of 50:50 to deposit an electron transport layer having a thickness of 35 nm on the light-emitting layer. After depositing compound El-1 as an electron injection layer having a thickness of 2 nm on the electron transport layer, an A₁ cathode having a thickness of 80 nm was deposited on the electron injection layer by another vacuum vapor deposition apparatus. Thus, an OLED was produced. Each compound was used after purification by vacuum sublimation under 10⁻⁶ torr for each material.

The results of the operating voltage, luminous efficiency, and luminous color at a luminance of 1,000 nit, and the time taken for luminance to decrease from 100% to 95% at a luminance of 5,500 nit (lifetime, T95) of the OLEDs produced in Device Examples 3-1 and 3-2 are provided in Table 3 below.

TABLE 3

| | | | Operating | Luminous | | |
|---|---|---|---|---|---|---|
| | First | | Voltage | Efficiency | | |
| | Host | Second Host | [V] | [cd/A] | Luminous Color | Lifetime (T95) [hr] |
| Device | C-36 | H-2 | 3.0 | 30.8 | Red | 308 |
| Example 3-1 | | | | | | |
| Device | C-32 | H-2 | 3.1 | 33.2 | Red | 705 |
| Example 3-2 | | | | | | |

From Tables 2 and 3, it can be confirmed that the OLEDs comprising a specific combination of compounds according to the present disclosure as a host material have significantly improved efficiency and lifetime compared to conventional OLEDs.

The compounds used in the Device Examples and the Comparative Examples are shown in Table 4 below.

TABLE 4

| | |
|---|---|
| Hole injection Layer/ Hole Transport Layer | 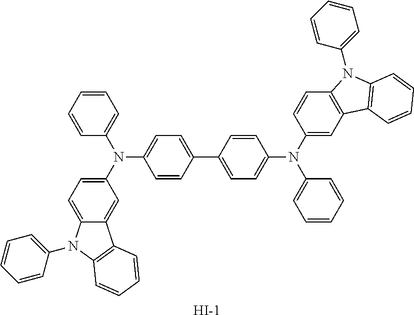 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 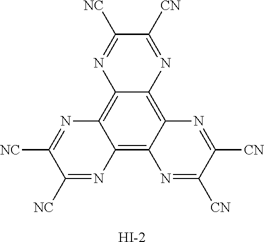 |
| | 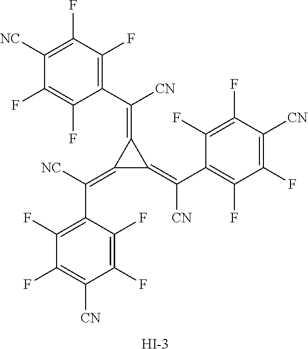 |
| | 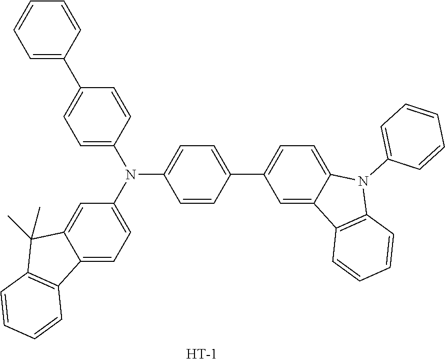 |
| | 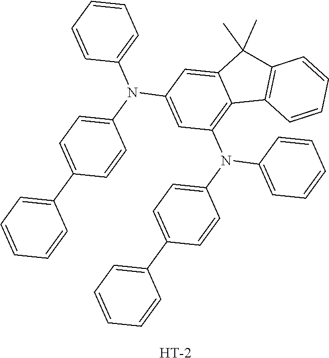 |
| Light- Emitting Layer | 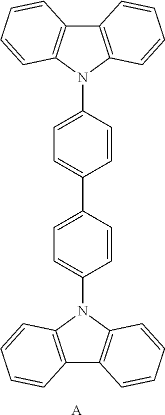 |
| | 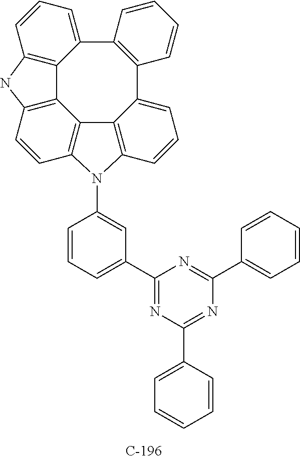 [SMILES: c1ccc(-c2nc(-c3ccccc3)nc(-c3cccc(-n4c5cccc6c7ccccc7c7cccc8[nH]c9ccc4c(c9c87)c65)c3)n2)cc1] |
| | C-1 |
| | 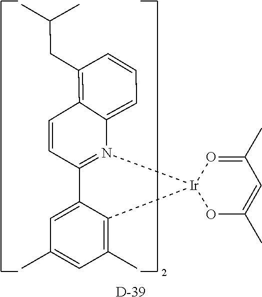 |
| | C-29 |
| | 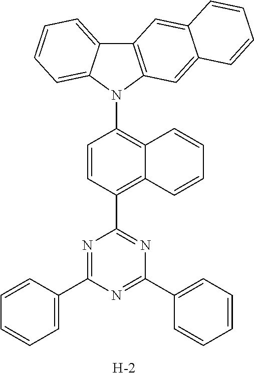 |
| | 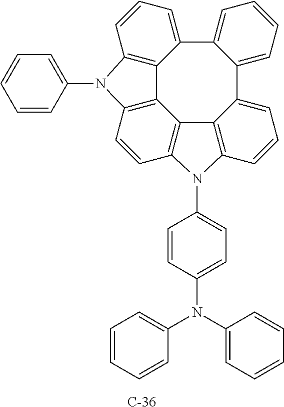 |
| | 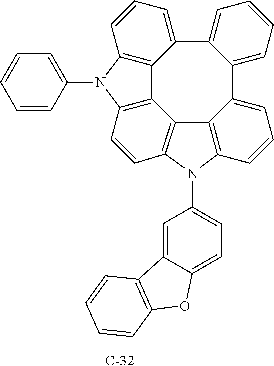 |
| Electron Transport Layer/ Electron Injection Layer | 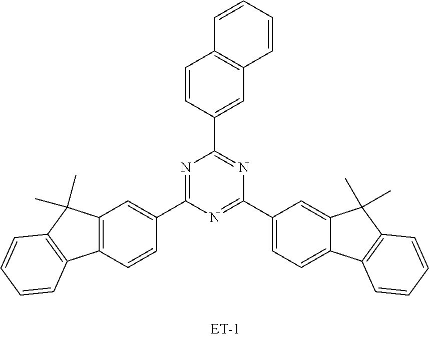 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 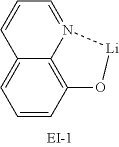 |

1. An organic electroluminescent compound represented by the following formula 1: 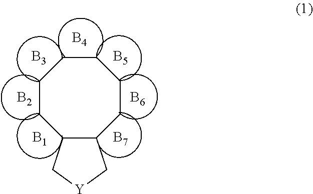
wherein
B₁ to B₇, each independently, are not present or represent a substituted or unsubstituted (C5-C20) ring, in which the carbon atom of the ring may be replaced with one or more heteroatoms selected from nitrogen, oxygen and sulfur; with the proviso that at least five of B₁ to B₇ are present, and the adjacent rings of B₁ to B₇ are fused with each other;
Y represents —N-L₁-(Ar₁)ₙ, —O—, —S—, or —CR₁R₂;
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ represents a substituted or unsubstituted (C6-C30)aryl, a substituted or unsubstituted (3- to 30-membered)heteroaryl, or —NR₃R₄;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to an adjacent substituent(s) to form a ring(s); and
n represents an integer of 1 or 2; where if n represents 2, each of Ar₁ may be the same as or different from each other.

2. The organic electroluminescent compound according to claim 1, wherein B₁ to B₇, each independently, are not present or represent a substituted or unsubstituted benzene ring, a substituted or unsubstituted naphthalene ring, a substituted or unsubstituted pyrrole ring, a substituted or unsubstituted furan ring, a substituted or unsubstituted thiophene ring, a substituted or unsubstituted cyclopentadiene ring, a substituted or unsubstituted fluorene ring, a substituted or unsubstituted pyridine ring, or a substituted or unsubstituted dibenzofuran ring, with the proviso that at least five of B₁ to B₇ are present, and the adjacent rings of B₁ to B₇ are fused with each other.

3. The organic electroluminescent compound according to claim 1, wherein the formula 1 is represented by any one of the following formulas 1-1 to 1-5: 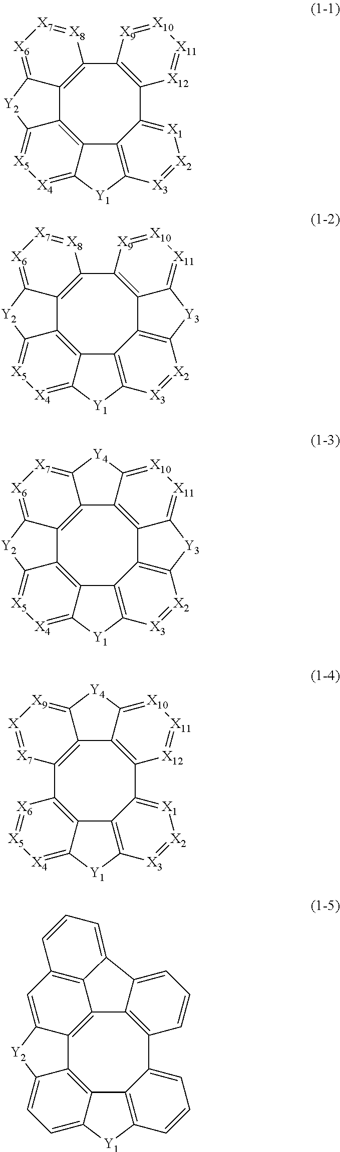
wherein
Y₁, Y₂, Y₃, and Y₄, each independently, are the same as the definition of Y in claim 1, and where if a plurality of Ar₁ is present, each of Ar₁ may be the same as or different from each other;
X₁ to X₁₂, each independently, represent —N═ or —C(Rₐ)=; and
Rₐ, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or adjacent Rₐ's may be linked to each other to form a ring(s); and where if a plurality of Rₐ is present, each of Rₐ may be the same as or different from each other.

4. The organic electroluminescent compound according to claim 3, wherein at least one of Ar₁(s) and Rₐ(s) represents any one selected from those listed in the following Group 1: 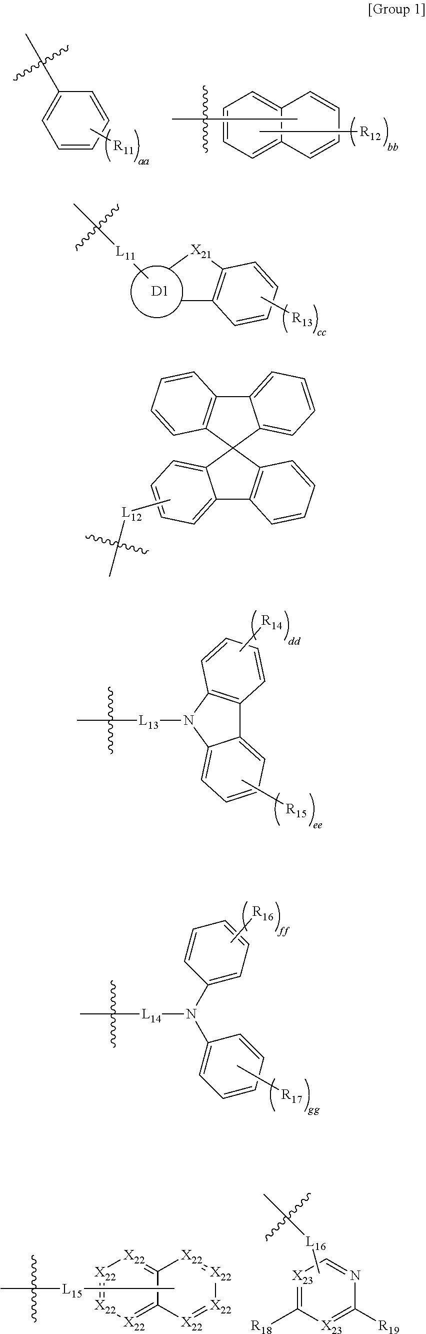 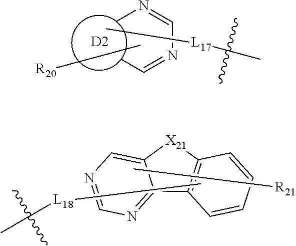
in Group 1,
D1 and D2, each independently, represent a benzene ring or a naphthalene ring;
X₂₁ represents O, S, NRs, or CR₆R₇;
X₂₂, each independently, represent CR₈ or N; with the proviso that at least one of X₂₂ represents N;
X₂₃, each independently, represent CR₉ or N;
L₁₁ to L₁₈, each independently, represent a single bond, a substituted or unsubstituted (C6-C30)arylene, or a substituted or unsubstituted (3- to 30-membered)heteroarylene;
R₁₁ to R₂₁, and R₅ to R₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to an adjacent substituent(s) to form a ring(s);
aa, ff, and gg, each independently, represent an integer of 1 to 5; bb represents an integer of 1 to 7; and cc, dd, and ee, each independently, represent an integer of 1 to 4.

5. The organic electroluminescent compound according to claim 3, wherein the substituents of the substituted (C1-C30)alkyl(ene), the substituted (C6-C30)aryl(ene), the substituted (3- to 30-membered)heteroaryl(ene), and the substituted (C3-C30)cycloalkyl(ene) in Ar₁, L₁, R₁ to R₄, and Rₐ, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one selected from the group consisting of deuterium and a (3- to 30-membered)heteroaryl(s); a (3- to 30-membered)heteroaryl unsubstituted or substituted with a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

6. The organic electroluminescent compound according to claim 3, wherein at least one of Ar₁(s) and Rₐ(s) represents any one selected from those listed in the following Groups 2 and 3: 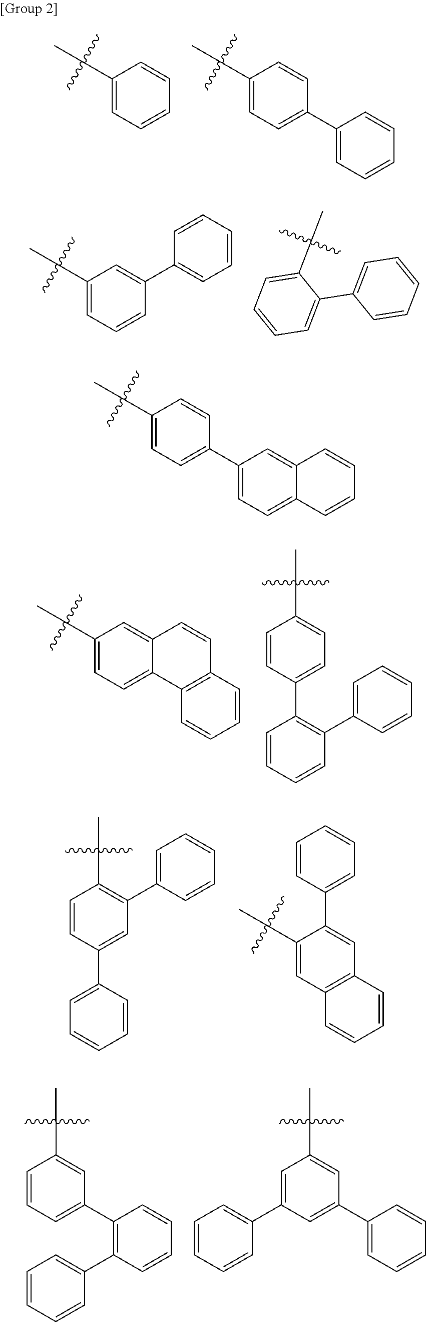 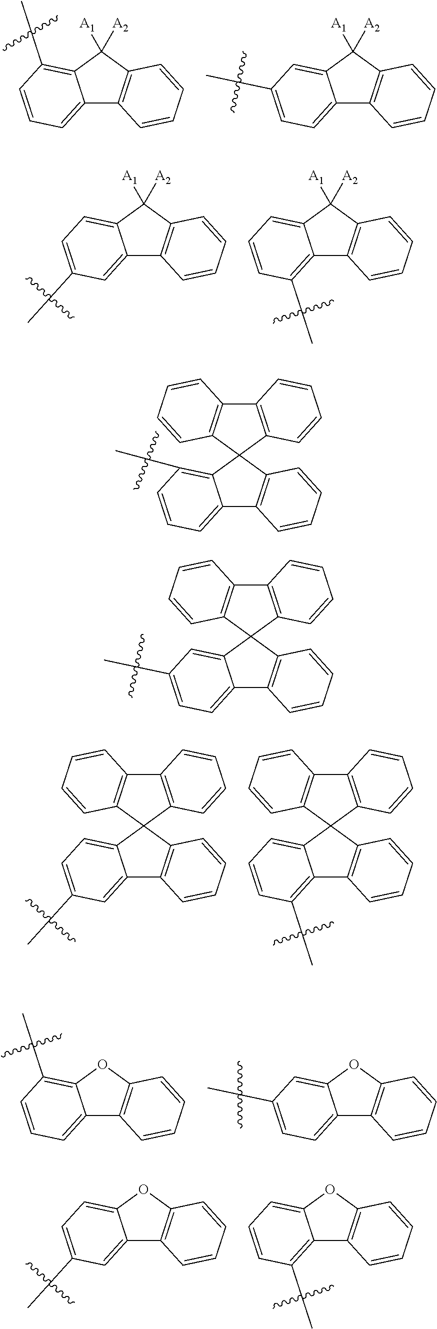 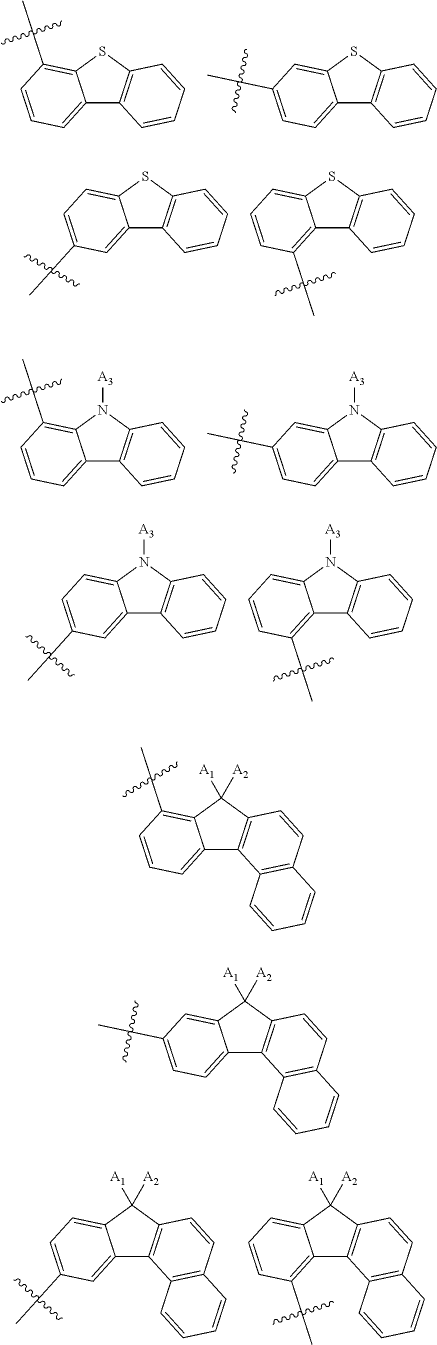 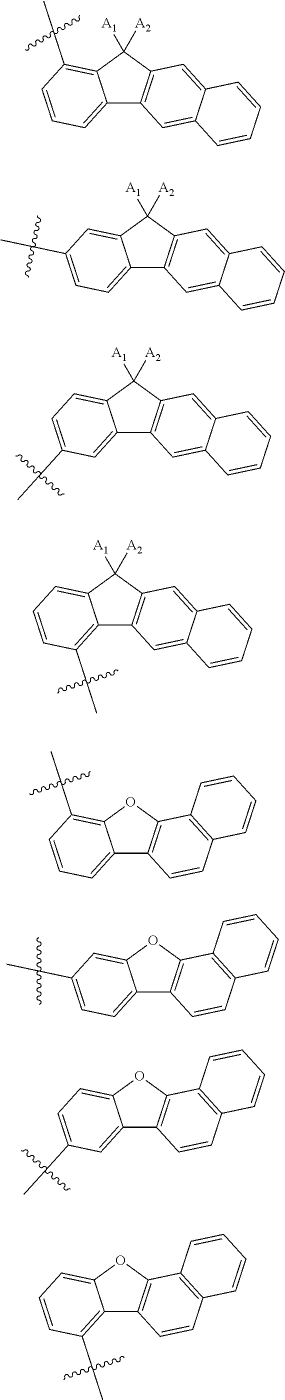 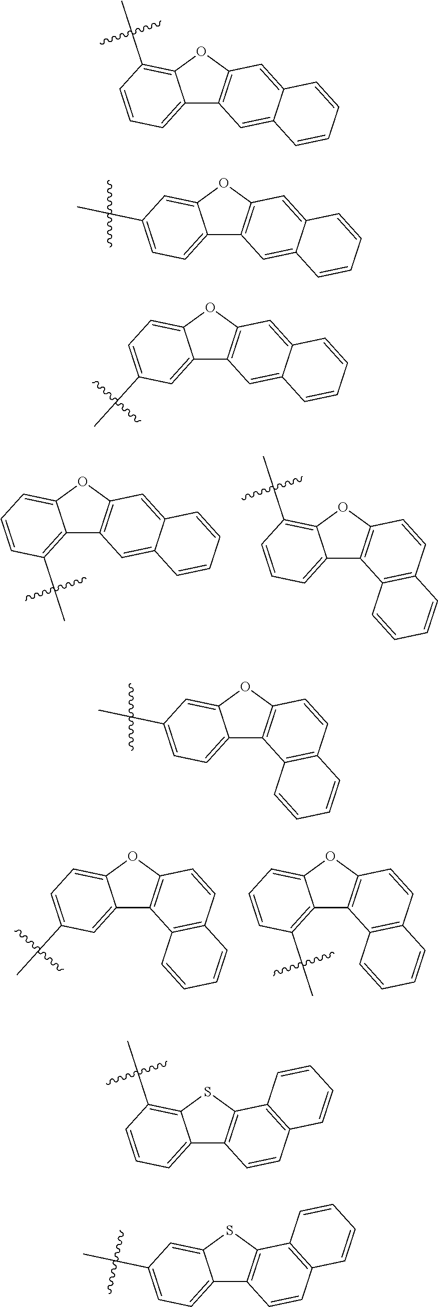 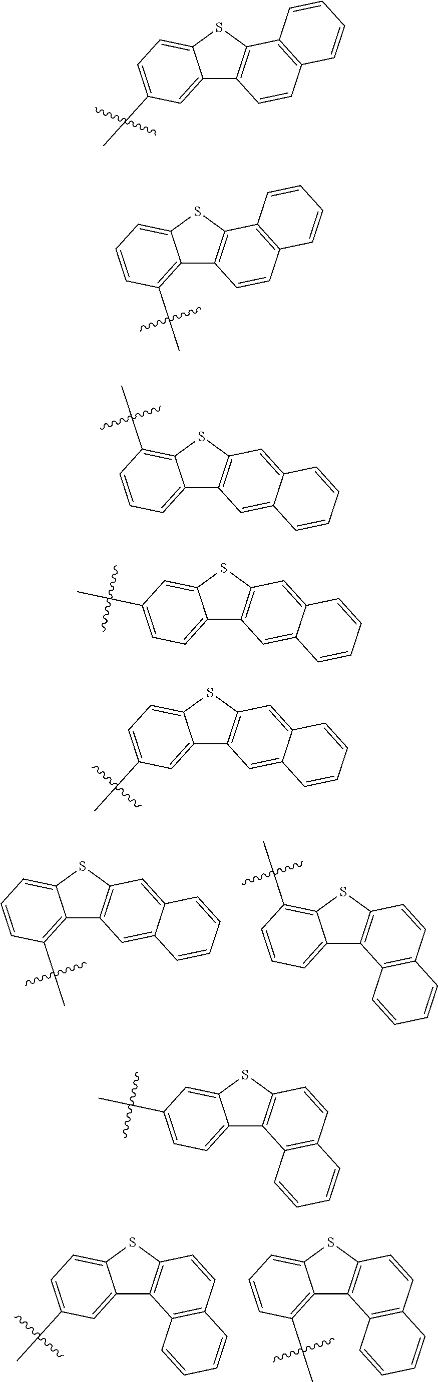 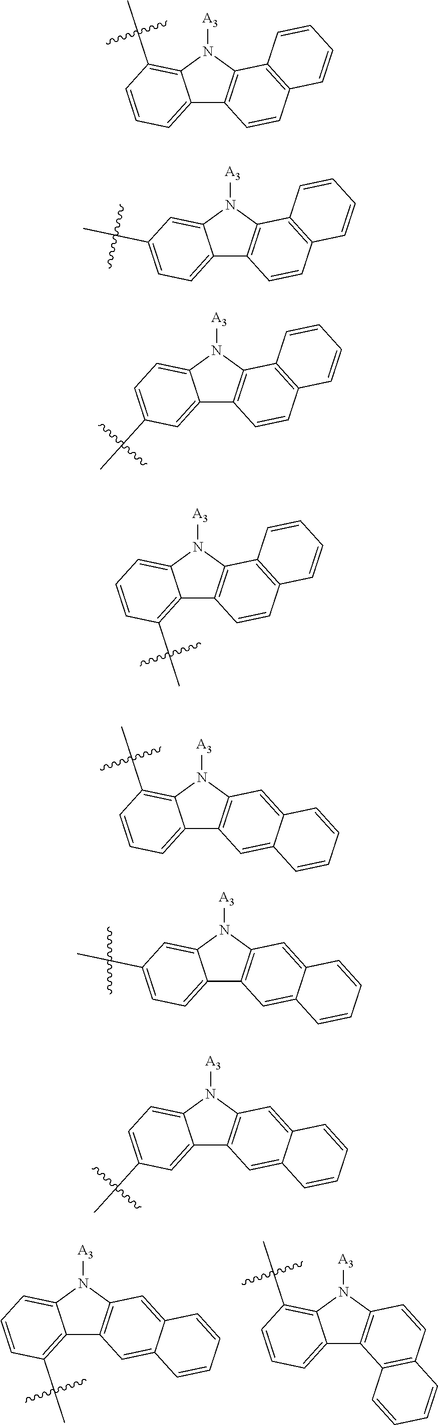 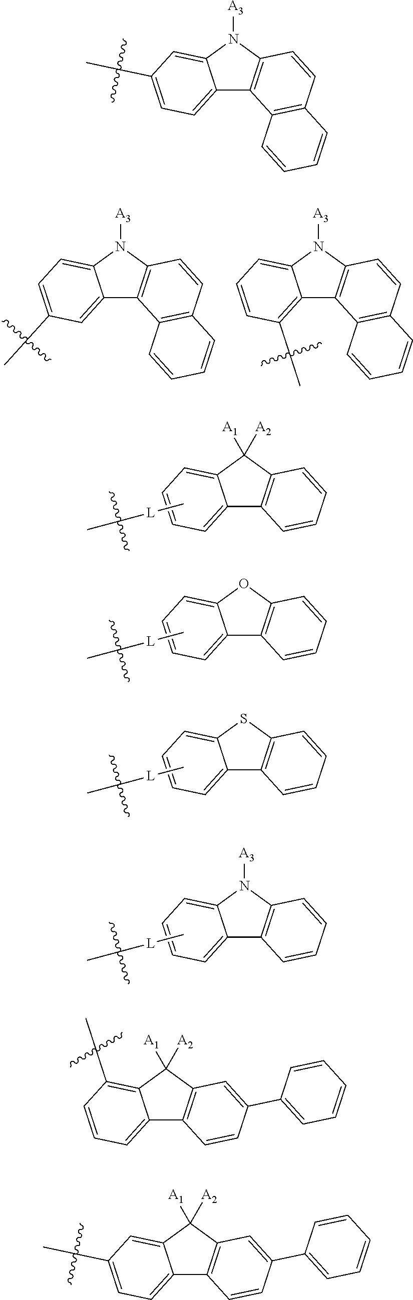 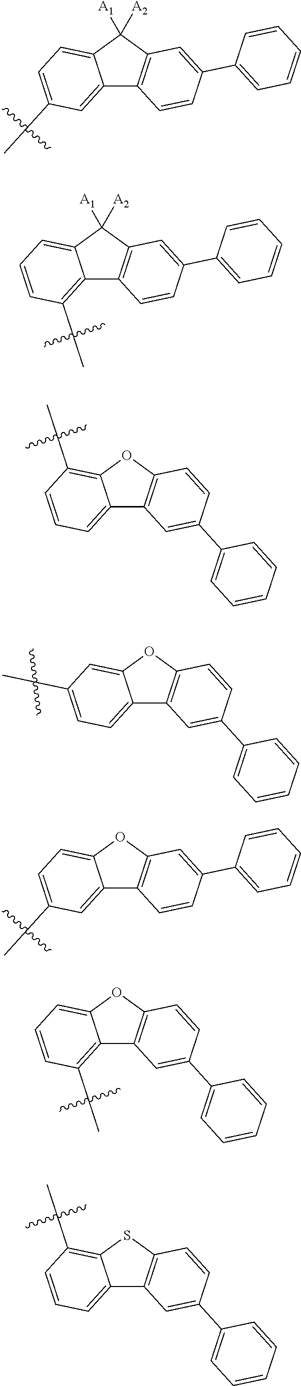 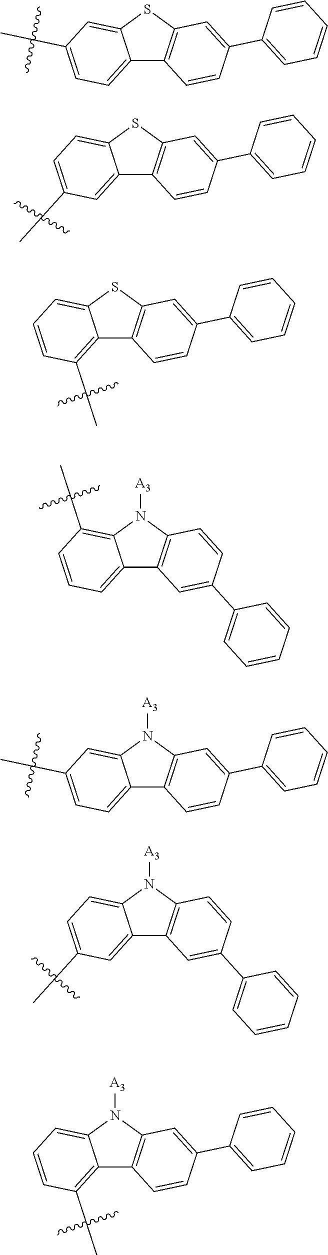 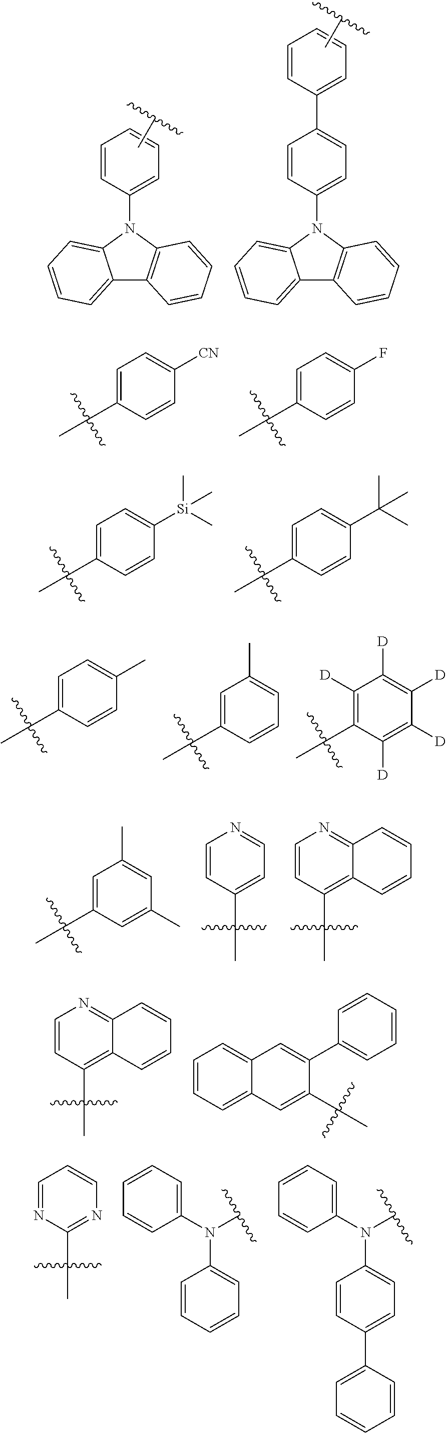 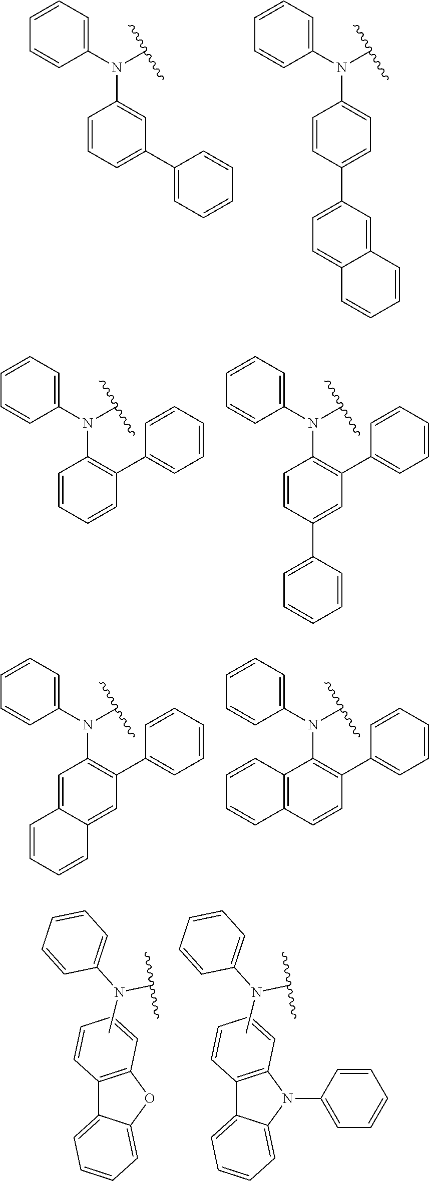 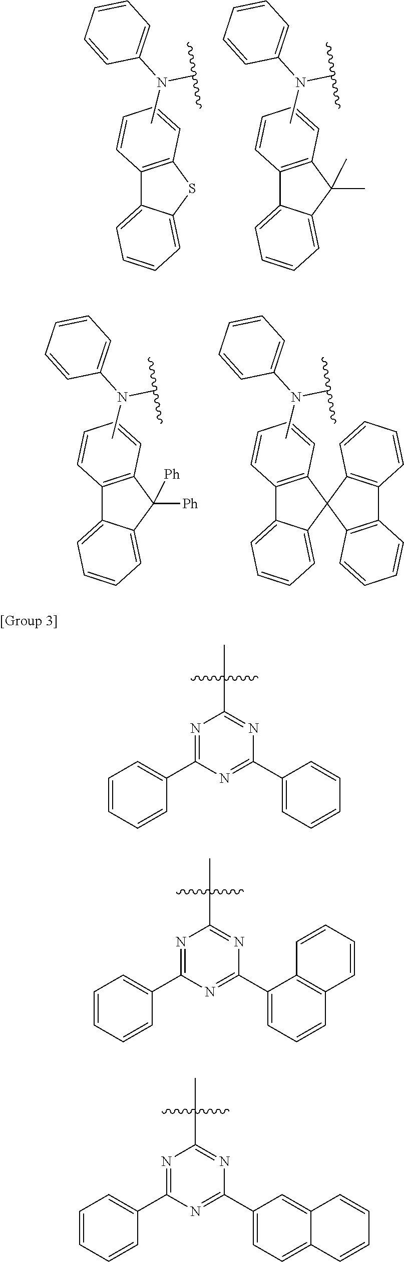 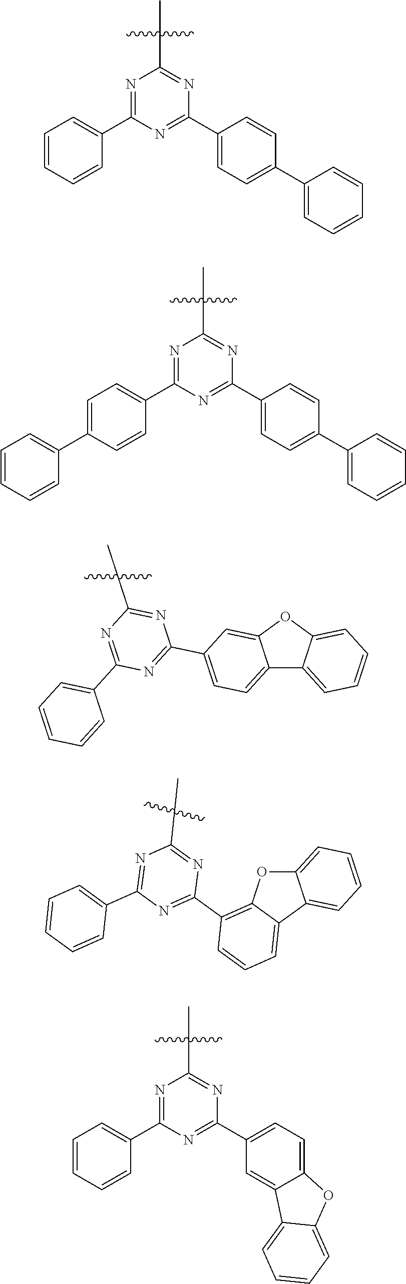 [SMILES: *c1nc(-c2ccccc2)nc(-c2ccc(-c3ccccc3)cc2)n1 ; *c1nc(-c2ccc(-c3ccccc3)cc2)nc(-c2ccc(-c3ccccc3)cc2)n1 ; *c1nc(-c2ccccc2)nc(-c2ccc3c(c2)oc2ccccc23)n1 ; *c1nc(-c2ccccc2)nc(-c2cccc3c2oc2ccccc23)n1 ; *c1nc(-c2ccccc2)nc(-c2ccc3oc4ccccc4c3c2)n1] 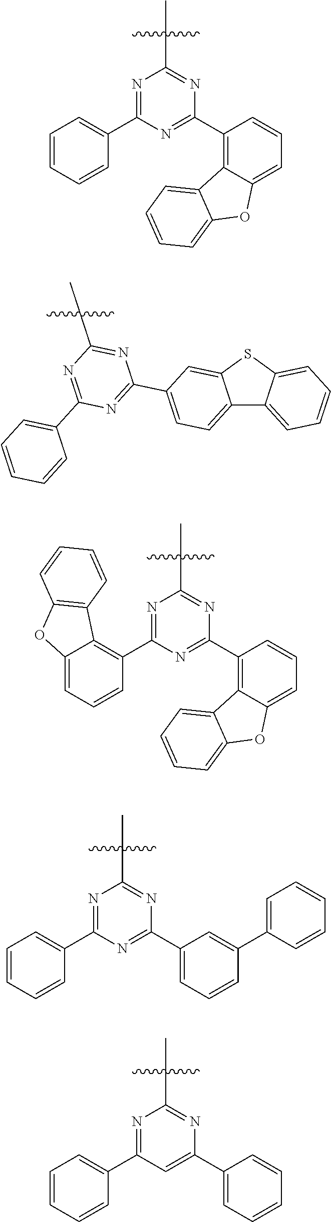 [SMILES: *c1nc(-c2ccccc2)nc(-c2cccc3oc4ccccc4c23)n1 ; *c1nc(-c2ccccc2)nc(-c2ccc3c(c2)sc2ccccc23)n1 ; *c1nc(-c2cccc3oc4ccccc4c23)nc(-c2cccc3oc4ccccc4c23)n1 ; *c1nc(-c2ccccc2)nc(-c2cccc(-c3ccccc3)c2)n1 ; *c1nc(-c2ccccc2)cc(-c2ccccc2)n1] 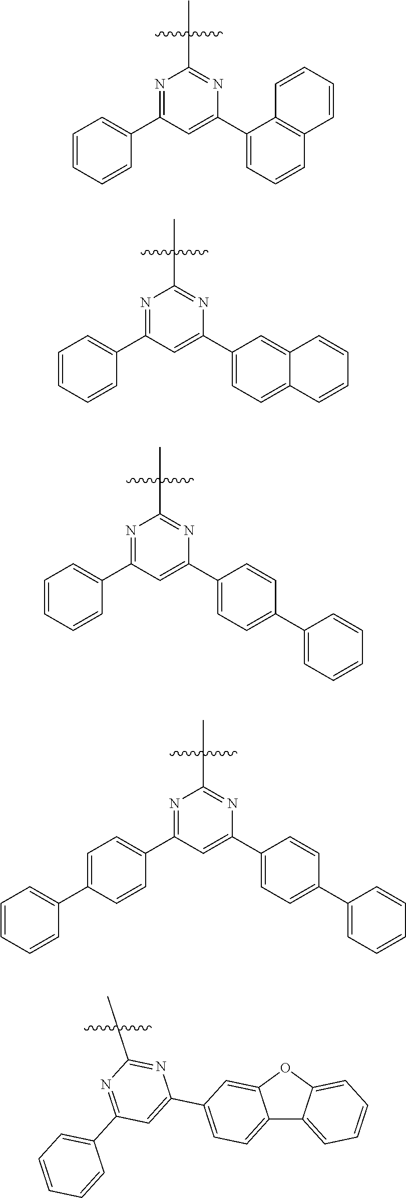 [SMILES: *c1nc(-c2ccccc2)cc(-c2cccc3ccccc23)n1 ; *c1nc(-c2ccccc2)cc(-c2ccc3ccccc3c2)n1 ; *c1nc(-c2ccccc2)cc(-c2ccc(-c3ccccc3)cc2)n1 ; *c1nc(-c2ccc(-c3ccccc3)cc2)cc(-c2ccc(-c3ccccc3)cc2)n1 ; *c1nc(-c2ccccc2)cc(-c2ccc3c(c2)oc2ccccc23)n1] 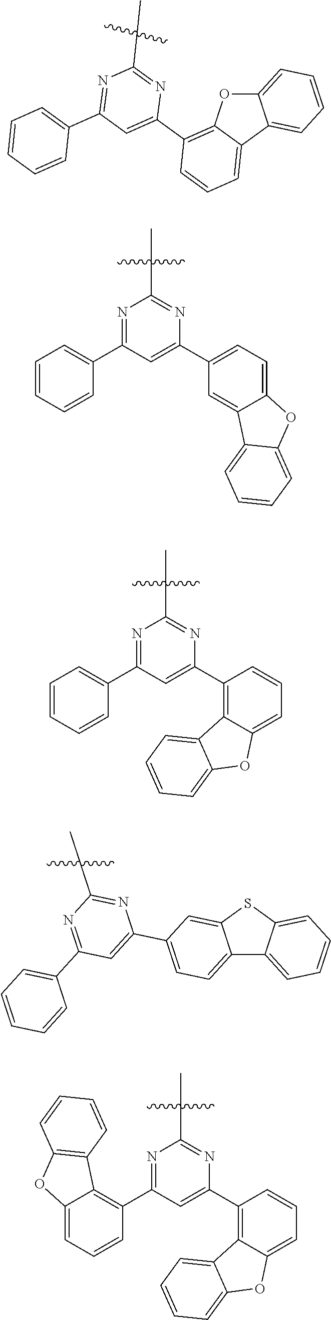 [SMILES: *c1nc(-c2ccccc2)cc(-c2cccc3c2oc2ccccc23)n1 ; *c1nc(-c2ccccc2)cc(-c2ccc3oc4ccccc4c3c2)n1 ; *c1nc(-c2ccccc2)cc(-c2cccc3oc4ccccc4c23)n1 ; *c1nc(-c2ccccc2)cc(-c2ccc3c(c2)sc2ccccc23)n1 ; *c1nc(-c2cccc3oc4ccccc4c23)cc(-c2cccc3oc4ccccc4c23)n1] 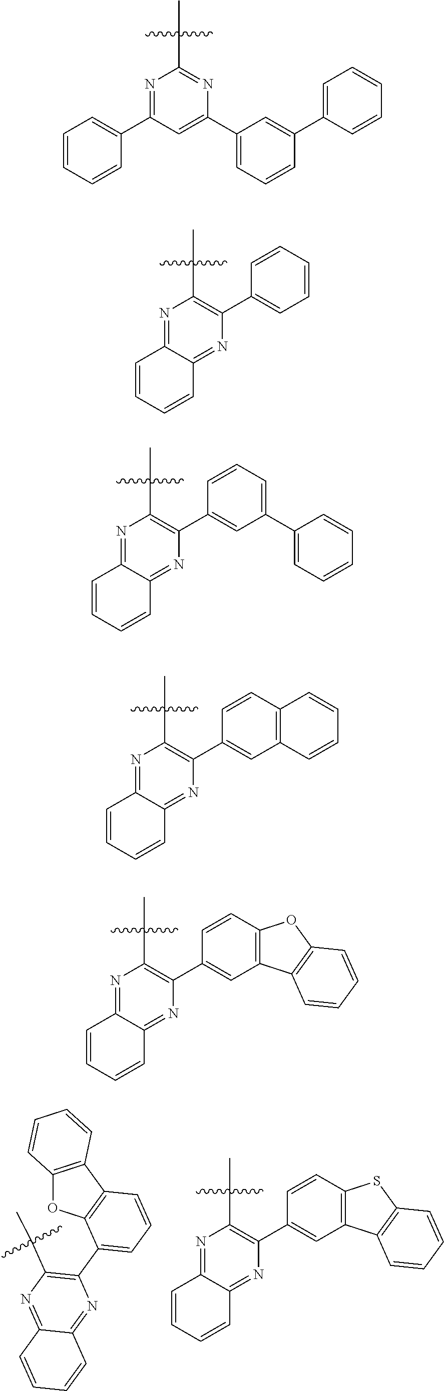 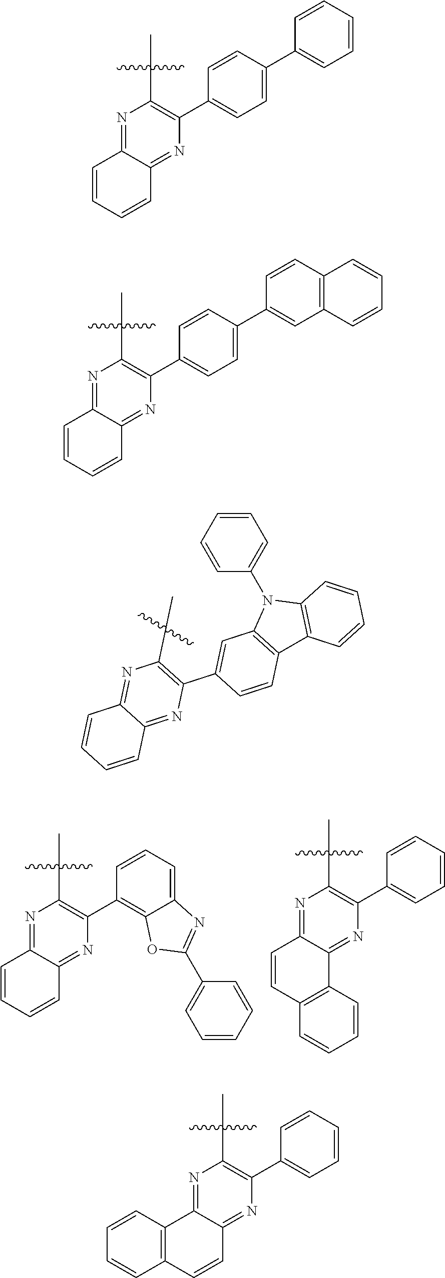 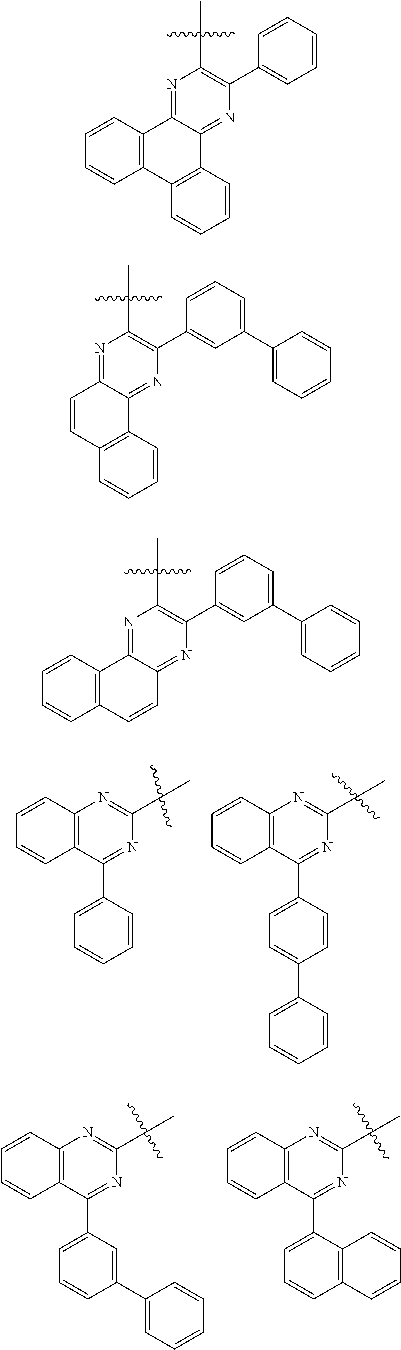 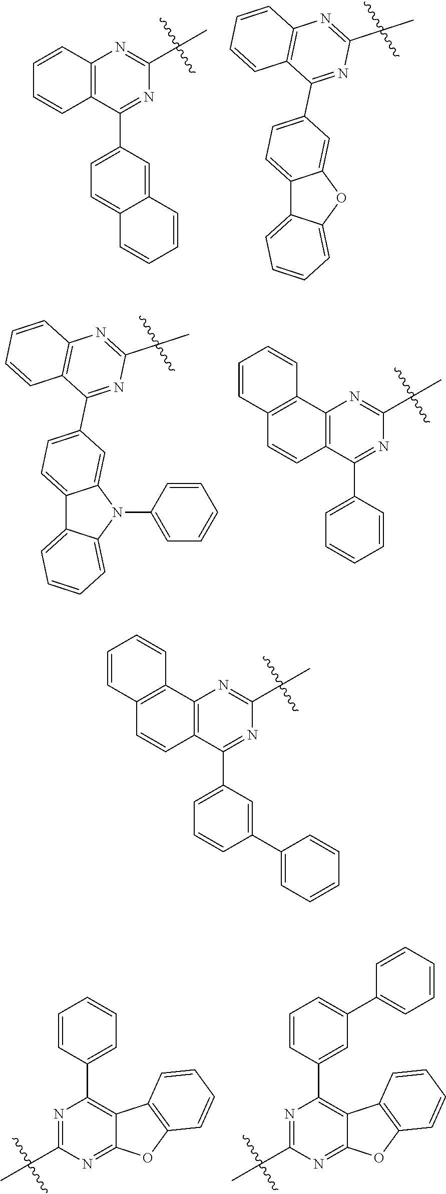 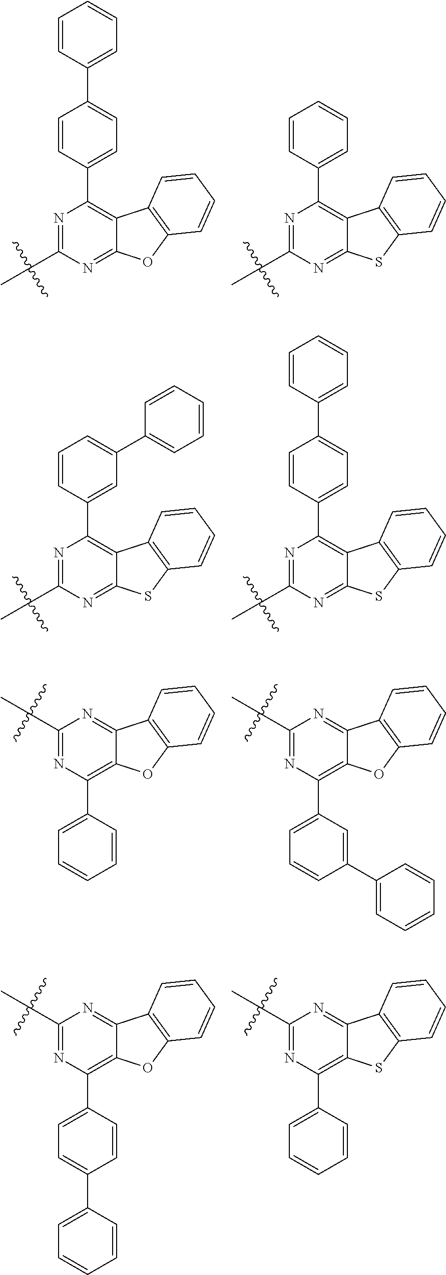 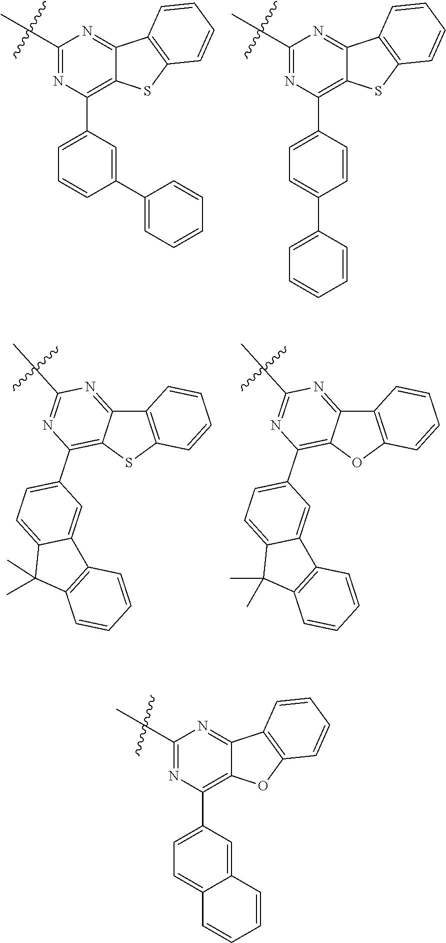
in Group 2,
L represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; and
A₁ to A₃, each independently, represent a substituted or unsubstituted (C1-C30)alkyl, or a substituted or unsubstituted (C6-C30)aryl.

7. The organic electroluminescent compound according to claim 1, wherein the compound represented by formula 1 is selected from the group consisting of the following compounds:

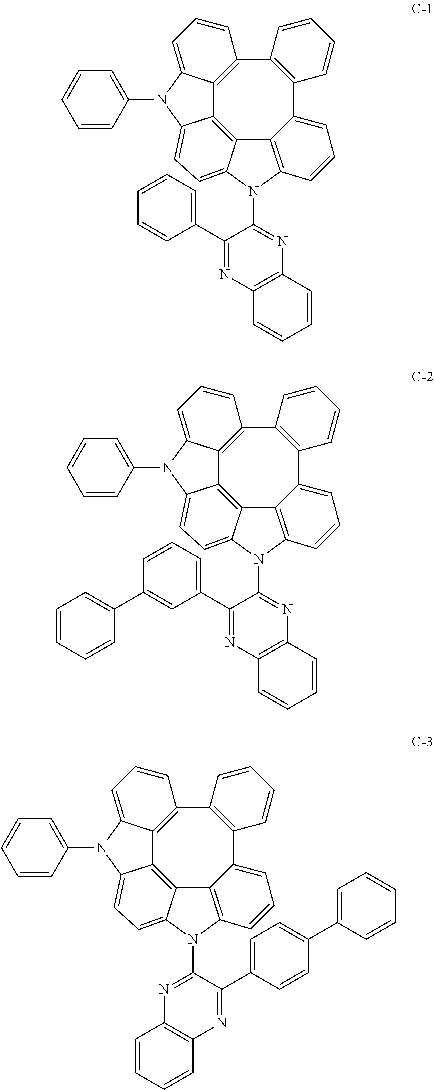 [SMILES: c1ccc(-c2nc3ccccc3nc2-n2c3cccc4c5ccccc5c5cccc6c5c5c(c43)c2ccc5n6-c2ccccc2)cc1 ; c1ccc(-c2cccc(-c3nc4ccccc4nc3-n3c4cccc5c6ccccc6c6cccc7c6c6c(c54)c3ccc6n7-c3ccccc3)c2)cc1 ; c1ccc(-c2ccc(-c3nc4ccccc4nc3-n3c4cccc5c6ccccc6c6cccc7c6c6c(c54)c3ccc6n7-c3ccccc3)cc2)cc1]

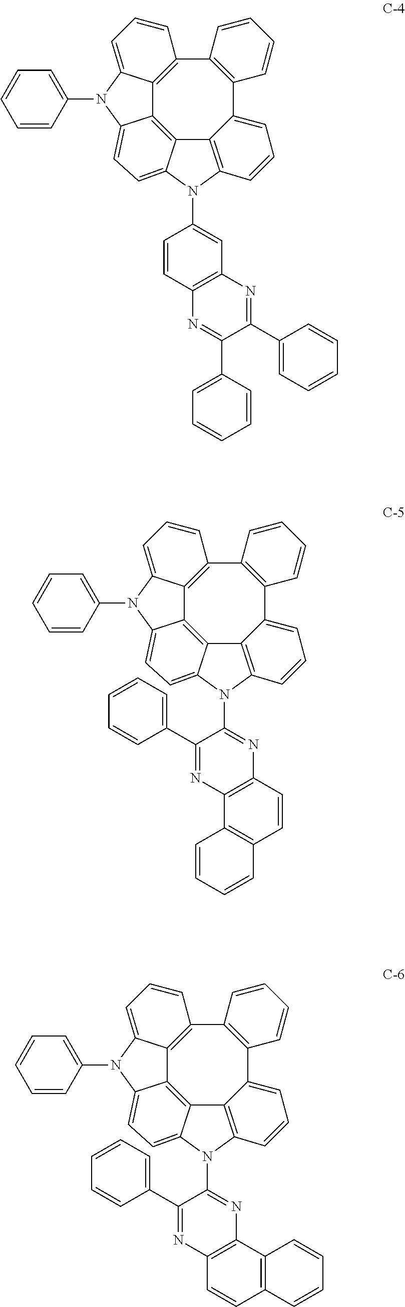 [SMILES: c1ccc(-c2nc3ccc(-n4c5cccc6c7ccccc7c7cccc8c7c7c(c65)c4ccc7n8-c4ccccc4)cc3nc2-c2ccccc2)cc1 ; c1ccc(-c2nc3c(ccc4ccccc43)nc2-n2c3cccc4c5ccccc5c5cccc6c5c5c(c43)c2ccc5n6-c2ccccc2)cc1 ; c1ccc(-c2nc3ccc4ccccc4c3nc2-n2c3cccc4c5ccccc5c5cccc6c5c5c(c43)c2ccc5n6-c2ccccc2)cc1]

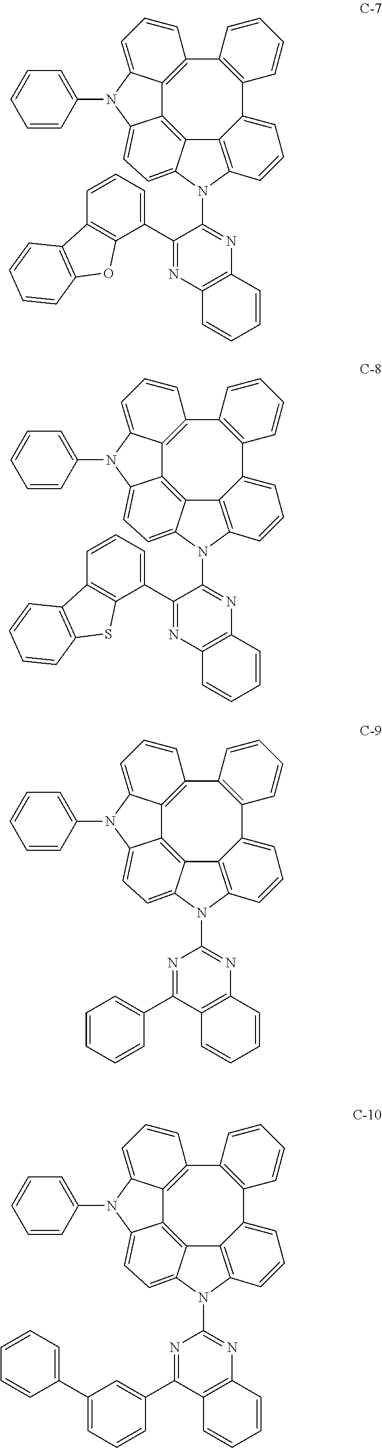

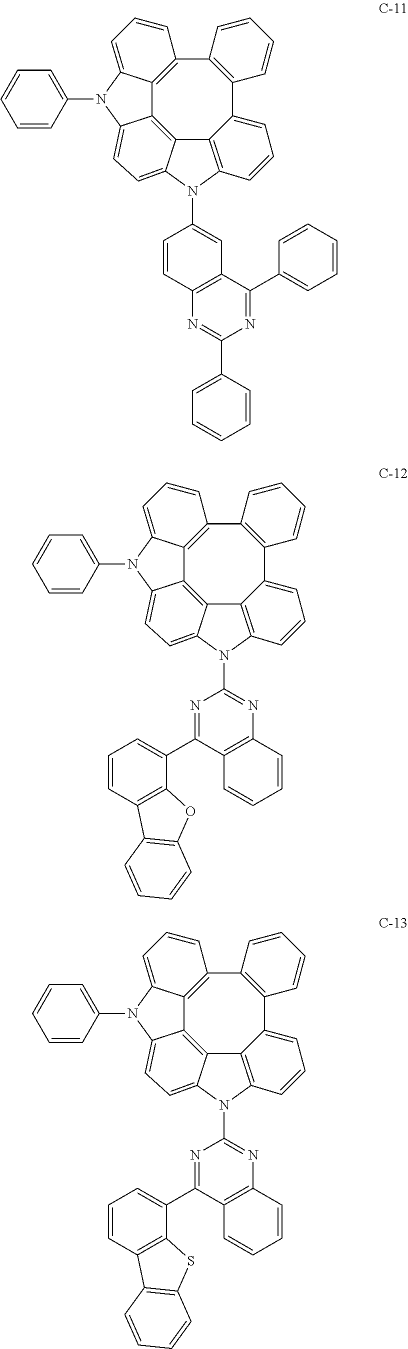

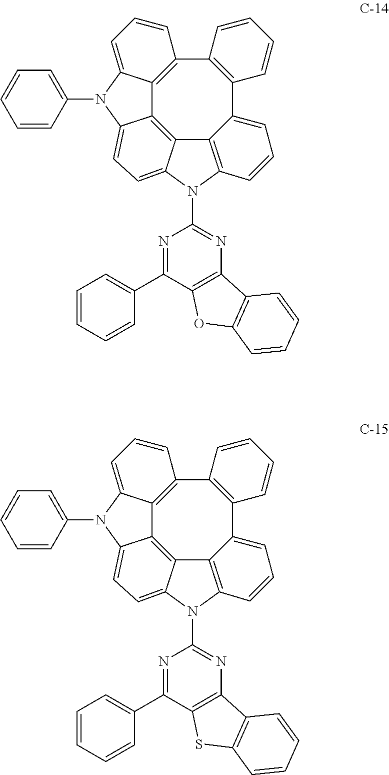 [SMILES: c1ccc(-c2nc(-n3c4cccc5c6ccccc6c6cccc7c6c6c(c54)c3ccc6n7-c3ccccc3)nc3c2oc2ccccc23)cc1 ; c1ccc(-c2nc(-n3c4cccc5c6ccccc6c6cccc7c6c6c(c54)c3ccc6n7-c3ccccc3)nc3c2sc2ccccc23)cc1]

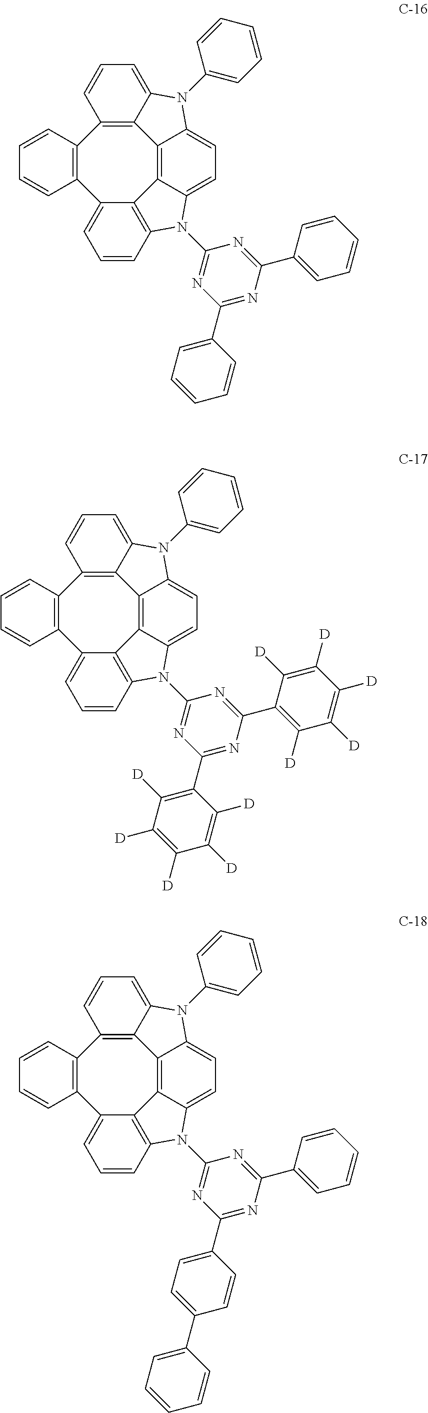 [SMILES: c1ccc(-c2nc(-c3ccccc3)nc(-n3c4cccc5c6ccccc6c6cccc7c6c6c(c54)c3ccc6n7-c3ccccc3)n2)cc1 ; [2H]c1c([2H])c([2H])c(-c2nc(-c3c([2H])c([2H])c([2H])c([2H])c3[2H])nc(-n3c4cccc5c6ccccc6c6cccc7c6c6c(c54)c3ccc6n7-c3ccccc3)n2)c([2H])c1[2H] ; c1ccc(-c2ccc(-c3nc(-c4ccccc4)nc(-n4c5cccc6c7ccccc7c7cccc8c7c7c(c65)c4ccc7n8-c4ccccc4)n3)cc2)cc1]

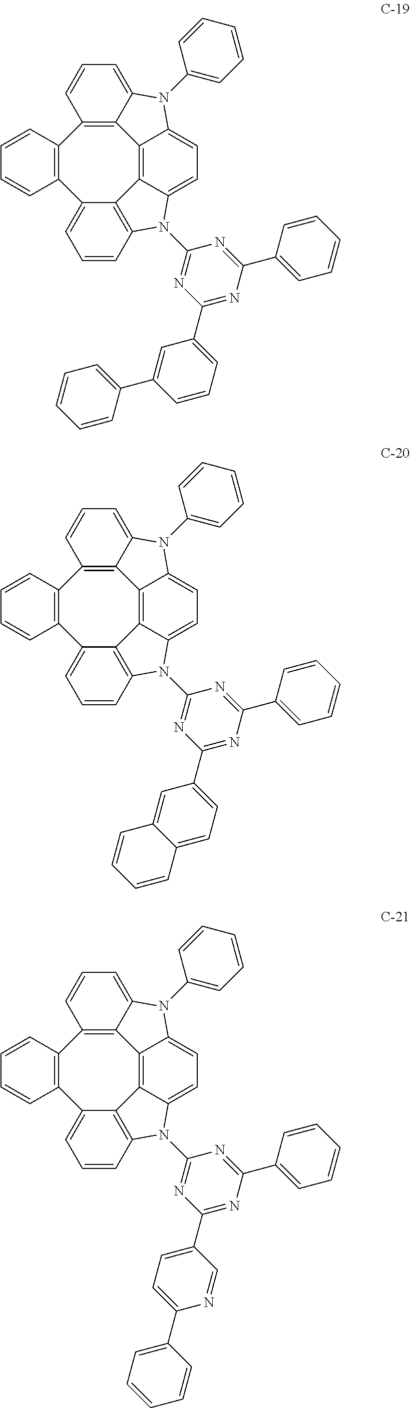 [SMILES: c1ccc(-c2cccc(-c3nc(-c4ccccc4)nc(-n4c5cccc6c7ccccc7c7cccc8c7c7c(c65)c4ccc7n8-c4ccccc4)n3)c2)cc1 ; c1ccc(-c2nc(-c3ccc4ccccc4c3)nc(-n3c4cccc5c6ccccc6c6cccc7c6c6c(c54)c3ccc6n7-c3ccccc3)n2)cc1 ; c1ccc(-c2ccc(-c3nc(-c4ccccc4)nc(-n4c5cccc6c7ccccc7c7cccc8c7c7c(c65)c4ccc7n8-c4ccccc4)n3)cn2)cc1]

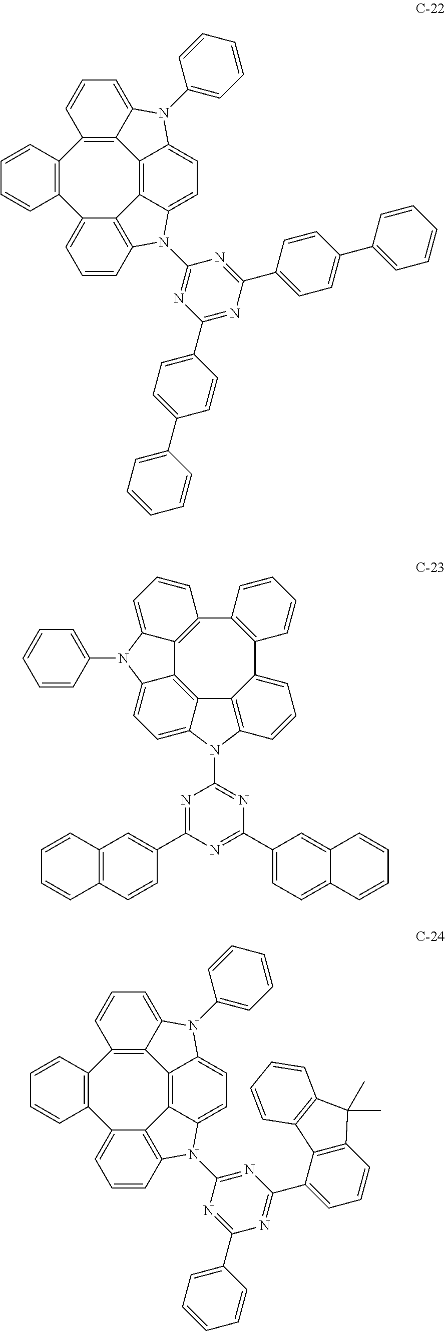 [SMILES: c1ccc(-c2ccc(-c3nc(-c4ccc(-c5ccccc5)cc4)nc(-n4c5cccc6c7ccccc7c7cccc8c7c7c(c65)c4ccc7n8-c4ccccc4)n3)cc2)cc1 ; c1ccc(-n2c3cccc4c5ccccc5c5cccc6c5c5c(c43)c2ccc5n6-c2nc(-c3ccc4ccccc4c3)nc(-c3ccc4ccccc4c3)n2)cc1 ; CC1(C)c2ccccc2-c2c(-c3nc(-c4ccccc4)nc(-n4c5cccc6c7ccccc7c7cccc8c7c7c(c65)c4ccc7n8-c4ccccc4)n3)cccc21]

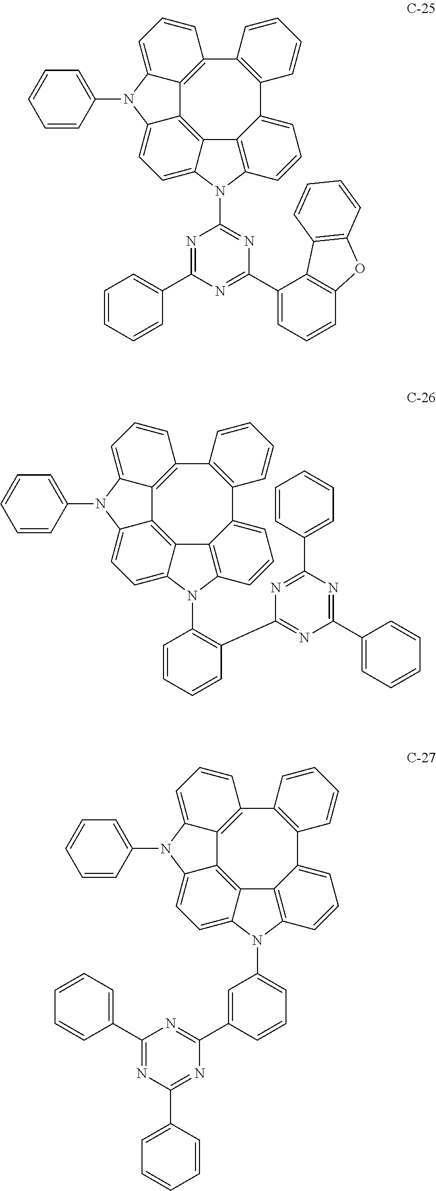 [SMILES: c1ccc(-c2nc(-c3cccc4oc5ccccc5c34)nc(-n3c4cccc5c6ccccc6c6cccc7c6c6c(c54)c3ccc6n7-c3ccccc3)n2)cc1 ; c1ccc(-c2nc(-c3ccccc3)nc(-c3ccccc3-n3c4cccc5c6ccccc6c6cccc7c6c6c(c54)c3ccc6n7-c3ccccc3)n2)cc1 ; c1ccc(-c2nc(-c3ccccc3)nc(-c3cccc(-n4c5cccc6c7ccccc7c7cccc8c7c7c(c65)c4ccc7n8-c4ccccc4)c3)n2)cc1]

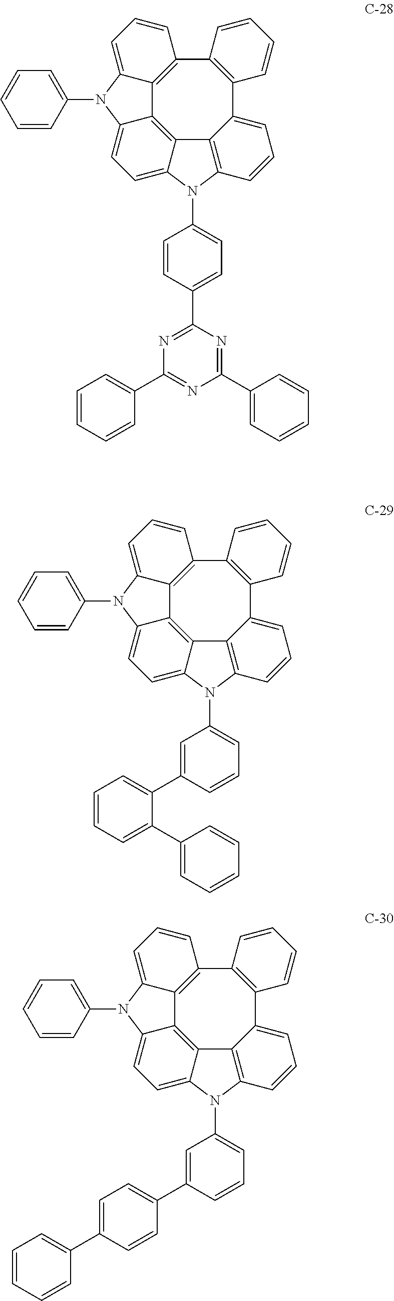 [SMILES: c1ccc(-c2nc(-c3ccccc3)nc(-c3ccc(-n4c5cccc6c7ccccc7c7cccc8c7c7c(c65)c4ccc7n8-c4ccccc4)cc3)n2)cc1 ; c1ccc(-c2ccccc2-c2cccc(-n3c4cccc5c6ccccc6c6cccc7c6c6c(c54)c3ccc6n7-c3ccccc3)c2)cc1 ; c1ccc(-c2ccc(-c3cccc(-n4c5cccc6c7ccccc7c7cccc8c7c7c(c65)c4ccc7n8-c4ccccc4)c3)cc2)cc1]

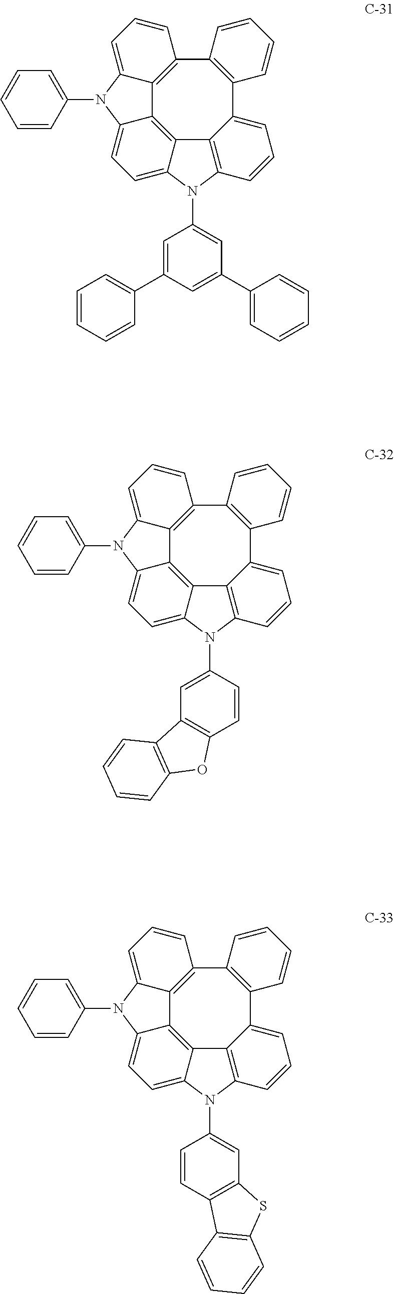 [SMILES: c1ccc(-c2cc(-c3ccccc3)cc(-n3c4cccc5c6ccccc6c6cccc7c6c6c(c54)c3ccc6n7-c3ccccc3)c2)cc1 ; c1ccc(-n2c3cccc4c5ccccc5c5cccc6c5c5c(c43)c2ccc5n6-c2ccc3oc4ccccc4c3c2)cc1 ; c1ccc(-n2c3cccc4c5ccccc5c5cccc6c5c5c(c43)c2ccc5n6-c2ccc3c(c2)sc2ccccc23)cc1]

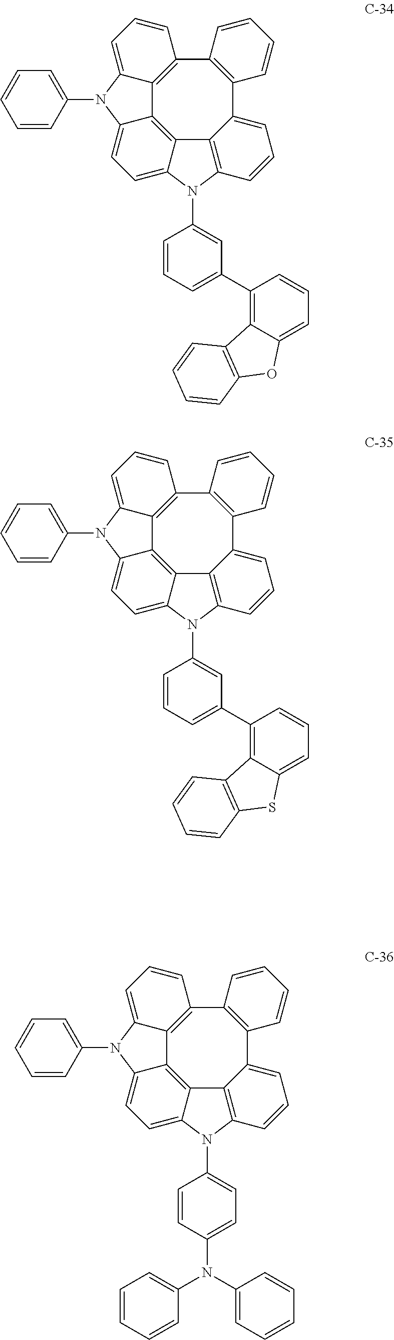 [SMILES: c1ccc(-n2c3cccc4c5ccccc5c5cccc6c5c5c(c43)c2ccc5n6-c2cccc(-c3cccc4oc5ccccc5c34)c2)cc1 ; c1ccc(-n2c3cccc4c5ccccc5c5cccc6c5c5c(c43)c2ccc5n6-c2cccc(-c3cccc4sc5ccccc5c34)c2)cc1 ; c1ccc(N(c2ccccc2)c2ccc(-n3c4cccc5c6ccccc6c6cccc7c6c6c(c54)c3ccc6n7-c3ccccc3)cc2)cc1]

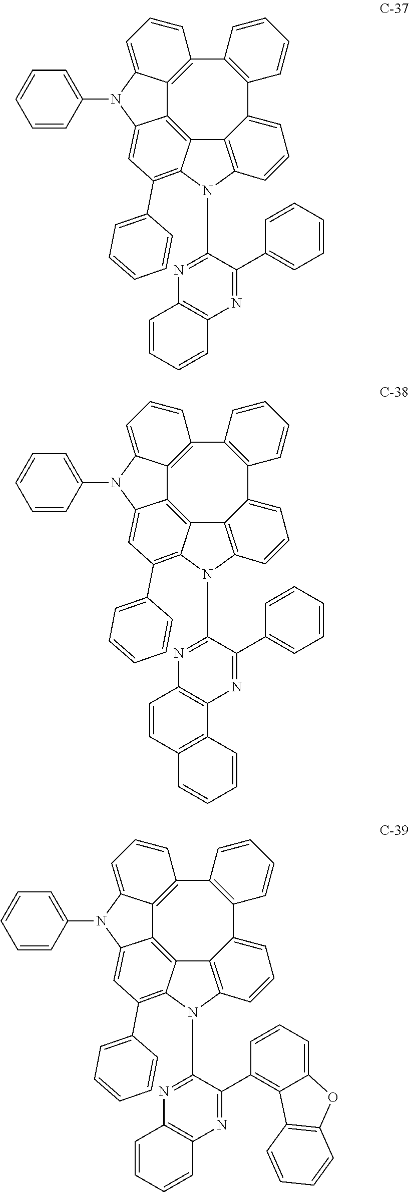

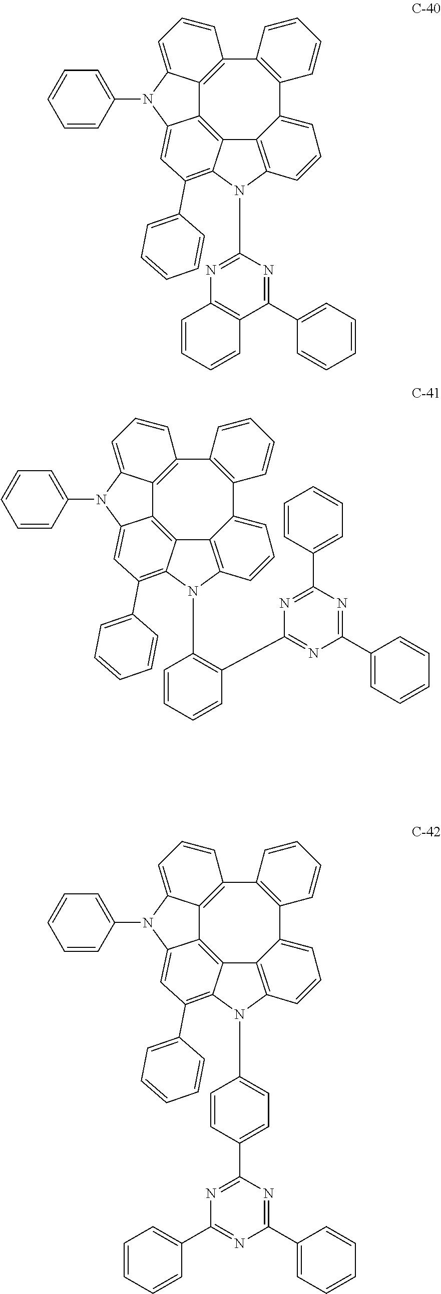 [SMILES: c1ccc(-c2nc(-n3c4cccc5c6ccccc6c6cccc7c6c6c(c54)c3c(-c3ccccc3)cc6n7-c3ccccc3)nc3ccccc23)cc1 ; c1ccc(-c2nc(-c3ccccc3)nc(-c3ccccc3-n3c4cccc5c6ccccc6c6cccc7c6c6c(c54)c3c(-c3ccccc3)cc6n7-c3ccccc3)n2)cc1 ; c1ccc(-c2nc(-c3ccccc3)nc(-c3ccc(-n4c5cccc6c7ccccc7c7cccc8c7c7c(c65)c4c(-c4ccccc4)cc7n8-c4ccccc4)cc3)n2)cc1]

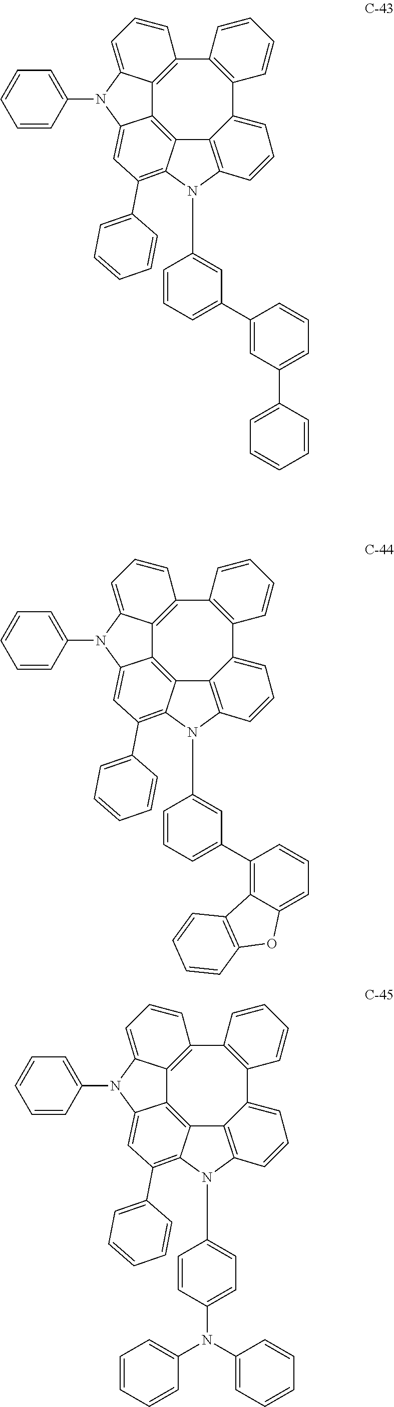

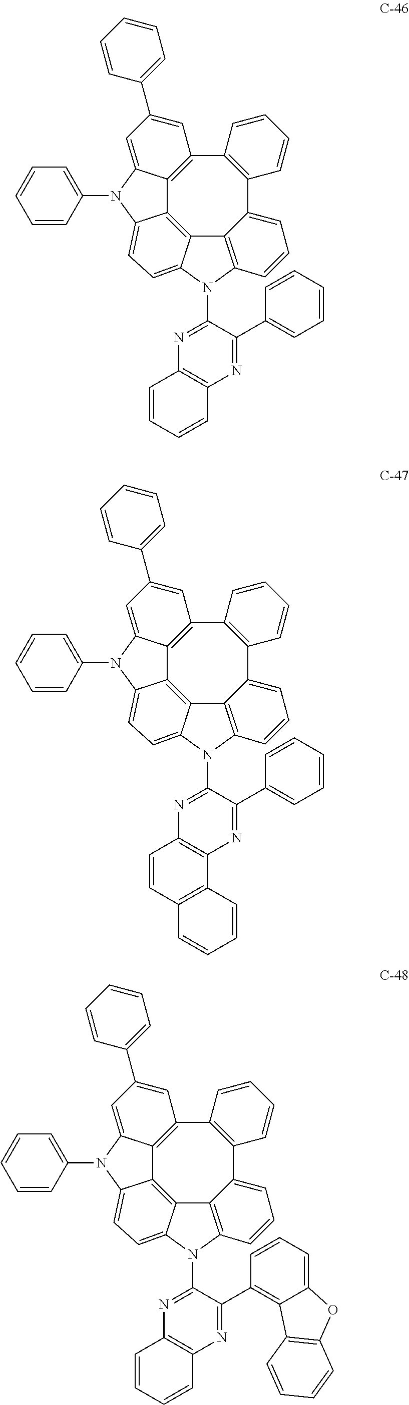 [SMILES: c1ccc(-c2cc3c4ccccc4c4cccc5c4c4c6c3c(c2)n(-c2ccccc2)c6ccc4n5-c2nc3ccccc3nc2-c2ccccc2)cc1 ; c1ccc(-c2cc3c4ccccc4c4cccc5c4c4c6c3c(c2)n(-c2ccccc2)c6ccc4n5-c2nc3ccc4ccccc4c3nc2-c2ccccc2)cc1 ; c1ccc(-c2cc3c4ccccc4c4cccc5c4c4c6c3c(c2)n(-c2ccccc2)c6ccc4n5-c2nc3ccccc3nc2-c2cccc3oc4ccccc4c23)cc1]

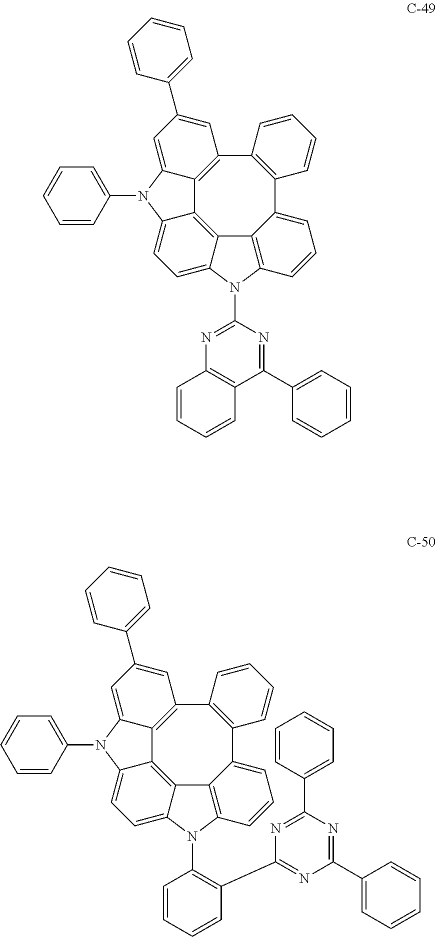 [SMILES: c1ccc(-c2cc3c4ccccc4c4cccc5c4c4c6c3c(c2)n(-c2ccccc2)c6ccc4n5-c2nc(-c3ccccc3)c3ccccc3n2)cc1 ; c1ccc(-c2cc3c4ccccc4c4cccc5c4c4c6c3c(c2)n(-c2ccccc2)c6ccc4n5-c2ccccc2-c2nc(-c3ccccc3)nc(-c3ccccc3)n2)cc1]

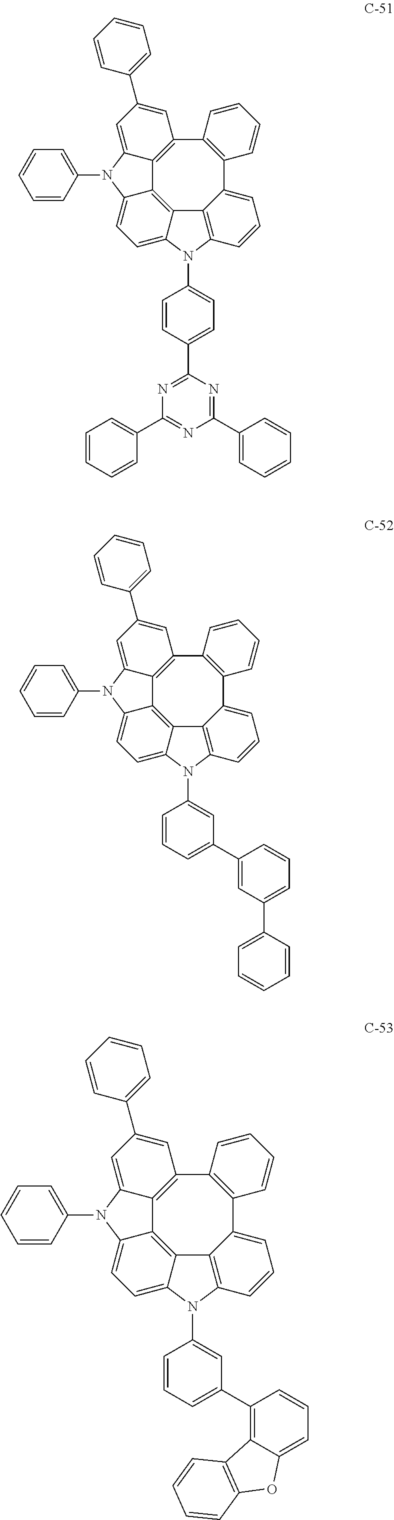 [SMILES: c1ccc(-c2cc3c4ccccc4c4cccc5c4c4c6c3c(c2)n(-c2ccccc2)c6ccc4n5-c2ccc(-c3nc(-c4ccccc4)nc(-c4ccccc4)n3)cc2)cc1 ; c1ccc(-c2cccc(-c3cccc(-n4c5cccc6c7ccccc7c7cc(-c8ccccc8)cc8c7c7c(c65)c4ccc7n8-c4ccccc4)c3)c2)cc1 ; c1ccc(-c2cc3c4ccccc4c4cccc5c4c4c6c3c(c2)n(-c2ccccc2)c6ccc4n5-c2cccc(-c3cccc4oc5ccccc5c34)c2)cc1]

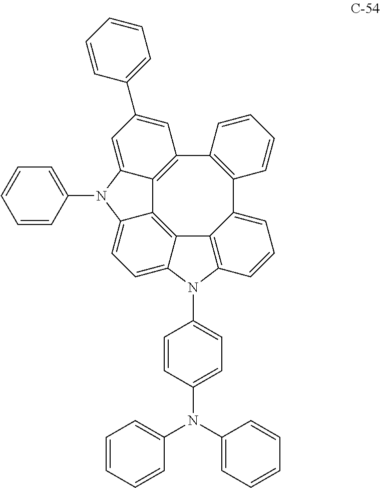 [SMILES: c1ccc(-c2cc3c4ccccc4c4cccc5c4c4c6c3c(c2)n(-c2ccccc2)c6ccc4n5-c2ccc(N(c3ccccc3)c3ccccc3)cc2)cc1]

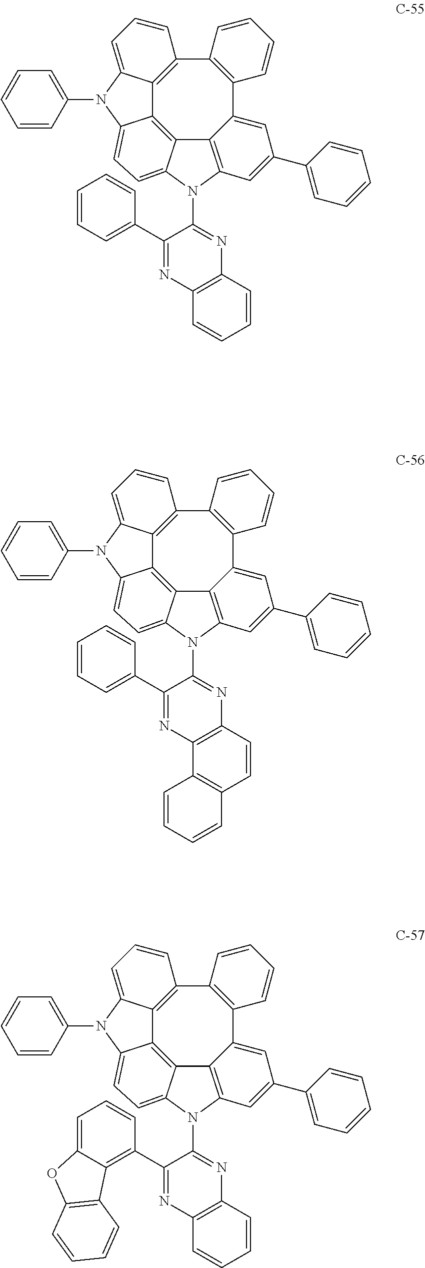 [SMILES: c1ccc(-c2cc3c4ccccc4c4cccc5c4c4c6c3c(c2)n(-c2nc3ccccc3nc2-c2ccccc2)c6ccc4n5-c2ccccc2)cc1 ; c1ccc(-c2cc3c4ccccc4c4cccc5c4c4c6c3c(c2)n(-c2nc3ccc7ccccc7c3nc2-c2ccccc2)c6ccc4n5-c2ccccc2)cc1 ; c1ccc(-c2cc3c4ccccc4c4cccc5c4c4c6c3c(c2)n(-c2nc3ccccc3nc2-c2cccc3oc7ccccc7c23)c6ccc4n5-c2ccccc2)cc1]

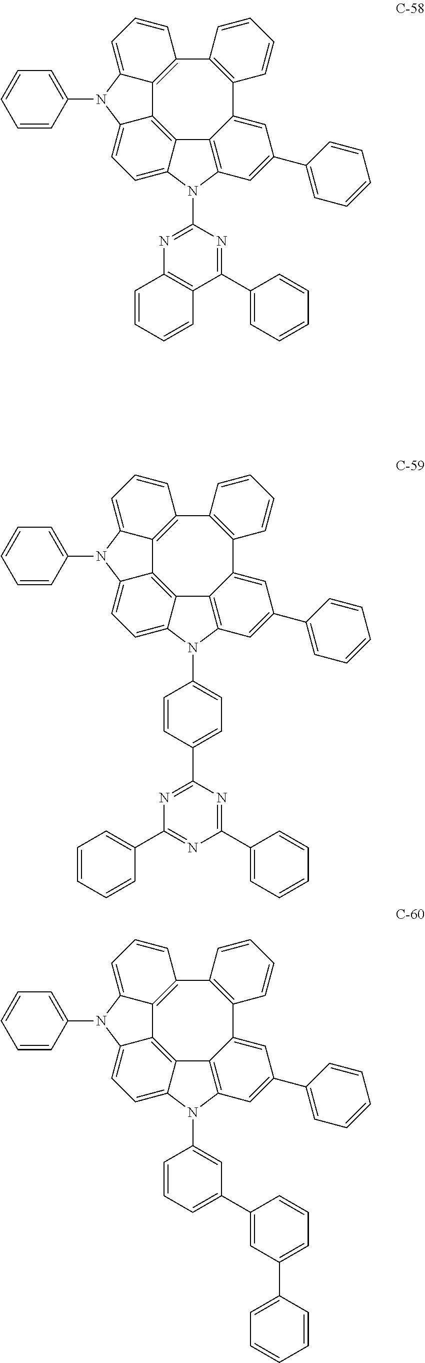 [SMILES: c1ccc(-c2cc3c4ccccc4c4cccc5c4c4c6c3c(c2)n(-c2nc(-c3ccccc3)c3ccccc3n2)c6ccc4n5-c2ccccc2)cc1 ; c1ccc(-c2cc3c4ccccc4c4cccc5c4c4c6c3c(c2)n(-c2ccc(-c3nc(-c7ccccc7)nc(-c7ccccc7)n3)cc2)c6ccc4n5-c2ccccc2)cc1 ; c1ccc(-c2cccc(-c3cccc(-n4c5cc(-c6ccccc6)cc6c7ccccc7c7cccc8c7c7c(c65)c4ccc7n8-c4ccccc4)c3)c2)cc1]

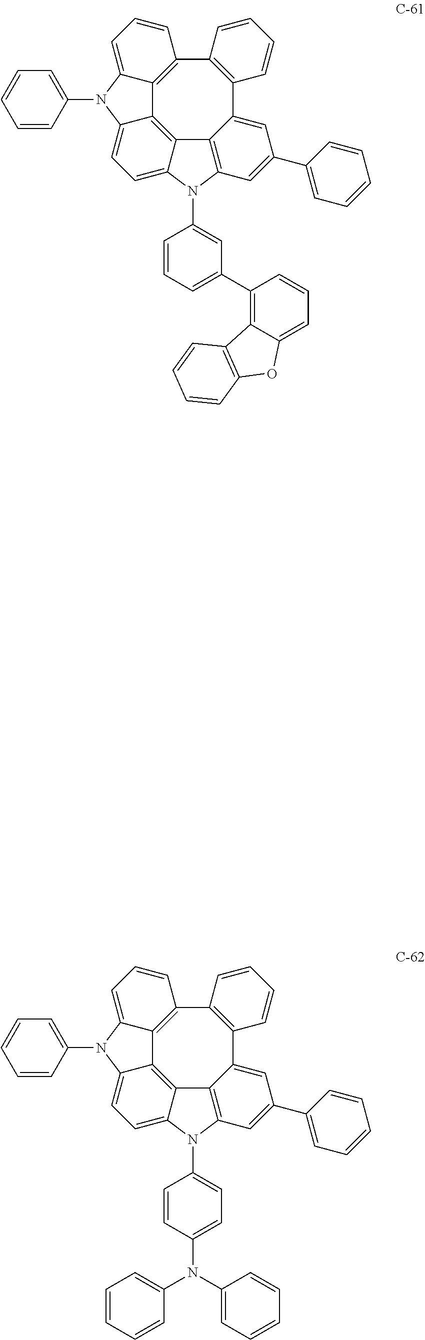 [SMILES: c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1]

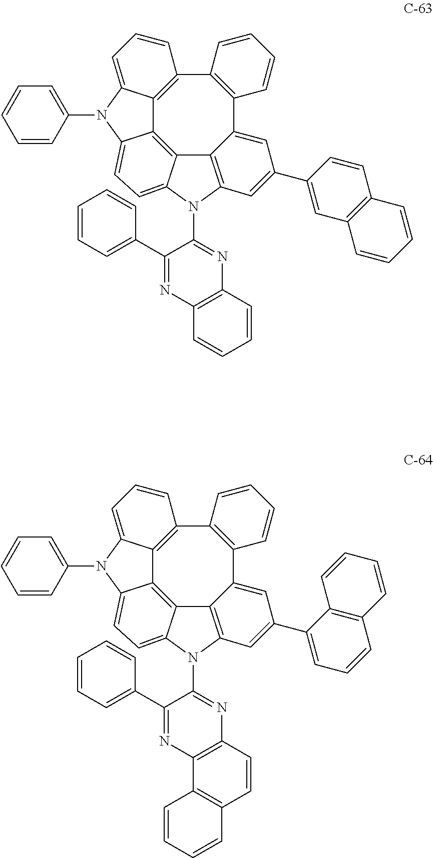 [SMILES: c1ccc(-c2nc3ccccc3nc2-n2c3cc(-c4ccc5ccccc5c4)cc4c5ccccc5c5cccc6c5c5c(c43)c2ccc5n6-c2ccccc2)cc1 ; c1ccc(-c2nc3c(ccc4ccccc43)nc2-n2c3cc(-c4cccc5ccccc45)cc4c5ccccc5c5cccc6c5c5c(c43)c2ccc5n6-c2ccccc2)cc1]

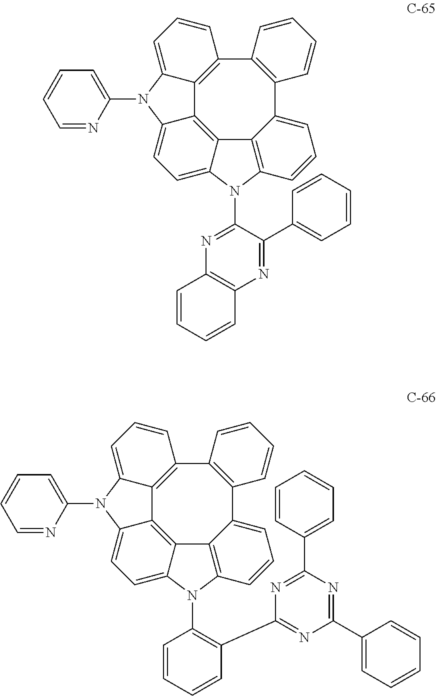 [SMILES: c1ccc(-c2nc3ccccc3nc2-n2c3cccc4c5ccccc5c5cccc6c5c5c(c43)c2ccc5n6-c2ccccn2)cc1 ; c1ccc(-c2nc(-c3ccccc3)nc(-c3ccccc3-n3c4cccc5c6ccccc6c6cccc7c6c6c(c54)c3ccc6n7-c3ccccn3)n2)cc1]

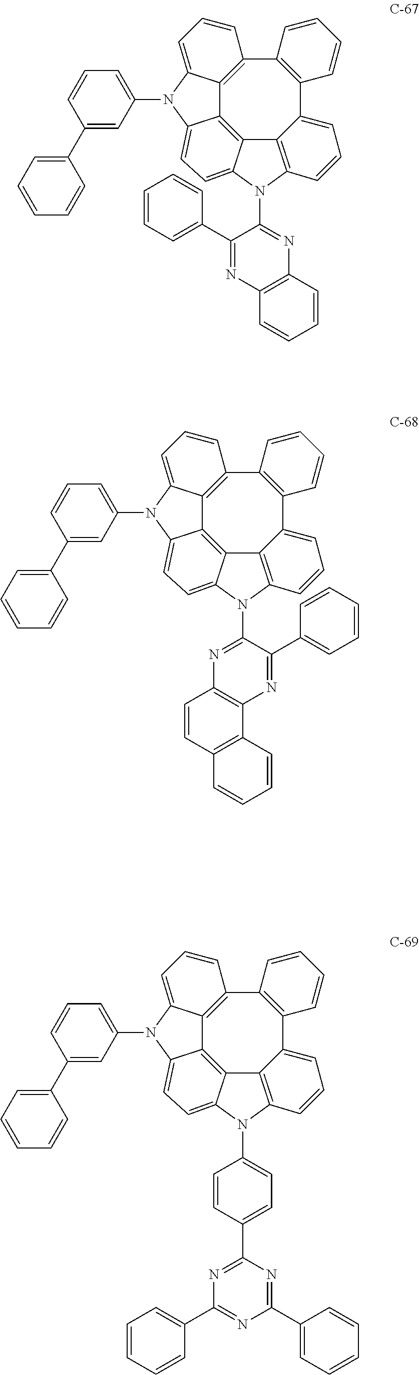 [SMILES: c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3nc4ccc5ccccc5c4nc3-c3ccccc3)c2)cc1 ; c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1]

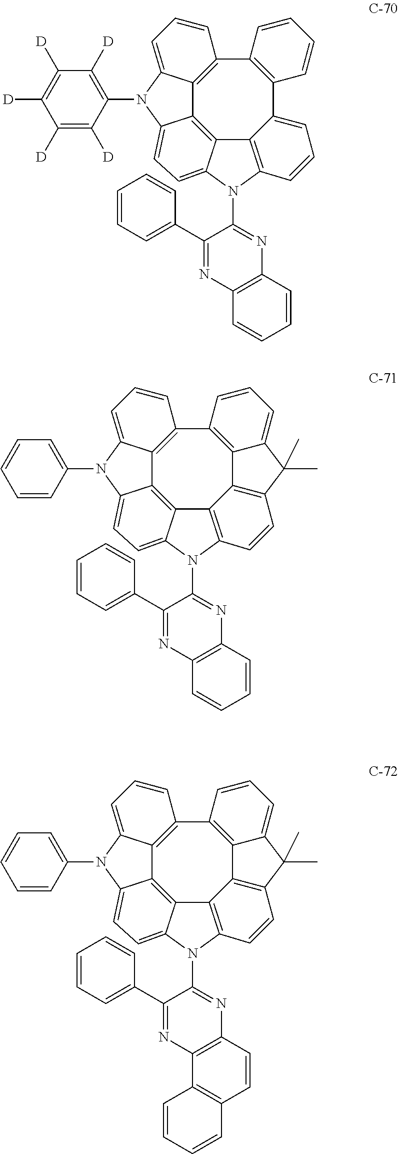 [SMILES: [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H] ; CC1(C)c2cccc3c4cccc5c4c4c6c7c(c1ccc7n(-c1nc7ccccc7nc1-c1ccccc1)c6ccc4n5-c1ccccc1)c23 ; CC1(C)c2cccc3c4cccc5c4c4c6c7c(c1ccc7n(-c1nc7ccc8ccccc8c7nc1-c1ccccc1)c6ccc4n5-c1ccccc1)c23]

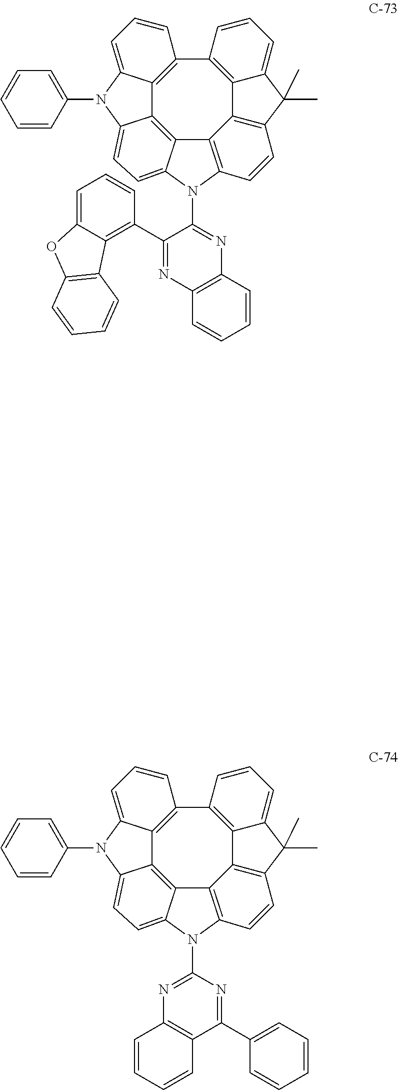 [SMILES: CC1(C)c2cccc3c4cccc5c4c4c6c7c(c1ccc7n(-c1nc7ccccc7nc1-c1cccc7oc8ccccc8c17)c6ccc4n5-c1ccccc1)c23 ; CC1(C)c2cccc3c4cccc5c4c4c6c7c(c1ccc7n(-c1nc(-c7ccccc7)c7ccccc7n1)c6ccc4n5-c1ccccc1)c23]

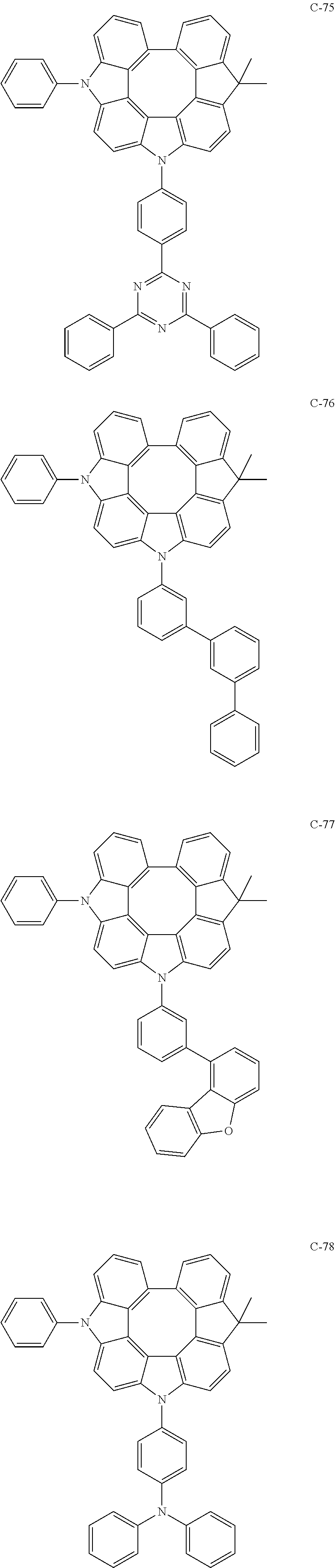 [SMILES: CC1(C)c2cccc3c4cccc5c4c4c6c7c(c1ccc7n(-c1ccc(-c7nc(-c8ccccc8)nc(-c8ccccc8)n7)cc1)c6ccc4n5-c1ccccc1)c23 ; CC1(C)c2cccc3c4cccc5c4c4c6c7c(c1ccc7n(-c1cccc(-c7cccc(-c8ccccc8)c7)c1)c6ccc4n5-c1ccccc1)c23 ; CC1(C)c2cccc3c4cccc5c4c4c6c7c(c1ccc7n(-c1cccc(-c7cccc8oc9ccccc9c78)c1)c6ccc4n5-c1ccccc1)c23 ; CC1(C)c2cccc3c4cccc5c4c4c6c7c(c1ccc7n(-c1ccc(N(c7ccccc7)c7ccccc7)cc1)c6ccc4n5-c1ccccc1)c23]

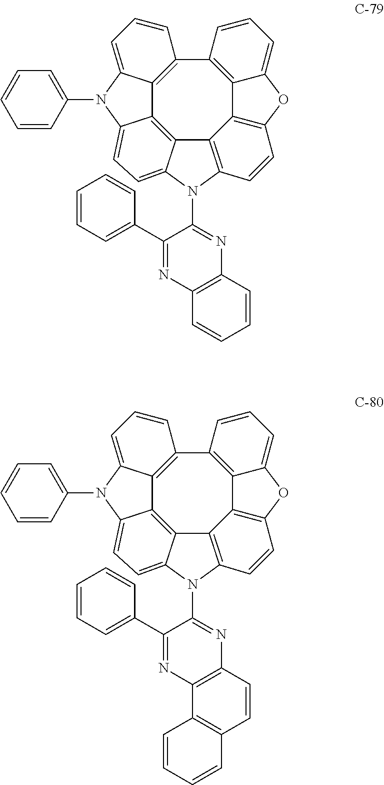 [SMILES: c1ccc(-c2nc3ccccc3nc2-n2c3ccc4oc5cccc6c7cccc8c7c7c(c3c4c56)c2ccc7n8-c2ccccc2)cc1 ; c1ccc(-c2nc3c(ccc4ccccc43)nc2-n2c3ccc4oc5cccc6c7cccc8c7c7c(c3c4c56)c2ccc7n8-c2ccccc2)cc1]

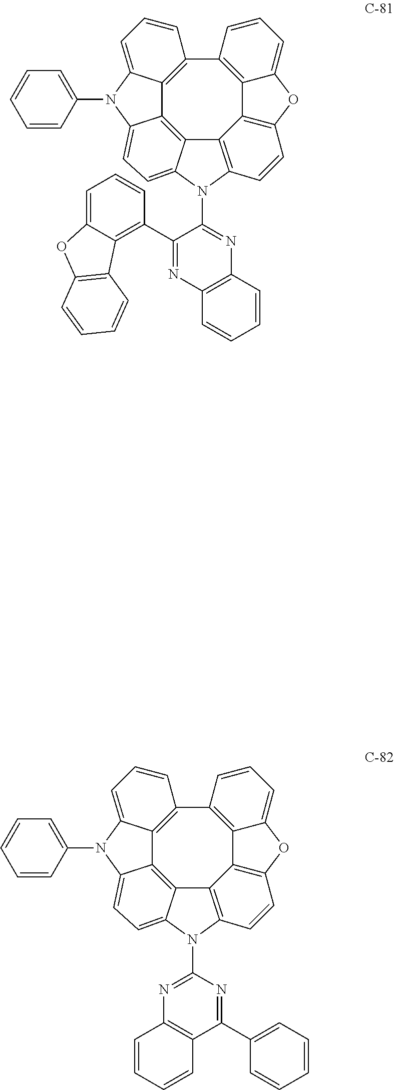 [SMILES: c1ccc(-n2c3cccc4c5cccc6oc7ccc8c(c7c65)c5c(c43)c2ccc5n8-c2nc3ccccc3nc2-c2cccc3oc4ccccc4c23)cc1 ; c1ccc(-c2nc(-n3c4ccc5oc6cccc7c8cccc9c8c8c(c4c5c67)c3ccc8n9-c3ccccc3)nc3ccccc23)cc1]

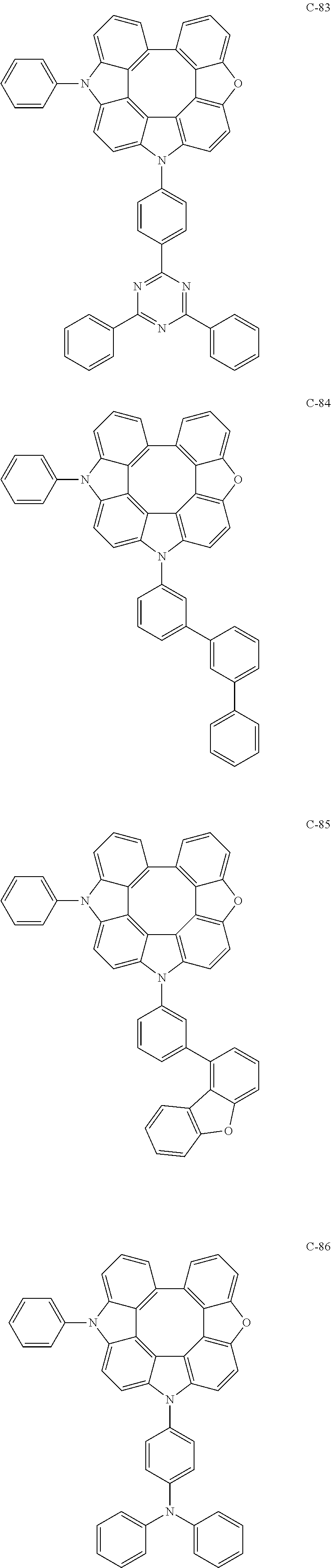 [SMILES: c1ccc(-c2nc(-c3ccccc3)nc(-c3ccc(-n4c5ccc6oc7cccc8c9cccc%10c9c9c(c5c6c78)c4ccc9n%10-c4ccccc4)cc3)n2)cc1 ; c1ccc(-c2cccc(-c3cccc(-n4c5ccc6oc7cccc8c9cccc%10c9c9c(c5c6c78)c4ccc9n%10-c4ccccc4)c3)c2)cc1 ; c1ccc(-n2c3cccc4c5cccc6oc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5oc6cccc7c8cccc9c8c8c(c4c5c67)c3ccc8n9-c3ccccc3)cc2)cc1]

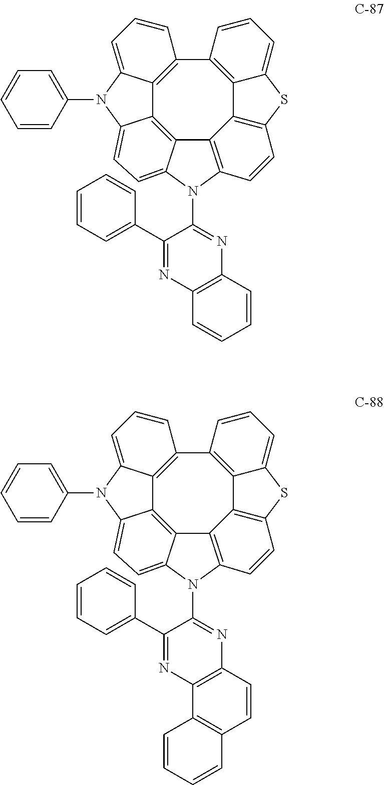 [SMILES: c1ccc(-c2nc3ccccc3nc2-n2c3ccc4sc5cccc6c7cccc8c7c7c(c3c4c56)c2ccc7n8-c2ccccc2)cc1 ; c1ccc(-c2nc3c(ccc4ccccc43)nc2-n2c3ccc4sc5cccc6c7cccc8c7c7c(c3c4c56)c2ccc7n8-c2ccccc2)cc1]

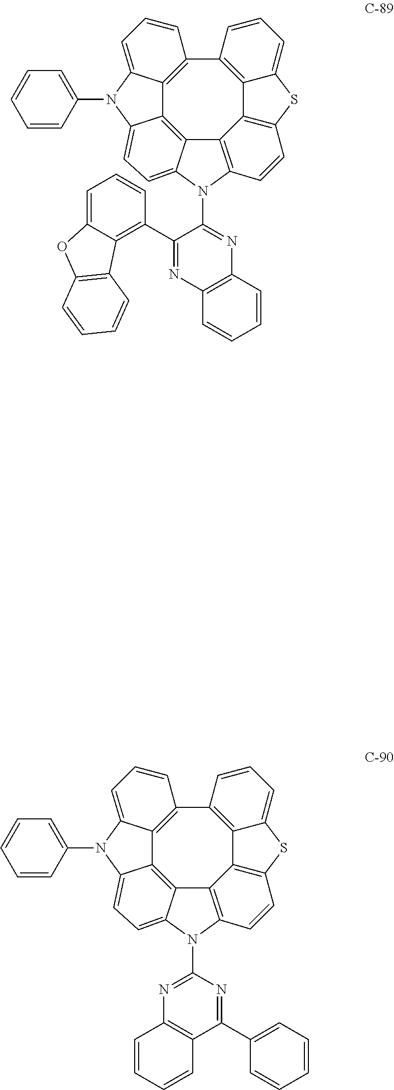 [SMILES: c1ccc(-n2c3cccc4c5cccc6sc7ccc8c(c7c65)c5c(c43)c2ccc5n8-c2nc3ccccc3nc2-c2cccc3oc4ccccc4c23)cc1 ; c1ccc(-c2nc(-n3c4ccc5sc6cccc7c8cccc9c8c8c(c4c5c67)c3ccc8n9-c3ccccc3)nc3ccccc23)cc1]

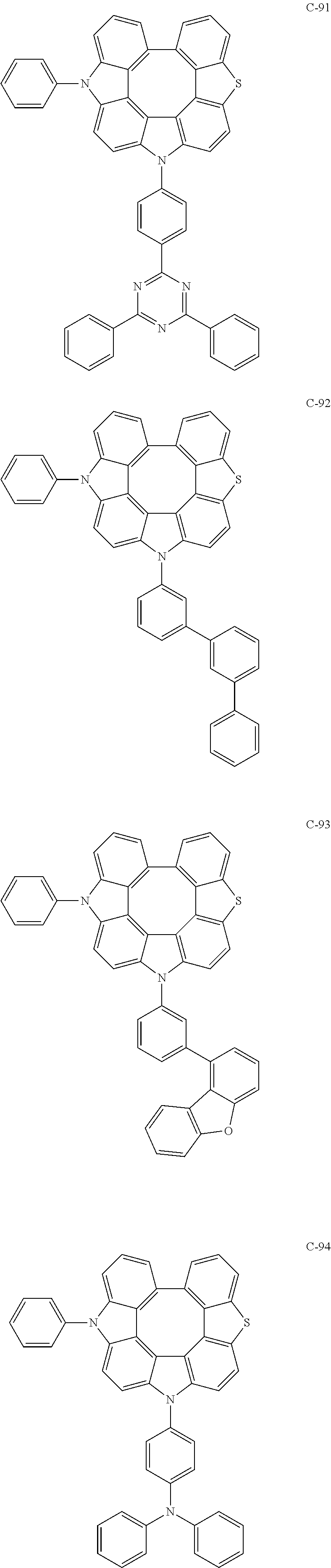 [SMILES: c1ccc(-c2nc(-c3ccccc3)nc(-c3ccc(-n4c5ccc6sc7cccc8c9cccc%10c9c9c(c5c6c78)c4ccc9n%10-c4ccccc4)cc3)n2)cc1 ; c1ccc(-c2cccc(-c3cccc(-n4c5ccc6sc7cccc8c9cccc%10c9c9c(c5c6c78)c4ccc9n%10-c4ccccc4)c3)c2)cc1 ; c1ccc(-n2c3cccc4c5cccc6sc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5sc6cccc7c8cccc9c8c8c(c4c5c67)c3ccc8n9-c3ccccc3)cc2)cc1]

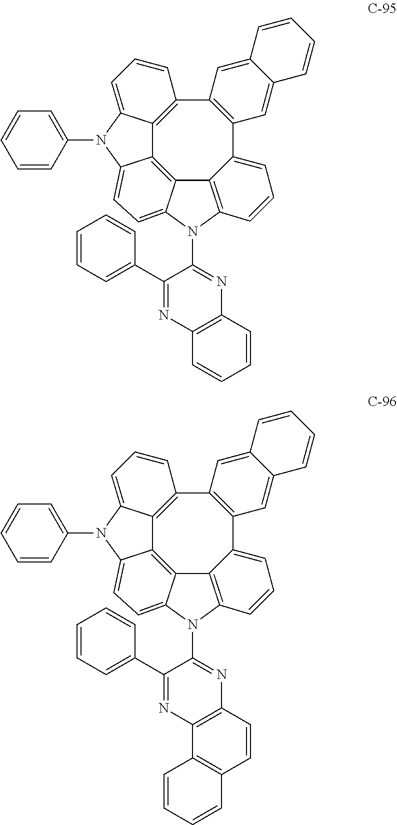 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1]

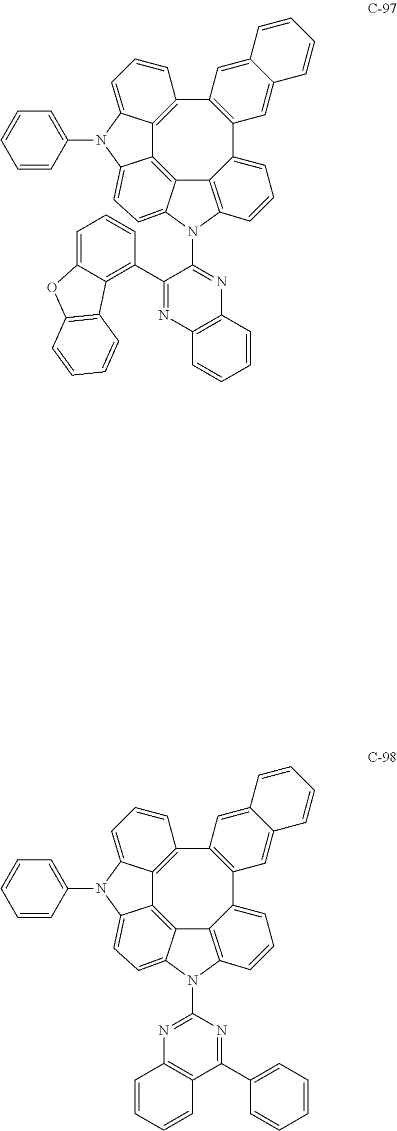 [SMILES: c1ccc(-n2c3cccc4c5cc6ccccc6cc5c5cccc6c5c5c(c43)c2ccc5n6-c2nc3ccccc3nc2-c2cccc3oc4ccccc4c23)cc1 ; c1ccc(-c2nc(-n3c4cccc5c6cc7ccccc7cc6c6cccc7c6c6c(c54)c3ccc6n7-c3ccccc3)nc3ccccc23)cc1]

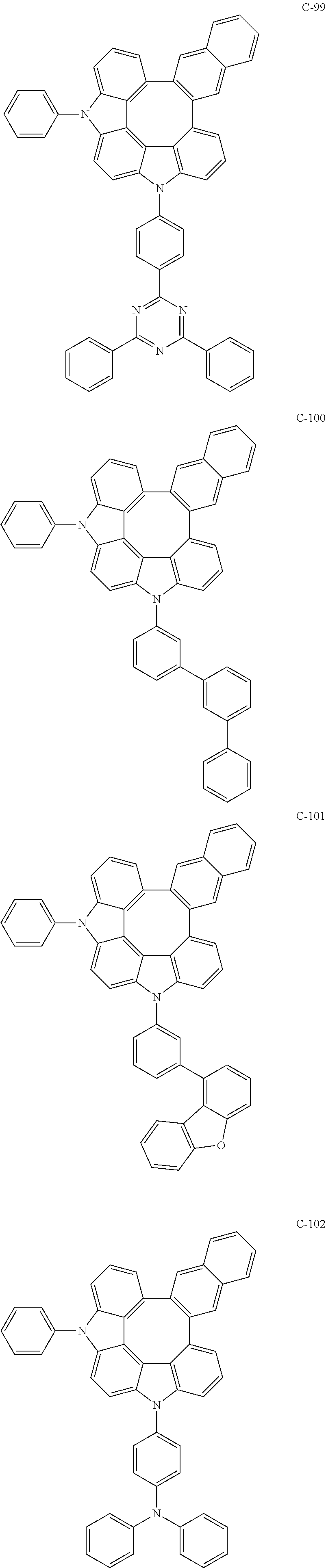

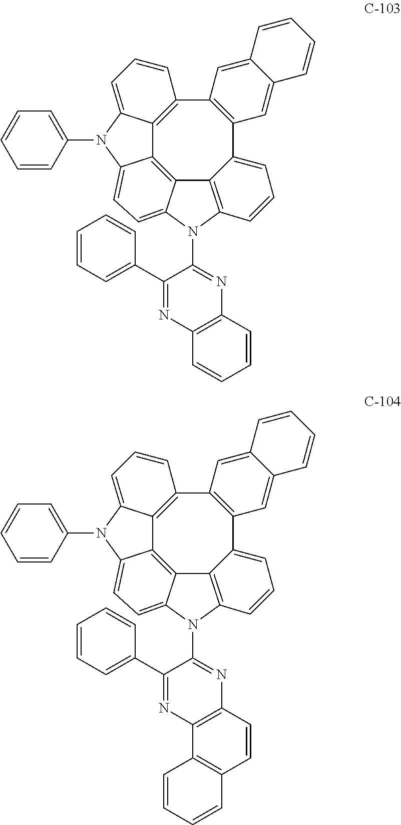 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1]

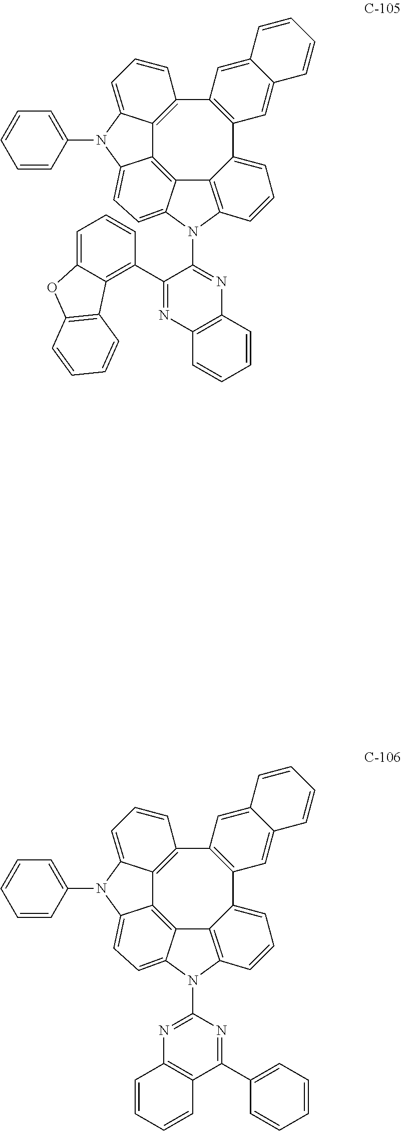 [SMILES: c1ccc(-n2c3cccc4c5cc6ccccc6cc5c5cccc6c5c5c(c43)c2ccc5n6-c2nc3ccccc3nc2-c2cccc3oc4ccccc4c23)cc1 ; c1ccc(-c2nc(-n3c4cccc5c6cc7ccccc7cc6c6cccc7c6c6c(c54)c3ccc6n7-c3ccccc3)nc3ccccc23)cc1]

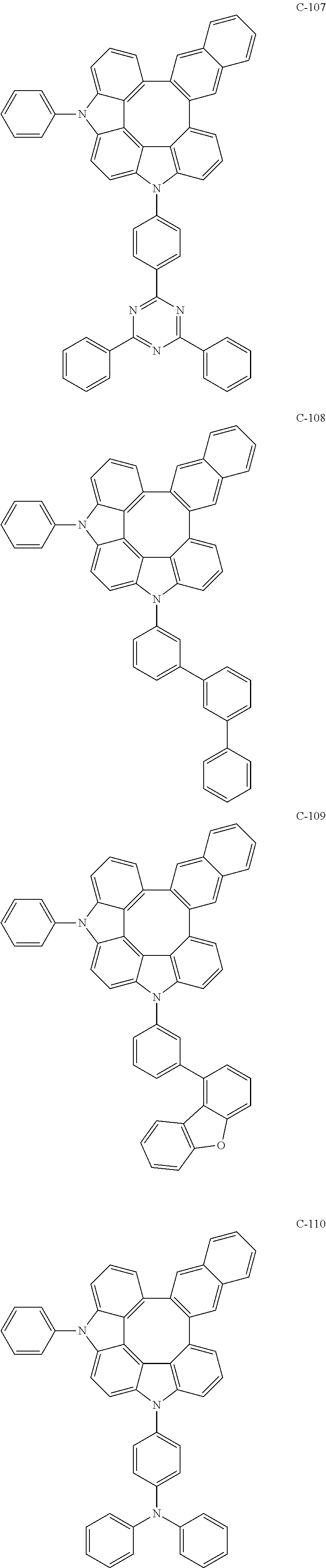

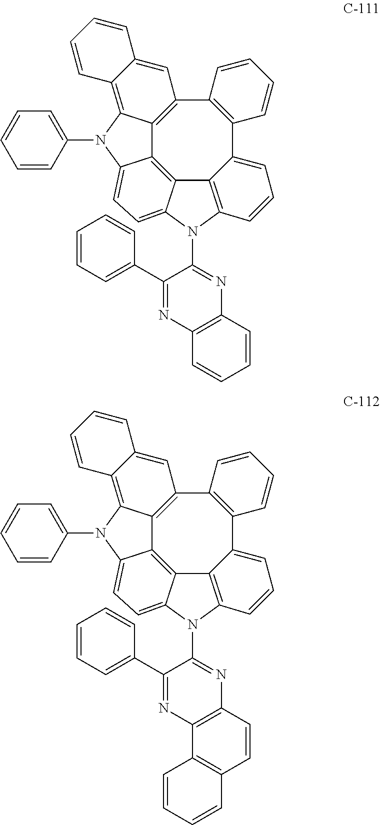 [SMILES: c1ccc(-c2nc3ccccc3nc2-n2c3cccc4c5ccccc5c5cc6ccccc6c6c5c5c(c43)c2ccc5n6-c2ccccc2)cc1 ; c1ccc(-c2nc3c(ccc4ccccc43)nc2-n2c3cccc4c5ccccc5c5cc6ccccc6c6c5c5c(c43)c2ccc5n6-c2ccccc2)cc1]

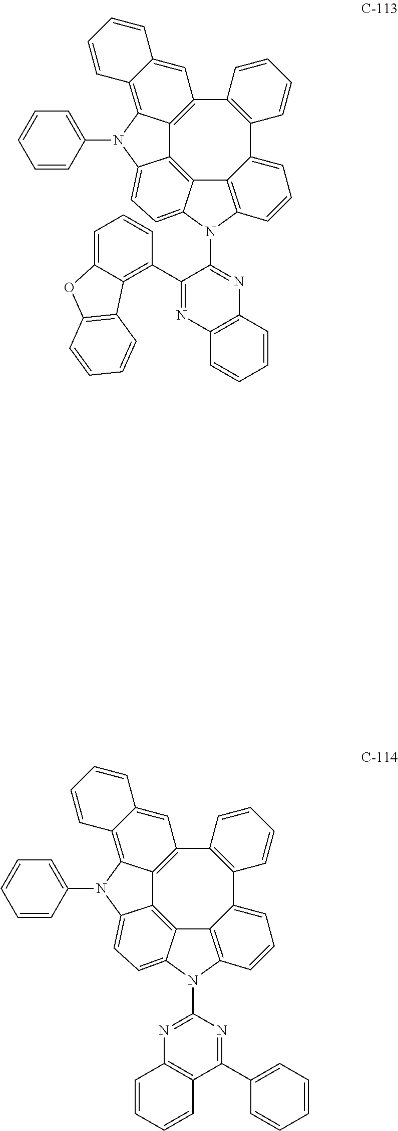 [SMILES: c1ccc(-n2c3ccc4c5c6c(cccc6n4-c4nc6ccccc6nc4-c4cccc6oc7ccccc7c46)c4ccccc4c4cc6ccccc6c2c4c53)cc1 ; c1ccc(-c2nc(-n3c4cccc5c6ccccc6c6cc7ccccc7c7c6c6c(c54)c3ccc6n7-c3ccccc3)nc3ccccc23)cc1]

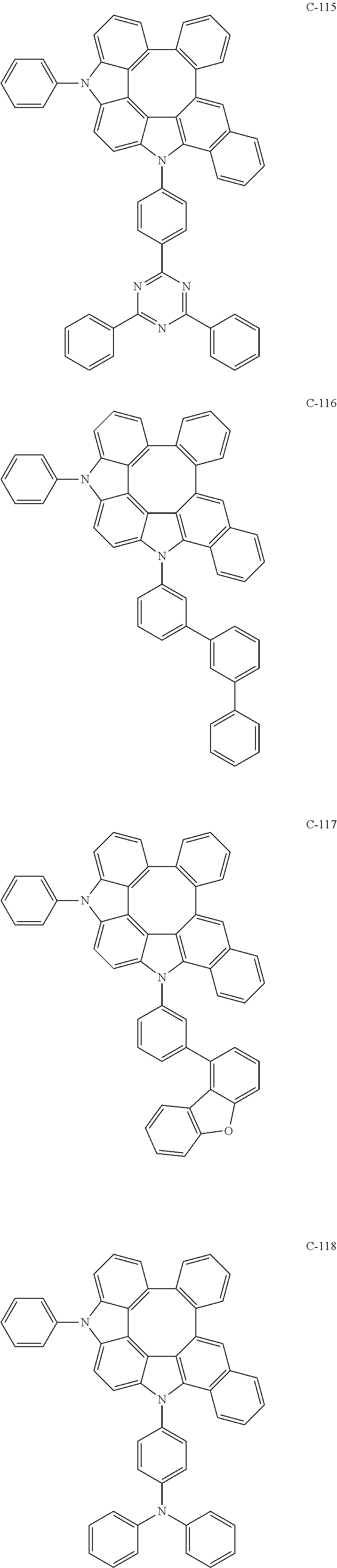 [SMILES: c1ccc(-c2nc(-c3ccccc3)nc(-c3ccc(-n4c5ccc6c7c8c(cccc8n6-c6ccccc6)c6ccccc6c6cc8ccccc8c4c6c75)cc3)n2)cc1 ; c1ccc(-c2cccc(-c3cccc(-n4c5ccc6c7c8c(cccc8n6-c6ccccc6)c6ccccc6c6cc8ccccc8c4c6c75)c3)c2)cc1 ; c1ccc(-n2c3cccc4c5ccccc5c5cc6ccccc6c6c5c5c(c43)c2ccc5n6-c2cccc(-c3cccc4oc5ccccc5c34)c2)cc1 ; c1ccc(N(c2ccccc2)c2ccc(-n3c4ccc5c6c7c(cccc7n5-c5ccccc5)c5ccccc5c5cc7ccccc7c3c5c64)cc2)cc1]

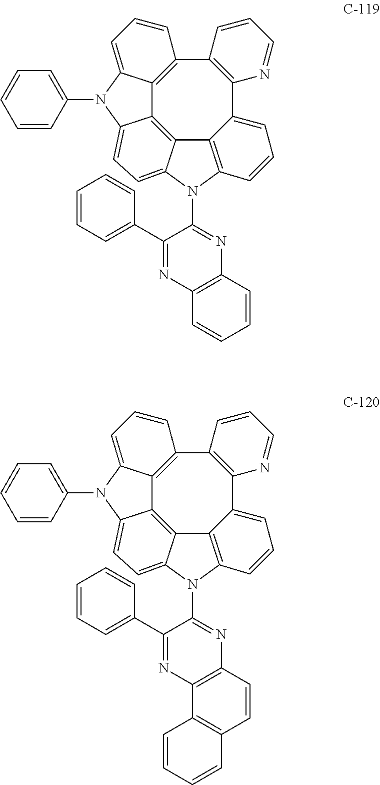 [SMILES: c1ccc(-c2nc3ccccc3nc2-n2c3cccc4c5ncccc5c5cccc6c5c5c(c43)c2ccc5n6-c2ccccc2)cc1 ; c1ccc(-c2nc3c(ccc4ccccc43)nc2-n2c3cccc4c5ncccc5c5cccc6c5c5c(c43)c2ccc5n6-c2ccccc2)cc1]

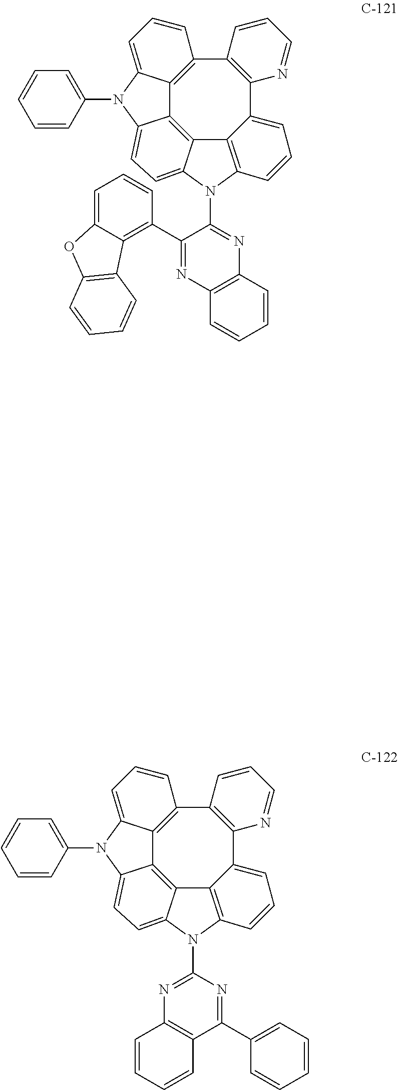 [SMILES: c1ccc(-n2c3cccc4c5cccnc5c5cccc6c5c5c(c43)c2ccc5n6-c2nc3ccccc3nc2-c2cccc3oc4ccccc4c23)cc1 ; c1ccc(-c2nc(-n3c4cccc5c6ncccc6c6cccc7c6c6c(c54)c3ccc6n7-c3ccccc3)nc3ccccc23)cc1]

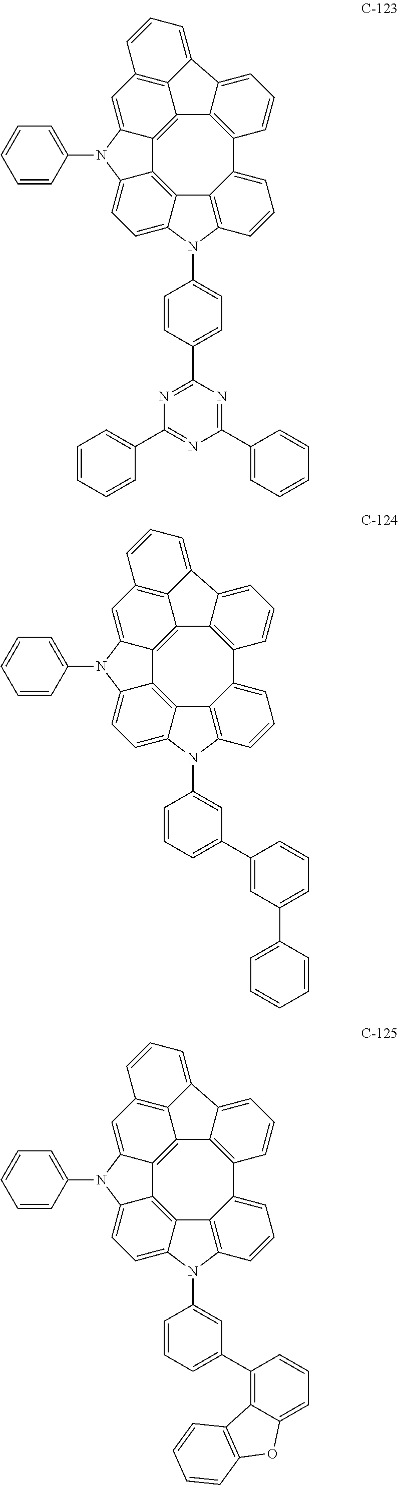 [SMILES: c1ccc(-c2nc(-c3ccccc3)nc(-c3ccc(-n4c5cccc6c7cccc8c7c7c9c-8cccc9cc8c7c7c(c65)c4ccc7n8-c4ccccc4)cc3)n2)cc1 ; c1ccc(-c2cccc(-c3cccc(-n4c5cccc6c7cccc8c7c7c9c-8cccc9cc8c7c7c(c65)c4ccc7n8-c4ccccc4)c3)c2)cc1 ; c1ccc(-n2c3cc4cccc5c4c4c6c-5cccc6c5cccc6c5c5c(c43)c2ccc5n6-c2cccc(-c3cccc4oc5ccccc5c34)c2)cc1]

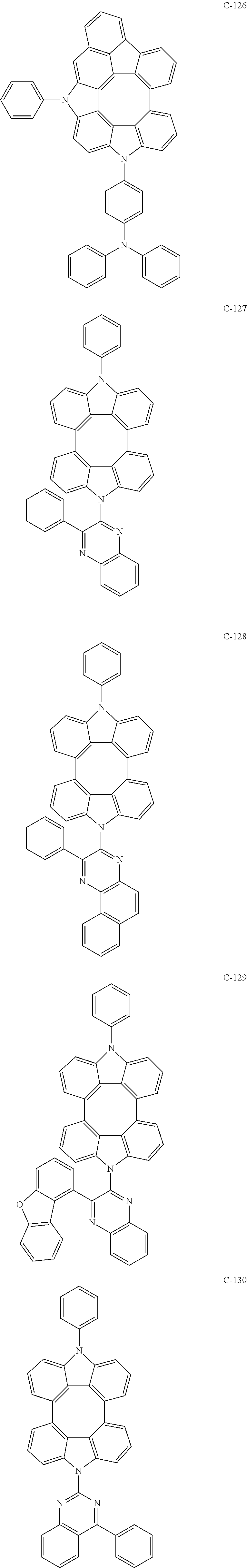

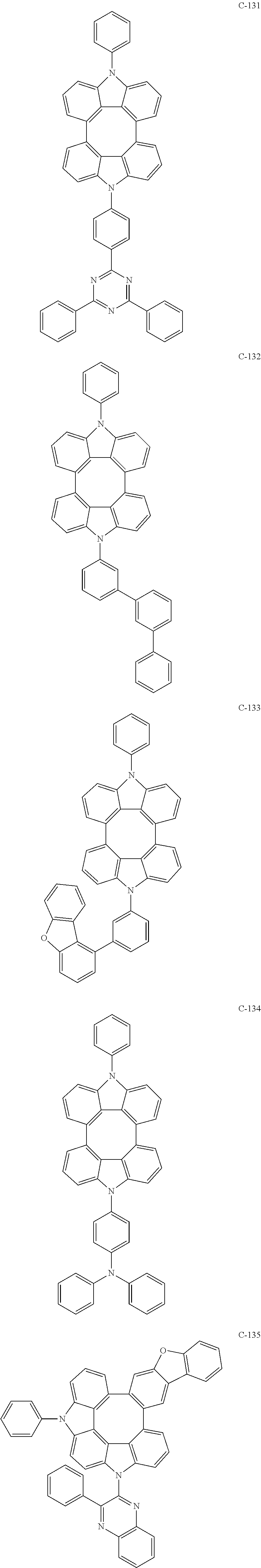 [SMILES: c1ccc(-c2nc(-c3ccccc3)nc(-c3ccc(-n4c5cccc6c7cccc8c7c7c(cccc7n8-c7ccccc7)c7cccc4c7c65)cc3)n2)cc1 ; c1ccc(-c2cccc(-c3cccc(-n4c5cccc6c7cccc8c7c7c(cccc7n8-c7ccccc7)c7cccc4c7c65)c3)c2)cc1 ; c1ccc(-n2c3cccc4c5cccc6c5c5c(cccc5n6-c5cccc(-c6cccc7oc8ccccc8c67)c5)c5cccc2c5c43)cc1 ; c1ccc(N(c2ccccc2)c2ccc(-n3c4cccc5c6cccc7c6c6c(cccc6n7-c6ccccc6)c6cccc3c6c54)cc2)cc1 ; c1ccc(-c2nc3ccccc3nc2-n2c3cccc4c5cc6c(cc5c5cccc7c5c5c(c43)c2ccc5n7-c2ccccc2)oc2ccccc26)cc1]

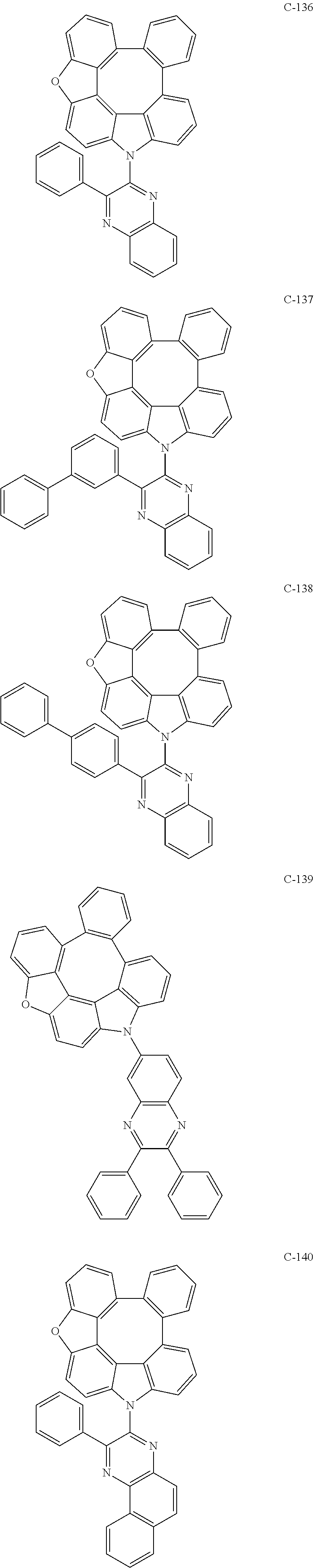

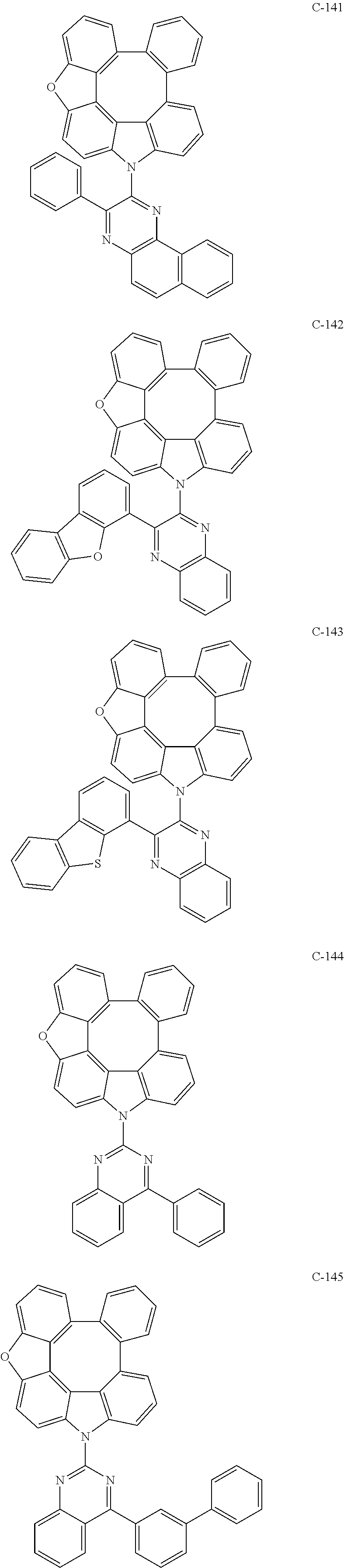

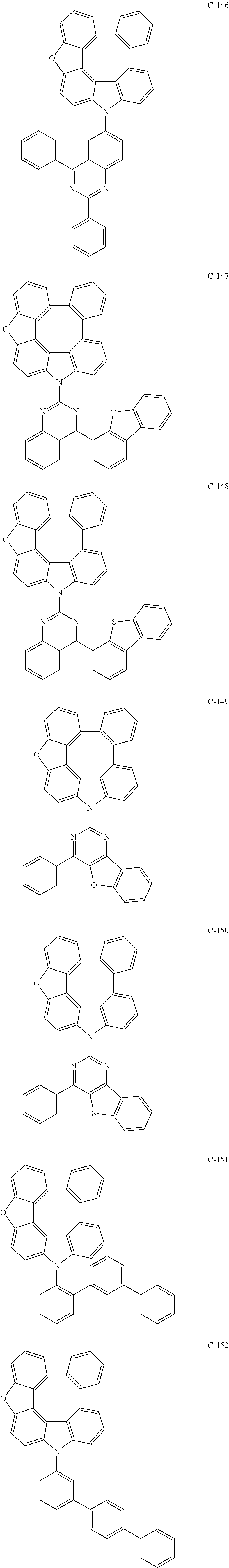

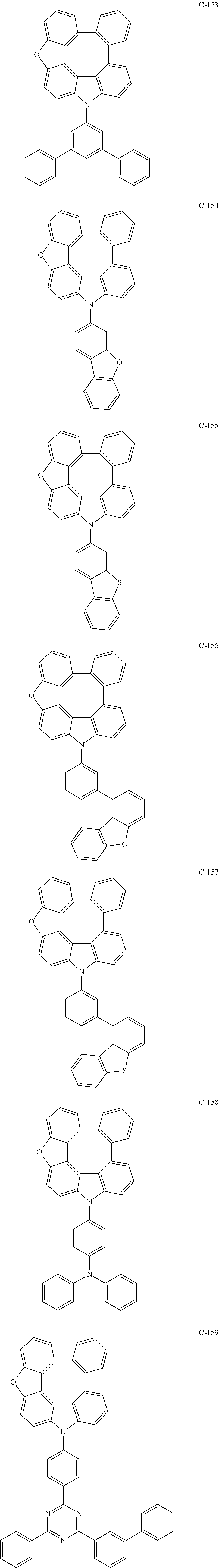

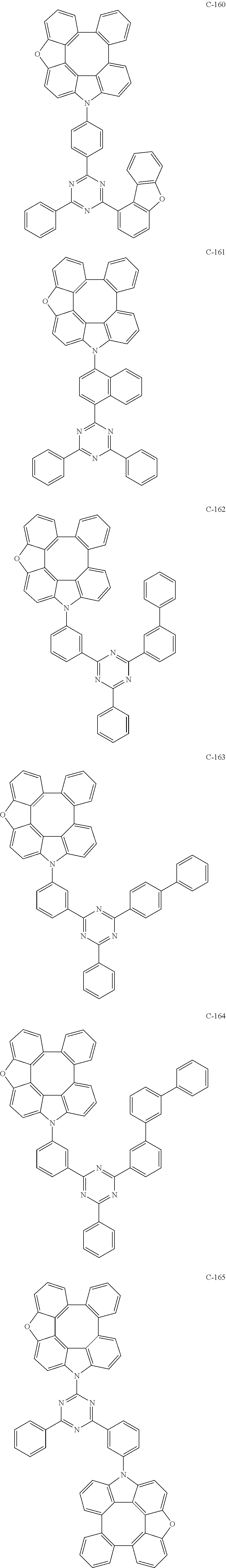

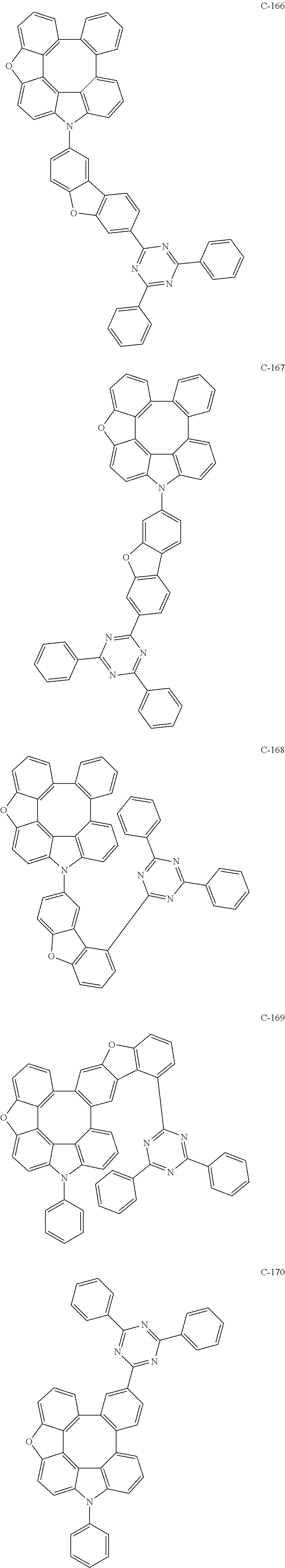

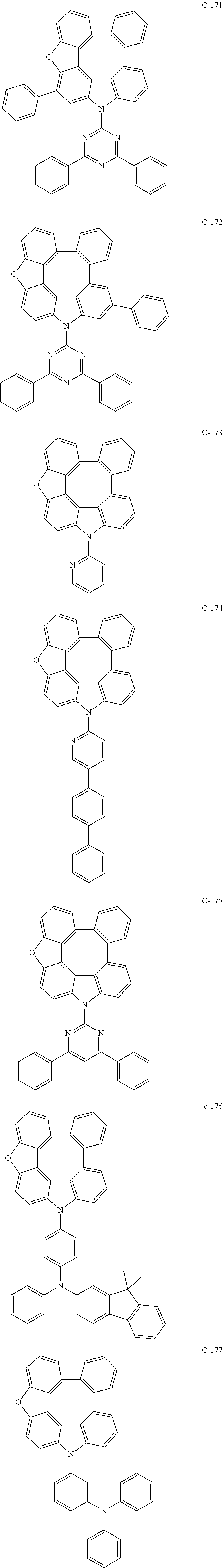

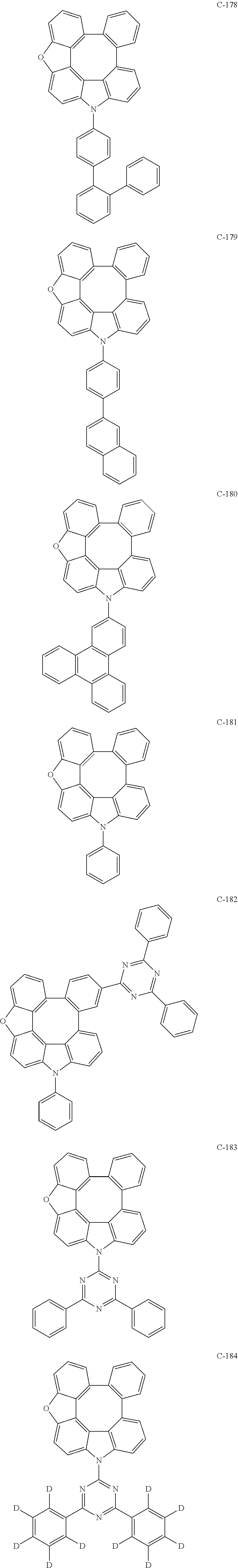

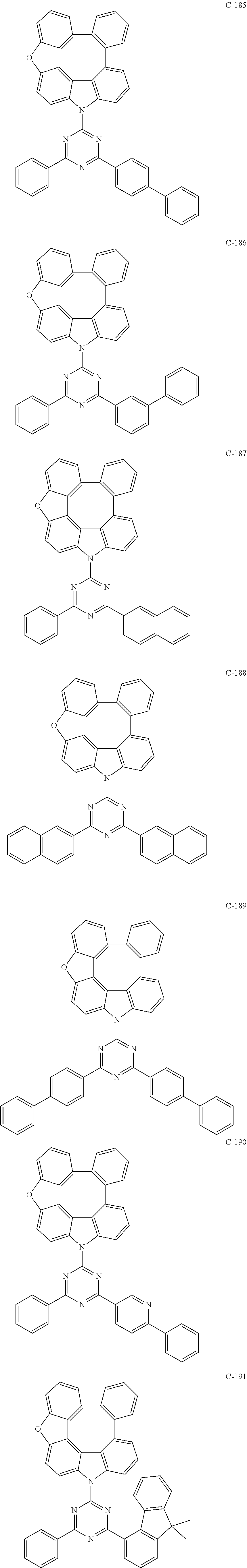

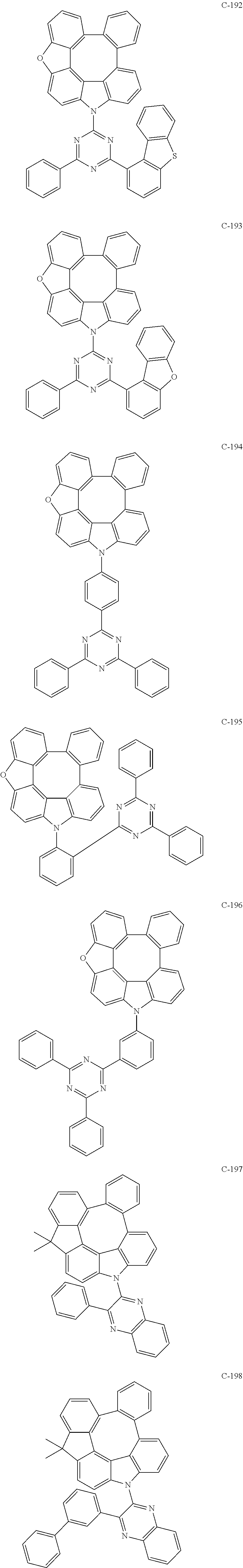

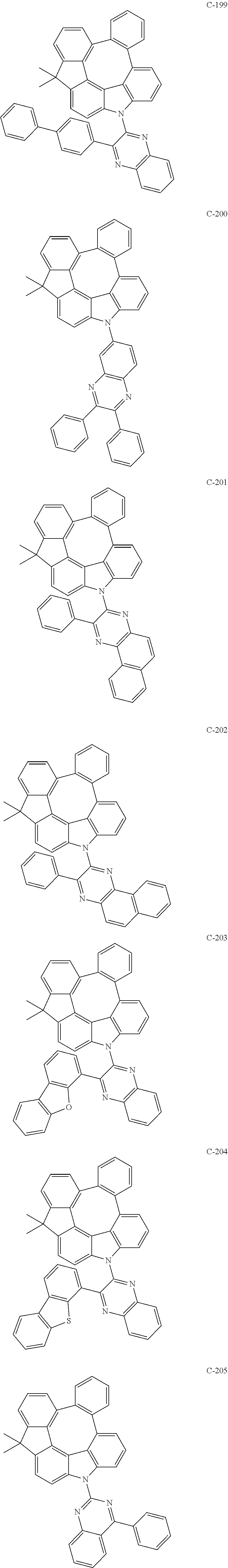

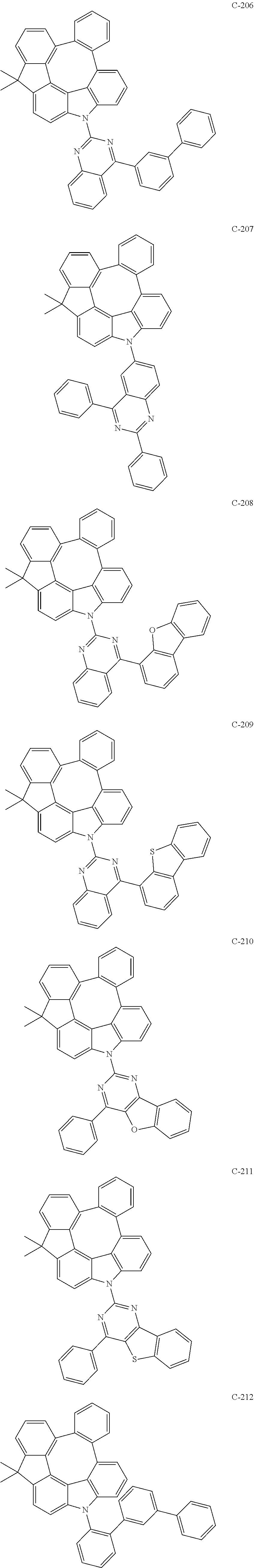

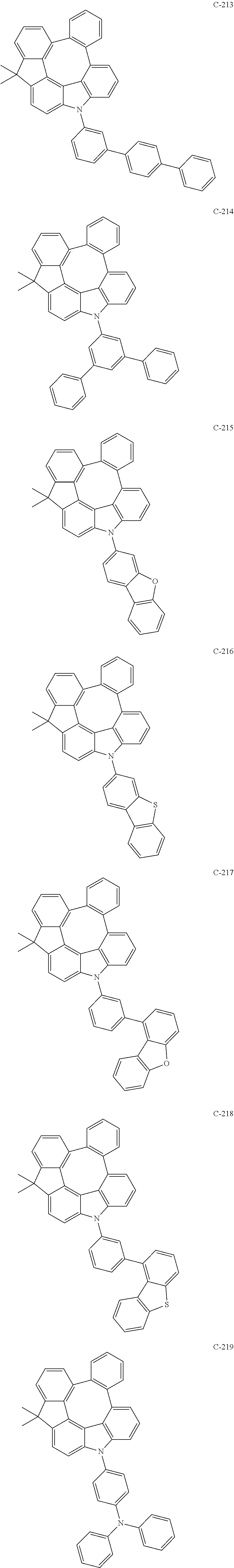

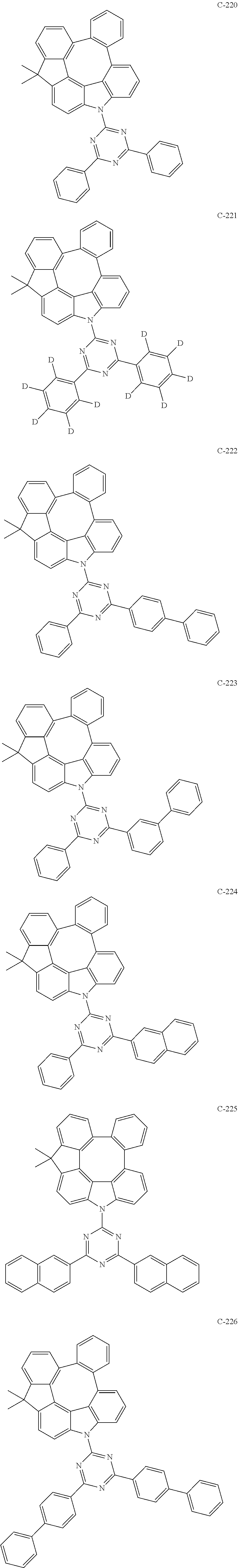

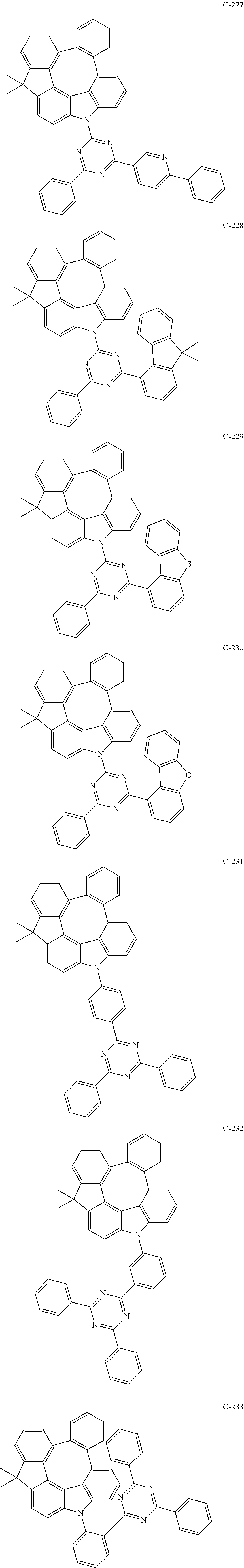

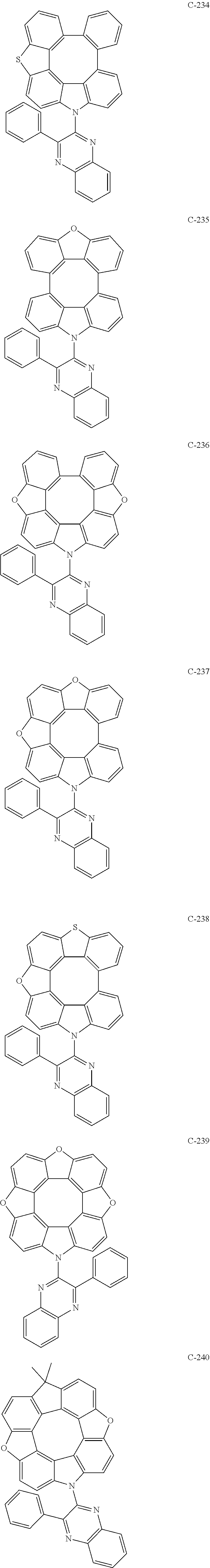

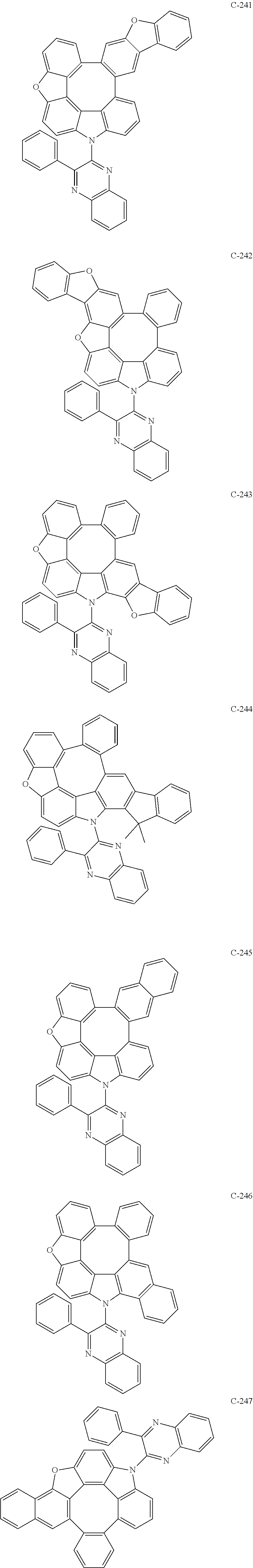

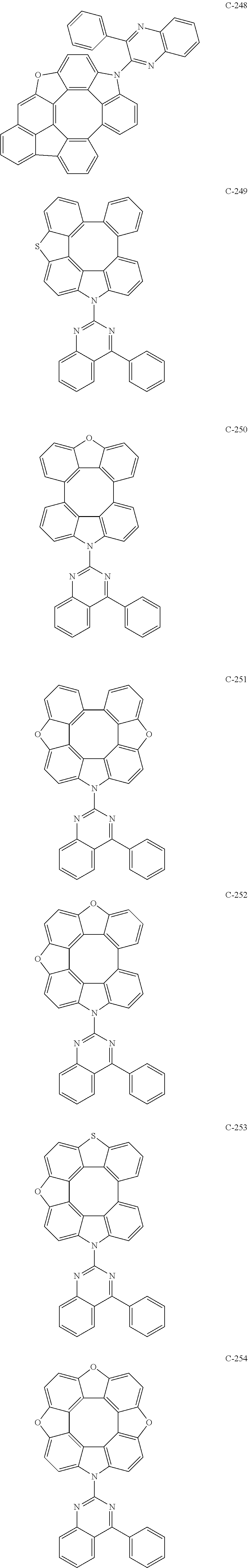

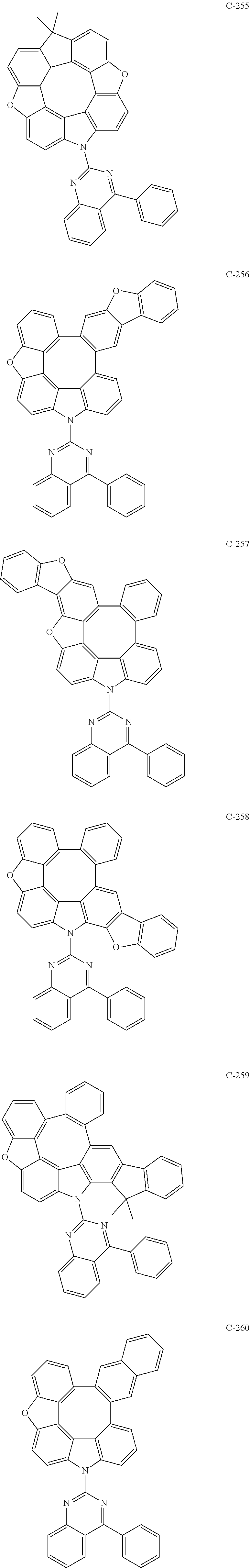

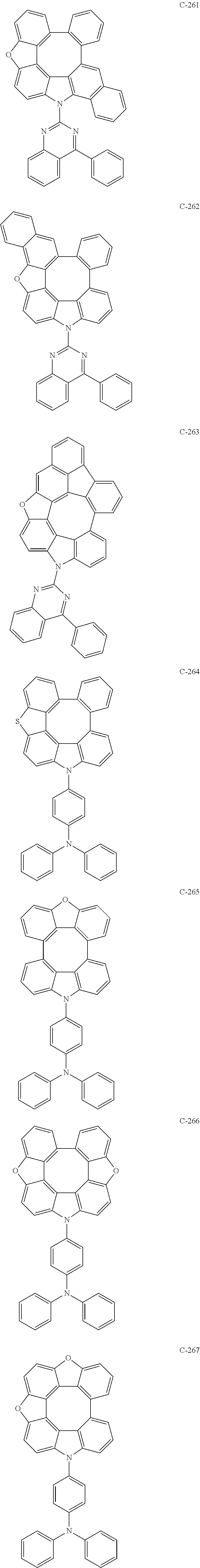

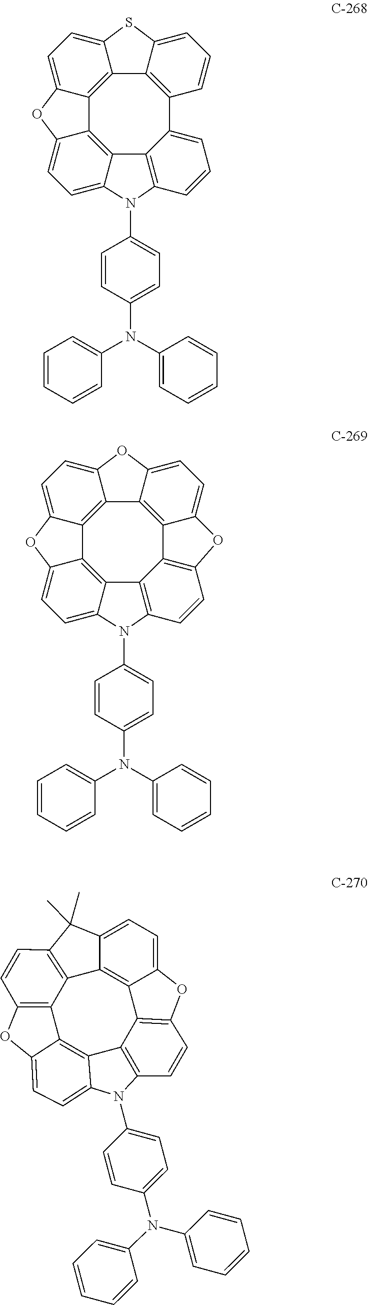 [SMILES: c1ccc(N(c2ccccc2)c2ccc(-n3c4cccc5c6cccc7sc8ccc9oc%10ccc3c(c%10c9c8c76)c54)cc2)cc1 ; c1ccc(N(c2ccccc2)c2ccc(-n3c4ccc5oc6ccc7oc8ccc9oc%10ccc3c3c%10c9c8c7c6c5c34)cc2)cc1 ; CC1(C)c2ccc3oc4ccc5c6c4c3c2c2c1ccc1oc3ccc(c6c3c12)n5-c1ccc(N(c2ccccc2)c2ccccc2)cc1]

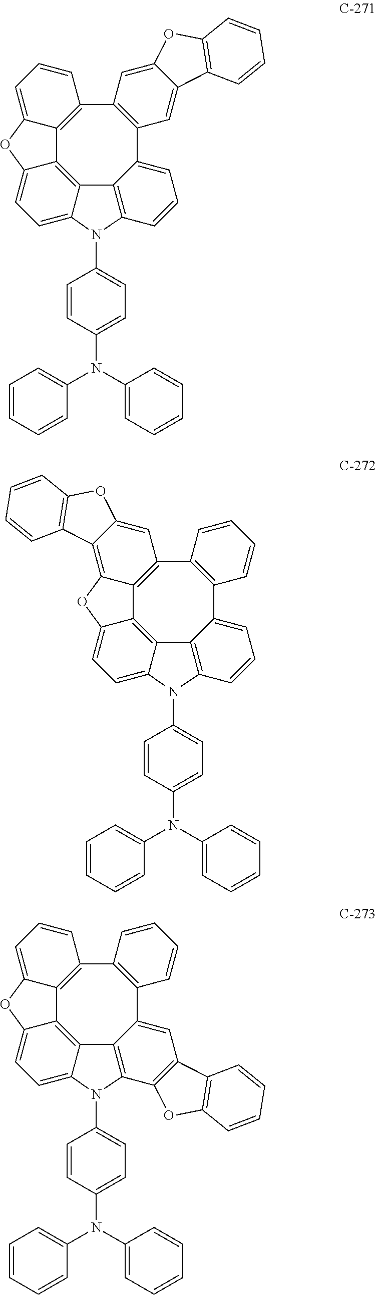

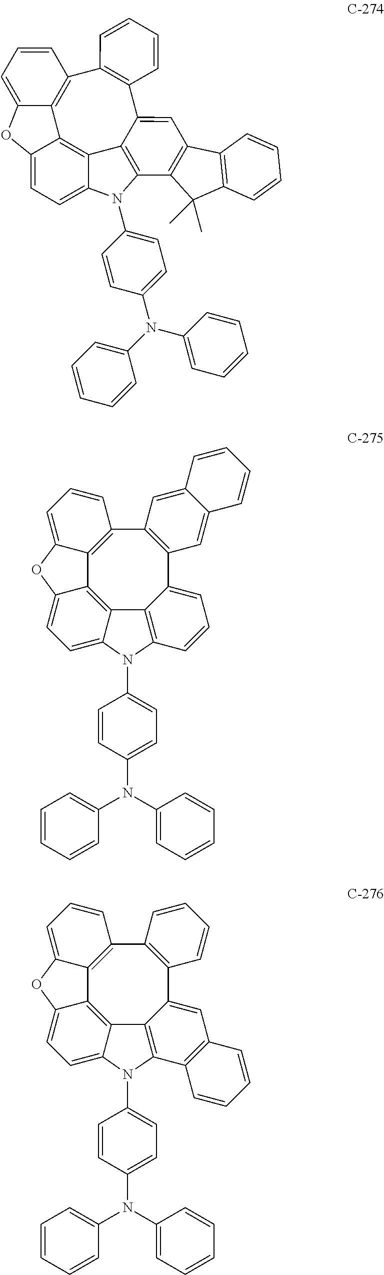

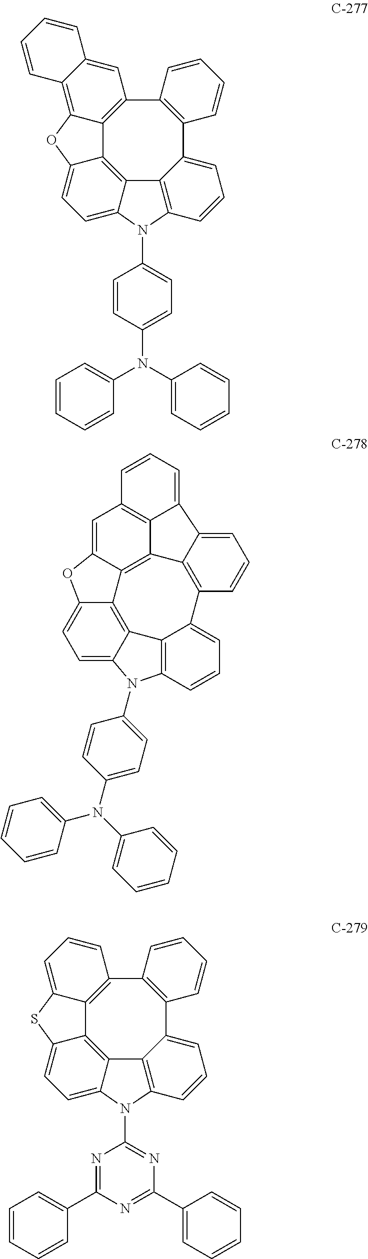

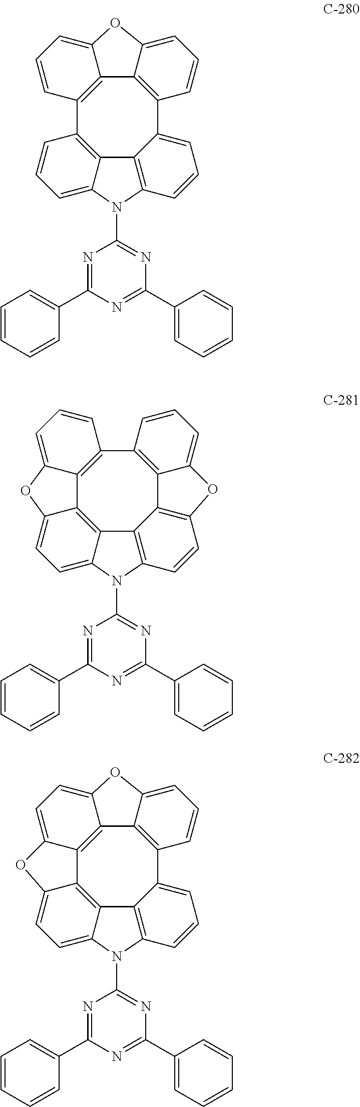 [SMILES: c1ccc(-c2nc(-c3ccccc3)nc(-n3c4cccc5c6cccc7oc8cccc(c9cccc3c9c54)c8c76)n2)cc1 ; c1ccc(-c2nc(-c3ccccc3)nc(-n3c4ccc5oc6cccc7c8cccc9oc%10ccc3c(c%10c98)c4c5c67)n2)cc1 ; c1ccc(-c2nc(-c3ccccc3)nc(-n3c4cccc5c6cccc7oc8ccc9oc%10ccc3c(c%10c9c8c76)c54)n2)cc1]

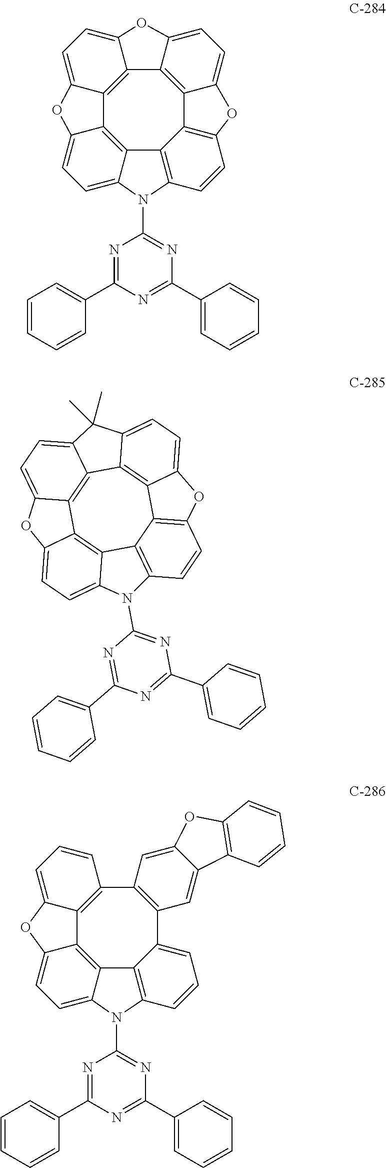 [SMILES: c1ccc(-c2nc(-c3ccccc3)nc(-n3c4ccc5oc6ccc7oc8ccc9oc%10ccc3c3c%10c9c8c7c6c5c34)n2)cc1 ; CC1(C)c2ccc3oc4ccc5c6c4c3c2c2c1ccc1oc3ccc(c6c3c12)n5-c1nc(-c2ccccc2)nc(-c2ccccc2)n1 ; c1ccc(-c2nc(-c3ccccc3)nc(-n3c4cccc5c6cc7c(cc6c6cccc8oc9ccc3c(c9c86)c54)oc3ccccc37)n2)cc1]

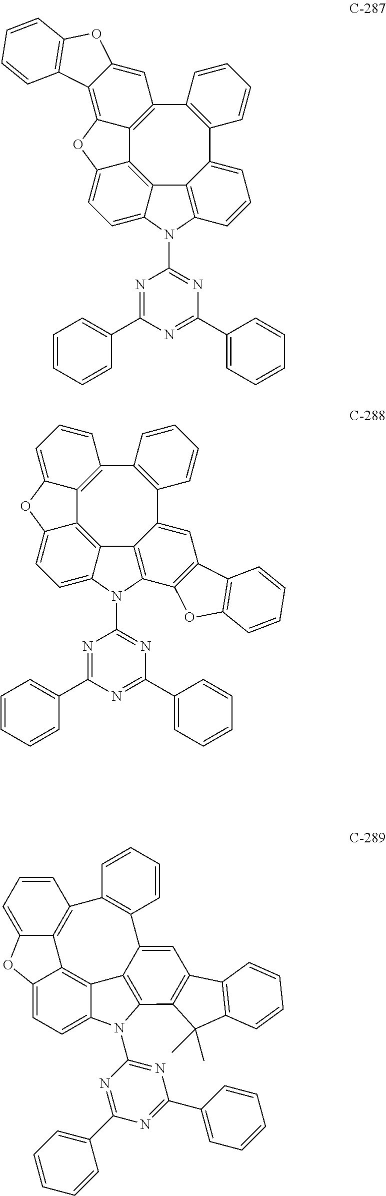

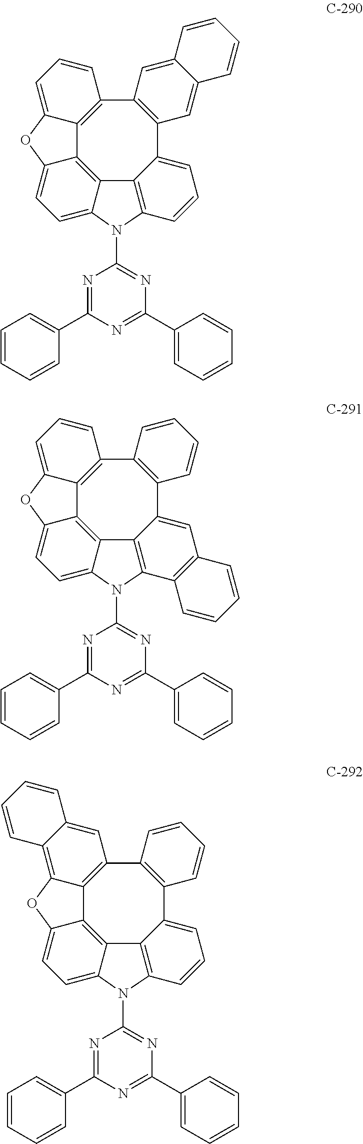

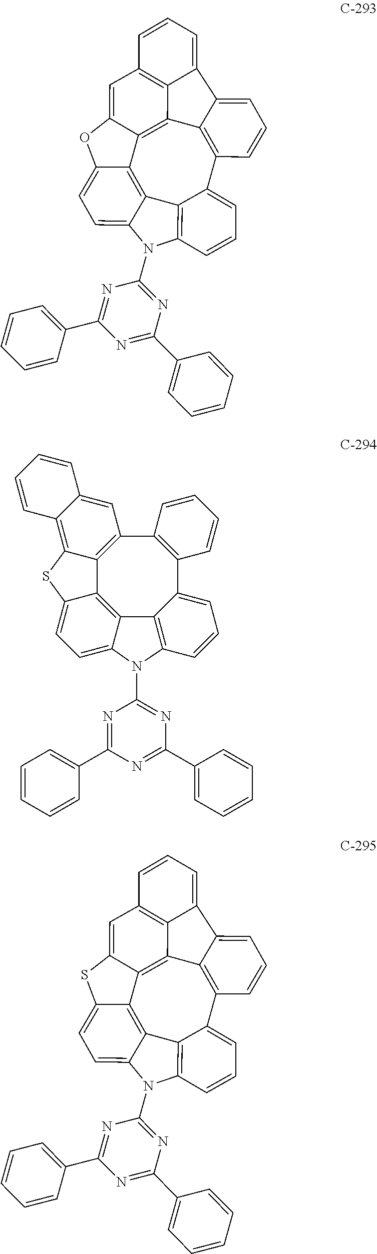 [SMILES: c1ccc(-c2nc(-c3ccccc3)nc(-n3c4cccc5c6cccc7c6c6c8c-7cccc8cc7oc8ccc3c(c8c76)c54)n2)cc1 ; c1ccc(-c2nc(-c3ccccc3)nc(-n3c4cccc5c6ccccc6c6cc7ccccc7c7sc8ccc3c(c8c67)c54)n2)cc1 ; c1ccc(-c2nc(-c3ccccc3)nc(-n3c4cccc5c6cccc7c6c6c8c-7cccc8cc7sc8ccc3c(c8c76)c54)n2)cc1]

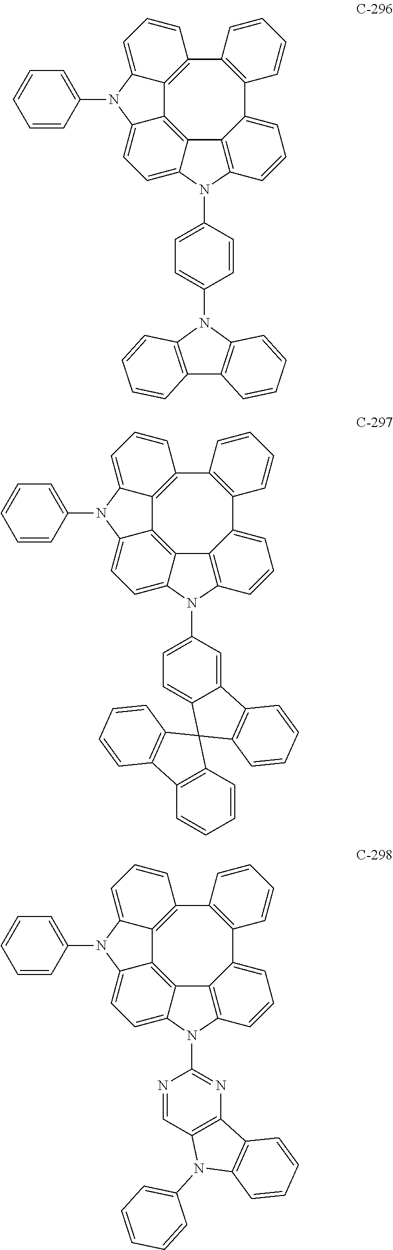

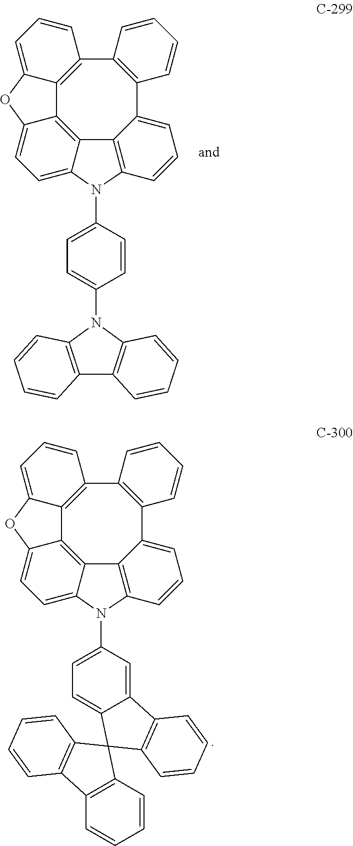

8. A plurality of host materials comprising a first host material and a second host material, wherein the first host material comprises the compound represented by formula 1 according to claim 1, and the second host material comprises an organic electroluminescent compound other than the compound represented by formula 1.

9. The plurality of host materials according to claim 8, wherein the second host material comprises a compound represented by the following formula 11: 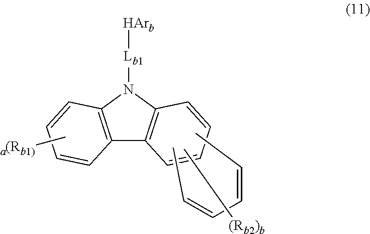
wherein
HAr_{b} represents a substituted or unsubstituted (3- to 30-membered)heteroaryl;
L_{b1} represents a single bond, a substituted or unsubstituted (C6-C30)arylene, or a substituted or unsubstituted (3- to 30-membered)heteroarylene;
R_{b1} and R_{b2}, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or may be linked to an adjacent substituent(s) to form a ring(s);
a represents an integer of 1 to 4; and b represents an integer of 1 to 6; where if a and b, each independently, represent an integer of 2 or more, each of R_{b1} and each of R_{b2} may be the same as or different from each other.

10. An organic electroluminescent device comprising the organic electroluminescent compound according to claim 1.
